(19) United States
(12) Patent Application Publication
(10) Pub. No.: US 2020/0290978 A1
(43) Pub. Date: Sep. 17, 2020

(54) COMPOSITIONS AND METHODS FOR TREATING CANCER

(71) Applicant: The Regents of the University of California, Oakland, CA (US)

(72) Inventors: David A. Nathanson, Los Angeles, CA (US); Wilson X. Mai, Los Angeles, CA (US); Michael E. Jung, Los Angeles, CA (US); Peter M. Clark, Los Angeles, CA (US); Timothy F. Cloughesy, Calabasas, CA (US); Gyudong Kim, Seoul (KR); Jonathan Tsang, Los Angeles, CA (US); Lorenz Urner, Los Angeles, CA (US)

(73) Assignee: The Regents of the University of California, Oakland, CA (US)

(21) Appl. No.: 16/651,256

(22) PCT Filed: Sep. 26, 2018
(86) PCT No.: PCT/US2018/052858

§ 371 Date: Mar. 26, 2020

Related U.S. Application Data
(60) Provisional application No. 62/563,373, filed on Sep. 26, 2017.
(60) Provisional application No. 62/589,972, filed on Nov. 22, 2017.

(51) Int. Cl. C07D 239/95; G01N 33/574; A61K 31/517; C07D 491/056; A61K 31/519; C07D 405/10; G01N 33/50; A61K 31/496; A61K 31/40; A61K 45/06; A61P 35/00

(52) U.S. Cl. CPC C07D 239/95; G01N 33/57407; A61K 31/517; C07D 491/056; A61K 31/519; A61P 35/00; G01N 33/5011; A61K 31/496; A61K 31/40; A61K 45/06; C07D 405/10

(57) ABSTRACT

The present disclosure relates to compounds that are capable penetrating to the blood brain barrier to modulate the activity of EGFR tyrosine kinase. The disclosure further relates to methods of treating glioblastoma and other EGFR mediated cancers. The disclosure further relates to methods of treating glioblastoma and other EGFR mediated cancers that have been determined to have altered glucose metabolism in the presence of inhibitors. The present disclosure also provides methods of administering to a subject a glucose metabolism inhibitor and a cytoplasmic p53 stabilizer.

### RELATED APPLICATIONS

This application claims the benefit of U.S. Provisional Patent Application No. 62/589,972, filed on Nov. 22, 2017, and U.S. Provisional Patent Application No. 62/563,373, filed on Sep. 26, 2017. The contents of each of these applications are hereby incorporated by reference in its entirety.

### STATEMENT OF GOVERNMENT SUPPORT

This invention was made with government support under Grant Numbers CA151819, CA211015, and CA213133, awarded by the National Institutes of Health. The government has certain rights in the invention.

### BACKGROUND

Glioblastoma (glioblastoma multiforme; GBM) accounts for the majority of primary malignant brain tumors in adults. Amplification and mutation of the epidermal growth factor receptor (EGFR) gene is a signature genetic abnormality encountered in GBM (Sugawa, et al. (1990) Proc. Natl. Acad. Sci. 87: 8602-8606; Ekstrand, et al. (1992) Proc. Natl. Acad. Sci. 89: 4309-4313). A range of potential therapies that target EGFR or its mutant constitutively active form, ΔEGFR, including tyrosine kinase inhibitors (TKIs), monoclonal antibodies, vaccines, and RNA-based agents, are currently in development or in clinical trials for the treatment of GBM. However, to date their efficacy in the clinic has so far been limited by both upfront and acquired drug resistance (Taylor, et al. (2012) Curr. Cancer Drug Targets. 12:197-209). A major limitation is that current therapies such as erlotinib, lapatinib, gefitinib and afatinib are poorly brain penetrant (Razier, et al. (2010) Neuro-Oncology 12:95-103; Reardon, et al. (2015) Neuro-Oncology 17:430-439; Thiessen, et al. (2010) Cancer Chemother. Pharmacol. 65:353-361).

Molecular targeted therapies have revolutionized cancer treatment and paved the path for modern precision medicine. However, despite well-defined actionable genetic alterations, targeted drugs have failed in glioblastoma (GBM) patients. This is in large part due to insufficient CNS penetration of most targeted agents to levels necessary for tumor kill; potentially evoking robust adaptive mechanisms to drive therapeutic resistance. While drug combinations that inhibit both the primary lesion and the compensatory signaling pathway(s) are appealing, these combination therapy strategies have been hampered by enhanced toxicities leading to subthreshold dosing of each drug.

An alternative therapeutic approach targets an oncogenic driver to modify an important functional property for tumor survival, rendering cells vulnerable to an orthogonal second hit⁶. This “synthetic lethal” strategy may be particularly attractive when the oncogene-regulated functional network(s) intersect with tumor cell death pathways. In a certain example, oncogenic signaling drives glucose metabolism to suppress intrinsic apoptosis and promote survival. Inhibition of oncogenic drivers with targeted therapies can trigger the intrinsic apoptotic machinery as a direct consequence of attenuated glucose consumption. The intertwined nature of these tumorigenic pathways may present therapeutic opportunities for rational combination treatments, however, this has yet to be investigated.

In view of the foregoing, there remains a clinical need for brain penetrant chemotherapeutics for the treatment of glioblastoma and other cancers.

### SUMMARY OF THE INVENTION

The present disclosure provides compounds of formula I-a or I-b:

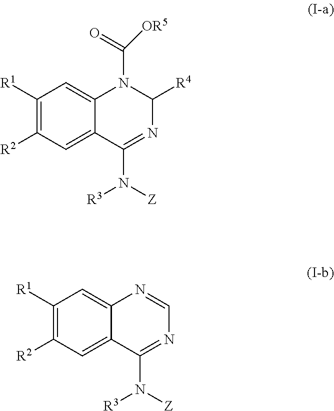

or a pharmaceutically acceptable salt thereof, wherein:
Z is aryl or heteroaryl, and is optionally substituted with one or more R⁶;
R¹ is hydrogen, alkyl, halo, CN, NO₂, OR⁷, cycloalkyl, heterocyclyl, aryl or heteroaryl;
R² is hydrogen, alkyl, halo, CN, NO₂, OR⁸, cycloalkyl, heterocyclyl, aryl or heteroaryl; or R¹ and R² complete a carbocyclic or heterocyclic ring;
R³ is hydrogen, alkyl, or acyl;
R⁴ is alkoxy;
R⁵ is alkyl;
each instance of R⁶ is independently selected from alkyl, alkoxy, OH, CN, NO₂, halo, alkenyl, alkynyl, aralkyloxy, cycloalkyl, heterocyclyl, aryl, or heteroaryl; and
R⁷ and R⁸ are, each independently, selected from hydrogen, alkyl, such as alkoxyalkyl, aralkyl, or arylacyl.

In certain aspects, the present disclosure provides methods of inhibiting EGFR or ΔEGFR, comprising administering to a subject an effective amount of a compound of formula I-a or I-b.

In certain aspects, the present disclosure provides methods of treating cancer comprising administering to a subject in need of a treatment for cancer an effective amount of a compound of formula I-a or I-b. In some embodiments, the cancer is glioblastoma multiforme.

In certain aspects, the present disclosure provides methods of treating cancer comprising administering to a subject a glucose metabolism inhibitor and a cytoplasmic p53 stabilizer. In some embodiments, the cancer is glioblastoma multiforme. In some embodiments, the glucose metabolism inhibitor is a compound of formula I-a or I-b.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

It is contemplated that any method or composition described herein can be implemented with respect to any other method or composition described herein and that different embodiments may be combined.

### BRIEF DESCRIPTION OF THE DRAWINGS

FIG. 1 depicts the oral pharmacokinetics of JGK005 at 10 mg/kg and those of erlotinib at 25 mg/kg. JGK005 has good CNS penetration compared to erlotinib.

FIG. 2 depicts the activity of erlotinib (left columns) and JGK005 (right columns) against EGFR mutant glioblastomas HK301 and GBM39, respectively. JGK005 has lower activity than erlotinib in both cases.

FIG. 3 depicts the cell free EGFR kinase activities of erlotinib and JGK010. Both compounds have an IC₅₀ of approximately 8 nM.

FIG. 4 depicts the potencies of erlotinib (left columns), JGK005 (center columns), and JGK010 (right columns) against HK301 and GBM39 cells.

FIG. 5 shows the oral pharmacokinetics of JGK005 at 10 mg/kg and of JGK010 at 10 mg/kg.

FIG. 6 depicts comparisons of EGFR inhibitors in multiple primary glioblastoma cell lines. Columns 1-4: GBM39 (EGFRvIII), 5-8: GS100 (EGFRwt/EGFRvIII), 9-12: GS017 (A289T), 13-16: GS024 (EGFR polysomy).

FIG. 7A depicts JGK010 activity in EGFR altered lung cancer. FIG. 7B depicts JGK010 activity in EGFR Amp epidermoid carcinoma.

FIG. 8A depicts JGK010 oral pharmacokinetics at 6 mg/kg. FIG. 8B depicts JGK010 oral pharmacokinetics at 10 mg/kg. FIG. 8C depicts JGK010 IV pharmacokinetics at 6 mg/kg. FIG. 8D depicts JGK010 IP pharmacokinetics at 6 mg/kg.

FIG. 9 depicts the activities of erlotinib and exemplary compounds of the disclosure against EGFR Amp WT+vIII HK301.

FIG. 10 depicts the activities of erlotinib and exemplary compounds of the disclosure against EGFR vIII Amp GBM 39.

FIG. 11 depicts the activities of erlotinib and exemplary compounds of the disclosure against HK301 cells.

FIG. 12 depicts the activities of erlotinib and exemplary compounds of the disclosure against GBM 39 cells.

FIG. 13A depicts the phosphor-EGFR vIII inhibition of erlotinib and exemplary compounds of the disclosure. FIG. 13B depicts the phosphor-EGFR vIII inhibition of erlotinib and exemplary compounds of the disclosure.

FIG. 14A depicts the pharmacokinetics of JGK005. FIG. 14B depicts the pharmacokinetics of JGK005.

FIG. 15A depicts the pharmacokinetics of JGK038. FIG. 15B depicts the pharmacokinetics of JGK038.

FIG. 16A depicts the pharmacokinetics of JGK010. FIG. 16B depicts the pharmacokinetics of JGK010.

FIG. 17A depicts the pharmacokinetics of JGK037. FIG. 17B depicts the pharmacokinetics of JGK037.

FIG. 18A depicts a comparison of mouse brain/blood pharmacokinetics between Erlotinib and JGK037. FIG. 18B depicts a comparison of mouse brain/blood pharmacokinetics between Erlotinib and JGK037.

FIG. 19 depicts the brain penetration of erlotinib and exemplary compounds of the disclosure.

FIG. 20 depicts the effect of treatment with either a vehicle or JGK037 on RLU change.

FIG. 21 depicts the inhibition of EGFR-driven glucose metabolism induces minimal cell death but primes GBM cells for apoptosis. FIG. 21A depicts percent change in ¹⁸F-FDG uptake after 4 hours of erlotinib treatment relative to vehicle in 19 patient-derived GBM gliomaspheres. “Metabolic responders” (blue) are samples that show a significant decrease in ¹⁸F-FDG uptake relative to vehicle, whereas “non-responders” (red) show no significant decrease. FIG. 21B depicts percent change in glucose consumption and lactate production with 12 hours of erlotinib treatment relative to vehicle. Measurements are made using Nova Biomedical BioProfile Analyzer. FIG. 21C depicts Annexin V staining of metabolic responders (blue, n=10) or non-responders (red, n=9) after treatment with erlotinib for 72 hours. FIG. 21D depicts the percent change, relative to vehicle control, in priming as determined by cytochrome c release following exposure to each BH3 peptide (BIM, BID, or PUMA) in metabolic responders or non-responders treated with erlotinib for 24 hours. FIG. 21E depicts Left: Immunoblot of whole cell lysate of HK301 cells overexpressing GFP control or GLUT1 and GLUT3 (GLUT1/3). Right: Changes in glucose consumption or lactate production of HK301-GFP or HK301-GLUT1/3 after 12 hours of erlotinib treatment. Values are relative to vehicle control. FIG. 21F depicts using HK301-GFP or HK301-GLUT1/3 cells. Erlotinib concentration for all experiments was 1 μM. Comparisons were made using two-tailed unpaired Student's t-test. Data represent means±s.e.m. values of three independent experiments. *p<0.05, **p<0.01, ***p<0.001, ****p<0.0001.

FIG. 22 depicts Cytoplasmic p53 links EGFR to intrinsic apoptosis. FIG. 22A depicts immunoblot of indicated proteins in two responders (HK301 and HK336) expressing CRISPR/CAS9 protein with control guide RNA (sgCtrl) or p53 guide RNA (p53KO). FIG. 22B depicts the percent change, relative to vehicle control, in priming as determined by cytochrome c release following exposure to BIM peptide in sgCtrl and p53KO cells treated with erlotinib for 24 hours. FIG. 22C depicts immunoblot of indicated proteins in HK301 sgCtrl, p53KO, p53KO+p53^{cyto}, and p53KO+p53^{wt}. FIG. 22D depicts that immunofluorescence of p53 protein combined with DAPI staining to reveal protein localization in HK301 sgCtrl, p53KO+p53^{cyto}, and p53KO+p53^{wt} (scale bars=20 μm). Gliomaspheres were first disassociated to single cell and adhered to the 96-well plates using Cell-Tak (Corning) according to manufacturer instructions. Adhered cells were then fixed with ice-cold methanol for 10 min then washed three times with PBS. Cells were then incubated with blocking solution containing 10% FBS and 3% BSA in PBS for 1 hr and subsequently incubated with p53 (Santa Cruz, SC-126, dilution of 1:50) antibody overnight at 4° C. The following day, cells were incubated with secondary antibody (Alexa Fluor 647, dilution 1:2000) for an hour and DAPI staining for 10 min, then imaged using a Nikon TI Eclipse microscope equipped with a Cascade II fluorescent camera (Roper Scientific). Cells were imaged with emissions at 461 nM and 647 nM and then processed using NIS-Elements AR analysis software. FIG. 22E depicts changes in indicated mRNA levels following 100 nM doxorubicin treatment for 24 hrs in HK301 sgCtrl, p53KO, p53KO+p53^{cyto}, and p53KO+p53^{wt}. Levels were normalized to respective DMSO treated cells. FIG. 22F depicts similar data to 22B but in HK301 sgCtrl, p53KO, p53KO+p53^{cyto}, and p53KO+p53^{wt}. FIG. 22G depicts similar data to 22E but in HK301 sgCtrl, p53KO, p53KO+p53^{R175H}, p53KO+p53^{R273H}, and p53KO+p53^{NES}. FIG. 22H depicts similar data to 22B and 22F but in HK301 sgCtrl, p53KO, p53KO+p53R^{175H}, p53KO+p53^{R273H}, and p53KO+p53^{NES}. Erlotinib concentration for all experiments was 1 μM. Comparisons were made using two-tailed unpaired Student's I-test. Data represent means±s.e.m. values of three independent experiments. *p<0.05, **p<0.01, ***p<0.001, ****p<0.0001.

FIG. 23 depicts Bcl-xL prevents GBM cell death by binding to and sequestering cytoplasmic p53 in EGFRi-metabolic responders. FIG. 23A depicts the immunoprecipitation of p53 in two metabolic responders (HK301 and GBM39) following 24 hours of erlotinib treatment. The immunoprecipitate was probed with the indicated antibodies. Below are respective pre-immunoprecipitation lysates (input). FIG. 23B depicts data similar to 23A but in two non-responders (HK393 and HK254). FIG. 23C depicts data similar to 23A and 23B but in HK301-GFP and HK301-GLUT/3. To the right are immunoblots for indicated inputs. FIG. 23D depicts HK301 was treated for 24 hours with erlotinib, WEHI-539, or both and immunoprecipitation and immunoblotting was performed as described previously. FIG. 23E depicts annexin V staining of two responders (GBM39 and HK301) and a non-responder (HK393) following 72 hours of treatment with erlotinib, WEHI-539, or both. FIG. 23F depicts annexin V staining of HK301-GFP and HK301-GLUT1/3 following 72 hours of treatment with erlotinib, wehi-539, or both. Erlotinib and WEHI-539 concentrations for all experiments were 1 μM and 5 μM, respectively. Comparisons were made using two-tailed unpaired Student's t-test. Data represent means±s.e.m. values of three independent experiments. *p<0.05, **p<0.01.

FIG. 24 depicts the synergistic lethality of combined targeting of EGFR and p53. FIG. 24A depicts a summary of alterations in EGFR and genes involved in p53 regulation across 273 GBM samples. Genetic alterations in EGFR (amp/mutation) are mutually exclusive to those in p53. As shown, EGFR alterations are on the left side of the table while most alterations in p53 are on the right. FIG. 24B depicts a table indicating the significant associations between alterations in EGFR and genes involved in the p53 pathway. FIG. 24C depicts Annexin V staining of a metabolic responder (left: HK301) and non-responder (right: GS017) treated with varying concentrations of erlotinib, nutlin, and in combination represented as a dose-titration matrix. FIG. 24D depicts the dose-titration of erlotinib and nutlin as described in 24C was conducted across 10 metabolic responders and 6 non-responders, and the synergy score was calculated (see Materials and Methods). FIG. 24E depicts Annexin V staining of HK301-GFP and HK301 GLUT1/3 following 72 hours of treatment with erlotinib, nutlin, or both. FIG. 24F depicts the same as 24E but in HK301-sgCtrl and HK301-p53KO. FIG. 24G depicts HK301 that was treated for 24 hours with erlotinib, nutlin, or in combination. Immunoprecipitation was performed with immunoglobulin G control antibody or anti-p53 antibody, and the immunoprecipitate was probed with the indicated antibodies. Below are respective pre-immunoprecipitation lysates (input). All data are representative of at least n=3 independent experiments, mean±SEM. Unless indicated, erlotinib and nutlin concentrations for all experiments were 1 μM and 2.5 μM, respectively. ** p<0.01, *** p<0.001, **** p<0.0001

FIG. 25 depicts the modulation of glucose metabolism primes EGFRi non-responders for p53-mediated cell death. FIG. 25A depicts the percentage change in ¹⁸F-FDG uptake after 4 hours of erlotinib, 2DG, or pictilisib treatment relative to vehicle in HK393 and HK254. FIG. 25B depicts the percentage change, relative to vehicle control, in priming as determined by cytochrome c release following exposure to BIM peptide in HK393 and HK254 following erlotinib, 2DG, or pictilisib for 24 hours. FIG. 25C depicts data similar to 25B but in HK393 sgCtrl and p53KO. FIG. 25D depicts the immunoprecipitation of p53 in HK393 and HK254 following 24 hours of 2DG or pictilisib treatment. The immunoprecipitate was probed with the indicated antibodies. Below are respective pre-immunoprecipitation lysates (input). FIG. 25E depicts the synergy score of various drugs (erlotinib, 2DG, and pictilisib) in combination with nutlin in HK393 and HK254. FIG. 25F depicts Annexin V staining of HK393 sgCtrl and HK393 p53KO following 72 hours of treatment with 2DG, pictilisib, 2DG+nutlin, or pictilisib+nutlin. Unless indicated, erlotinib, 2DG, pictilisib, and nutlin concentrations for all experiments were 1 μM, 1 mM, 1 μM and 2.5 μM, respectively. Comparisons were made using two-tailed unpaired Student's 1-test. Data represent means±s.e.m. values of three independent experiments. *p<0.05, **p<0.01, ***p<0.001.

FIG. 26 depicts the combined targeting of EGFR-driven glucose uptake and p53 suppresses tumor growth in vivo. FIG. 26A depicts the ¹⁸F-FDG PET/CT imaging of GBM39 intracranial xenografts before and after 15 hours erlotinib treatment (75 mg/kg). FIG. 26B depicts GBM39 intracranial xenografts that were treated with vehicle (n=5), 75 mg/kg erlotinib (n=7), 50 mg/kg Idasanutlin (n=5), or in combination daily (n=12), and tumor burden was assessed at indicated days using secreted gaussia luciferase (see Materials and Methods). FIG. 26C depicts data similar to 26A but in HK393 intracranial xenografts. FIG. 26D depicts data similar to 26B, but in HK393 intracranial xenografts (n=7 for all groups). FIG. 25E depicts the percent survival of 26B. FIG. 26F depicts the percent survival of 26C. FIG. 26G depicts the percent survival of metabolic responder HK336 following indicated treatments for 25 days and then released from drug (n=7 for all groups). FIG. 26H depicts the percent survival of non-responder GS025 following indicated treatments for 25 days and then released from drug (n=9 for all groups). Comparisons for 26B and 26D used data sets from the last measurements and were made using two-tailed unpaired t-test. Data represent means±s.e.m. values. **p<0.01.

FIG. 27 depicts the characterization of GBM cell lines following EGFR inhibition. FIG. 27A depicts the percent change in ¹⁸F-FDG uptake at indicated times of erlotinib treatment relative to vehicle in two metabolic responders (HK301 and GBM39). FIG. 27B depicts an immunoblot of indicated proteins of a metabolic responder (HK301) and non-responder (HK217) following genetic knockdown of EGFR with siRNA. FIG. 27C depicts the percent change in ¹⁸F-FDG uptake in HK301 and HK217 following genetic knockdown of EGFR. FIG. 27D depicts the change in glucose consumption with 12 hours of erlotinib treatment in three metabolic responders (HK301, GBM39, HK390) and three non-responders (HK393, HK217, HK254). Measurements are made using Nova Biomedical BioProfile Analyzer. FIG. 27E depicts the change in and lactate production with 12 hours of erlotinib treatment in three metabolic responders (HK301, GBM39, HK390) and three non-responders (HK393, HK217, HK254). Measurements are made using Nova Biomedical BioProfile Analyzer. FIG. 27F depicts basal ECAR measurements of two responders (HK301 and GBM39, in blue) and two non-responders (HK217 and HK393, in red) following 12 hours of erlotinib treatment. FIG. 27G depicts change in glutamine consumption following 12 hours of erlotinib treatment, as measured by Nova Biomedical BioProfile Analyzer. Erlotinib concentrations for all experiments were 1 μM. Comparisons were made using two-tailed unpaired Student's t-test. Data represent means±s.e.m. values of three independent experiments. *p<0.05, **p<0.01, ***p<0.001, ****p<0.0001.

FIG. 28 depicts alterations in downstream signaling following EGFR inhibition correlate with metabolic response. FIG. 28A depicts an immunoblot of indicated proteins following 4 hours of erlotinib treatment in metabolic responders. FIG. 28B depicts an immunoblot of indicated proteins following 4 hours of erlotinib treatment in metabolic non-responders.

FIG. 29 depicts the genetic characterization of patient-derived GBM cell lines. FIG. 29A depicts the genetic background across a panel of GBM lines. FIG. 29B depicts fluorescence in situ hybridization (FISH) of HK390, HK336, HK254, and HK393 showing polysomy of EGFR. Fluorescence in situ hybridization (FISH) was performed using commercially available fluorescently labeled dual-color EGFR (red)/CEP 7(green) probe (Abbott-Molecular). FISH hybridization and analyses were performed on cell lines, following the manufacturer's suggested protocols. The cells were counterstained with DAPI and the fluorescent probe signals were imaged under a Zeiss (Axiophot) Fluorescent Microscope equipped with dual- and triple-color filters.

FIG. 30 depicts EGFR inhibition shifts the apoptotic balance in metabolic responders. FIG. 30A depicts an immunoblot of indicated proteins following 24 hours of erlotinib treatment in metabolic responders (GBM39, HK301, and HK336) and non-responders (HK217, HK393, and HK254). FIG. 30B depicts example of dynamic BH3 profiling analysis in a metabolic responder (HK301). Left: Percent cytochrome c release is measured following exposure to various peptides at indicated concentrations. Right: The difference in cytochrome c release between vehicle treated cells and erlotinib treated cells is calculated to obtain the percent priming. Erlotinib concentrations for all experiments was 1 μM.

FIG. 31 depicts GLUT1/3 overexpression rescues attenuated glucose metabolism caused by EGFR inhibition. FIG. 31A depicts the change in glucose consumption and lactate production with 12 hours of erlotinib treatment in HK301-GFP and HK301 GLUT1/3. Measurements are made using Nova Biomedical BioProfile Analyzer. FIG. 31B depicts Left: Immunoblot of whole cell lysate of GBM39 cells overexpressing GFP control or GLUT1 and GLUT3 (GLUT1/3). Right: Changes in glucose consumption or lactate production of GBM39-GFP or GBM39-GLUT1/3 after 12 hours of erlotinib treatment. Values are relative to vehicle control. FIG. 31C depicts data similar to 35A but in GBM39-GFP and GBM39-GLUT1/3. Erlotinib concentrations for all experiments was 1 μM. Comparisons were made using two-tailed unpaired Student's 1-test. Data represent means±s.e.m. values of three independent experiments. *p<0.05, **p<0.01, ***p<0.001, ****p<0.0001.

FIG. 32 depicts cytoplasmic p53 is required for EGFRi-mediated apoptopic priming. FIG. 32A depicts the percent change in ¹⁸F-FDG uptake following 4 hours of erlotinib treatment in HK301 sgCtrl and p53 KO cells (mean±s.d., n=3). FIG. 32B depicts relative mRNA levels of p53-regulated genes following 24 hours 1 μM erlotinib treatment in or 100 nM doxorubicin treatment in HK301 (metabolic responder). FIG. 32C depicts HK301 cells infected with a p53-luciferase reporter system and p53 activity was measured following 24 hours of 1 μM erlotinib treatment (mean±s.d., n=3). Results are representative of two independent experiments. FIG. 32D depicts Immunoblot of indicated proteins in HK336 sgCtrl, p53KO, p53KO+p53⁻¹, and p53KO+p53^{wt}. FIG. 32E depicts the immunofluorescence of p53 protein combined with DAPI staining to reveal protein localization in HK336 sgCtrl, p53KO+p53^{cyto}, and p53KO+p53^{wt} (scale bars=20 μm). Immunofluorescence was performed as previously described. FIG. 32F depicts changes in indicated mRNA levels following 100 nM doxorubicin treatment for 24 hrs in HK336 sgCtrl, p53KO, p53KO+p53^{cyto}, and p53KO+p53^{wt} (mean±s.d., n=3). Levels were normalized to respective DMSO treated cells. FIG. 32G depicts the percent change, relative to vehicle control, in apoptotic priming—as determined by cytochrome c release following exposure to BIM peptide—in HK336 sgCtrl, p53KO, p53KO+p53^{cyto}, and p53KO+p53^{wt} cells treated with erlotinib for 24 hours. (mean±s.d., n=2). Results are representative of two independent experiments. FIG. 32H depicts an immunoblot of indicated proteins in HK301 sgCtrl, p53KO, p53KO+p53^{R175H}, p53KO+p53^{R273H}, and p53KO+p53^{NES}. FIG. 32I depicts the percent change in priming in HK301 following 24 hours of erlotinib treatment with or without PFTμ pre-treatment (10 μM for 2 hours) (mean±s.d., n=2). Results are representative of two independent experiments.

FIG. 33 depicts the inhibition of EGFR-driven glucose metabolism induces a Bcl-xL dependency through cytoplasmic p53 functions. FIG. 33A depicts the percent change, relative to vehicle control, in priming as determined by cytochrome c release following exposure to BAD and HRK peptides in metabolic responders (HK301 and HK336) or non-responder (HK229) treated with erlotinib. FIG. 33B depicts Left: Immunoprecipitation of p53 in GBM39-GFP and GBM39-GLUT1/3 following 24 hours of erlotinib treatment. The immunoprecipitate was probed with the indicated antibodies. Right: respective pre-immunoprecipitation lysates (input). FIG. 33C depicts Annexin V staining of HK301 (left) and HK336 (right) sgCtrl, p53KO, p53 KO+p53^{cyto}, and p53KO+p53^{wt} following 72 hours of treatment with erlotinib, WEHI-539, or combination. FIG. 33D depicts data similar to 33C but in GBM39-GFP and GBM39-GLUT1/3. Erlotinib and WEHI-539 concentrations for all experiments were 1 μM. Comparisons were made using two-tailed unpaired Student's t-test. Data represent means±s.e.m. values of three independent experiments. *p<0.05, **p<0.01, ***p<0.001.

FIG. 34 depicts the inhibition of EGFR-regulated glucose metabolism and p53 activation promote intrinsic apoptosis in GBM. FIG. 34A depicts the immunoblot of indicated proteins following 24 hours of erlotinib, nutlin or in combination in two metabolic responders (HK301 and GBM39). FIG. 34B depicts Annexin V staining in HK301 and HK217 following genetic knockdown of EGFR and subsequent nutlin treatment for 72 hours. FIG. 34C depicts the detection of BAX oligomerization in HK301-GFP and HK301-GLUT1/GLUT3. Following 24 hours of indicated treatment, cells were harvested and incubated in 1 mM BMH to promote protein cross-linking and immunoblotted with indicated antibodies. Below BAX is immunoblot for cytosolic cytochrome c following cellular fractionation. FIG. 34D depicts the Top: Immunoblot of indicated proteins in HK301-GFP and HK301-HA-BclxL. Bottom: Annexin V staining in HK301-GFP and HK301-HA-BclxL following 72 hours of treatment with erlotinib, nutlin, or combination. FIG. 34E depicts Annexin V staining of HK301 following 72 hours of erlotinib, nutlin or the combination+/−PFTμ pretreatment (10 μM for 2 hours). FIG. 34F depicts Annexin V staining of HK301 sgCtrl, p53KO, p53KO+p53^{R175H}, p53KO+p53^{R273H}, and p53KO+p53^{NES} following 72 hours of treatment with erlotinib, nutlin, or combination. FIG. 34G depicts data similar to 34F but in HK301 sgCtrl, p53KO, p53KO+p53^{cyto}, and p53KO+p53^{wt}. Drug concentrations for all experiments are as follows: erlotinib (1 μM), nutlin (2.5 μM). Comparisons were made using two-tailed unpaired Student's t-test. Data represent means±s.e.m. values of three independent experiments. *p<0.05, **p<0.01, ***p<0.001, ****p<0.0001. FIG. 34H depicts data similar to 34G but in HK336 sgCtrl, p53KO, p53KO+p53^{cyto}, and p53KO+p53^{cyto}. Drug concentrations for all experiments are as follows: eriotinib (1 μM), nutlin (2.5 μM). Comparisons were made using two-tailed unpaired Student's t-test. Data represent means±s.e.m. values of three independent experiments. *p<0.05, **p<0.01, ***p<0.001, ****p<0.0001.

FIG. 35 depicts the inhibition of glucose metabolism in metabolic responders and non-responders promotes intrinsic apoptosis. FIG. 35A depicts the percent change, relative to vehicle control, in priming as determined by cytochrome c release following exposure to BIM peptides in metabolic responder HK301 following 24 hours of erlotinib or 2DG treatment. FIG. 35B depicts Left: Immunoprecipitation of p53 in HK301 following 24 hours of 2DG treatment. The immunoprecipitate was probed with the indicated antibodies. Right: respective pre-immunoprecipitation lysates (input). FIG. 35C depicts OCR and ECAR measurements of HK301 cells following exposure to oligomycin and rotenone. FIG. 35D depicts Annexin V staining in HK301 following 72 hours of treatment with nutlin, erlotinib, 2DG, oligomycin, rotenone as individual agents or in combination with nutlin. FIG. 35E depicts an immunoblot of indicated proteins following 4 hours of erlotinib or pictilisib treatment in two non-responders (HK254 and HK393). FIG. 35F depicts the Immunoprecipitation of p53 in HK254 following 24 hours of pictilisib or 2DG treatment. The immunoprecipitate was probed with the indicated antibodies. Below are respective pre-immunoprecipitation lysates (input). Drug concentrations for all experiments are as follows: erlotinib (1 μM), nutlin (2.5 μM), 2DG (3 mM for HK301 and 1 mM for HK254), oligomycin (1 μM), rotenone (1 μM), and pictilisib (1 μM). Comparisons were made using two-tailed unpaired Student's t-test. Data represent means±s.e.m. values of three independent experiments. ****p<0.0001.

FIG. 36 depicts the In vivo efficacy of EGFR inhibition and p53 activation. FIG. 36A depicts the brain and plasma concentrations of Idasanutlin at indicated time points (n=2 mice/time point) in non-tumor bearing mice. FIG. 36B depicts the immunohistochemistry (IHC) analysis of p53 expression in intracranial tumor-bearing xenografts following 36 hours Idasanutlin (50 mg/kg) treatment. FIG. 36C depicts the percent change in ¹⁸F-FDG uptake following 15 hours of erlotinib treatment in GBM39 (n=3) and HK393 (n=5) intracranial xenografts. FIG. 36D depicts the change in mice body weight following daily treatment with erlotinib (75 mg/kg) or combined erlotinib (75 mg/kg) and Idasanutlin (50 mg/kg). All treatments were done orally. Data represent means±s.e.m. values of three independent experiments. *p<0.05.

FIG. 37A depicts that direct inhibition of glycolysis with 2DG (hexokinase inhibitor) or cytochalasin B (a glucose transporter inhibitor) unexpectedly synergizes with p53 activation (with nutlin). FIG. 37B depicts low glucose (0.25 mM) leads to synergistic cell kill with BCL-xL inhibition with navitoclax (ABT-263). FIG. 37C depicts low glucose (0.25 mM) leads to synergistic cell kill with BCL-xL inhibition with nutlin.

FIG. 38 depicts a comparison between metabolic responders to EGFRi inhibitor, erlotinib, and metabolic non-responders. The combination of erlotinib and nutlin leads to an unexpected synergistic synthetic lethality in metabolic responders but not in non-responders.

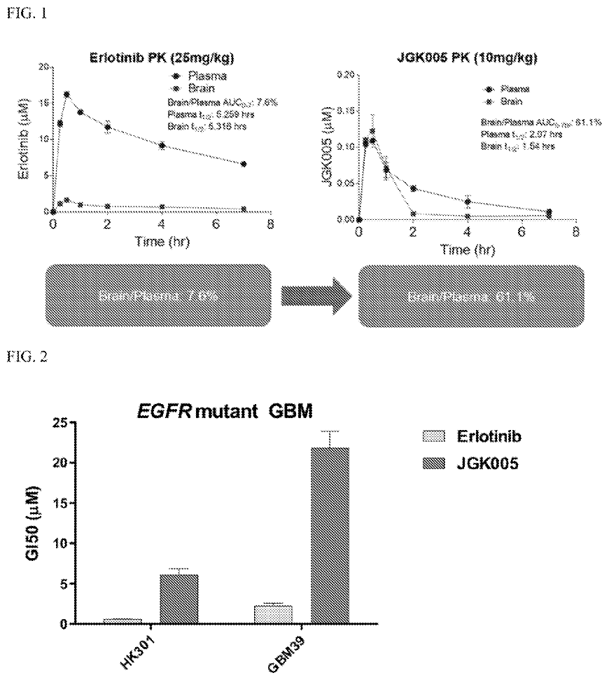

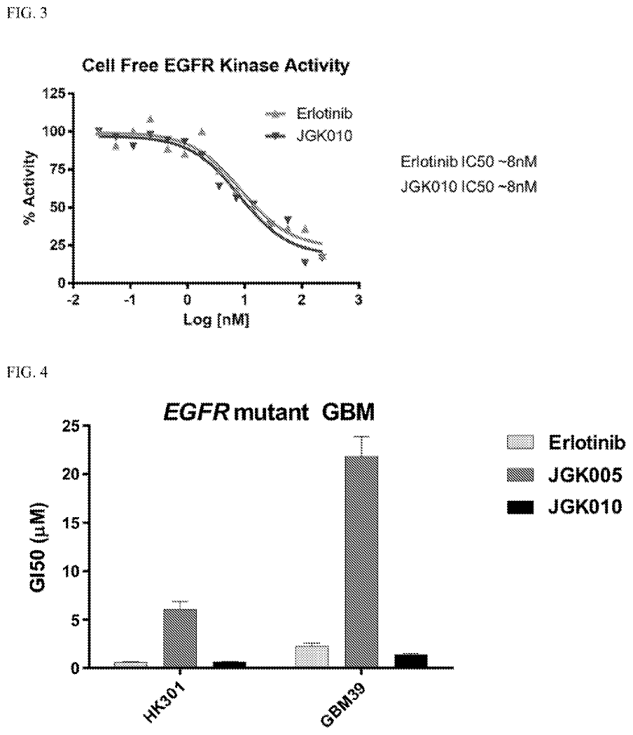

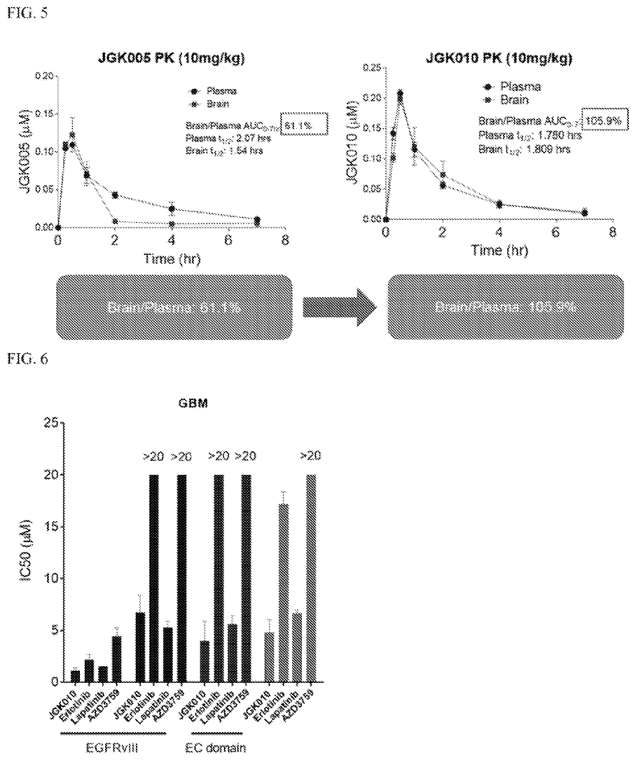

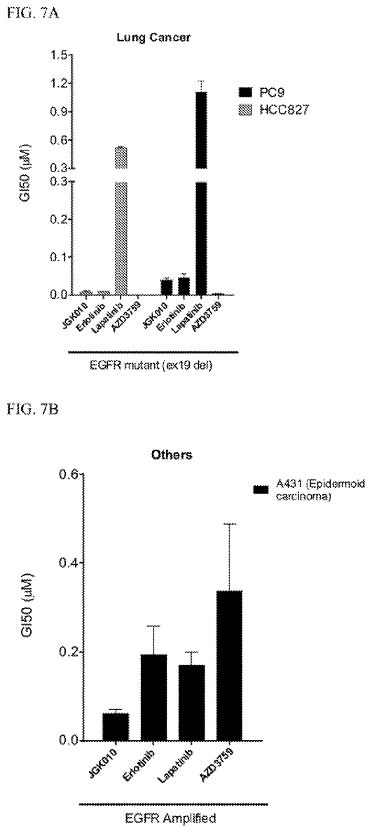

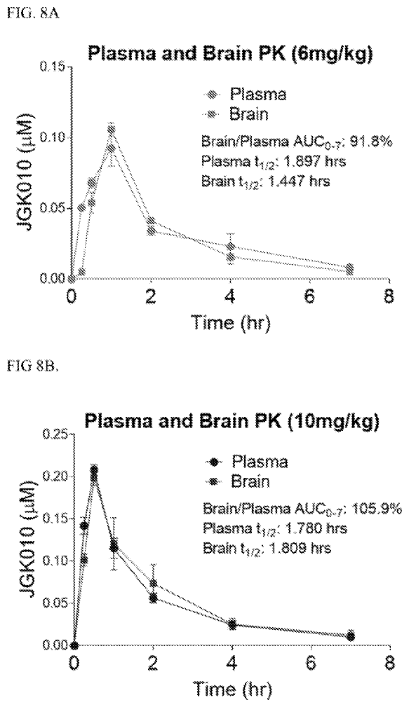

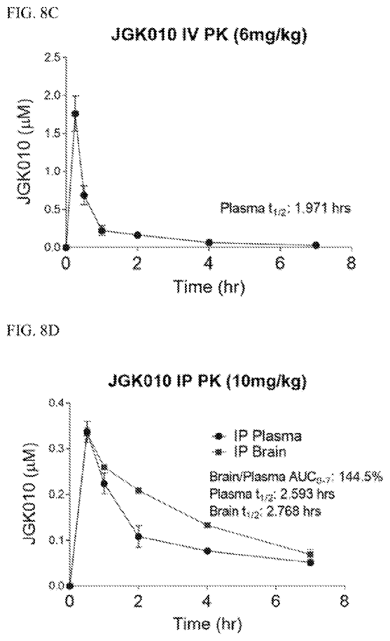

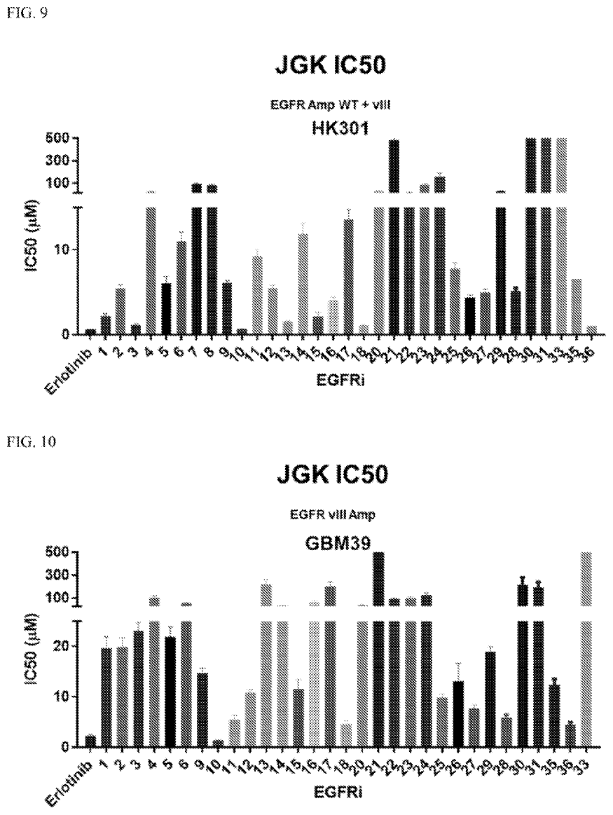

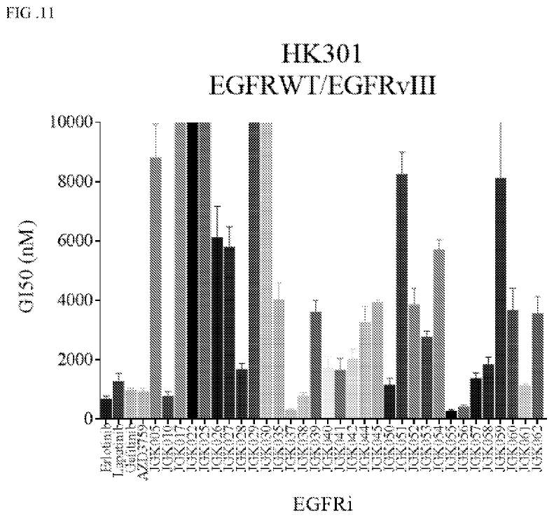

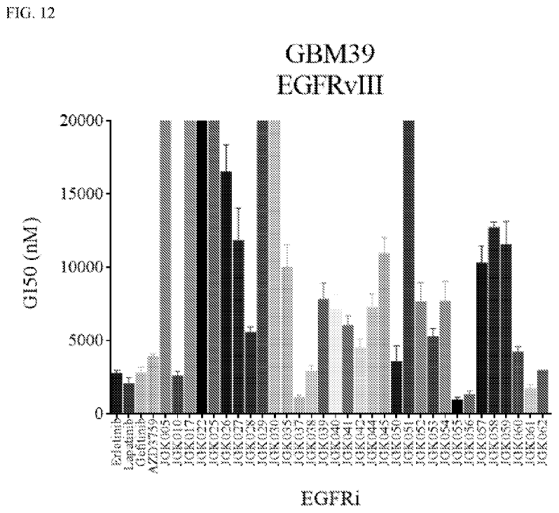

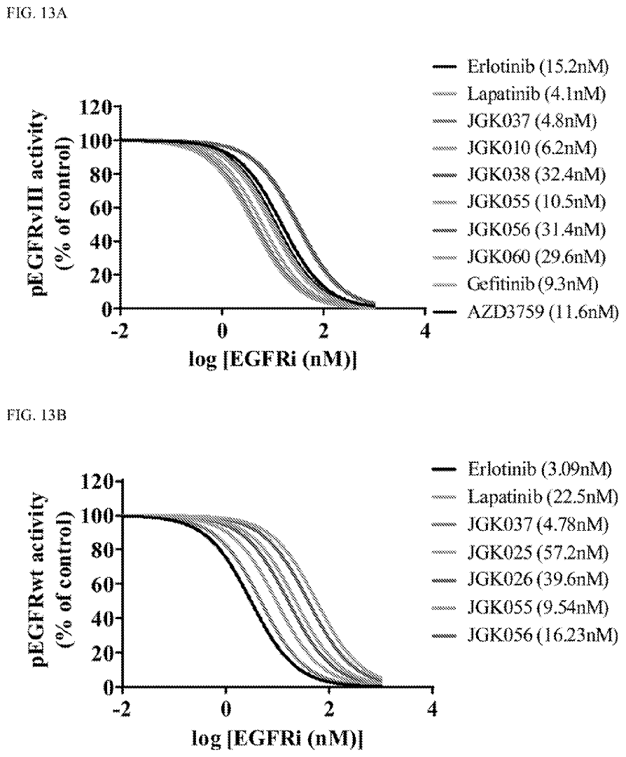

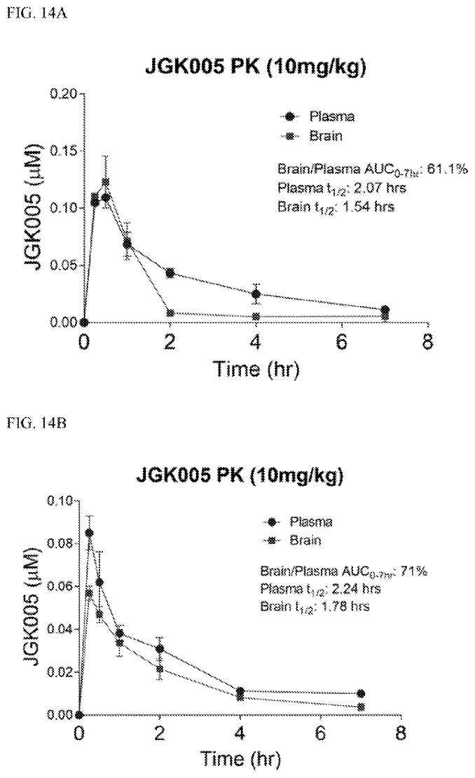

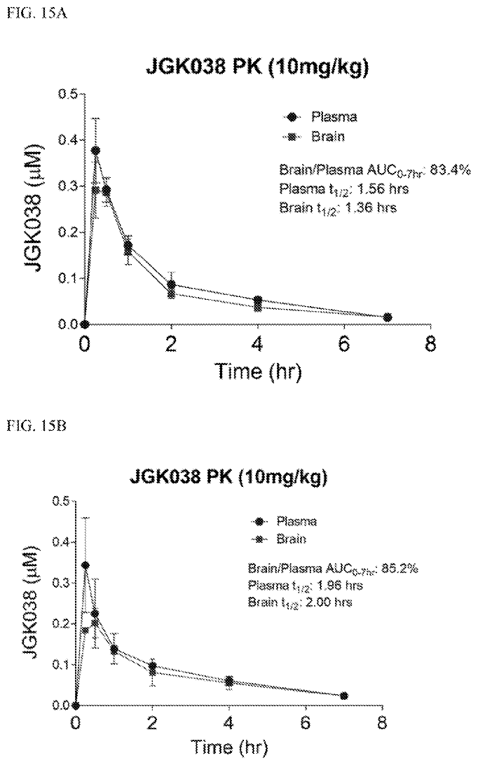

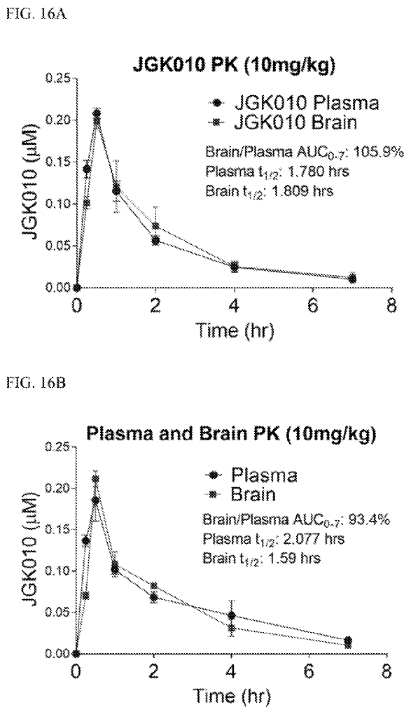

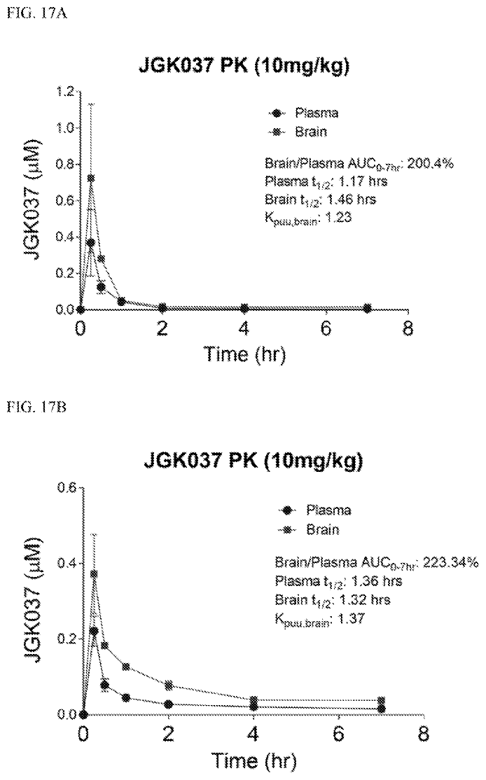

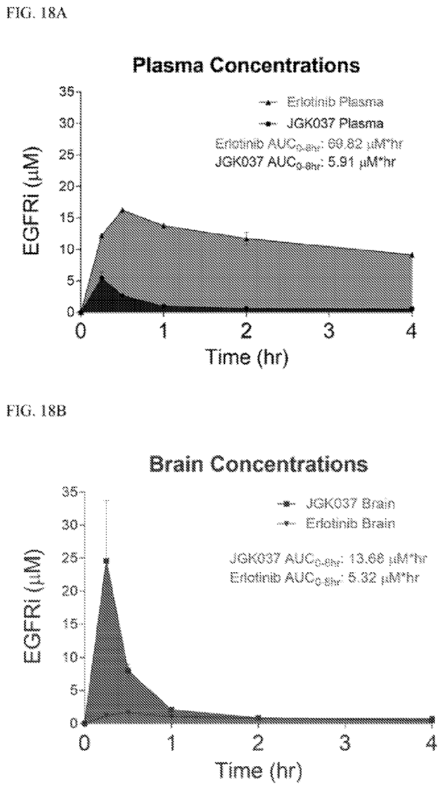

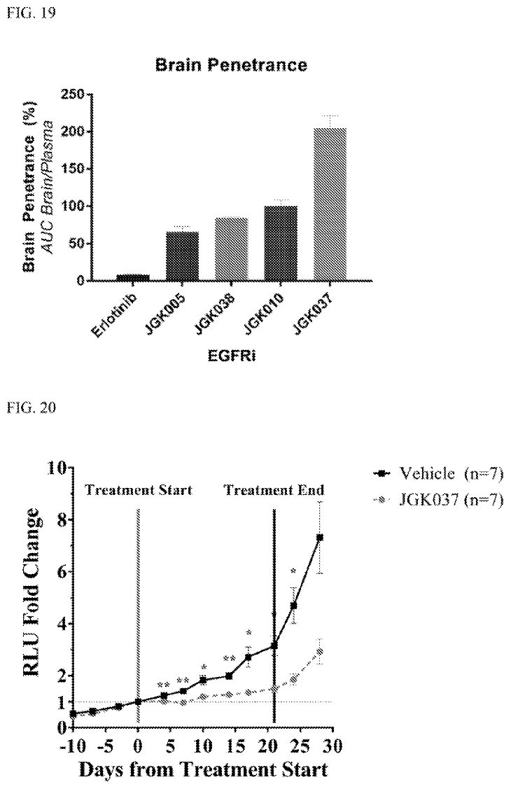

### DETAILED DESCRIPTION OF THE INVENTION

Gliomas are the most commonly occurring form of brain tumor, with glioblastoma multiforme (GBM) being most malignant form, causing 3-4% of all cancer-related deaths (Louis et al. (2007) Acta. Neuropathol. 114: 97-109.). The World Health Organization defines GBM as a grade IV cancer characterized as malignant, mitotically active, and predisposed to necrosis. GBM has a very poor prognosis with a 5-year survival rate of 4-5% with the median survival rate of GBM being 12.6 months (McLendon et al. (2003) Cancer. 98:1745-1748.). This can attributed to unique treatment limitations such as a high average age of onset, tumor location, and poor current understandings of the tumor pathophysiology (Louis et al. (2007) Acta. Neuropathol. 114: 97-109).The standard current standard of care for GBM includes tumor resection with concurrent radiotherapy and chemotherapy and in recent years there have been few marked improvements that increase survival rates (Stewart, et al. (2002) Lancet. 359:1011-1018.).

The standard for GBM chemotherapy is temozolomide (TMZ), which is a brain-penetrant alkylating agent that methylates purines (A or G) in DNA and induces apoptosis (Stupp, et al. (2005) N. Engl. J. Med. 352:987-996). However, TMZ use has drawbacks in that significant risk arises from DNA damage in healthy cells and that GBM cells can rapidly develop resistance towards the drug (Carlsson, et al. (2014) EMBO. Mol. Med. 6: 1359-1370). As such, additional chemotherapy options are urgently required.

EGFR is a member of the HER superfamily of receptor tyrosine kinases together with ERBB2, ERBB3, and ERBB4. A common driver of GBM progression is EGFR amplification, which is found in nearly 40% of all GBM cases (Hynes et al. (2005) Nat. Rev. Cancer. 5: 341-354; Hatanpaa et al. (2010) Neoplasia. 12:675-684). Additionally, EGFR amplification is associated with the presence of EGFR protein variants: in 68% of EGFR mutants; there is a deletion in the N-terminal ligand-binding region between amino acids 6 and 273. These deletions in the ligand-binding domains of EGFR can lead to ligand-independent activation of EGFR (Yamazaki et al. (1990) Jpn. J. Cancer Res. 81: 773-779.).

Small molecule tyrosine kinase inhibitors (TKIs) are the most clinically advanced of the EGFR-targeted therapies, and both reversible and irreversible inhibitors are in clinical trials. Examples of the reversible inhibitors and irreversible inhibitors include erlotinib. gefitinib, lapatinib, PKI1166, canertinib and pelitinib (Mischel et al. (2003) Brain Pathol. 13: 52-61). Mechanistically, these TKIs compete with ATP for binding to the tyrosine kinase domain of EGFR, however, these EGFR-specific tyrosine kinase inhibitors have been relatively ineffective against gliomas, with response rates only reaching as high as 25% in the case of erlotinib (Mischel et al. (2003) Brain Pathol. 13: 52-61; Gan et al. (2009) J. Clin. Neurosci. 16: 748-54). Although TKIs are well tolerated and display some antitumor activity in GBM patients, the recurrent problem of resistance to receptor inhibition limits their efficacy (Learn et al. (2004) Clin. Cancer. Res. 10: 3216-3224; Rich et al. (2004) Nat. Rev. Drug Discov. 3: 430-446). Additionally, recent studies have shown that brain plasma concentrations of gefitinib and erlotinib following therapy were only 6-11% of the starting dose, suggesting that these compounds may be failing to cross the blood-brain barrier as illustrated in table 1 (Karpel-Massler et al. (2009) Mol. Cancer Res. 7:1000-1012). Thus, insufficient delivery to the target may be another cause of the disappointing clinical results.

TABLE 1
Brain Penetration Rates of the Current Standard of Care Drugs

| | | | | | Brain |
|---|---|---|---|---|---|
| | | | | | Penetration |
| | | Daily | Plasma | CSF | rate |
| Compound | Primary | Dose | (ng/ml) | (ng/ml) | (%) |
| Afatinib | EGFR- | 50 | 66.7 | 0.46 | 0.7 |
| | mutant | | | | |
| | NSCLC | | | | |
| Alectinib | ALK- | 1200 | 1.5 (unbound | 1.3 | 86.7 |
| | mutant | | conc.) | | |
| | NSCLC | | | | |
| Crizotinib | ALK- | 500 | 237 | 0.616 | 0.26 |
| | mutant | | | | |
| | NSCLC | | | | |
| Erlotinib | EGFR- | 150 | 1140 ± | 28.7 ± | 2.77 ± |
| | mutant | | 937 | 16.8 | 0.45 |
| | NSCLC | | | | |
| | EGFR- | 1500 | 4445.9 | 51.1 | 1.2 |
| | mutant | (weekly) | | | |
| | NSCLC | | | | |
| Gefitinib | EGFR- | 250 | 326 ± | 3.7 ± | 1.13 ± |
| | mutant | | 116 | 1.9 | 0.36 |
| | NSCLC | | | | |
| | EGFR- | 750- | 1345.9- | 14.7- | 1.07- |
| | mutant | 1000 | 5094.4 | 143.1 | 3.58 |
| | NSCLC | | | | |
| Lapatinib | HER2 + | 1250 | 1515, 3472 | 1.3, 4.5 | 0.09, 0.13 |
| | breast | | | | |
| | cancer | | | | |

In light of this evidence, there remains an unmet clinical need for potent tyrosine kinase inhibitors that have the ability to cross the blood brain barrier and treat inhibit EGFR and its isoforms.

Furthermore, cross-talk among oncogenic signaling and metabolic pathways is shown by the inventors to create opportunities for novel combination therapies in GBM. More specifically, the inventors have discovered that acute inhibition of EGFR-driven glucose uptake induces minimal cell death, yet lowers the apoptotic threshold in patient-derived GBM cells and “primes” cells for apoptosis. Unexpectedly, mechanistic studies, by the inventors, revealed that Bcl-xL blocks cytoplasmic p53 from triggering intrinsic apoptosis, leading to tumor survival. Pharmacological stabilization of p53 (such as for example, with the brain-penetrant small molecule, Idasanutlin) enables p53 to engage the intrinsic apoptotic machinery, promoting synergistic lethality with targeting EGFR-driven glucose uptake in GBM xenografts. Notably, the inventors also discovered that rapid changes in ¹⁸F-fluorodeoxyglucose (¹⁸F-FDG) uptake using, for example, non-invasive positron emission tomography could predict sensitivity to the combination in vivo.

The inventors, inter alia, identify a critical link between oncogene signaling, glucose metabolism, and cytoplasmic p53, which could be exploited for combination therapy in GBM and other malignancies

### COMPOUNDS OF THE DISCLOSURE

The present disclosure provides compounds of formula I-a or I-b:

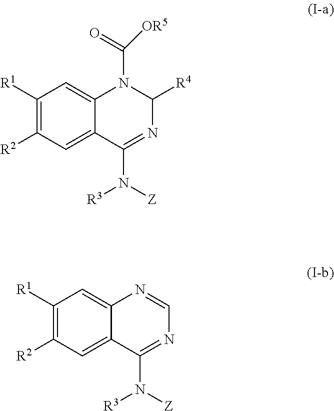

or a pharmaceutically acceptable salt thereof, wherein:
Z is aryl or heteroaryl, and is optionally substituted with one or more R⁶;
R¹ is hydrogen, alkyl, halo, CN, NO₂, OR⁷, cycloalkyl, heterocyclyl, aryl or heteroaryl;
R² is hydrogen, alkyl, halo, CN, NO₂, OR⁸, cycloalkyl, heterocyclyl, aryl or heteroaryl; or R¹ and R² complete a carbocyclic or heterocyclic ring;
R³ is hydrogen, alkyl, or acyl;
R⁴ is alkoxy;
R⁵ is alkyl;
each instance of R⁶ is independently selected from alkyl, alkoxy, OH, CN, NO₂, halo, alkenyl, alkynyl, aralkyloxy, cycloalkyl, heterocyclyl, aryl, or heteroaryl; and
R⁷ and R⁸ are, each independently, selected from hydrogen, alkyl, such as alkoxyalkyl, aralkyl, or arylacyl.

In certain embodiments of Formula I-a or Formula I-b, if R⁷ and R⁸ are alkoxyalkyl and R³ is hydrogen, then Z is not 3-ethynylphenyl.

In certain embodiments of Formula I-a or Formula I-b, Z is optionally substituted with R⁶ selected from alkyl, alkoxy, OH, CN, NO₂, halo, alkenyl, aralkyloxy, cycloalkyl, heterocyclyl, aryl, and heteroaryl.

In certain embodiments of Formula I-a or Formula I-b, R⁷ and R⁸ are, each independently, selected from hydrogen, aralkyl, or arylacyl; each instance of R⁶ is independently selected from alkyl, alkoxy, OH, CN, NO₂, halo, alkenyl, aralkyloxy, cycloalkyl, heterocyclyl, aryl, or heteroaryl; or R¹ and R² taken together complete a carbocyclic or heterocyclic ring.

In certain embodiments of Formula I-a or Formula I-b, if R⁷ and R⁸ are combined to form a heterocylic ring and R³ is hydrogen, then Z is not 2-fluoro,4-bromophenyl, 3-bromophenyl, 3-methylphenyl, 3-trifluoromethylphenyl, or 3-chloro,4-fluorophenyl.

In certain embodiments of Formula I-a or Formula I-b, the compound is a compound of Formula (II-a) or formula (II-b):

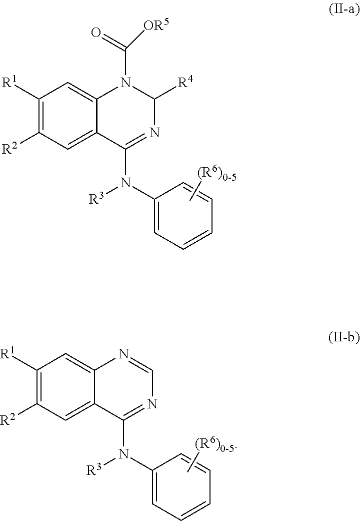

In certain embodiments of Formula I-a, Formula I-b, Formula II-a, or Formula II-b, R¹ is hydrogen. In other embodiments, R¹ is OR⁷.

In certain embodiments of Formula I-a, Formula I-b, Formula II-a, or Formula II-b, R⁷ is hydrogen. In certain embodiments, R⁷ is alkyl. In certain embodiments, R⁷ is alkoxyalkyl. In certain embodiments, R⁷ is arylacyl.

In certain embodiments of Formula I-a, Formula I-b, Formula II-a, or Formula II-b, R² is heteroaryl, such as furanyl. In certain embodiments, the heteroaryl of R² is substituted with alkyl, alkoxy, OH, CN, NO₂, halo,

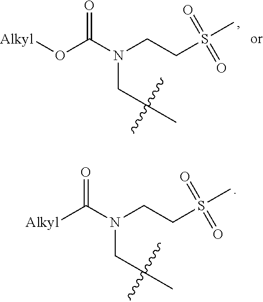

In other embodiments, R² is OR⁸.

In certain embodiments of Formula I-a, Formula I-b, Formula II-a, or Formula II-b, R⁸ is hydrogen. In certain embodiments, R⁸ is alkoxyalkyl. In certain embodiments, R⁸ is alkyl substituted with

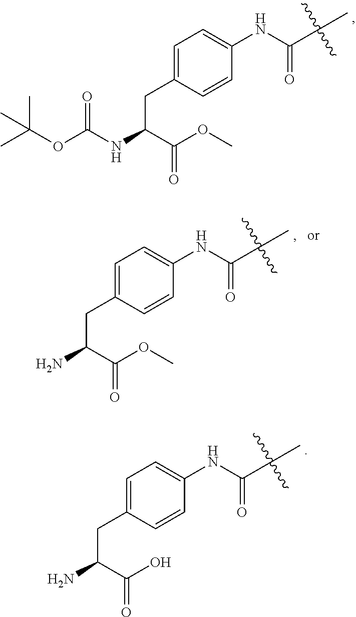 [SMILES: *C(=O)Nc1ccc(C[C@H](NC(=O)OC(C)(C)C)C(=O)OC)cc1 ; *C(=O)Nc1ccc(C[C@H](N)C(=O)OC)cc1 ; *C(=O)Nc1ccc(C[C@H](N)C(=O)O)cc1]

In certain embodiments, R⁸ is acyl. In certain embodiments, R⁸ is arylacyl.

In certain preferred embodiments of Formula I-a, Formula I-b, Formula II-a, or Formula II-b, R¹ and R² combine to form a carbocylic or heterocyclic ring, such as a 5-member, 6-member, or 7-member carbocyclic or heterocyclic ring. In certain embodiments, the carbocyclic or heterocyclic ring is substituted with hydroxyl, alkyl (e.g., methyl), or alkenyl (e.g., vinyl). In certain embodiments, the compound is the compound is

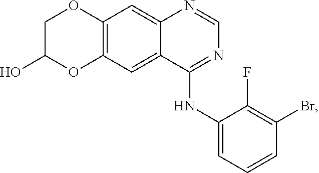 [SMILES: OC1COc2cc3ncnc(Nc4cccc(Br)c4F)c3cc2O1]

In certain embodiments, the carbocyclic or heterocyclic ring is substituted with alkyl (e.g., methyl) and the alkyl moieties are trans relative to each other. In certain embodiments, the compound is the compound is

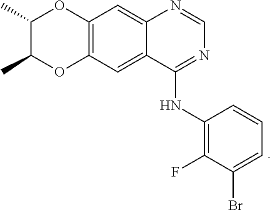 [SMILES: C[C@@H]1Oc2cc3ncnc(Nc4cccc(Br)c4F)c3cc2O[C@H]1C]

In certain embodiments, the carbocyclic or heterocyclic ring is substituted with alkyl (e.g., methyl) and the alkyl moieties are cis relative to each other. In certain embodiments, the compound is the compound is

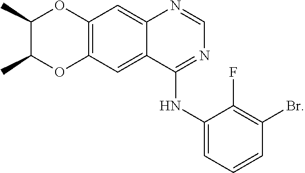 [SMILES: C[C@@H]1Oc2cc3c(Nc4cccc(Br)c4F)ncnc3cc2O[C@@H]1C]

In even more preferred embodiments of Formula I-a, Formula I-b, Formula II-a, or Formula II-b, the compound is a compound of Formula (III-a), (III-b), (III-c), (III-d), (III-e), or (III-f):

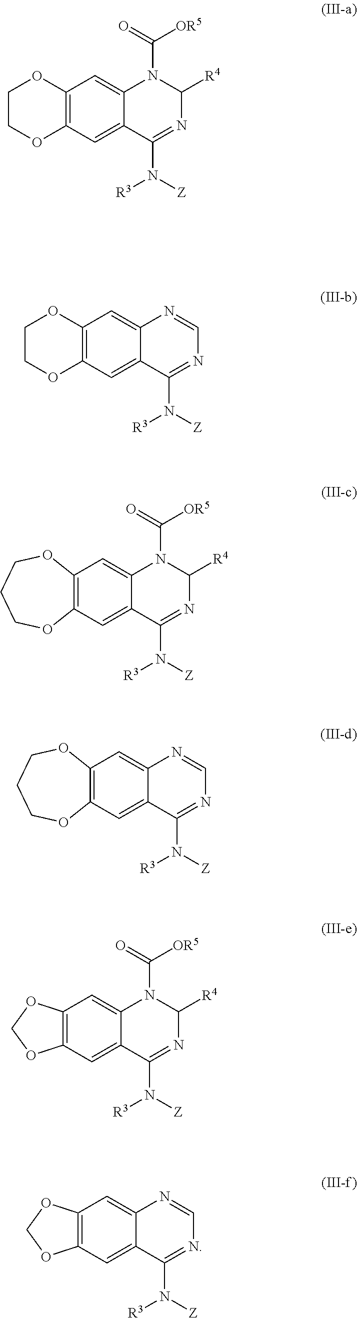

In certain embodiments of Formula I-a, Formula I-b, Formula II-a, Formula II-b, Formula III-a, Formula III-b, Formula III-c, Formula III-d, Formula III-e, or Formula III-f, R³ is hydrogen. In certain embodiments, R³ is acyl. In certain embodiments, R³ is alkylacyl. In certain embodiments, R³ is alkyloxyacyl. In certain embodiments, R³ is acyloxyalkyl. In certain embodiments, R³ is

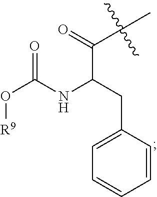

and R⁹ is alkyl.

In certain embodiments of Formula I-a, Formula I-b, Formula II-a, Formula II-b, Formula III-a, Formula III-b, Formula III-c, Formula III-d, Formula III-e, or Formula III-f, Z is aryl or heteroaryl optionally substituted with one or more R⁶; and each instance of R⁶ is independently selected from alkyl, alkoxy, OH, CN, NO₂, halo, alkenyl, alkynyl, aralkyloxy, cycloalkyl, heterocyclyl, aryl, or heteroaryl. In certain preferred embodiments, Z is phenyl substituted with 1, 2, 3, 4, or 5 R⁶. In certain embodiments, each R⁶ is independently selected from halo, alkyl, alkynyl, or arylalkoxy. In even more preferred embodiments, Z is 2-fluoro-3-chlorophenyl, 2-fluorophenyl, 2,3-difluorophenyl, 2,4-difluorophenyl, 2,5-difluorophenyl, 2,6-difluorophenyl, 2,4,6-trifluorophenyl, pentafluorophenyl, 2-fluoro-3-bromophenyl, 2-fluoro-3-ethynylphenyl, and 2-fluoro-3-(trifluoromethyl)phenyl. In other even more preferred embodiments, Z is 3-ethynylphenyl. In yet other even more preferred embodiments, Z is 3-chloro-4-((3-fluorobenzyl)oxy)benzene. In yet other even more preferred embodiments, Z is 3-chloro-2-(trifluoromethyl)phenyl. In yet other even more preferred embodiments, Z is 3-bromophenyl. In yet other even more preferred embodiments, Z is 2-fluoro,5-bromophenyl. In yet other even more preferred embodiments, Z is 2,6-difluoro,5-bromophenyl. In certain embodiments, Z is substituted with one R⁶ selected from

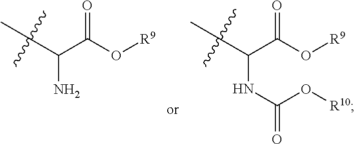

and R⁹ and R¹⁰ are independently selected from alkyl.

In certain embodiments of Formula I-a, Formula I-b, Formula II-a, Formula II-b, Formula III-a, Formula III-b, Formula III-c, Formula III-d, Formula III-e, or Formula III-f, the compound is a compound of Formula (IV-a):

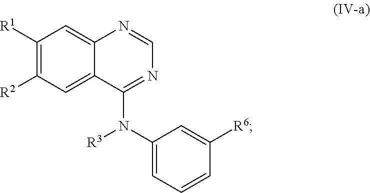

and each R⁶ is independently selected from fluoro, chloro, or bromo.

In certain embodiments of Formula I-a, Formula I-b, Formula II-a, Formula II-b, Formula III-a, Formula III-b, Formula III-c, Formula III-d, Formula III-e, or Formula III-f, the compound is a compound of Formula (IV-b):

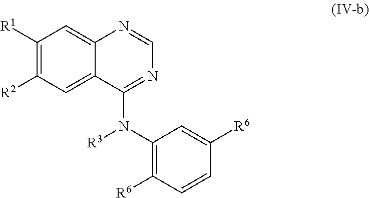

and each R⁶ is independently selected from fluoro, chloro, or bromo.

In certain embodiments of Formula I-a, Formula I-b, Formula II-a, Formula II-b, Formula III-a, Formula III-b, Formula III-c, Formula III-d, Formula III-e, or Formula III-f, the compound is a compound of Formula (IV-c):

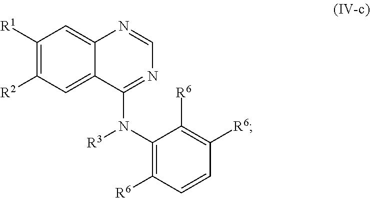

and each R⁶ is independently selected from fluoro, chloro, or bromo.

In certain embodiments of Formula I-a, Formula I-b, Formula II-a, Formula II-b, Formula In-a, Formula III-b, Formula III-c, Formula i-d, Formula III-e, Formula III-f, Formula IV-a, Formula IV-b, or Formula IV-c, the compound is a compound of Formula (V-a):

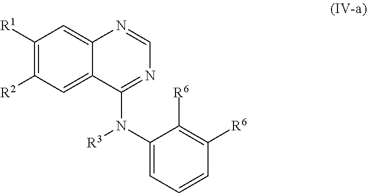

and each R⁶ is independently selected from fluoro, chloro, or bromo.

In certain embodiments of Formula I-a, Formula I-b, Formula II-a, Formula II-b, Formula III-a, Formula III-b, Formula III-c, Formula III-d, Formula III-e, Formula III-f, Formula IV-a, Formula IV-b, or Formula IV-c, the compound is a compound of Formula (V-b):

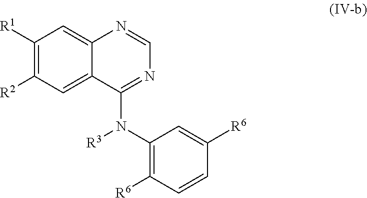

and each R⁶ is independently selected from fluoro, chloro, or bromo.

In certain embodiments of Formula I-a, Formula I-b, Formula II-a, Formula II-b, Formula III-a, Formula III-b, Formula III-c, Formula III-d, Formula III-e, Formula III-f, Formula IV-a, Formula IV-b, or Formula IV-c, the compound is a compound of Formula (V-b):

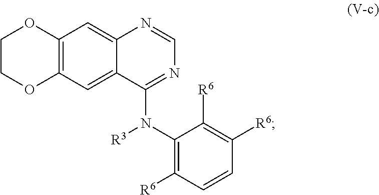

and each R⁶ is independently selected from fluoro, chloro, or bromo.

In certain embodiments, the compound of Formula I-a or I-b is selected from a compound in Table 2.

TABLE 2
Exemplary Compounds of the Present Invention

| Number | Structure |
|---|---|
| JGK001 | 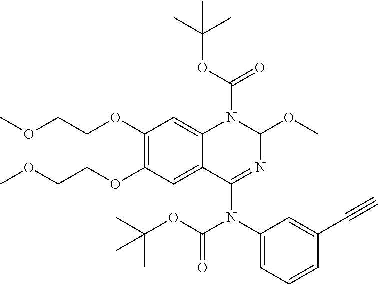 [SMILES: C#Cc1cccc(N(C(=O)OC(C)(C)C)C2=NC(OC)N(C(=O)OC(C)(C)C)c3cc(OCCOC)c(OCCOC)cc32)c1] |
| JGK002 | 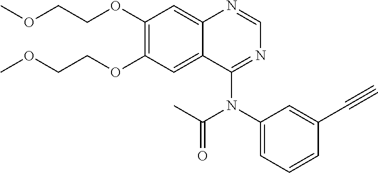 [SMILES: C#Cc1cccc(N(C(C)=O)c2ncnc3cc(OCCOC)c(OCCOC)cc23)c1] |
| JGK003 | 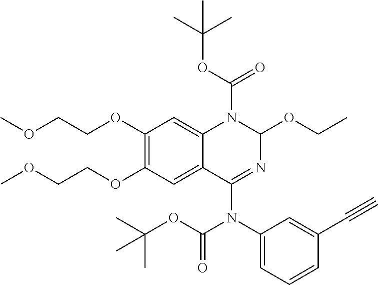 [SMILES: C#Cc1cccc(N(C(=O)OC(C)(C)C)C2=NC(OCC)N(C(=O)OC(C)(C)C)c3cc(OCCOC)c(OCCOC)cc32)c1] |
| JGK004 | 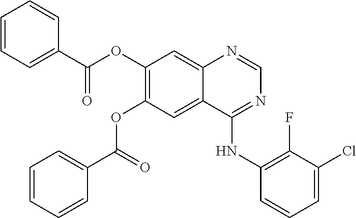 [SMILES: O=C(Oc1cc2ncnc(Nc3cccc(Cl)c3F)c2cc1OC(=O)c1ccccc1)c1ccccc1] |
| JGK005 | 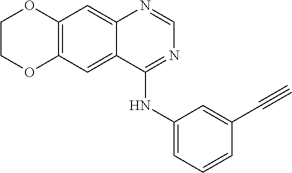 [SMILES: C#Cc1cccc(Nc2ncnc3cc4c(cc23)OCCO4)c1] |
| JGK006 | 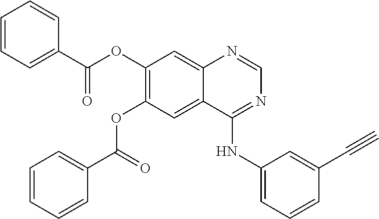 [SMILES: C#Cc1cccc(Nc2ncnc3cc(OC(=O)c4ccccc4)c(OC(=O)c4ccccc4)cc23)c1] |
| JGK007 | 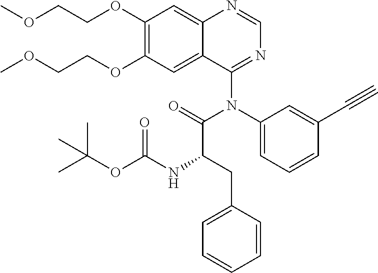 [SMILES: C#Cc1cccc(N(C(=O)[C@H](Cc2ccccc2)NC(=O)OC(C)(C)C)c2ncnc3cc(OCCOC)c(OCCOC)cc23)c1] |
| JGK008 | 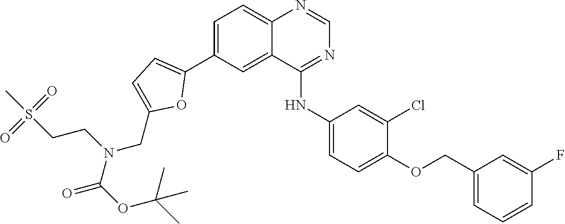 [SMILES: CC(C)(C)OC(=O)N(CCS(C)(=O)=O)Cc1ccc(-c2ccc3ncnc(Nc4ccc(OCc5cccc(F)c5)c(Cl)c4)c3c2)o1] |
| JGK009 | 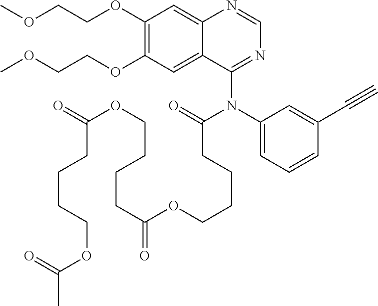 [SMILES: C#Cc1cccc(N(C(=O)CCCCOC(=O)CCCCOC(=O)CCCCOC(C)=O)c2ncnc3cc(OCCOC)c(OCCOC)cc23)c1] |
| JGK010 | 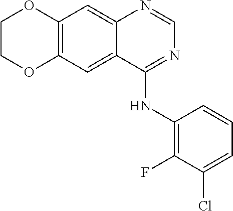 [SMILES: Fc1c(Cl)cccc1Nc1ncnc2cc3c(cc12)OCCO3] |
| JGK011 | 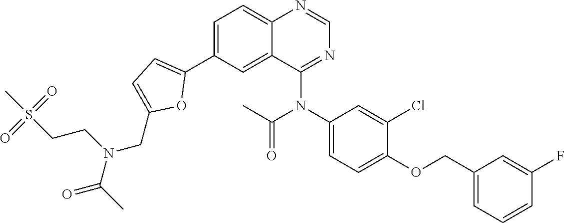 [SMILES: CC(=O)N(CCS(C)(=O)=O)Cc1ccc(-c2ccc3ncnc(N(C(C)=O)c4ccc(OCc5cccc(F)c5)c(Cl)c4)c3c2)o1] |
| JGK0012 | 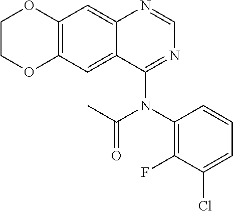 [SMILES: CC(=O)N(c1cccc(Cl)c1F)c1ncnc2cc3c(cc12)OCCO3] |
| JGK013 | 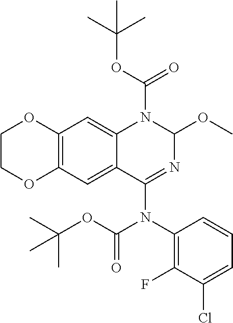 [SMILES: COC1N=C(N(C(=O)OC(C)(C)C)c2cccc(Cl)c2F)c2cc3c(cc2N1C(=O)OC(C)(C)C)OCCO3] |
| JGK014 | 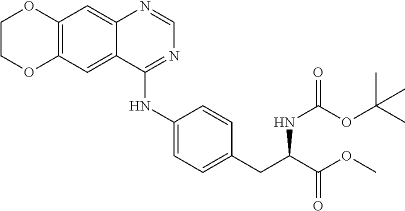 [SMILES: COC(=O)[C@@H](Cc1ccc(Nc2ncnc3cc4c(cc23)OCCO4)cc1)NC(=O)OC(C)(C)C] |
| JGK015 | 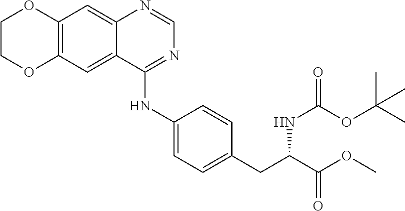 [SMILES: COC(=O)[C@H](Cc1ccc(Nc2ncnc3cc4c(cc23)OCCO4)cc1)NC(=O)OC(C)(C)C] |
| JGK016 | 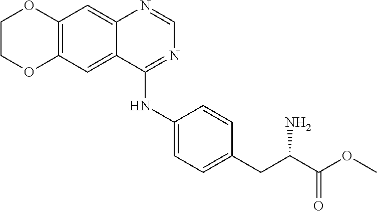 [SMILES: COC(=O)[C@@H](N)Cc1ccc(Nc2ncnc3cc4c(cc23)OCCO4)cc1] |
| JGK017 | 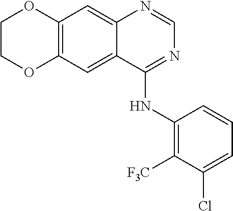 [SMILES: FC(F)(F)c1c(Cl)cccc1Nc1ncnc2cc3c(cc12)OCCO3] |
| JGK018 | 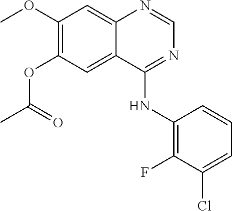 [SMILES: COc1cc2ncnc(Nc3cccc(Cl)c3F)c2cc1OC(C)=O] |
| JGK019 | 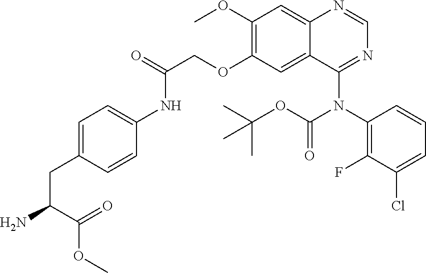 [SMILES: COC(=O)[C@@H](N)Cc1ccc(NC(=O)COc2cc3c(N(C(=O)OC(C)(C)C)c4cccc(Cl)c4F)ncnc3cc2OC)cc1] |
| JGK020 | 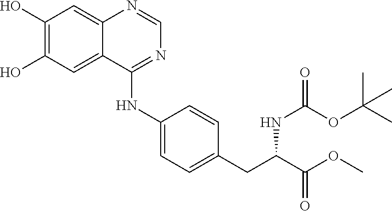 [SMILES: COC(=O)[C@H](Cc1ccc(Nc2ncnc3cc(O)c(O)cc23)cc1)NC(=O)OC(C)(C)C] |
| JGK021 | 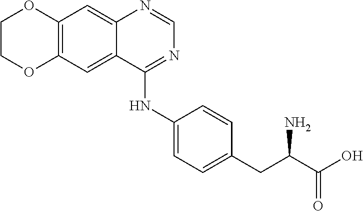 [SMILES: N[C@H](Cc1ccc(Nc2ncnc3cc4c(cc23)OCCO4)cc1)C(=O)O] |
| JGK022 | 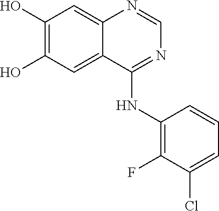 [SMILES: Oc1cc2ncnc(Nc3cccc(Cl)c3F)c2cc1O] |
| JGK023 | 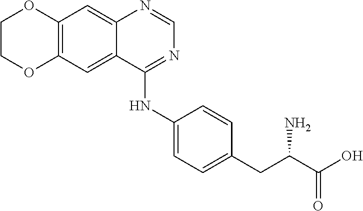 [SMILES: N[C@@H](Cc1ccc(Nc2ncnc3cc4c(cc23)OCCO4)cc1)C(=O)O] |
| JGK024 | 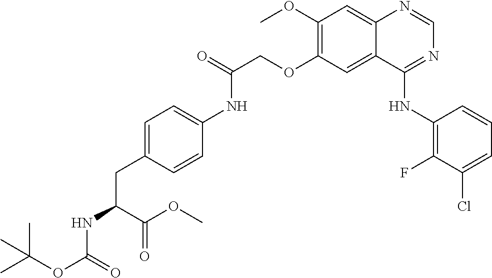 [SMILES: COC(=O)[C@H](Cc1ccc(NC(=O)COc2cc3c(Nc4cccc(Cl)c4F)ncnc3cc2OC)cc1)NC(=O)OC(C)(C)C] |
| JGK025 | 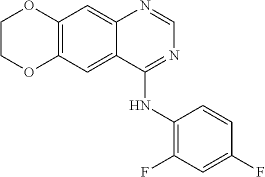 [SMILES: Fc1ccc(Nc2ncnc3cc4c(cc23)OCCO4)c(F)c1] |
| JGK026 | 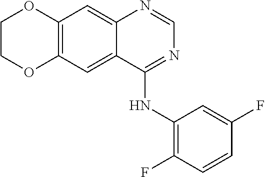 [SMILES: Fc1ccc(F)c(Nc2ncnc3cc4c(cc23)OCCO4)c1] |
| JGK027 | 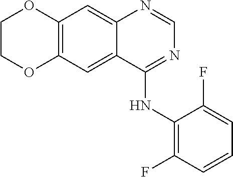 [SMILES: Fc1cccc(F)c1Nc1ncnc2cc3c(cc12)OCCO3] |
| JGK028 | 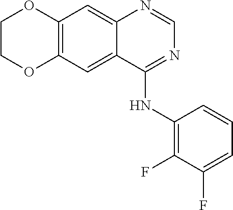 [SMILES: Fc1cccc(Nc2ncnc3cc4c(cc23)OCCO4)c1F] |
| JGK029 | 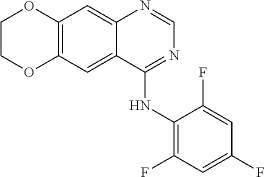 [SMILES: Fc1cc(F)c(Nc2ncnc3cc4c(cc23)OCCO4)c(F)c1] |
| JGK030 | 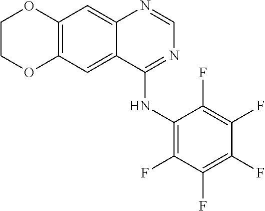 [SMILES: Fc1c(F)c(F)c(Nc2ncnc3cc4c(cc23)OCCO4)c(F)c1F] |
| JGK031 | 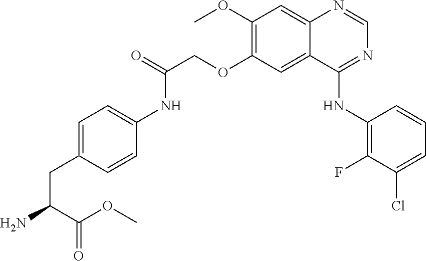 [SMILES: COC(=O)[C@@H](N)Cc1ccc(NC(=O)COc2cc3c(Nc4cccc(Cl)c4F)ncnc3cc2OC)cc1] |
| JGK032 | 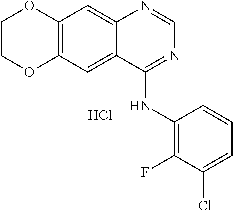 [SMILES: Fc1c(Cl)cccc1Nc1ncnc2cc3c(cc12)OCCO3 ; Cl] |
| JGK033 | 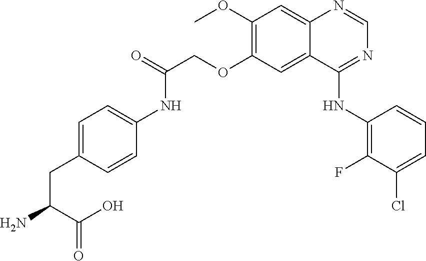 [SMILES: COc1cc2ncnc(Nc3cccc(Cl)c3F)c2cc1OCC(=O)Nc1ccc(C[C@H](N)C(=O)O)cc1] |
| JGK035 | 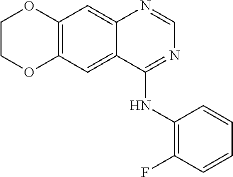 [SMILES: Fc1ccccc1Nc1ncnc2cc3c(cc12)OCCO3] |
| JGK036 | 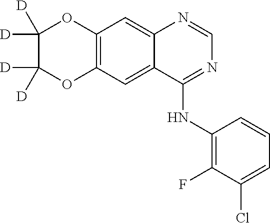 [SMILES: [2H]C1([2H])Oc2cc3ncnc(Nc4cccc(Cl)c4F)c3cc2OC1([2H])[2H]] |
| JGK037 | 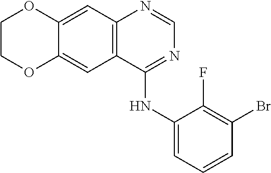 [SMILES: Fc1c(Br)cccc1Nc1ncnc2cc3c(cc12)OCCO3] |
| JGK038 | 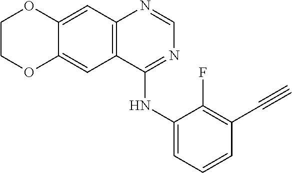 [SMILES: C#Cc1cccc(Nc2ncnc3cc4c(cc23)OCCO4)c1F] |
| JGK039 | 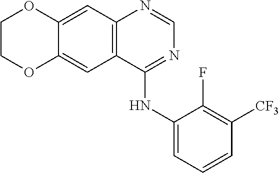 [SMILES: Fc1c(Nc2ncnc3cc4c(cc23)OCCO4)cccc1C(F)(F)F] |
| JGK040 | 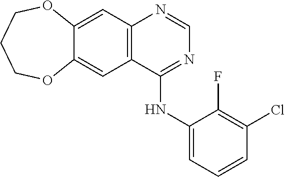 [SMILES: Fc1c(Cl)cccc1Nc1ncnc2cc3c(cc12)OCCCO3] |
| JGK041 | 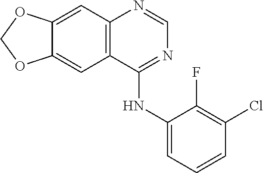 [SMILES: Fc1c(Cl)cccc1Nc1ncnc2cc3c(cc12)OCO3] |
| JGK042 | 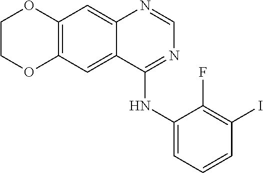 [SMILES: Fc1c(I)cccc1Nc1ncnc2cc3c(cc12)OCCO3] |
| JGK043 | 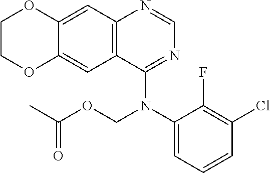 [SMILES: CC(=O)OCN(c1cccc(Cl)c1F)c1ncnc2cc3c(cc12)OCCO3] |
| JGK044 | 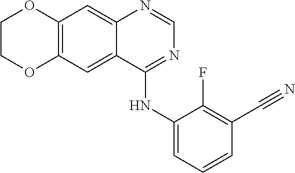 [SMILES: N#Cc1cccc(Nc2ncnc3cc4c(cc23)OCCO4)c1F] |
| JCK045 | 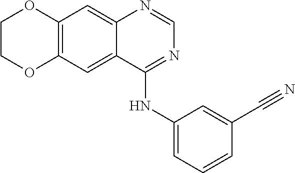 [SMILES: N#Cc1cccc(Nc2ncnc3cc4c(cc23)OCCO4)c1] |
| JGK047 | 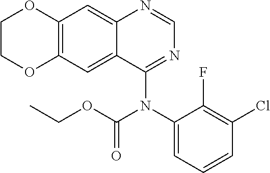 [SMILES: CCOC(=O)N(c1cccc(Cl)c1F)c1ncnc2cc3c(cc12)OCCO3] |
| JGK050 | 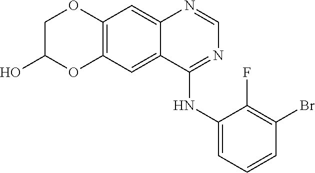 [SMILES: OC1COc2cc3ncnc(Nc4cccc(Br)c4F)c3cc2O1] |
| JGK051 | 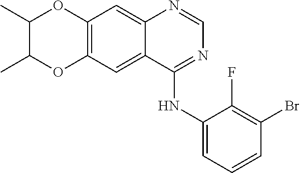 [SMILES: CC1Oc2cc3ncnc(Nc4cccc(Br)c4F)c3cc2OC1C] |
| JGK052 | 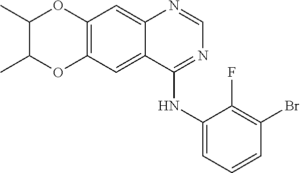 [SMILES: CC1Oc2cc3ncnc(Nc4cccc(Br)c4F)c3cc2OC1C] |
| JGK053 | 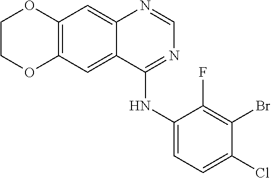 [SMILES: Fc1c(Nc2ncnc3cc4c(cc23)OCCO4)ccc(Cl)c1Br] |
| JGK054 | 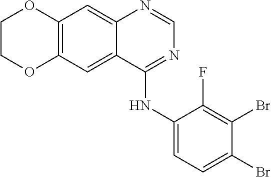 [SMILES: Fc1c(Nc2ncnc3cc4c(cc23)OCCO4)ccc(Br)c1Br] |
| JGK055 | 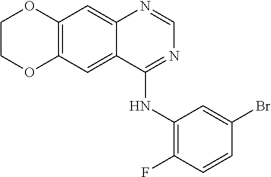 [SMILES: Fc1ccc(Br)cc1Nc1ncnc2cc3c(cc12)OCCO3] |
| JGK056 | 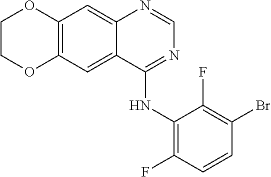 [SMILES: Fc1ccc(Br)c(F)c1Nc1ncnc2cc3c(cc12)OCCO3] |
| JGK057 | 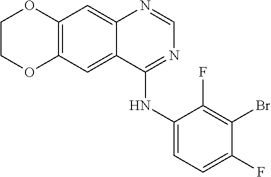 [SMILES: Fc1ccc(Nc2ncnc3cc4c(cc23)OCCO4)c(F)c1Br] |
| JGK058 | 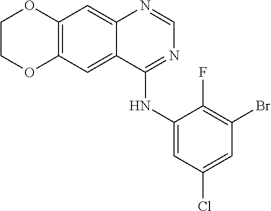 [SMILES: Fc1c(Br)cc(Cl)cc1Nc1ncnc2cc3c(cc12)OCCO3] |
| JGK059 | 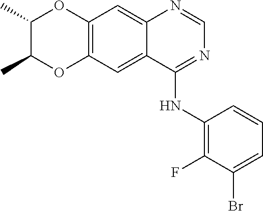 [SMILES: C[C@@H]1Oc2cc3ncnc(Nc4cccc(Br)c4F)c3cc2O[C@H]1C] |
| JGK060 | 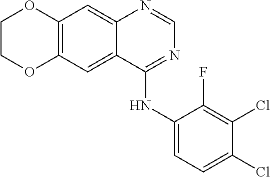 [SMILES: Fc1c(Nc2ncnc3cc4c(cc23)OCCO4)ccc(Cl)c1Cl] |
| JGK061 | 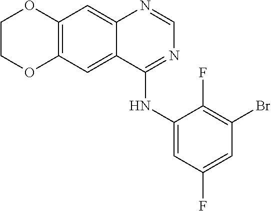 [SMILES: Fc1cc(Br)c(F)c(Nc2ncnc3cc4c(cc23)OCCO4)c1] |
| JGK062 | 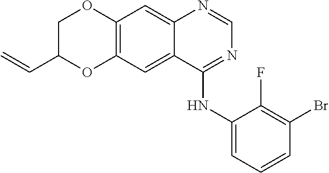 [SMILES: C=CC1COc2cc3ncnc(Nc4cccc(Br)c4F)c3cc2O1] |

### Methods of Treatment

In certain aspects, the present disclosure provides methods of inhibiting EGFR or ΔEGFR, comprising administering to a subject an effective amount of a compound of formula I-a or I-b.

In certain aspects, the present disclosure provides methods of treating cancer comprising of administering to a subject in need of a treatment for cancer an effective amount of a compound of formula I-a or I-b. In some embodiments, the cancer is glioblastoma multiforme.

In certain aspects, the present disclosure provides methods of treating glioblastoma in a subject, the method comprising administering to the subject an amount of a glucose uptake inhibitor and a cytoplasmic p53 stabilizer.

In certain aspects, the present disclosure provides methods of reducing glioblastoma proliferation in a subject, the method comprising administering to the subject an amount of an EGFR inhibitor and a MDM2 inhibitor.

In certain aspects, the present disclosure provides methods of treating cancer or reducing cancer cell proliferation in a subject, comprising administering to the subject an amount of a glucose metabolism inhibitor and a p53 stabilizer.

In certain aspects, the present disclosure provides methods of treating malignant glioma or glioblastoma in a subject, the method comprising administering to the subject an amount of the glucose metabolism inhibitor and a cytoplasmic p53 stabilizer.

In certain aspects, the present disclosure provides methods of treating cancer or reducing cancer cell proliferation in a subject, comprising administering to the subject an amount of a glucose metabolism inhibitor and a p53 stabilizer.

In certain aspects, the present disclosure provides methods of treating cancer comprising of administering to a subject in need of a treatment for cancer an effective amount of a glucose metabolism inhibitor and a cytoplasmic p53 stabilizer. In some embodiments, the cancer is glioblastoma multiforme. In some embodiments, the subject's tumor has been determined to be sensitive to the glucose metabolism inhibitor.

In some embodiments, the present disclosure provides methods to suppress GBM growth or proliferation by administering to a subject that has been previously determined to be qualified for such a treatment, a glucose metabolism inhibitor and a cytoplasmic p53 stabilizer. In some embodiments, the inhibition of glucose metabolism and the stabilization of cytoplasmic p53 can be concomitant or sequential.

In some embodiments, the present disclosure provides methods of treating GBM, methods of reducing or inhibiting GBM in a subject. In some embodiments, the present disclosure provides methods of inhibiting growth of a GBM cell. In some embodiments, the present disclosure provides methods for treating GBM patient with a glucose metabolism inhibitor and a cytoplasmic p53 stabilizer. In some embodiments, the present disclosure provides methods for improving the prognosis of GMB patients. In some embodiments, the present disclosure provides methods for reducing the risk of GBM. In some embodiments, the present disclosure provides methods of classifying patients with GBM. In some embodiments, the present disclosure provides methods of assessing the response of a patient to treatment. In some embodiments, the present disclosure provides methods of priming a GBM tumor cell to apoptosis. In some embodiments, the present disclosure provides methods of conditioning a GBM patient to treatment. In some embodiments, the present disclosure provides methods of reducing risk of ineffective therapy. In some embodiments, the present disclosure provides methods of ameliorating the symptoms of GBM. In some embodiments, the present disclosure provides methods for reducing the chances of a tumor survival. In some embodiments, the present disclosure provides methods for increasing the vulnerability of tumor cells to therapy. The steps and embodiments discussed in this disclosure are contemplated as part of any of these methods. Moreover, compositions for use in any of these methods are also contemplated.

In certain aspects, the present disclosure provides methods of treating malignant glioma or GBM in a subject, the method comprising administering to the subject after the subject has been determined to be susceptible to a glucose metabolism inhibitor an amount of the glucose metabolism inhibitor and a cytoplasmic p53 stabilizer.

In some embodiments, the subject has been determined to be susceptible to a glucose metabolism inhibitor by a method comprising obtaining a tumor biopsy from the subject, measuring the level of glucose uptake by the tumor cells in the presence of any glucose metabolism inhibitor, comparing the level of glucose uptake by the tumor cells obtained to the level of glucose uptake by a control; and determining that the subject is susceptible to the glucose metabolism inhibitor if the level of glucose uptake by the tumor cells is attenuated compared to the control. In some embodiments, the glucose uptake is measured by the uptake of radio-labelled glucose 2-deoxy-2-[fluorine-18]fluoro-D-glucose (¹⁸F-FDG). In further embodiments, detecting the ¹⁸F-FDG is by positron emission tomography (PET). In some embodiments, the biopsy is taken from a GBM tumor.

In some embodiments, the subject has been determined to be susceptible to the glucose metabolism inhibitor by a method comprising obtaining a first blood sample from the subject, placing the subject on a ketogenic diet for a period of time, obtaining a second blood sample from the subject after being placed on a ketogenic diet, measuring glucose level in the first and in the second blood sample; comparing the glucose level in the second blood sample with the glucose level in the first blood sample, and determining that the subject is susceptible if the glucose level in the second blood sample is reduced as compared to glucose levels in the first blood sample. In some embodiments, the reduction in the glucose level is between the second blood sample and the control blood sample is about or greater than 0.15 mM, is about or greater than 0.20 mM, is in the range of 0.15 mM-2.0 mM or is in the range of 0.25 mM-1.0 mM. In some embodiments, the reduction in the glucose levels is about, at least about, or at most about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0 mM (or any range derivable therein).

In certain aspects, the present disclosure provides methods of classifying a subject diagnosed with glioma or GBM, the method comprising obtaining a biological sample from the subject, treating the biological sample with glucose metabolism inhibitor(s) and determining if glucose metabolism is attenuated by the glucose metabolism inhibitor.

Determining attenuation of glucose metabolism comprises determining change in glucose level, and/or change in rate of glycolysis, and/or change in glucose uptake, and/or change in extracellular acidification rate (ECAR), and/or measuring the activity of hexokinase, or phosphofructokinase, or pyruvate kinase before and after administration of the glucose metabolism inhibitor. In some embodiments, determining changes in glycolysis comprises direct measurement of pyruvate and/or lactate. In certain embodiments, the biological sample comprises cancer cells from a GBM tumor. In other embodiments, the method further comprises comparing the level of glucose attenuation to a control. In some embodiments, the method further comprises classifying the subject as a metabolic responder if glucose metabolism is attenuated by the glucose metabolism inhibitor in the biological sample. In further embodiments, the method further comprises treating the subject classified as a metabolic responder with a composition comprising a glucose metabolism inhibitor and a cytoplasmic p53 stabilizer.

In certain aspects, the present disclosure provides methods of assessing the sensitivity of cancer cells or tumor to treatment with a glucose metabolism inhibitor and a cytoplasmic p53 stabilizer, the method comprising measuring or detecting the level of glucose uptake by the cancer cells and comparing the level of glucose uptake with a control. The glucose can be radio-labelled such as for example, 2-deoxy-2-[fluorine-18]fluoro-D-glucose (¹⁸F-FDG). In some embodiments, the measuring and detecting of the radio labeled glucose uptake is done by positron emission tomography (PET).

In certain aspects the present disclosure provides methods of treating glioblastoma in a subject, the method comprising administering to the subject a therapeutically effective amount of a glucose uptake inhibitor and a cytoplasmic p53 stabilizer after determining that the subject is susceptible to reduced glucose metabolism by an EGFR inhibitor.

In certain aspects the present disclosure provides methods of reducing glioblastoma proliferation in a subject, the method comprising administering to the subject an effective amount of an EGFR inhibitor and a MDM2 inhibitor after determining that the subject is susceptible to EGFR inhibitors.

In some embodiments the glucose metabolism inhibitor and the cytoplasmic p53 stabilizer are administered in the same composition. In other embodiments, the glucose metabolism inhibitor and the cytoplasmic p53 are administered in separate compositions. For example, in some embodiments the glucose metabolism inhibitor and the cytoplasmic p53 stabilizer are administered within 24, 23, 22, 21, 20, 19, 18, 17, 16, 15, 14, 13, 12, 11, 10, 9, 8, 7, 6, 5, 4, 3, 2, 1 hours or within 30 min of each other or any fraction of hours or minutes in between. Yet in further embodiments, the glucose metabolism inhibitor and the p53 stabilizer are administered at the same time to the subject.

In certain embodiments of the current methods, the use of a control is contemplated such as comparing the level of glucose (or change in glucose or glucose attenuation) in a sample from a subject with a control sample. The control can comprise a non-cancerous sample, a cancerous sample with a different phenotype, a cancer sample with a wildtype EGFR expression level or any other control sample that is either taken from the patient's non-cancer cells or is not taken from the patient. In certain embodiments, the control is from a sample taken from the patient before the sample is subjected to glucose metabolism inhibitors.

In certain aspects the present disclosure provides methods for treating cancer or reducing cancer cell proliferation in a subject that has been determined to have cancer that is responsive to glucose metabolism inhibitors, comprising administering to a cancer patient an effective amount of a glucose metabolism inhibitor and a cytoplasmic p53 stabilizer. In some embodiments, the cancer is a Central Nervous System (CNS) cancer for example CNS metastases. In some embodiments, the cancer is a non-CNS cancer. In some embodiments, the cancer is glioblastoma multiforme, glioma, low-grade astrocytoma, mixed oligoastrocytoma, pilocytic astrocytoma, pleomorphic xanthoastrocytoma, subependymal giant cell astrocytoma, anaplastic astrocytoma, lung cancer or others.

In embodiments of the methods herein, the subject has been diagnosed with glioblastoma multiforme. In some embodiments, the subject has been previously treated for glioblastoma with a prior treatment. Yet is some embodiments, the subject has been determined to be resistant to the prior treatment.

In certain embodiments of the current methods, the method further comprises administration of an additional therapy. In some embodiments, the additional therapy is radiation therapy, chemotherapy, targeted therapy, immunotherapy, surgery. In some embodiments, the additional therapy comprises one or more therapies described herein.

### Malignanl Gliomas and Glioblastomas

### Types and Stages of Gliomas

Primary malignant brain tumors are tumors that start in the brain or spine are known collectively as gliomas. Gliomas are not a specific type of cancer but are a term used to describe tumors that originate in glial cells. Examples of primary malignant brain tumors include astrocytomas, pilocytic astrocytomas, pleomorphic xanthoastrocytomas, diffuse astrocytomas, anaplastic astrocytomas, GBMs, gangliogliomas, oligodendrogliomas, ependymomas. According to the WHO classification of brain tumors, astrocytomas have been categorized into four grades, determined by the underlying pathology. The characteristics that are used to classify gliomas include mitoses, cellular or nuclear atypia, and vascular proliferation and necrosis with pseudopalisading features. Malignant (or high-grade) gliomas include anaplastic glioma (WHO grade III) as well as glioblastoma multiforme (GBM; WHO grade IV). These are the most aggressive brain tumors with the worst prognosis.

GBMs is the most common, complex, treatment resistant, and deadliest type of brain cancer, accounting for 45% of all brain cancers, with nearly 11,000 men, women, and children diagnosed each year. GBM (also known as grade-4 astrocytoma and glioblastoma multiforme) are the most common types of malignant (cancerous) primary brain tumors. They are extremely aggressive for a number of reasons. First, glioblastoma cells multiply quickly, as they secrete substances that stimulate a rich blood supply. They also have an ability to invade and infiltrate long distances into the normal brain by sending microscopic tendrils of tumor alongside normal cells. Two types of glioblastomas are known. Primary GBM are the most common form; they grow quickly and often cause symptoms early. Secondary glioblastomas are less common, accounting for about 10 percent of all GBMs. They progress from low-grade diffuse astrocytoma or anaplastic astrocytoma, and are more often found in younger patients. Secondary GBM are preferentially located in the frontal lobe and carry a better prognosis.

GBM is usually treated by combined multi-modal treatment plan including surgical removal of the tumor, radiation and chemotherapy. First, as much tumor as possible is removed during surgery. The tumor's location in the brain often determines how much of it can be safely removed. After surgery, radiation and chemotherapy slow the growth of remaining tumor cells. The oral chemotherapy drug, temozolomide, is most often used for six weeks, and then monthly thereafter. Another drug, bevacizumab (known as Avastin®), is also used during treatment. This drug attacks the tumor's ability to recruit blood supply, often slowing or even stopping tumor growth.

Novel investigational treatments are also used and these may involve adding treatments to the standard therapy or replacing one part of the standard therapy with a different treatment that may work better. Some of these treatments include immunotherapy such as vaccine immunotherapies, or low-dose pulses of electricity to the area of the brain where the tumor exists and nano therapies involving spherical nucleic acids (SNAs) such as NU-0129. In some embodiments, the methods of the current disclosure are used in combination with one or more of the aforementioned therapies.

Embodiments of the methods and compositions discussed herein are also contemplated to be applicable to other types of cancers, including but not limited to lung cancer, non-CNS cancers, CNS cancers, and CNS metastases such as brain metastases, leptomeningeal metastases, choroidal metastases, spinal cord metastases, and others.

### Glucose Metabolism Inhibitors

In certain aspects, the methods and compositions of the current disclosure comprise glucose metabolism inhibitor(s). The inhibitor can be a glucose uptake inhibitor, a glucose transporter inhibitor, a glycolysis inhibitor, a hexokinase inhibitor, an epidermal growth factor receptor (EGFR) inhibitor, any inducer of intracellular glucose deprivation or any combination thereof.

### EGFR Inhibitors

In some embodiments, the glucose metabolism inhibitor is an EGFR inhibitor. The EGFR signaling pathway is activated in several cancers, including gliomas and GBMs. Activation may occur by multiple mechanisms, including activating mutations in the EGFR protein, or EGFR overexpression, typically due to increased EGFR gene copy number. EGFR gene amplification and overexpression are a particularly striking feature of glioblastoma (GBM), observed in approximately 40% of tumors. EGFR inhibitors can be small molecule tyrosine kinase inhibitors or antibodies. For example, monoclonal antibodies.

### Small Molecule Inhibitors

In some embodiments, the EGFR inhibitor is erlotinib, a small molecule targeted therapy that binds in a reversible fashion to the ATP binding site of the EGFR receptor, thereby blocking the receptor's ability to form phosphotyrosine residues on the EGFR homodimers and preventing the transduction of signal cascades to activate other cellular biochemical processes. The current disclosure also contemplates that other EGFR inhibitors can also be used with the methods and compositions described herein, such as for example, gefitinib, lapatinib, cetuximab, panitumumab, vandetanib, necitumumab, or osimertinib. In some embodiments, the compositions of the disclosure excludes one or more EGFR inhibitor. In certain preferred embodiments, the EGFR inhibitor is a compound of formula I-a or I-b.

### Antibodies

In certain embodiments, the methods and compositions comprise EGFR inhibitors that are antibodies. EGFR antibodies in clinical use include but are not limited to, cetuximab (ERBITUX™) and panitumumab (VECTIBIX™) that bind to the extracellular domain of the EGFR. The extracellular receptor domain includes the ligand binding site and these antibodies are believed to block ligand binding; thereby, disrupting EGFR signaling. Many studies have focused on the production of antibodies (or other binding molecules) specific for the EGFR extracellular domain (see, e.g., U.S. Pat. Nos. 5,459,061, 5,558,864, 5,891,996, 6,217,866, 6,235,883, 6,699,473, and 7,060,808; European Pat. Nos. EP0359282 and EP0667165 all of which are hereby incorporated by reference in their entirety.

In some embodiments, the antibody is a monoclonal antibody or a polyclonal antibody. In some embodiments, the antibody is a chimeric antibody, an affinity matured antibody, a humanized antibody, or a human antibody. In some embodiments, the antibody is an antibody fragment. In some embodiments, the antibody is a Fab, Fab′, Fab′-SH, F(ab′)2, or scFv. In one embodiment, the antibody is a chimeric antibody, for example, an antibody comprising antigen binding sequences from a non-human donor grafted to a heterologous non-human, human or humanized sequence (e.g., framework and/or constant domain sequences). In one embodiment, the non-human donor is a mouse. In one embodiment, an antigen binding sequence is synthetic, e.g, obtained by mutagenesis (e.g., phage display screening, etc.). In one embodiment, a chimeric antibody has murine V regions and human C region. In one embodiment, the murine light chain V region is fused to a human kappa light chain or a human IgG1 C region.

Examples of antibody fragments include, without limitation: (i) the Fab fragment, consisting of VL, VH, CL and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multi specific fragments constructed by gene fusion (U.S. Patent Pub. 2005/0214860) Fv, scFv or diabody molecules may be stabilized by the incorporation of disulfide bridges linking the VH and VL domains. Minibodies comprising a scFv joined to a CH3 domain may also be made (Hu et al, 1996).

A monoclonal antibody is a single species of antibody wherein every antibody molecule recognizes the same epitope because all antibody producing cells are derived from a single B-lymphocyte cell line. Hybridoma technology involves the fusion of a single B lymphocyte from a mouse previously immunized with an antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced. However, in therapeutic applications a goal of hybridoma technology is to reduce the immune reaction in humans that may result from administration of monoclonal antibodies generated by the non-human (e.g., mouse) hybridoma cell line.

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

By known means as described herein, polyclonal or monoclonal antibodies, binding fragments and binding domains and CDRs (including engineered forms of any of the foregoing), may be created that are specific to a protein described herein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. Particularly, the antibodies may be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by this reference. These techniques are further described in: Marks (1992); Stemmer (1994); Gram et al. (1992); Barbas et al. (1994); and Schier et al. (1996).

Methods for producing polyclonal antibodies in various animal species, as well as for producing monoclonal antibodies of various types, including humanized, chimeric, and fully human, are well known in the art. Methods for producing these antibodies are also well known. For example, the following U.S. patents and patent publications provide enabling descriptions of such methods and are herein incorporated by reference; U.S. Patent publication Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855, 4,703,003; 4,742,159; 4,767,720; 4,816,567; 4,867,973; 4,938,948, 4,946,778; 5,021,236; 5,164,296, 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent publications, and other publications cited herein and therein are hereby incorporated by reference in the present application.

It is fully expected that antibodies against EGFR will have the ability to neutralize or counteract the effects of the protein regardless of the animal species, monoclonal cell line or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into binding fragments having the binding domain or CDR. Removal of the Fc portion reduces the likelihood that the antigen binding fragment will elicit an undesirable immunological response and, thus, antibodies without Fc may be particularly useful for prophylactic or therapeutic treatments. As described above, antibodies may also be constructed so as to be chimeric, partially or fully human, so as to reduce or eliminate the adverse immunological consequences resulting from administering to an animal an antibody that has been produced in, or has sequences from, other species. In some embodiments, the inhibitor is a peptide, polypeptide, or protein inhibitor. In some embodiments, the inhibitor is an antagonistic antibody.

### PI3K Inhibitors

In certain embodiments, the methods and compositions comprise a glucose metabolism inhibitor thats is a phosphatidylinositol 3-kinase PI3K inhibitor. Signaling through the mitogen-activated protein (MAP) kinase and phosphatidylinositol 3-kinases (PI3Ks)/AKT pathway is triggered by extracellular stimulation and regulates a variety of biological processes, such as proliferation, differentiation and cell death. Phosphatidylinositol 3-kinases (PI3Ks) are critical coordinators of intracellular signaling in response to such extracellular stimulation. They comprise a family of lipid kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). The current methods and compositions also contemplate the use of isoform-selective inhibitors to the different PI3K isoforms, each of which play divergent roles in cellular signaling and cancer. Inhibitors targeting individual isoforms may be able to achieve greater therapeutic efficacy.

Exemplary PI3K inhibitors include, pictilisib, dactolisib, wortmannin, LY294002, Idelalisib (CAL-101, GS-1101), duvelisib, buparlisib, IPI-549, SP2523, GDC-0326, TGR-1202, VPS34 inhibitor 1, GSK2269557 (Nemiralisib), GDC-0084, SAR405, AZD8835, LY3023414, PI-103, TGX-221, NU7441 (KU-57788), IC-87114, wortmannin, XL147 analogue, ZSTK474, Alpelisib (BYL719), PIK-75 HCl, A66, AS-605240, 3-Methyladenine (3-MA), PIK-93, PIK-90, AZD64822, PF-04691502, Apitolisib (GDC-0980, RG7422), GSK1059615, Duvelisib (IPI-145, INK1197), Gedatolisib (PF-05212384, PKI-587), TG100-115, AS-252424, BGT226 (NVP-BGT226), CUDC-907, AS-604850, PIK-294, GSK2636771, Copanlisib (BAY 80-6946), YM201636, CH5132799, CAY10505, PIK-293, PKI-402, TG100713, VS-5584 (SB2343), Taselisib (GDC 0032), CZC24832, AMG319, GSK2292767, HS-173, Quercetin, Voxtalisib (SAR245409, XL765), PIK-93, Omipalisib (GSK2126458, GSK458), PIK-90, GNE-317, Pilaralisib (XL147), PF-4989216, AZD8186, 740 Y-P, Vps34-IN1, PIK-III, PI-3065 or any other PI3K inhibitor or analogs thereof.

### Glucose Uptake Inhibitors/Hexokinase Inhibitors

In some embodiments, the glucose metabolism inhibitor is a glucose uptake or glycolysis inhibitor. In some embodiments, the inhibitor is a hexokinase inhibitor. Exemplary hexokinase inhibitors include, but are not limited to, 2-deoxyglucose (2DG), brompyruvic acid, lonidamine mitochondrial hexokinase inhibitor and PKM2 modulators.

### Glucose Transporter Inhibitors

In some embodiments, the methods of treating GBMs or cancer comprise administering to a subject an effective amount of a glucose transporter inhibitor and a cytoplasmic p53 stabilizer. Exemplary inhibitors of the glucose transporter family of molecules include several members of the flavonoid family. For example, forskolin, phloretin (a flavonoid-like compound) and cytochalasin B are known to inhibit GLUT1. Quercetin, a flavonol, has been shown to inhibit GLUT2-mediated glucose transport (Song et al., J. Biol. Chem. 277: 15252-15260, 2002). Oestradiol and the isoflavone phytoestrogen Genistein, are also inhibitors of GLUT1-mediated glucose transport (Afzal et al., Biochem J. 365: 707-719, 2002). The glucose transporter inhibitors forskolin, dipyridamole and isobutylmethylxanthine (IBMX) bind to both GLUT1 and GLUT4 (Hellwig & Joost, Mol. Pharmacol. 40:383-389, 1991). Cytochalasin B also binds GLUT4 (Wandel et al., Biochim. Biophys. Acta 1284:56-62, 1996). Accordingly, in some embodiments of the current methods and compositions, the glucose metabolism inhibitor is forskolin, quercetin, genistein, oestradiol, dipyridamole, isobutylmethylxanthine or cytochalasin B.

In addition to these known inhibitors, a person skilled in the art will appreciate that there are a number of assays known to identify inhibitors of glucose transporters. For example, the effect of inhibitors on a glucose transporter can be assessed by expressing the GLUT of interest, preferably glucose transporter 8, in cells such as Xenopus laevis oocytes or CHO, measuring glucose uptake in the presence or absence of the inhibitor, and determining whether the inhibitor is competitive or non-competitive. Once the sequence of a given GLUT isoform is known, its sensitivity to a large number of molecules can be readily tested to identify drug candidates.

### Cytoplasmic p53 Stabilizers

The inventors have demonstrated that the pharmacological p53 stabilization, such as with a CNS-penetrant small molecule, for example, was synergistically lethal with the inhibition of EGFR-driven glucose uptake in patient-derived, primary GBM models. The inventors have demonstrated, for the first time, that the non-transcriptional functions of p53 can have a critical role in stimulating intrinsic apoptosis in metabolic responders. Accordingly, the methods of treatment described herein comprise the administration of cytoplasmic p53 stabilizer(s) in combination with glucose metabolism inhibitors. Cytoplasmic p53 stabilizer(s) and glucose metabolism inhibitors can be administered in the same or in different compositions, cocomitantly or sequentially. It is contemplated that in some embodiments a single p53 stabilizer is used and in other embodiments more than on p53 stabilizer is used. For example, the combination of nutlin with ABT 737 (which binds BCL-2 and BCL-XL) is reported to synergistically target the balance of pro-apoptotic and anti-apotptoic proteins at the mitochondrial level, thereby promoting cell death. (Hoe et al. 2014. Nature Reviews. Vol. 13. pp. 217) As intended herein, a cytoplasmic p53 stabilizer is any small molecule, antibody, peptide, protein, nucleic acid or derivatives thereof that can pharmacologically stabilize or activate p53 directly or indirectly. The stabilization of cytoplasmic p53 leads to priming cells, such as cancer cells, for apoptosis.

### MDM2 Antagonists

Protein levels of p53 within cells are tightly controlled and kept low by its negative regulator, the E3 ubiquitin protein ligase MDM2. In embodiments of the methods or composition of the current disclosure, the cytoplasmic p53 stabilizer is an MDM2 antagonist/inhibitor. In some embodiments, the MDM2 antagonist is a nutlin. In further embodiments, the nutlin is nutlin-3 or idasanutlin. In other embodiments, the MDM2 antagonist is RO5045337 (also known as RG7112), RO5503781, RO6839921, SAR405838 (also known as MI-773), DS-3032, DS-3032b, or AMG-232 or any other MDM2 inhibitor.

Other compounds within the scope of the current methods known to bind MDM-2 include Ro-2443, MI-219, MI-713, MI-888, DS-3032b, benzodiazepinediones (for example, TDP521252), sulphonamides (for example, NSC279287), chromenotriazolopyrimidine, morpholinone and piperidinones (AM-8553), terphenyls, chalcones, pyrazoles, imidazoles, imidazole-indoles, isoindolinone, pyrrolidinone (for example, PXN822), priaxon, piperidines, naturally derived prenylated xanthones, SAH-8 (stapled peptides) sMTide-02, sMTide-02a (stapled peptides), ATSP-7041 (stapled peptide), spiroligomer (α-helix mimic). Other compounds that are known to cause protein folding of MDM2 include PRIMA-IMET (also known as APR-246) Aprea 102-105, PK083, PK5174, PK5196, PK7088, benzothiazoles, stictic acid and NSC319726.

### BCL-2 Inhibitors

In further embodiments of the current methods or compositions, the cytoplasmic p53 stabilizer is a BCL-2 inhibitor. In some embodiments, the BCL-2 inhibitor is, for example, antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, APG-1252 or any other BCL-2 inhibitor.

### Bcl-xL Inhibitors

In yet further embodiments of the current methods or compositions, the cytoplasmic p53 stabilizer is a Bcl-xL inhibitor. In some embodiments, the Bcl-xL inhibitor is, for example, WEHI 539, ABT-263, ABT-199, ABT-737, sabutoclax, AT101, TW-37, APG-1252, gambogic acid or any other Bcl-xL inhibitor.

### Additional Methods

In certain aspects, the present disclosure provides methods of inhibiting EGFR or ΔEGFR, comprising of administering to a subject an effective amount of a compound of formula I-a or I-b.

In certain aspects, the present disclosure provides methods of treating cancer comprising administering to a subject in need of a treatment for cancer an effective amount of a compound of formula I-a or I-b. In some embodiments, the cancer is glioblastoma multiforme.

In certain aspects, the present disclosure provides methods of treating cancer comprising of administering to a subject in need of a treatment for cancer an effective amount of a glucose metabolism inhibitor and a cytoplasmic p53 stabilizer. In some embodiments, the cancer is glioblastoma multiforme. In some embodiments, the subject's tumor has been determined to be sensitive to the glucose metabolism inhibitor.

In some embodiments, the present disclosure provides methods to suppress GBM growth or proliferation by administering to a subject that has been previously determined to be qualified for such a treatment, a glucose metabolism inhibitor and a cytoplasmic p53 stabilizer. In some embodiments, the inhibition of glucose metabolism and the stabilization of cytoplasmic p53 can be concomitant or sequential.

In some embodiments, the present disclosure provides methods of treating GBM, methods of reducing or inhibiting GBM in a subject. In some embodiments, the present disclosure provides methods of inhibiting growth of a GBM cell. In some embodiments, the present disclosure provides methods for treating GBM patient with a glucose metabolism inhibitor and a cytoplasmic p53 stabilizer. In some embodiments, the present disclosure provides methods for improving the prognosis of GMB patients. In some embodiments, the present disclosure provides methods for reducing the risk of GBM. In some embodiments, the present disclosure provides methods of classifying patients with GBM. In some embodiments, the present disclosure provides methods of assessing the response of a patient to treatment. In some embodiments, the present disclosure provides methods of priming a GBM tumor cell to apoptosis. In some embodiments, the present disclosure provides methods of conditioning a GBM patient to treatment. In some embodiments, the present disclosure provides methods of reducing risk of ineffective therapy. In some embodiments, the present disclosure provides methods of ameliorating the symptoms of GBM. In some embodiments, the present disclosure provides methods for reducing the chances of a tumor survival. In some embodiments, the present disclosure provides methods for increasing the vulnerability of tumor cells to therapy. The steps and embodiments discussed in this disclosure are contemplated as part of any of these methods. Moreover, compositions for use in any of these methods are also contemplated.

In certain aspects, the present disclosure provides methods of treating malignant glioma or GBM in a subject, the method comprising administering to the subject after the subject has been determined to be susceptible to a glucose metabolism inhibitor an amount of the glucose metabolism inhibitor and a cytoplasmic p53 stabilizer.

In some embodiments, the subject has been determined to be susceptible to a glucose metabolism inhibitor by a method comprising obtaining a tumor biopsy from the subject, measuring the level of glucose uptake by the tumor cells in the presence of any glucose metabolism inhibitor, comparing the level of glucose uptake by the tumor cells obtained to the level of glucose uptake by a control; and determining that the subject is susceptible to the glucose metabolism inhibitor if the level of glucose uptake by the tumor cells is attenuated compared to the control. In some embodiments, the glucose uptake is measured by the uptake of radio-labelled glucose 2-deoxy-2-[fluorine-18]fluoro-D-glucose (¹⁸F-FDG). In further embodiments, detecting the ¹⁸F-FDG is by positron emission tomography (PET). In some embodiments, the biopsy is taken from a GBM tumor.

In some embodiments, the subject has been determined to be susceptible to the glucose metabolism inhibitor by a method comprising obtaining a first blood sample from the subject, placing the subject on a ketogenic diet for a period of time, obtaining a second blood sample from the subject after being placed on a ketogenic diet, measuring glucose level in the first and in the second blood sample; comparing the glucose level in the second blood sample with the glucose level in the first blood sample, and determining that the subject is susceptible if the glucose level in the second blood sample is reduced as compared to glucose levels in the first blood sample. In some embodiments, the reduction in the glucose level is between the second blood sample and the control blood sample is about or greater than 0.15 mM, is about or greater than 0.20 mM, is in the range of 0.15 mM-2.0 mM or is in the range of 0.25 mM-1.0 mM. In some embodiments, the reduction in the glucose levels is about, at least about, or at most about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0 mM (or any range derivable therein).

In certain aspects, the present disclosure provides methods of classifying a subject diagnosed with glioma or GBM, the method comprising obtaining a biological sample from the subject, treating the biological sample with glucose metabolism inhibitor(s) and determining if glucose metabolism is attenuated by the glucose metabolism inhibitor. Determining attenuation of glucose metabolism comprises determining change in glucose level, and/or change in rate of glycolysis, and/or change in glucose uptake, and/or change in extracellular acidification rate (ECAR), and/or measuring the activity of hexokinase, or phosphofructokinase, or pyruvate kinase before and after administration of the glucose metabolism inhibitor. In some embodiments, determining changes in glycolysis comprises direct measurement of pyruvate and/or lactate. In certain embodiments, the biological sample comprises cancer cells from a GBM tumor. In other embodiments, the method further comprises comparing the level of glucose attenuation to a control. In some embodiments, the method further comprises classifying the subject as a metabolic responder if glucose metabolism is attenuated by the glucose metabolism inhibitor in the biological sample. In further embodiments, the method further comprises treating the subject classified as a metabolic responder with a composition comprising a glucose metabolism inhibitor and a cytoplasmic p53 stabilizer.

In certain aspects, the present disclosure provides methods of assessing the sensitivity of cancer cells or tumor to treatment with a glucose metabolism inhibitor and a cytoplasmic p53 stabilizer, the method comprising measuring or detecting the level of glucose uptake by the cancer cells and comparing the level of glucose uptake with a control. The glucose can be radio-labelled such as for example, 2-deoxy-2-[fluorine-18]fluoro-D-glucose (¹⁸F-FDG). In some embodiments, the measuring and detecting of the radio labeled glucose uptake is done by positron emission tomography (PET).

In certain embodiments, the glucose metabolism inhibitors comprise one or more of a glucose uptake inhibitor, a glucose transporter inhibitor, a glycolysis inhibitor, a hexokinase inhibitor, an epidermal growth factor receptor (EGFR) inhibitor, any inducer of intracellular glucose deprivation or any combination thereof. In some embodiments, the EGFR inhibitor is erlotinib, gefitinib, lapatinib, cetuximab, panitumumab, vandetanib, necitumumab, or osimertinib. In further embodiments, the glucose inhibitor is a phosphatidylinositol 3-kinase PI3K inhibitor. The PI3K inhibitor can be, for example, pictilisib, dactolisib, wortmannin, LY294002, Idelalisib, duvelisib, buparlisib, IPI-549, SP2523, GDC-0326, TGR-1202, VPS34 inhibitor 1, GSK2269557, GDC-0084, SAR405, AZD8835, LY3023414, PI-103, TGX-221, NU7441, IC-87114, wortmannin, XL147 analogue, ZSTK474, Alpelisib, PIK-75 HCl, A66, AS-605240, 3-Methyladenine (3-MA), PIK-93, PIK-90, AZD64822, PF-04691502, Apitolisib, GSK1059615, Duvelisib, Gedatolisib, TG100-115, AS-252424, BGT226, CUDC-907, AS-604850, PIK-294, GSK2636771, Copanlisib, YM201636, CH5132799, CAY10505, PIK-293, PKI-402, TG100713, VS-5584, Taselisib, CZC24832, AMG319, GSK2292767, HS-173, Quercetin, Voxtalisib, PIK-93, Omipalisib, PIK-90, GNE-317, Pilaralisib, PF-4989216, AZD8186, 740 Y-P, Vps34-IN1, PIK-III, PI-3065 or analogs thereof. In some embodiments, the glucose metabolism inhibitor is 2-deoxyglucose (2DG) or cytochalasin B.

In some embodiments, the cytoplasmic p53 stabilizer is MDM2 antagonist/inhibitor. In some embodiments, the MDM2 antagonist is a nutlin. In further embodiments, the nutlin is nutlin-3 or idasanutlin. In other embodiments, the MDM2 antagonist is RO5045337, RO5503781, RO6839921, SAR405838, DS-3032, DS-3032b, or AMG-232 or any other MDM2 inhibitor.

In some embodiments, the cytoplasmic p53 stabilizer is a BCL-2 inhibitor. In some embodiments, the BCL-2 inhibitor is, for example, antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, APG-1252 or any other BCL-2 inhibitor.

In some embodiments, the cytoplasmic p53 stabilizer is a Bcl-xL inhibitor. In some embodiments, the Bcl-xL inhibitor is, for example, WEHI 539, ABT-263, ABT-199, ABT-737, sabutoclax, AT101, TW-37, APG-1252, gambogic acid or any other Bcl-xL inhibitor.

In some embodiments, the glucose metabolism inhibitor is erlotinib and the cytoplasmic p53 stabilizer is idasanutlin. In some embodiments the subject is administered any dose between about 1 mg to 250 mg of erlotinib. In some embodiments, the subject is administered 1, 5, 10, 15, 20, 21, 22, 23, 24, 25, 26, 27, 28, 29, 30, 21, 32, 33, 34, 35, 40, 45, 50, 55, 60, 65, 70, 75, 80, 85, 90, 95, 96, 97, 98, 99, 100, 101, 102, 103, 104, 105, 106, 107, 108, 109, 110, 115, 120, 125, 130, 135, 140, 145, 146, 147, 148, 149, 150, 151, 152, 153, 154, 155, 160, 170, 175, 180, 185, 190, 195, 200, 205, 210, 215, 220, 225, 230, 235, 240, 245, and 250 mg of erlotinib or any dose derivable in between. In some embodiments, the subject is administered any dose in between 50 mg to 1600 mg idasanutlin. In some embodiment, the subject is administered 100, 150, 300, 400, 450, or 600 mg of idasanutlin. In some embodiments the subject is administered 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1125, 1150, 1175, 1200, 1225, 1250, 1275, 1300, 1325, 1350, 1375, 1400, 1425, 1450, 1475, 1500, 1525, 1550, 1575, 1600, 1625 and 1650 mg or any dose derivable therein.

In other embodiments, the glucose metabolism inhibitor is a compound of formula I-a or I-b. In some embodiments thee glucose metabolism inhibitor is a compound of formula I-a or I-b and the cytoplasmic p53 stabilizer is idasanutlin.

In certain aspects the present disclosure provides methods of treating glioblastoma in a subject, the method comprising administering to the subject a therapeutically effective amount of a glucose uptake inhibitor and a cytoplasmic p53 stabilizer after determining that the subject is susceptible to reduced glucose metabolism by an EGFR inhibitor.

In certain aspects the present disclosure provides methods of reducing glioblastoma proliferation in a subject, the method comprising administering to the subject an effective amount of an EGFR inhibitor and a MDM2 inhibitor after determining that the subject is susceptible to EGFR inhibitors.

In some embodiments the glucose metabolism inhibitor and the cytoplasmic p53 stabilizer are administered in the same composition. In other embodiments, the glucose metabolism inhibitor and the cytoplasmic p53 are administered in separate compositions. For example, in some embodiments the glucose metabolism inhibitor and the cytoplasmic p53 stabilizer are administered within 24, 23, 22, 21, 20, 19, 18, 17, 16, 15, 14, 13, 12, 11, 10, 9, 8, 7, 6, 5, 4, 3, 2, 1 hours or within 30 min of each other or any fraction of hours or minutes in between. Yet in further embodiments, the glucose metabolism inhibitor and the p53 stabilizer are administered at the same time to the subject.

In certain embodiments of the current methods, the use of a control is contemplated such as comparing the level of glucose (or change in glucose or glucose attenuation) in a sample from a subject with a control sample. The control can comprise a non-cancerous sample, a cancerous sample with a different phenotype, a cancer sample with a wildtype EGFR expression level or any other control sample that is either taken from the patient's non-cancer cells or is not taken from the patient. In certain embodiments, the control is from a sample taken from the patient before the sample is subjected to glucose metabolism inhibitors.

In certain aspects the present disclosure provides methods for treating cancer or reducing cancer cell proliferation in a subject that has been determined to have cancer that is responsive to glucose metabolism inhibitors, comprising administering to a cancer patient an effective amount of a glucose metabolism inhibitor and a cytoplasmic p53 stabilizer. In some embodiments, the cancer is a Central Nervous System (CNS) cancer for example CNS metastases. In some embodiments, the cancer is a non-CNS cancer. In some embodiments, the cancer is glioblastoma multiforme, glioma, low-grade astrocytoma, mixed oligoastrocytoma, pilocytic astrocytoma, pleomorphic xanthoastrocytoma, subependymal giant cell astrocytoma, anaplastic astrocytoma, lung cancer or others.

In embodiments of the methods herein, the subject has been diagnosed with glioblastoma multiforme. In some embodiments, the subject has been previously treated for glioblastoma with a prior treatment. Yet is some embodiments, the subject has been determined to be resistant to the prior treatment.

In certain embodiments of the current methods, the method further comprises administration of an additional therapy. In some embodiments, the additional therapy is radiation therapy, chemotherapy, targeted therapy, immunotherapy, surgery. In some embodiments, the additional therapy comprises one or more therapies described herein.

In some embodiments, the compositions and/or methods of the disclosure excludes one or more of gefitinib, lapatinib, cetuximab, panitumumab, vandetanib, necitumumab, or osimertinib, pictilisib, dactolisib, LY294002, duvelisib, or buparlisib, 2-deoxyglucose (2DG), cytochalasin B, APR-246, RO5045337, RO5503781, RO6839921, SAR405838, DS-3032, DS-3032b, or AMG-232, antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252, sabutoclax, AT101, TW-37, APG-1252, or gambogic acid. In some embodiments, the compositions and/or methods exclude pictilisib, dactolisib, wortmannin, LY294002, Idelalisib, duvelisib, buparlisib, IPI-549, SP2523, GDC-0326, TGR-1202, VPS34 inhibitor 1, GSK2269557, GDC-0084, SAR405, AZD8835, LY3023414, PI-103, TGX-221, NU7441, IC-87114, wortmannin, XL147 analogue, ZSTK474, Alpelisib, PIK-75 HCl, A66, AS-605240, 3-Methyladenine (3-MA), PIK-93, PIK-90, AZD64822, PF-04691502, Apitolisib, GSK1059615, Duvelisib, Gedatolisib, TG100-115, AS-252424, BGT226, CUDC-907, AS-604850, PIK-294, GSK2636771, Copanlisib, YM201636, CH5132799, CAY10505, PIK-293, PKI-402, TG100713, VS-5584, Taselisib, CZC24832, AMG319, GSK2292767, HS-173, Quercetin, Voxtalisib, PIK-93, Omipalisib, PIK-90, GNE-317, Pilaralisib, PF-4989216, AZD8186, 740 Y-P, Vps34-IN1, PIK-III, PI-3065 or analogs thereof.

Use of the one or more compositions may be employed based on methods described herein. Use of one or more compositions may be employed in the preparation of medicaments for treatments according to the methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

### Methods of Assessment

### Glucose Uptake Tests

In embodiments of the methods and compositions of the current disclosure, the subject with GBM or cancer is classified to be either a “metabolic responder” or a “metabolic non-responder” i.e. determined to be susceptible to glucose metabolism inhibitors. In certain embodiments, the classification of the subject is prior to administering to the subject a treatment comprising a glucose metabolism inhibitor and a cytoplasmic p53 stabilizer. Accordingly, the current disclosure provides for methods for assessing a cancer, classifying a subject, determining the susceptibility of a subject to treatments involve analysis of glucose metabolism, glycolysis or glucose uptake. Methods to classify a subject as metabolic responder is described in details in Example 1. Techniques to monitor glycolysis and glucose uptake is provided by T. TeSlaa and M. A. Teitell. 2014. Methods in Enzymology, Volume 542, pp. 92-114, incorporated herein by reference.

Glycolysis is the intracellular biochemical conversion of one molecule of glucose into two molecules of pyruvate with the concurrent generation of two molecules of ATP. Pyruvate is a metabolic intermediate with several potential fates including entrance into the tricarboxylic acid (TCA) cycle within mitochondria to produce NADH and FADH₂. Alternatively, pyruvate can be converted into lactate in the cytosol by lactate dehydrogenase with concurrent regeneration of NAD⁺ from NADH. An increased flux through glycolysis supports the proliferation of cancer cells by providing, for example, additional energy in the form of ATP as well as glucose-derived metabolic intermediates for nucleotide, lipid, and protein biosynthesis. Warburg (Oncologia. 1956; 9(2):75-83) first observed that proliferating tumor cells augment aerobic glycolysis, the conversion of glucose to lactate in the presence of oxygen, in contrast to nonmalignant cells that mainly respire when oxygen is available. This mitochondrial bypass, called the Warburg effect, occurs in rapidly proliferating cells including cancer cells, activated lymphocytes, and pluripotent stem cells. The Warburg effect has been exploited for clinical diagnostic tests that use positron emission tomography (PET) scanning to identify increased cellular uptake of fluorinated glucose analogs such as ¹⁸F-deoxyglucose.

Thus, glycolysis represent a target for therapeutic and diagnostic methods. In the context of the current methods, the measurement of glucose uptake and lactate excretion by malignant cells may be useful to detect shifts in glucose catabolism and/or susceptibility to glucose metabolism inhibitors. Detecting such shifts is important for methods of treating GBM, methods of reducing the risk of ineffective therapy, methods for reducing the chances of tumor survival. For the purposes of this disclosure, ¹⁸F-deoxyglucose PET serves in certain embodiments as a rapid non-invasive functional biomarker to predict sensitivity to p53 activation. This non-invasive anlaysis could be particularly valuable for malignant brain tumors where pharmacokinetic/pharmacodynamics assessment is extremely difficult and impractical. In some cases, delayed imaging protocols (41) and parametric response maps (PRMs) with MRI fusion can be useful for quantifying the changes in tumore ¹⁸F-FDG uptake (42).

In certain aspects, the methods can relate to measuring glucose uptake and lactate production. For cells in culture, glycolytic flux can be quantified by measuring glucose uptake and lactate excretion. Glucose uptake into the cell is through glucose transporters (Glut1-Glut4), whereas lactate excretion is through monocarboxylate transporters (MCT1-MCT4) at the cell membrane.

### Extracellular Glucose and Lactate

Methods to detect glucose uptake and lactate excretion include, for example, extracellular glucose or lactate kit, extracellular bioanalyzer, ECAR measurement, [3H]-2-DG or [14C]-2-DG uptake ¹⁸FDG uptake or 2-NBDG uptake.

Commercially available kits and instruments are available to quantify glucose and lactate levels within cell culture media. Kit detection methods are usually colorimetric or fluorometric and are compatible with standard lab equipment such as spectrophotometers. BioProfile Analyzers (such as Nova Biomedical) or Biochemistry Analyzers (such as for example YSI Life Sciences) can measure levels of both glucose and lactate in cell culture media. GlucCell (Cesco BioProducts) can measure only glucose levels in cell culture media. While each commercial method has a different detection protocol, the collection of culture media for analysis is the same.

### Extracellular Acidification Rate

Glycolysis can also be determined through measurements of the extracellular acidification rate (ECAR) of the surrounding media, which is predominately from the excretion of lactic cid per unit time after its conversion from pyruvate. The Seahorse extracellular flux (XF) analyzer (Seahorse Bioscience) is a tool for measuring glycolysis and oxidative phosphorylation (through oxygen consumption) simultaneously in the same cells.

### Glucose Analog Uptake

Certain embodiments of the methods of the current disclosure include the use of glucose analogs. As would be familiar to a person skilled in the art, to determine the glucose uptake rate by cells, a labeled isoform of glucose can be added to the cell culture media and then measured within cells after a given period of time. Exemplary types of glucose analogs for these studies include but are not limited to radioactive glucose analogs, such as 2-deoxy-D-[1,2-3H]-glucose, 2-deoxy-D-[1-14C]-glucose, or 2-deoxy-2-(¹⁸F)-fluoro-D-glucose (¹⁸FDG), or fluorescent glucose analogs, such as 2-[N-(7-nitrobenz-2-oxa-1,3-diaxol-4-yl)amino]-2-deoxyglucose (2-NBDG). Measurements of radioactive glucose analog uptake require a scintillation counter, whereas 2-NBDG uptake is usually measured by flow cytometry or fluorescent microscopy. In some embodiments, the glucose uptake is measured by the uptake of radio-labelled glucose 2-deoxy-2-[fluorine-18]fluoro-D-glucose (¹⁸F-FDG). In further embodiments, detecting the ¹⁸F-FDG is by positron emission tomography (PET). In some embodiments, the biopsy is taken from a GBM tumor. A detailed description of an example of measuring ¹⁸F-FDG is provided in the examples below.

In certain aspects, the methods can relate to comparing glucose uptake of a biological sample such as a tumor sample with a control. Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression between a sample and a reference may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are with normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. In some embodiments, the levels can be relative to a control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some embodiments.

### Biological Sample Preparation

In certain aspects, methods involve obtaining a sample from a subject. Any biological sample from the patient that contains cancer cells may be used to evaluate the glucose uptake discussed herein. In some embodiments, a biological sample from a tumor is used. In other embodiments, a biological sample is blood or plasma. Evaluation of the sample may involve, though it need not involve, panning (enriching) for cancer cells, in vitro growth of cell line, or isolating the cancer cells.

In some embodiments, the subject has been determined to be susceptible to a glucose metabolism inhibitor by a method comprising obtaining a tumor biopsy from the subject, measuring the level of glucose uptake by the tumor cells in the presence of any glucose metabolism inhibitor, comparing the level of glucose uptake by the tumor cells obtained to the level of glucose uptake by a control; and determining that the subject is susceptible to the glucose metabolism inhibitor if the level of glucose uptake by the tumor cells is attenuated compared to the control.

The tumor biopsy may be but is not limited to GBMs. The methods of obtaining provided herein may include methods of biopsy such as needle aspiration, incisional biopsy, excisional biopsy, punch biopsy, or the like. The method of needle aspiration may further include fine needle aspiration, core needle biopsy, vacuum assisted biopsy, or large core biopsy. In some embodiments, multiple samples may be obtained by the methods herein to ensure a sufficient amount of biological material. In some cases, the fine needle aspirate sampling procedure may be guided by the use of an ultrasound, X-ray, or other imaging device.

In other embodiments the sample may be obtained from any of the tissues that include but are not limited to non-cancerous or cancerous tissue. General methods for obtaining biological samples are known in the art. Publications such as Ramzy, Ibrahim Clinical Cytopathology and Aspiration Biopsy 2001, which is herein incorporated by reference in its entirety, describes general methods for biopsy and cytological methods.

In other embodiments, the subject is determined to be susceptible to the glucose metabolism inhibitor by a method comprising obtaining a first blood sample from the subject, placing the subject on a ketogenic diet for a period of time, obtaining a second blood sample from the subject after being placed on a ketogenic diet, measuring glucose level in the first and in the second blood sample, comparing the glucose level in the second blood sample with the glucose level in the first blood sample, and determining that the subject is susceptible if the glucose level in the second blood sample is reduced as compared to glucose levels in the first blood sample. In some embodiments, the reduction in the glucose level is between the second blood sample and the control blood sample is about or greater than 0.15 mM, is about or greater than 0.20 mM, is in the range of 0.15 mM-2.0 mM or is in the range of 0.25 mM-1.0 mM.

The biological sample may be blood or plasma, may be a heterogeneous or homogeneous population of cells. The biological sample may be obtained using any method known to the art that can provide a sample suitable for the analytical methods described herein.

The sample may be obtained by methods known in the art. In some cases, the sample may be obtained, stored, or transported using components of a kit of the present methods. In some cases, multiple samples, such as multiple cancerous samples may be obtained for diagnosis by the methods described herein. In other cases, multiple samples, such as one or more samples from one tissue type and one or more samples from another tissue may be obtained for diagnosis by the methods. Samples may be obtained at different times are stored and/or analyzed by different methods.

### Pharmaceutical Compositions

In certain aspects, the present disclosure provides a pharmaceutical composition comprising a glucose metabolism inhibitor and a cytoplasmic p53 stabilizer as described herein.

In certain aspects, the compositions or agents for use in the methods described herein, such as therapeutic agents or inhibitors, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices, local administration, and the like.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrante (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

### Dosage

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

In certain embodiments, pharmaceutical compositions may comprise, for example, at least about 0.1% of an active agent, such as therapeutic agents or diagnostic agents. In other embodiments, the active agent may comprise between about 2% to about 75% of the weight of the unit, or between about 25% to about 60%, for example, and any range derivable therein. In embodiments, the compositions are administered orally. In embodiments, the compositions are administered sublingually In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

The dosage of the pharmaceutical compositions and formulations depends on the type of formulation and varies according to the size and health of the subject. Various combination and dosages are contemplated and are within the scope of the current invention and within the scope of “effective dose”, “therapeutically effective dose”, “pharmaceutically acceptable” or “pharmacologically acceptable” compositions, such as, by way of example, any dosage anywhere between 1-250 mg for erlotinib and between 50-450 mg for idasanutlin. In some embodiments, the subject is administered 150, 125, 100, 75, 50, 25 mg of erlotinib. In some embodiments, the subject is administered any dose in between 50 mg to 450 mg idasanutlin. In some embodiment, the subject is administered 100 or 150 mg of idasanutlin. Dosage of other therapeutics in accordance with the methods and compositions described herein are known in the medical community. The phrases “effective dose”, “therapeutically effective dose”, “pharmaceutically acceptable” or “pharmacologically acceptable” refer to molecular entities and compositions that do not produce an adverse, allergic, or other untoward reaction when administered to an animal, or human.

In certain embodiments, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

Upon formulation, solutions will be administered in a manner compatible with the dosage formulation and in such amount as is therapeutically or prophylactically effective.

The formulations are easily administered in a variety of dosage forms, such as the sublingual, buccal, and transdermal formulations described above. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

In certain embodiments, a subject is administered erlotinib in an amount of about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of an the condition have disappeared or been reduced or after 6, 12, 18, or 24 hours or 1, 2, 3, 4, or 5 days after symptoms have disappeared or been reduced.

In some embodiments, treatments of subjects may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months.

A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg (or any range derivable therein).

In some embodiments the glucose metabolism inhibitor and the cytoplasmic p53 stabilizer are administered in the same composition. In other embodiments, the glucose metabolism inhibitor and the cytoplasmic p53 are administered in separate compositions. For example, in some embodiments the glucose metabolism inhibitor and the cytoplasmic p53 stabilizer are administered within 24, 23, 22, 21, 20, 19, 18, 17, 16, 15, 14, 13, 12, 11, 10, 9, 8, 7, 6, 5, 4, 3, 2, 1 hours or within 30 min of each other or any fraction of hours or minutes in between. Yet in further embodiments, the glucose metabolism inhibitor and the p53 stabilizer are administered at the same time to the subject. In some embodiments, the glucose metabolism inhibitor and the p53 stabilizer are administered conjointly.

### Combination Therapies

In certain embodiments, compounds of the invention may be used alone or conjointly administered with another type of therapeutic agent.

In certain embodiments, the methods of the current disclosure are used in combination with additional therapies such as chemotherapy, therapeutic agents, surgical removal of cancerous cells, radiation therapy, and combinations thereof. In some aspects, the treatment regimen excludes one or more of chemotherapy, therapeutic agents, surgical removal of cancerous cells and/or radiation therapy. When administered concurrently, the combination cancer therapies may be administered in a single formulation or in separate formulations, and if separately, then optionally, by different modes of administration.

In further embodiments a combination of therapeutic treatment agents is administered to cancer cells. The therapeutic agents may be administered serially (within minutes, hours, or days of each other) or in parallel; they also may be administered to the patient in a pre-mixed single composition.

Various combinations of more than an anticancer modality, agent or compound (or a combination of such agents and/or compounds) may be employed, for example, a first anticancer modality, agent or compound is “A” and a second anticancer modality, agent or compound (or a combination of such modalities, agents and/or compounds) given as part of an anticancer therapy regime, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy.

Radiation therapy that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Alternative cancer therapy include any cancer therapy other than surgery, chemotherapy and radiation therapy, such as immunotherapy, targeted therapy, gene therapy, or a combination thereof. Subjects identified with poor prognosis using the present methods may not have favorable response to conventional treatment(s) alone and may be prescribed or administered one or more alternative cancer therapy per se or in combination with one or more conventional treatments.

### Pharmaceutically Acceptable Salts

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

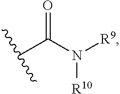

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

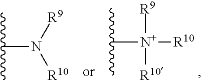

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

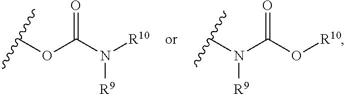

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

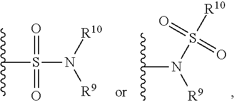

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

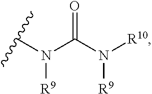

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Compounds described herein include all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified.

As used herein, “increased expression,” “increased level of expression,” “elevated expression,” “decreased expression,” or “decreased level of expression” refers to an expression level of a biomarker such as glucose uptake in the subject's sample as compared to a reference level or a control. In certain aspects, the reference level may be a reference level of expression from a non-cancerous tissue from the same subject. Alternatively, the reference level may be a reference level of expression from a different subject or group of subjects. For example, the reference level of expression may be an expression level obtained from a sample (e.g., a tissue, fluid or cell sample) of a subject or group of subjects without cancer, or an expression level obtained from a non-cancerous tissue of a subject or group of subjects with cancer. The reference level may be a single value or may be a range of values.

The reference level of expression can be determined using any method known to those of ordinary skill in the art. In some embodiments, the reference level is an average level of expression determined from a cohort of subjects with cancer or without cancer. The reference level may also be depicted graphically as an area on a graph. In certain embodiments, a reference level is a normalized level.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some embodiments it is contemplated that a numerical value discussed herein may be used with the term “about” or “approximately.”

The terms “protein”, “polypeptide” and “peptide” are, in certain embodiments, used interchangeably herein when referring to a gene product or functional protein. However, these terms are not limited to that usage and in certain embodiments convey their accepted meaning in the art.

The terms “attenuating”, “ameliorating,” “inhibiting,” or “reducing,” or any variation of these terms, when used in the claims and/or the specification includes any measurable decrease or complete inhibition to achieve a desired result.

The term “inhibitor” refers to a therapeutic agent that indirectly or directly inhibits the activity or expression of a protein, process (e.g. metabolic process), or biochemical pathway.

A person of ordinary skill in the art understands that an expression level from a test subject may be determined to have an elevated level of expression, a similar level of expression or a decreased level of expression compared to a reference level.

As used herein an “antagonist” describes a moiety that competitively binds to the receptor at the same site as an agonist, but does not activate the intracellular response initiated by the active form of the receptor and can thereby inhibit the intracellular responses by an agonist or partial agonist.

The term “inhibitor” refers to a therapeutic agen that indirectly or directly inhbitos the activity of expression of a protein, process (e. g. metabolic process), or biochemical pathway.

As used herein, in certain embodiments, “treating,” “treatment” or “therapy” is an approach for obtaining beneficial or desired clinical results. This includes: reduce the alleviation of symptoms, the reduction of inflammation, the inhibition of cancer cell growth, and/or the reduction of tumor size. In some embodiments, the term treatment refers to the inhibition or reduction of cancer cell proliferation in a subject having cancer. Furthermore, these terms are intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7): 1404-1411.

In certain embodiments, the term “pharmaceutical formulation” or “pharmaceutical composition” is intended to mean a composition or a mixture of compositions comprising at least one active ingredient; including but not limited to salts, solvates, and hydrates of compounds described herein.

The use of the word “a” or “an” when used in conjunction with the term “comprising” in the claims and/or the specification may mean “one,” but it is also consistent with the meaning of “one or more,” “at least one,” and “one or more than one.”

Throughout this application, in certain embodiments, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Compounds of the JGK Series

General Procedures:

All reactions were routinely carried out under an inert atmosphere of argon. Unless otherwise noted, materials were obtained from commercial suppliers and were used without purification. All solvents were purified and dried by standard techniques just before use. THF and Et₂O were freshly distilled from sodium and benzophenone. Methylene chloride, toluene, and benzene were purified by refluxing with CaH₂. Reactions were checked by thin layer chromatography (Kieselgel 60 F254, Merck). Spots were detected by viewing under a UV light, and by colorizing with charring after dipping in a p-anisaldehyde solution or phosphomolybdic acid solution. In aqueous work-up, all organic solutions were dried over anhydrous magnesium sulfate and filtered prior to rotary evaporation at water pump pressure. The crude compounds were purified by column chromatography on a silica gel (SilicaFlash P60, 230-400 mesh, SiliCycle Inc). Proton (¹H) and carbon (¹³C) NMR spectra were obtained on a Bruker AV 400 (400/100 MHz) or Bruker AV500 (500/125 MHz) spectrometer. Chemical shifts are reported in ppm units with Me₄Si or CHCl₃ as the internal standard. Splitting patterns are designated by: s, singlet; d, doublet; t, triplet; m, multiplet; b, broad. High resolution mass spectrometry data was obtained using a Thermo Fisher Scientific Exactive Plus with IonSense ID-CUBE DART source.

### Preparation of JGK001, JGK003

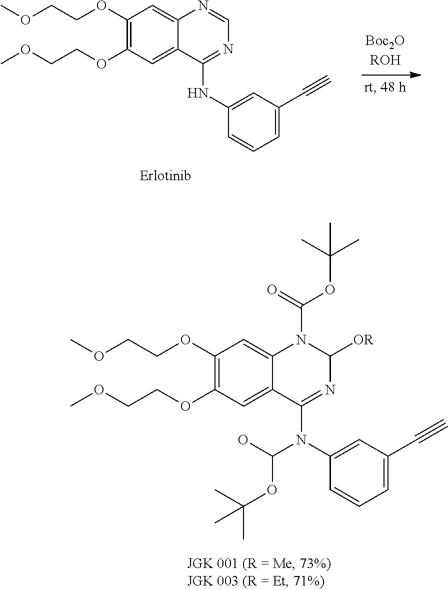

[JGK001]

To a solution of Eriotinib (134 mg, 0.3406 mmol) in anhydrous methanol (5.0 mL) was added Di-tert-butyl dicarbonate (228 mg, 1.7029 mmol) in one portion at room temperature. After being stirred at the same temperature for 48 h and concentrated in vacuo. The reaction mixture was diluted with H₂O (30 mL) and EtOAc (30 mL). The layers were separated, and the aqueous layer was extracted with EtOAc (2×30 mL). The combined organic layers were washed successively with H₂O and saturated brine, dried over anhydrous MgSO₄, filtered, and concentrated in vacuo. The residue was purified by column chromatography (silica gel, hexanes/EtOAc, 3/1) to give JGK001 (156 mg, 73%); ¹H NMR (400 MHz, CDCl₃) δ 7.86 (s, 1H), 7.43 (s, 1H), 7.20-7.23 (m, 2H), 7.16 (td, J=1.2, 7.6 Hz, 1H), 7.09 (d, J=8.0 Hz, 1H), 4.16-4.25 (m, 4H), 3.80 (t, J=5.2 Hz, 2H), 3.77 (t, J=5.2 Hz, 2H), 3.45 (s, 3H), 3.44 (s, 3H), 3.44 (s, 3H), 3.03 (s, 1H), 1.55 (s, 9H), 1.11 (s, 9H); ¹³C NMR (100 MHz, CDCl₃) δ 152.1, 151.5, 149.2, 148.8, 145.7, 142.7, 130.6, 128.9, 127.4, 125.3, 122.6, 121.5, 114.7, 111.4, 108.0, 90.7, 83.7, 83.3, 82.9, 76.8, 70.9, 70.7, 68.8, 68.7, 59.2, 59.1, 55.0, 28.2, 27.3; HRMS-ESI [M+H]⁺ found 626.3061 [calcd for C₃₃H₄₃N₃O₉ 625.2993].

[JGK003]

To a solution of Erlotinib (101 mg, 0.2567 mmol) in anhydrous ethanol (2.6 mL) was added Di-tert-butyl dicarbonate (172 mg, 1.2836 mmol) in one portion at room temperature. After being stirred at the same temperature for 48 h and concentrated in vacuo. The reaction mixture was diluted with H₂O (30 mL) and EtOAc (30 mL). The layers were separated, and the aqueous layer was extracted with EtOAc (2×30 mL). The combined organic layers were washed successively with H₂O and saturated brine, dried over anhydrous MgSO₄, filtered, and concentrated in vacuo. The residue was purified by column chromatography (silica gel, hexanes/EtOAc, 3/1) to give JGK003 (117 mg, 71%); ¹H NMR (400 MHz, CDCl₃) δ 7.86 (s, 1H), 7.43 (s, 1H), 7.34 (s, 1H), 7.21-7.24 (m, 2H), 7.16 (d, J=7.6 Hz, 1H), 7.10 (d, J=7.6 Hz, 1H), 4.17-4.25 (m, 4H), 3.61-3.82 (m, 6H), 3.46 (s, 3H), 3.45 (s, 3H), 3.02 (s, 1H), 1.55 (s, 9H), 1.23 (t, J=6.8 Hz, 3H), 1.12 (s, 9H); ¹³C NMR (100 MHz, CDCl₃) δ 152.0, 151.4, 149.2, 148.9, 145.5, 143.0, 130.8, 128.8, 127.3, 125.3, 122.5, 121.7, 114.7, 111.3, 107.9, 89.3, 83.7, 83.2, 82.8, 76.7, 70.9, 70.7, 68.8, 68.6, 63.0, 59.2, 59.1, 28.2, 27.4, 14.6; HRMS-ESI [M+H]⁺ found 640.3211 [calcd for C₃₄H₄₅N₃O₉ 639.3150].

### Preparation of JGK002

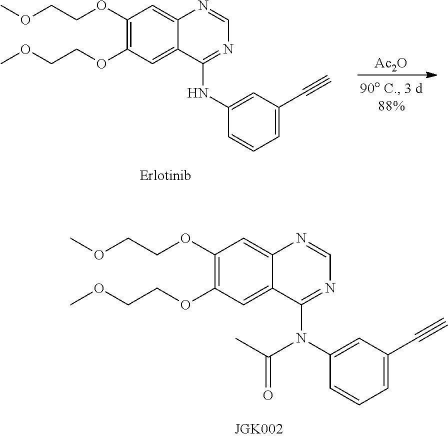

To a solid of erlotinib (165 mg, 0.4194 mmol) was added acetic anhydride (5.0 mL). After being heated at 90° C. (bath temperature) with stirring for 3 d, the reaction mixture was cooled to room temperature and neutralized with saturated aqueous NaHCO₃(20 mL), and diluted with EtOAc (20 mL). The layers were separated, and the aqueous layer was extracted with EtOAc (2×30 mL). The combined organic layers were washed successively with H₂O and saturated brine, dried over anhydrous MgSO₄, filtered, and concentrated in vacuo. The residue was purified by column chromatography (silica gel, hexanes/EtOAc, 1/1 to 1/3) to give JGK002 (161 mg, 88% isolated yield); ¹H NMR (400 MHz, CDCl₃) δ 9.06 (s, 1H), 7.45 (t, J=1.6 Hz, 1H), 7.37-7.39 (m, 2H), 7.36 (s, 1H), 7.30-7.34 (m, 1H), 7.15 (s, 1H), 4.32 (t, J=4.8 Hz, 2H), 4.16 (t, J=4.8 Hz, 2H), 3.86 (t, J=4.8 Hz, 2H), 3.79 (t, J=4.8 Hz, 2H), 3.46 (s, 3H), 3.45 (s, 3H), 3.06 (s, 1H), 2.14 (s, 3H); ¹³C NMR (100 MHz, CDCl₃) δ 170.5, 158.8, 156.1, 153.5, 151.1, 150.8, 141.0, 130.9, 130.3, 129.3, 127.5, 123.4, 117.2, 107.9, 103.1, 82.4, 78.4, 70.6, 70.3, 68.9, 68.7, 59.3, 59.3, 23.7; HRMS-ESI [M+H]⁺ found 436.1811 [calcd for C₂₄H₂₅N₃O₅ 435.1788].

Preparation of JGK010, JGK032—General Procedure for Substitution with Aniline Analogues

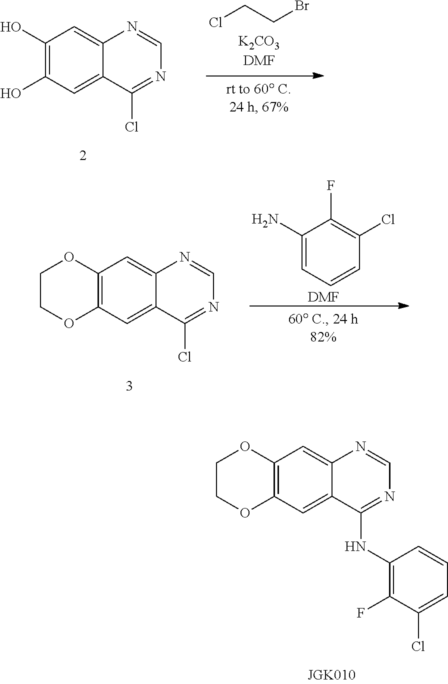

[Cyclization]

To a solution of diol 2 (530 mg, 2.6959 mmol) in DMF (13.5 mL, 0.2 M) was added in one portion potassium carbonate (1490 mg), followed by successive dropwise addition of 1-bromo-2-chloroethane (1.3 mL) at room temperature under Ar. After being heated at 60° C. (bath temperature) with stirring for 24 h, the reaction mixture was cooled to room temperature, quenched with H₂O (50 mL). The layers were separated, and the aqueous layer was extracted with EtOAc (50 mL). The combined organic layers were washed successively with H₂O and saturated brine, dried over anhydrous MgSO₄, filtered, and concentrated in vacuo. The residue was purified by column chromatography (silica gel, hexanes/EtOAc, 6/1 to 3/1) to give fused-chloroquinazoline 3 (404 mg, 67%); ¹H NMR (400 MHz, CDCl₃) δ 8.84 (s, 1H), 7.64 (s, 1H), 7.47 (s, 1H), 4.43-4.45 (m, 2H), 4.39-4.42 (m, 2H). [known compound; Chilin, A. et al. J. Med. Chem. 2010, 53, 1862-1866]

[JGK010]

To a solution of fused-chloroquinazoline 3 (114 mg, 0.5120 mmol) in DMF (2.6 mL) was dropwise added 3-chloro-2-fluoroaniline (0.10 mL) at room temperature. After being heated at 60° C. (bath temperature) with stirring for 24 h, the reaction mixture was cooled to room temperature and diluted with Et₂O (30.0 mL) to give white suspension. The resulting white solid were washed successively with Et₂O (2×50 mL) and collected to give JGK010 (140 mg, 82%); ¹H NMR (400 MHz, CDCl₃) δ 8.68 (s, 1H), 8.59 (ddd, J=3.2, 6.8, 6.8 Hz, 1H), 7.39 (s, 1H), 7.34 (s, 1H), 7.29 (s, 1H), 7.10-7.18 (m, 2H), 4.38-4.43 (m, 4H); ¹H NMR (500 MHz, DMSO-d₆) δ 11.78 (s, 1H), 8.79 (s, 1H), 8.45 (s, 1H), 7.62 (t, J=7.0 Hz, 1H), 7.50 (t, J=7.0 Hz, 1H), 7.43 (s, 1H), 7.34 (t, J=8.0 Hz, 1H), 4.46-4.53 (m, 2H), 4.40-4.52 (m, 2H); ¹³C NMR (125 MHz, DMSO-d₆) δ 159.8, 154.0, 152.2, 149.9, 145.7, 135.2, 129.9, 128.1, 126.4, 125.8, 120.9, 111.3, 108.1, 105.8, 65.5, 64.6; HRMS-ESI [M+H]⁺ found 332.0551 [calcd for C₁₆H₁₁ClFN₃O₂ 331.0518].

### Preparation of JGK005

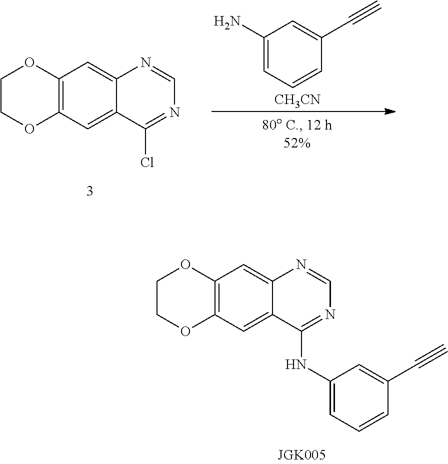

To a solution of fused-chloroquinazoline 3 (14 mg, 0.0628 mmol) in CH₃CN (2.0 mL) was dropwise added 3-ethynylaniline (0.05 mL) at room temperature. After being heated at 80° C. (bath temperature) with stirring for 12 h, the reaction mixture was cooled to room temperature, and concentrated in vacuo. The residue was purified by column chromatography (silica gel, hexanes/EtOAc, 3/1) to give JGK005 (10 mg, 52%); ¹H NMR (400 MHz, DMSO-d₆) δ 9.45 (s, 1H), 8.43 (s, 1H), 8.04-8.05 (m, 2H), 7.87-7.90 (m, 1H), 7.34 (t, J=7.9 Hz, 1H), 7.14-7.16 (m, 2H), 4.35-4.39 (m, 4H), 4.14 (s, 1H); ¹³C NMR (100 MHz, DMSO-d₆) δ 156.8, 153.3, 149.5, 146.5, 144.1, 140.2, 129.3, 126.7, 124.9, 122.7, 122.1, 113.0, 110.4, 108.8, 84.0, 80.9, 64.9, 64.6; HRMS-ESI [M+H]⁺ found 304.1079 [calcd for C₁₈H₁₃N₃O₂ 303.1002].

### JGK025

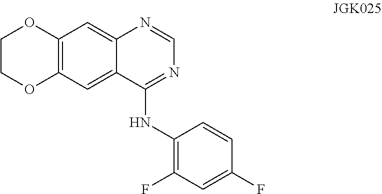 [SMILES: Fc1ccc(Nc2ncnc3cc4c(cc23)OCCO4)c(F)c1]

Preparation of JGK025 was followed by General Procedure; JGK025 (25%); ¹H NMR (400 MHz, DMSO-d₆) δ 11.30 (s, 1H), 8.73 (s, 1H), 8.28 (s, 1H), 7.51-7.58 (m, 1H), 7.41-7.48 (m, 1H), 7.35 (s, 1H), 7.17-7.23 (m, 1H), 4.44-4.50 (m, 2H), 4.39-4.44 (m, 2H); ¹³C NMR (125 MHz, MeOD) δ 161.6 (J=245.9 Hz), 160.0, 157.6 (J=249.6 Hz), 152.1, 150.1, 145.6, 135.4, 130.4, 121.4, 112.4, 110.9, 108.1, 108.1, 105.4, 65.5, 64.6; HRMS-ESI [M+H]⁺ found 316.0890 [calcd for C₁₆H₁₁F₂N₃O₂ 315.0813].

### JGK026

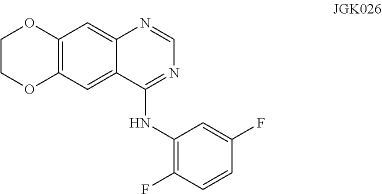 [SMILES: Fc1ccc(F)c(Nc2ncnc3cc4c(cc23)OCCO4)c1]

Preparation of JGK026 was followed by General Procedure; JGK026 (22%); ¹H NMR (400 MHz, DMSO-d₆) δ 11.09 (s, 1H), 8.74 (s, 1H), 8.18 (s, 1H), 7.39-7.51 (m, 2H), 7.30 (s, 1H), 7.23-7.29 (m, 1H), 4.45-4.49 (m, 2H), 4.40-4.44 (m, 2H); ¹³C NMR (125 MHz, MeOD) δ 159.8, 158.1 (J=239.6 Hz), 153.7 (J=243.2 Hz), 152.1, 150.1, 145.7, 135.7, 126.0, 117.9, 117.8, 116.0, 110.9, 108.2, 106.2, 65.5, 64.6; HRMS-ESI [M+H]⁺ found 316.0893 [calcd for C₁₆H₁₁F₂N₃O₂ 315.0813].

### JGK027

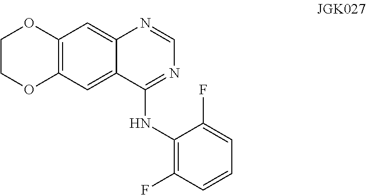 [SMILES: Fc1cccc(F)c1Nc1ncnc2cc3c(cc12)OCCO3]

Preparation of JGK027 was followed by General Procedure; JGK027 (6%); ¹H NMR (400 MHz, DMSO-d₆) δ 11.23 (bs, 1H), 8.75 (s, 1H), 8.29 (s, 1H), 7.91 (s, 1H), 7.46-7.55 (m, 1H), 7.29 (t, J=8.1 Hz, 2H), 4.46-4.50 (m, 2H), 4.40-4.46 (m, 2H); ¹³C NMR (125 MHz, MeOD) δ 163.6, 160.7, 160.4, 158.4, 152.5, 151.5, 146.2, 130.6, 115.6, 113.5, 113.4, 111.9, 109.3, 108.2, 66.2, 65.3; HRMS-ESI [M+H]⁺ found 316.0889 [calcd for C₁₆H₁₁F₂N₃O₂ 315.0813].

### JGK028

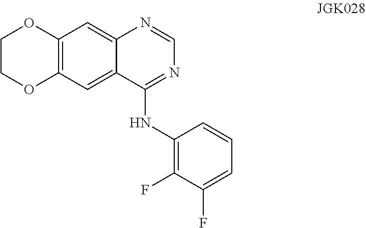 [SMILES: Fc1cccc(Nc2ncnc3cc4c(cc23)OCCO4)c1F]

Preparation of JGK028 was followed by General Procedure; JGK028 (41%); ¹H NMR (500 MHz, MeOD) δ 8.64 (s, 1H), 8.03 (s, 1H), 7.31-7.38 (m, 2H), 7.24-7.31 (m, 2H), 4.50-4.55 (m, 2H), 4.44-4.50 (m, 2H); ¹³C NMR (125 MHz, MeOD) δ 160.1, 152.8, 150.9 (J=245.6 Hz), 149.0, 146.2, 145.9 (J=249.7 Hz), 134.6, 126.0, 124.0, 123.1, 116.2, 109.8, 107.8, 105.0, 65.2, 64.2; HRMS-ESI [M+H]⁺ found 316.0884 [calcd for C₁₆H₁₁F₂N₃O₂ 315.0813].

### JGK029

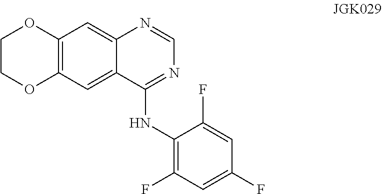 [SMILES: Fc1cc(F)c(Nc2ncnc3cc4c(cc23)OCCO4)c(F)c1]

Preparation of JGK029 was followed by General Procedure; JGK029 (52%); ¹H NMR (500 MHz, MeOD) δ 8.60 (s, 1H), 7.98 (s, 1H), 7.29 (s, 1H), 7.07-7.13 (m, 2H), 4.50-4.53 (m, 2H), 4.44-4.48 (n, 2H); ¹³C NMR (125 MHz, DMSO-d₆) δ 161.7, 160.3, 158.6, 158.1, 153.2, 150.3, 144.4, 143.7, 117.2, 113.8, 113.0, 112.3, 109.5, 101.5, 65.3, 64.5; HRMS-ESI [M+H]⁺ found 334.0794 [calcd for C₁₆H₁₀F₃N₃O₂ 333.0719].

### JGK017

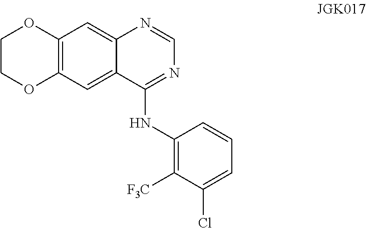 [SMILES: FC(F)(F)c1c(Cl)cccc1Nc1ncnc2cc3c(cc12)OCCO3]

Preparation of JGK017 was followed by General Procedure; JGK017 (5%); ¹H NMR (500 MHz, CDCl₃) δ 8.59 (s, 1H), 8.15 (d, J=8.3 Hz, 1H), 7.49 (t, J=8.1 Hz, 1H), 7.38 (s, 1H), 7.34 (d, J=7.9 Hz, 1H), 7.21 (s, 1H), 4.41-4.42 (m, 2H), 4.38-4.40 (m, 2H); ¹³C NMR (125 MHz, CDCl₃) δ 156.4, 153.2, 149.7, 146.7, 144.5, 138.4, 133.5, 132.2, 128.2, 125.4, 119.9, 119.7, 114.3, 110.4, 105.7, 64.5, 64.3.

Preparation of JGK004

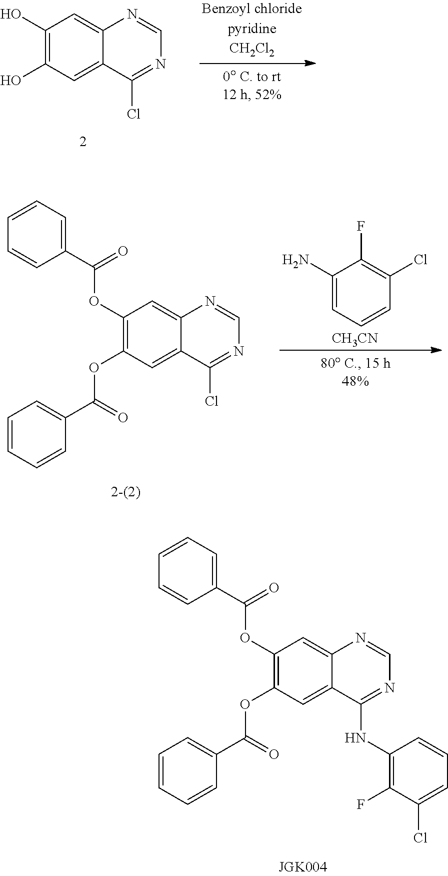

[Benzoylation]

To a cooled (0° C.) solution of diol 2 (205 mg, 1.0428 mmol) in anhydrous CH₂Cl₂ (5.2 mL, 0.2 M) was dropwise added successively pyridine (0.5 mL) and benzoyl chloride (0.7 mL) under Ar. After being stirred at the room temperature for 12 h, the reaction mixture was quenched with saturated aqueous NH₄Cl (20 mL), and diluted with CH₂Cl₂ (20 mL). The layers were separated, and the aqueous layer was extracted with CH₂Cl₂ (2×50 mL). The combined organic layers were washed successively with H₂O and saturated brine, dried over anhydrous MgSO₄, filtered, and concentrated in vacuo. The residue was purified by column chromatography (silica gel, hexanes/EtOAc, 10/1) to give benzoyl chloroquinazoline 2-(2) (220 mg, 52%); ¹H NMR (400 MHz, CDCl₃) δ 9.07 (s, 1H), 8.31 (s, 1H), 8.16 (s, 1H), 8.04-8.07 (m, 4H), 7.53-7.58 (m, 2H), 7.34-7.39 (m, 4H).

[JGK004]

To a solution of benzoyl chloroquinazoline 2-(2) (180 mg, 0.444 mmol) in CH₃CN (3.0 mL) was dropwise added 3-chloro-2-fluoroaniline (0.06 mL, 0.533 mmol) at room temperature. After being heated at 80° C. (bath temperature) with stirring for 15 h, the reaction mixture was cooled to room temperature, and concentrated in vacuo. The residue was purified by column chromatography (silica gel, hexanes/EtOAc, 3/1) to give JGK004 (109 mg, 48%); ¹H NMR (400 MHz, CDCl₃) δ 8.85 (s, 1H), 8.48-8.53 (m, 1H), 8.08 (t, J=7.2 Hz, 4H), 7.99 (d, J=3.2 Hz, 2H), 7.51-7.59 (m, 3H), 7.39 (dd, J=8.4, 16.0 Hz, 4H), 7.16-7.21 (m, 2H); ¹³C NMR (125 MHz, CDCl₃) δ 164.2, 163.7, 156.6, 155.1, 150.6, 149.1, 148.6, 147.5, 142.1, 134.1, 134.1, 130.3, 130.2, 128.6, 128.6, 128.1, 128.0, 127.9, 127.8, 125.3, 124.6, 124.5, 122.9, 121.6, 121.0, 120.9, 114.4, 113.3; HRMS-ESI [M+H]⁺ found 514.0963 [calcd for C₂₈H₁₇ClFN₃O₄ 513.0886].

### Preparation of JGK006

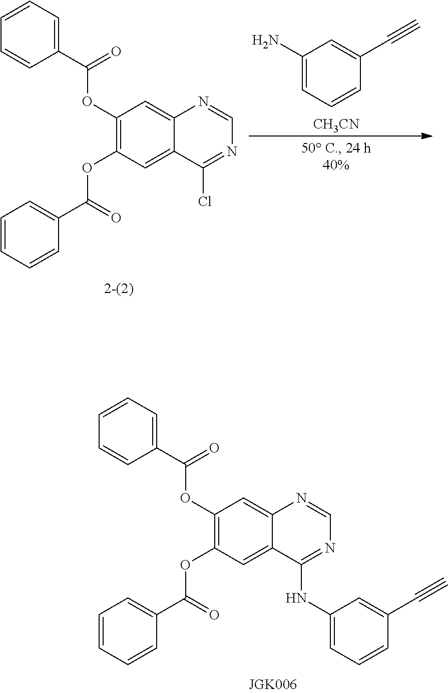

To a solution of benzoyl chloroquinazoline 2-(2) (100 mg, 0.247 mmol) in CH₃CN (3.0 mL) was dropwise added 3-ethynylaniline (0.05 mL, 0.430 mmol) at room temperature. After being heated at 50° C. (bath temperature) with stirring for 24 h, the reaction mixture was cooled to room temperature and concentrated in vacuo. The residue was purified by column chromatography (silica gel, hexanes/EtOAc, 4/1) to give JGK006 (48 mg, 40%); ¹H NMR (400 MHz, CDCl₃) δ 8.71 (s, 1H), 8.03 (d, J=8.0 Hz, 2H), 7.96 (s, 1H), 7.90-7.95 (m, 2H), 7.79 (s, 1H), 7.62-7.75 (m, 3H), 7.55 (t, J=7.3 Hz, 1H), 7.48 (t, J=7.5 Hz, 1H), 7.37 (t, J=7.4 Hz, 2H), 7.22-7.31 (m, 4H), 3.04 (s, 1H); ¹³C NMR (100 MHz, CDCl₃) δ 164.8, 163.9, 156.7, 155.3, 148.9, 146.9, 141.3, 138.1, 134.1, 134.0, 130.3, 130.1, 128.9, 128.6, 128.5, 128.1, 128.0, 127.9, 124.8, 122.7, 122.4, 122.1, 115.1, 113.1, 83.3; HRMS-ESI [M+H]⁺ found 486.1443 [calcd for C₃₀H₁₉N₃O₄ 485.1370]

### Preparation of JGK032

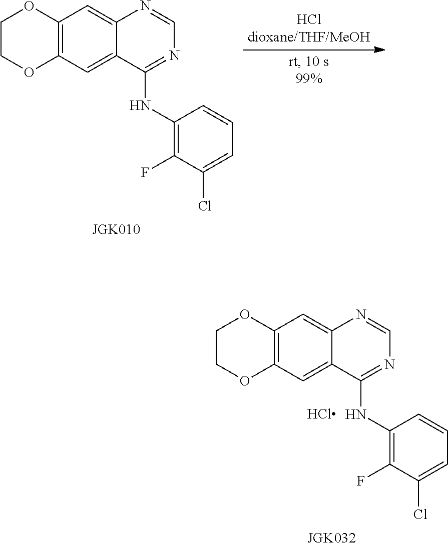 [SMILES: Fc1c(Cl)cccc1Nc1ncnc2cc3c(cc12)OCCO3 ; Fc1c(Cl)cccc1Nc1ncnc2cc3c(cc12)OCCO3]

1.0 M hydrogen chloride solution was generated by addition of hydrogen chloride solution (0.1 mL, 4.0 M in dioxane, 0.4 mmol) to THF (0.3 mL) at room temperature. To a solution of JGK010 (6.1 mg, 0.01839 mmol) in MeOH was dropwise added the above-generated hydrogen chloride solution (0.030 mL, 0.030 mmol) at room temperature. After being stirred at the same temperature for 10 seconds, the reaction mixture was concentrated in vacuo to give JGK032 (6.7 mg, 99%); ¹H NMR (500 MHz, DMSO-d₆) δ 11.63 (s, 1H), 8.81 (s, 1H), 8.36 (s, 1H), 7.63 (ddd, J=1.6, 6.9, 8.3 Hz, 1H), 7.39 (s, 1H), 7.35 (ddd, J=1.1, 8.1, 16.2 Hz, 1H), 4.49-4.51 (m, 2H), 4.43-4.45 (m, 2H).

### Preparation of JGK012

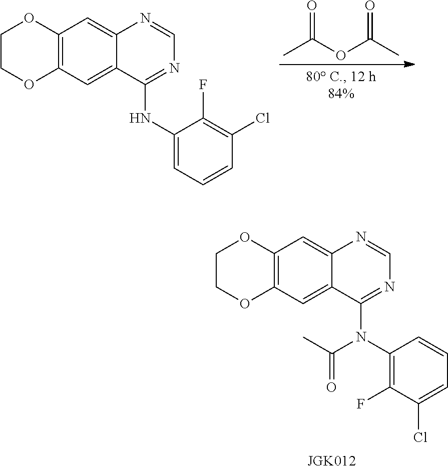

To a solid of JGK010 (39 mg, 0.1176 mmol) was added acetic anhydride (5.0 mL). After being heated at 80° C. (bath temperature) with stirring for 12 h, the reaction mixture was cooled to room temperature and neutralized with saturated aqueous NaHCO₃ (20 mL), and diluted with EtOAc (20 mL). The layers were separated, and the aqueous layer was extracted with EtOAc (2×30 mL). The combined organic layers were washed successively with H₂O and saturated brine, dried over anhydrous MgSO₄, filtered, and concentrated in vacuo. The residue was purified by column chromatography (silica gel, hexanes/EtOAc, 2/1 to 1/1) to give JGK012 (37 mg, 84% isolated yield); ¹H NMR (400 MHz, CDCl₃) δ 9.01 (s, 1H), 7.49 (s, 1H), 7.44 (s, 1H), 7.31-7.41 (m, 2H), 7.09 (t, J=8.0 Hz, 1H), 4.41-4.43 (m, 2H), 4.37-4.40 (m, 2H), 2.15 (s, 3H); ¹³C NMR (125 MHz, CDCl₃) δ 170.2, 159.2, 153.3, 151.3, 149.7, 145.8, 130.6, 129.8, 129.7, 124.7, 122.5, 122.4, 117.7, 113.6, 109.2, 64.5, 64.2, 22.9; HRMS-ESI [M+H]⁺ found 374.0701 [calcd for C₁₈H₁₃ClFN₃O₃ 373.0623].

### Preparation of JGK015

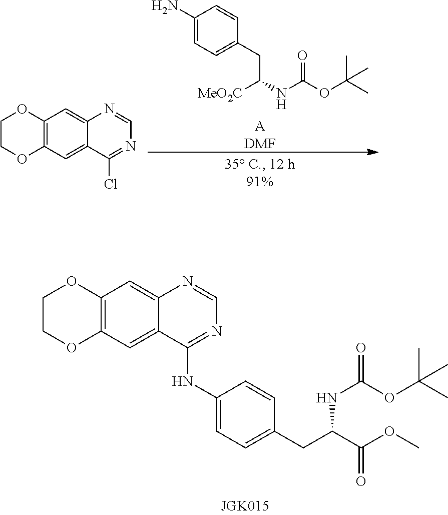

To a solution of L-amino acid analogue A (227 mg, 0.7712 mmol) in DMF (3.0 mL) was added in one portion fused-chloroquinazoline 3 (117 mg, 0.5932 mmol) at room temperature. After being heated at 35° C. (bath temperature) with stirring for 12 h, the reaction mixture was cooled to room temperature and diluted with saturated brine (30.0 mL) and EtOAc (30.0 mL) to give yellow suspension. The layers were separated, and the aqueous layer was extracted with EtOAc (2×50 mL). The combined organic layers were concentrated in vacuo. The residue was purified by column chromatography (silica gel, CH₂Cl₂/MeOH, 40/1 to 10/1) to give JGK015 (261 mg, 92%); ¹H NMR (500 MHz, CDCl₃) δ 8.57 (s, 1H), 7.64 (s, 1H), 7.61 (d, J=8.0 Hz, 2H), 7.37 (s, 1H), 7.27 (s, 1H), 7.08 (d, J=8.5 Hz, 2H), 5.09 (d, J=7.5 Hz, 1H), 4.54 (dd, J=6.0, 13.5 Hz, 1H), 4.31-4.33 (m, 2H), 4.27-4.29 (m, 2H), 3.68 (s, 3H), 3.06 (dd, J=5.6, 14.0 Hz, 1H), 3.00 (dd, J=6.1, 13.8 Hz, 1H), 1.39 (s, 9H); ¹³C NMR (125 MHz, CDCl₃) δ 172.4, 156.5, 155.2, 153.6, 149.1, 146.3, 143.8, 137.6, 131.6, 129.7, 121.7, 113.8, 110.3, 106.6, 80.0, 64.4, 64.2, 54.4, 52.2, 37.6, 28.3; HRMS-ESI [M+H]⁺ found 481.2082 [calcd for C₂₅H₂₈N₄O₆ 480.2003].

### Preparation of JGK016, JGK023

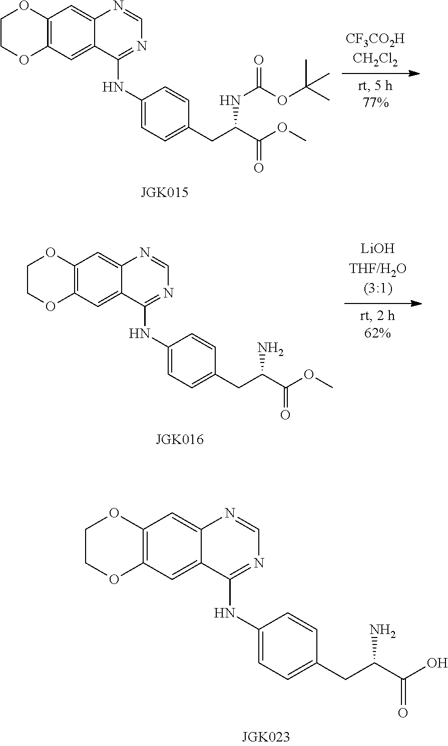 [SMILES: COC(=O)[C@H](Cc1ccc(Nc2ncnc3cc4c(cc23)OCCO4)cc1)NC(=O)OC(C)(C)C ; COC(=O)[C@@H](N)Cc1ccc(Nc2ncnc3cc4c(cc23)OCCO4)cc1 ; N[C@@H](Cc1ccc(Nc2ncnc3cc4c(cc23)OCCO4)cc1)C(=O)O]

[JGK016 (Boc Deprotection)]

To a solution of JGK015 (121 mg, 0.251 mmol) in anhydrous CH₂Cl₂ (5 mL, 0.05 M) was dropwise added trifluoroacetic acid (1.0 mL) at room temperature. After being stirred at the same temperature for 5 h, the reaction mixture was quenched with saturated aqueous NaHCO₃ (20 mL), and diluted with CH₂Cl₂ (20 mL). The layers were separated, and the aqueous layer was extracted with CH₂Cl2 (2×30 mL). The combined organic layers were washed successively with H₂O and saturated brine, dried over anhydrous MgSO₄, filtered, and concentrated in vacuo. The residue was purified by column chromatography (silica gel, CH₂Cl₂/MeOH, 20/1) to give JGK016 (74 mg, 77%); ¹H NMR (400 MHz, DMSO-d₆) δ 10.5 (s, 1H), 8.68 (s, 1H), 8.43 (s, 2H), 8.20 (s, 1H), 7.68 (d, J=8.4 Hz, 2H), 7.26 (d, J=8.4 Hz, 2H), 7.23 (s, 1H), 4.44-4.46 (m, 2H), 4.39-4.41 (m, 2H), 3.68 (s, 3H), 3.03-3.13 (m, 2H); ¹H NMR (400 MHz, CD₃OD) δ 8.26 (s, 1H), 7.73 (s, 1H), 7.60 (d, J=8.4 Hz, 2H), 7.17 (d, J=8.4 Hz, 2H), 7.08 (s, 1H), 4.31-4.35 (m, 4H), 3.72 (t, J=6.6 Hz, 1H), 3.68 (s, 3H), 3.27-3.29 (m, 1H), 3.01 (dd, J=5.9, 13.6 Hz, 1H), 2.89 (dd, J=7.0, 13.5 Hz, 1H); ¹³C NMR (100 MHz, CD₃OD) 174.4, 157.4, 152.6, 149.7, 145.0, 144.2, 137.5, 132.8, 129.2, 122.8, 122.3, 111.5, 110.1, 107.9, 64.5, 64.1, 55.2, 51.0, 39.5; HRMS-ESI [M+H]⁺ found 381.1553 [calcd for C₂₀H₂₀N₄O₄ 380.1479].

[JGK023 (Hydrolysis)]

To a cooled (0° C.) solution of JGK016 (42 mg, 0.1104 mmol) in THF/H₂O (3:1, total 4.0 mL) was added in one portion lithium hydroxide (14 mg). After being stirred at the room temperature for 2 h, the reaction mixture was neutralized with 1N HCl and diluted with EtOAc (20 mL). The layers were separated, and the aqueous layer was extracted with EtOAc (100 mL). The combined organic layers were washed successively with H₂O and saturated brine, dried over anhydrous MgSO₄, filtered, and concentrated in vacuo. The residue was purified by column chromatography (silica gel, CH₂Cl₂/MeOH, 30/1 to 15/1) to give JGK023 (25 mg, 62%); ¹H NMR (400 MHz, CDCl₃) δ 8.62 (s, 1H), 8.12 (s, 1H), 7.71 (d, J=8.4 Hz, 2H), 7.40 (d, J=8.4 Hz, 2H), 7.24 (s, 1H), 4.48-4.50 (m, 2H), 4.42-4.44 (m, 2H), 4.28 (t, J=6.8 Hz, 1H), 3.32-3.37 (m, 1H), 3.20 (dd, J=7.6, 14.8 Hz, 1H); ¹³C NMR (125 MHz, CDCl₃) δ 169.7, 159.1, 152.4, 148.8, 146.0, 136.1, 134.1, 133.1, 129.7, 129.7, 124.8, 124.8, 110.0, 108.1, 104.9, 65.2, 64.2, 53.6, 35.4; HRMS-ESI [M+H]⁺ found 367.1334 [calcd for C₁₉H₁₈N₄O₄ 366.1322].

### Preparation of JGK020

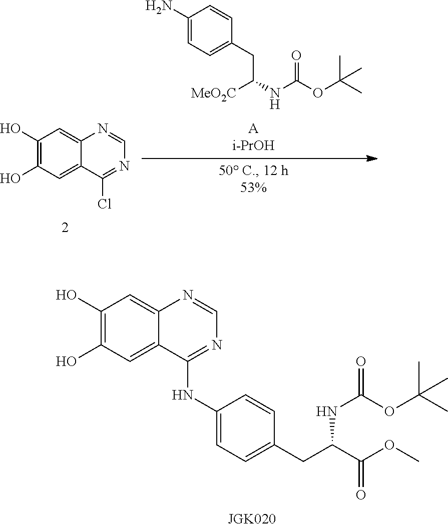 [SMILES: COC(=O)[C@H](Cc1ccc(N)cc1)NC(=O)OC(C)(C)C ; Oc1cc2ncnc(Cl)c2cc1O ; COC(=O)[C@H](Cc1ccc(Nc2ncnc3cc(O)c(O)cc23)cc1)NC(=O)OC(C)(C)C]

To a solution of chloroquinazoline 2 (104 mg, 0.5294 mmol) in isopropyl alcohol (5.3 mL) was dropwise added amino acid (187 mg) at room temperature. After being heated at 50° C. (bath temperature) with stirring for 12 h, the reaction mixture was cooled to room temperature and and concentrated in vacuo. The residue was purified by column chromatography (silica gel, hexanes/EtOAc, 5/1 to 3/1) to give JGK020 (128 mg, 53%); ¹H NMR (400 MHz, DMSO-d₆) δ 10.68 (s, 1H), 10.22 (br, 1H), 8.64 (s, 1H), 7.91 (s, 1H), 7.53 (d, J=8.4 Hz, 2H), 7.26-7.31 (m, 4H), 4.12-4.18 (m, 1H), 3.59 (s, 3H), 2.98 (dd, J=5.2, 14.0 Hz, 1H), 2.84 (dd, J=10.0, 13.2 Hz, 1H), 1.30 (s, 9H); ¹³C NMR (125 MHz, DMSO-d₆) δ 173.0, 158.2, 155.9, 155.6, 148.7, 148.4, 136.1, 135.8, 129.7, 124.7, 107.6, 107.3, 103.3, 78.8, 55.7, 52.3, 36.3, 28.6; HRMS-ESI [M+H]⁺ found 455.1920 [calcd for C₂₃H₂₆N₄O₆ 454.1846].

### JGK014

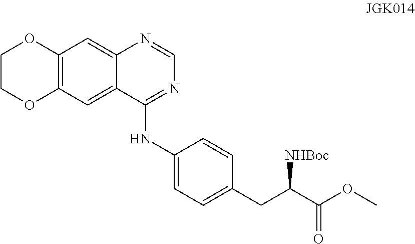 [SMILES: COC(=O)[C@@H](Cc1ccc(Nc2ncnc3cc4c(cc23)OCCO4)cc1)NC(=O)OC(C)(C)C]

JGK014 (26%); ¹H NMR (400 MHz, CDCl₃) δ 8.62 (s, 1H), 7.66 (d, J=8.4 Hz, 2H), 7.35 (s, 1H), 7.16 (d, J=8.4 Hz, 2H), 4.99 (d, J=7.6 Hz, 1H), 4.56-4.62 (m, 1H), 4.36-4.42 (m, 4H), 3.73 (s, 3H), 3.03-3.15 (m, 2H), 1.43 (s, 9H); ¹³C NMR (125 MHz, CDCl₃) δ 172.4, 156.5, 155.2, 153.6, 149.1, 146.3, 143.8, 137.6, 131.6, 129.7, 121.7, 113.8, 110.3, 106.6, 80.0, 64.4, 64.2, 54.4, 52.2, 37.6, 28.3; HRMS-ESI [M+H]⁺ found 481.2080 [calcd for C₂₅H₂₈N₄O₆ 480.2003].

### JGK021

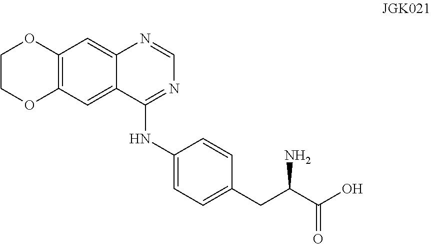 [SMILES: N[C@H](Cc1ccc(Nc2ncnc3cc4c(cc23)OCCO4)cc1)C(=O)O]

Preparation of JGK021 was followed by synthetic procedure of JGK023; ¹H NMR (400 MHz, CDCl₃) δ 8.62 (s, 1H), 8.12 (s, 1H), 7.71 (d, J=8.4 Hz, 2H), 7.40 (d, J=8.4 Hz, 2H), 7.24 (s, 1H), 4.48-4.50 (m, 2H), 4.42-4.44 (m, 2H), 4.28 (t, J=6.8 Hz, 1H), 3.32-3.37 (m, 1H), 3.20 (dd, J=7.6, 14.8 Hz, 1H); ¹³C NMR (125 MHz, CDCl₃) δ 169.7, 159.1, 152.4, 148.8, 146.0, 136.1, 134.1, 133.1, 129.7, 129.7, 124.8, 124.8, 110.0, 108.1, 104.9, 65.2, 64.2, 53.6, 35.4; HRMS-ESI [M+H]⁺ found 367.1347 [calcd for C₁₉H₁₈N₄O₄ 366.1322].

### Preparation of JGK022

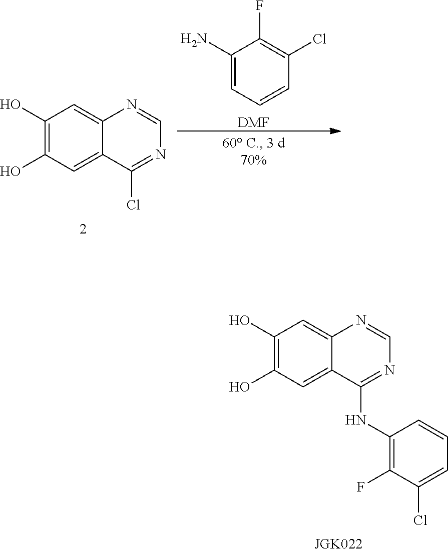

To a solution of diol X (121 mg, 0.6155 mmol) in DMF (3.0 mL) was dropwise added 3-chloro-2-fluoroaniline (0.14 mL) at room temperature. After being heated at 60° C. (bath temperature) with stirring for 3 d, the reaction mixture was cooled to room temperature and diluted with Et₂O (30.0 mL) to give white suspension. The resulting white solid were washed successively with Et₂O (3×50 mL) and CH₂Cl₂ (2×30 mL) and collected to give JGK022 (132 mg, 70%); ¹H NMR (400 MHz, DMSO-d₆) δ 11.16 (br, 1H), 10.43 (br, 1H), 8.68 (s, 1H), 7.91 (s, 1H), 7.58 (t, J=7.1 Hz, 1H), 7.48 (t, J=6.8 Hz, 1H), 7.40 (s, 1H), 7.30 (t, J=8.1 Hz, 1H); ¹³C NMR (125 MHz, DMSO-d₆) δ 159.1, 156.4, 154.1, 152.1, 149.1, 148.3, 135.1, 129.7, 128.1, 126.8, 125.7, 120.8, 107.4, 106.9, 102.9; HRMS-ESI [M+H]⁺ found 306.0437 [calcd for C₁₄H₉ClFN₃O₂ 305.0361].

### Preparation of JGK018

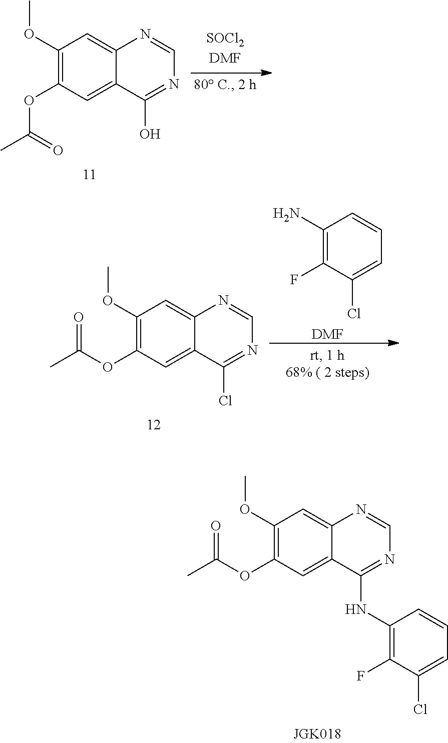

[Chlorination]

To a solution of 11 (500 mg, 2.134 mmol) in thionyl chloride (7.5 mL, 0.28 M) was dropwise added dimethylformamide (0.15 mL). After being heated at 80° C. (bath temperature) with stirring for 2 h, the reaction mixture was cooled to room temperature and concentrated in vacuo. The residue was washed successively with Et₂O (200 mL), and immediately used to the next step.

[Substitution]

To a solution of above generated chloroquinazoline 12 in anhydrous DMF (11 mL, 0.2 M) was dropwise added 3-chloro-2-fluoroaniline (0.50 mL, 4.548 mmol) at room temperature under Ar. After being stirred at the same temperature for 1 h, the reaction mixture was diluted with Et₂O (100.0 mL) to give white suspension. The resulting white solid were washed successively with Et₂O (2×50 mL) and collected to give JGK018 (525 mg, 68%); The spectroscopic data was matched with Zhang, X. et al J. Med. Chem. 2015, 58, 8200-8215.

### Preparation of 13

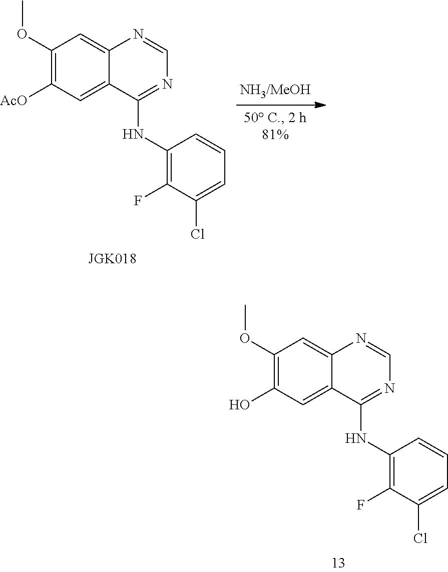 [SMILES: COc1cc2ncnc(Nc3cccc(Cl)c3F)c2cc1OC(C)=O ; COc1cc2ncnc(Nc3cccc(Cl)c3F)c2cc1O]

[Acetyl Deprotection]

To JGK018 (550 mg, 1.520 mmol) was dropwise added ammonia solution (8.0 mL, 7 N in methanol). After being heated at 50° C. (bath temperature) in sealed tube with stirring for 2 h, the reaction mixture was cooled to room temperature and concentrated in vacuo. The resulting white solid were washed successively with Et₂O (2×50 mL) and collected to give 13 (394 mg, 81%); The resulting spectroscopic data was matched with that of Zhang, X. et al J. Med. Chem. 2015, 58, 8200-8215.

### Preparation of 14

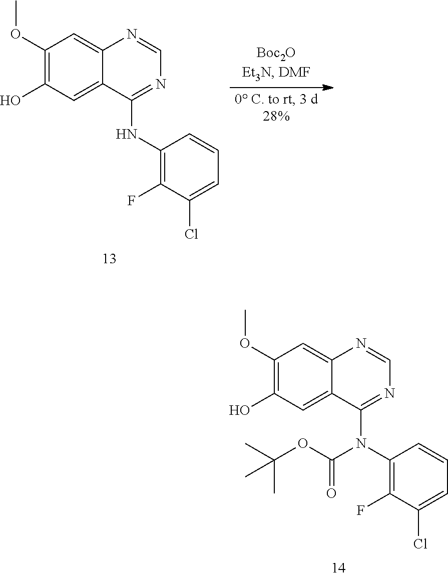 [SMILES: COc1cc2ncnc(Nc3cccc(Cl)c3F)c2cc1O ; COc1cc2ncnc(N(C(=O)OC(C)(C)C)c3cccc(Cl)c3F)c2cc1O]

To a cooled (0° C.) solution of 13 (113 mg, 0.353 mmol) in anhydrous DMF (2 mL) was dropwise added triethylamine (0.25 mL, 1.767 mmol) followed by dropwise addition of Di-tert-butyl dicarbonate (62 mg, 0.459 mmol) in anhydrous DMF (2 mL) under Ar. After being stirred at room temperature for 3 d, the reaction mixture was quenched with saturated aqueous H₂O (10 mL), and diluted with EtOAc (10 mL). The layers were separated, and the aqueous layer was extracted with EtOAc (2×50 mL). The combined organic layers were washed successively with H₂O and saturated brine, dried over anhydrous MgSO₄, filtered, and concentrated in vacuo. The residue was purified by column chromatography (silica gel, hexanes/EtOAc, 5/1 to 3/1) to give 14 (42 mg, 28% isolated yield); ¹H NMR (400 MHz, CDCl₃) δ 8.69 (s, 1H), 8.39-8.45 (m, 1H), 7.64 (s, 1H), 7.44 (s, 1H), 7.11-7.16 (m, 2H), 3.90 (s, 3H), 1.59 (s, 9H); ¹³C NMR (125 MHz, CDCl₃) δ 156.2 (J=39.5 Hz), 154.9, 151.3, 150.3, 149.5 (J=224.1 Hz), 140.4, 128.1 (J=9.6 Hz), 124.8, 124.4 (J=4.8 Hz), 121.5, 120.8 (J=51.2 Hz), 113.5, 109.0, 108.8, 84.6, 56.2, 27.6;

### Preparation of C

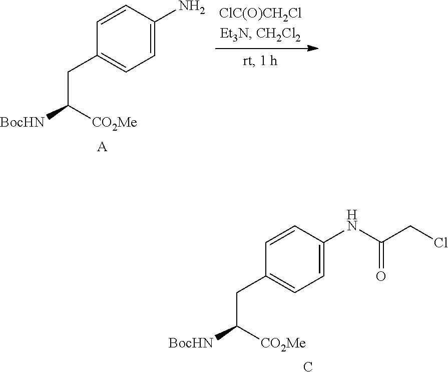

To a solution of A¹ (56 mg, 0.1904 mmol) in anhydrous CH₂Cl₂ (2 mL) was dropwise added triethylamine (0.08 mL, 0.5712 mmol) followed by addition of chloroacetyl chloride (0.05 mL, 0.6286 mmol) at room temperature under Ar. After being stirred at the same temperature for 1 h, the reaction mixture was quenched with saturated aqueous NH₄Cl (20 mL), and diluted with CH₂Cl₂ (20 mL). The layers were separated, and the aqueous layer was extracted with CH₂Cl₂ (2×30 mL). The combined organic layers were washed successively with H₂O and saturated brine, dried over anhydrous MgSO₄, filtered, and concentrated in vacuo. The crude product was used for the next step without further purification.

### Preparation of JGK031

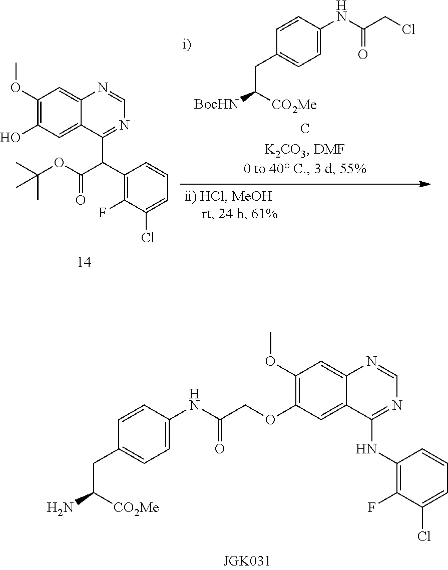

[Alkylation]

To a cooled (0° C.) solution of 14 (44 mg, 0.1058 mmol) in DMF (2.0 mL) was added in one portion potassium carbonate (73 mg, 0.528 mmol) followed by dropwise addition of above generated C (0.1904 mmol) in DMF (2.0 mL) at room temperature. After being heated at 40° C. (bath temperature) with stirring for 3 d, the reaction mixture was quenched with H₂O (10 mL), and diluted with EtOAc (10 mL). The layers were separated, and the aqueous layer was extracted with EtOAc (2×50 mL). The combined organic layers were washed successively with H₂O and saturated brine, dried over anhydrous MgSO₄, filtered, and concentrated in vacuo. The residue was purified by column chromatography (silica gel, hexanes/EtOAc, 100/1 to 30/1) to give alkylated product 14-(2) (43 mg, 55% isolated yield); ¹H NMR (400 MHz, CDCl₃) δ 8.16 (s, 1H), 7.48 (d, J=8.4 Hz, 2H), 7.44 (s, 1H), 6.98-7.13 (m, 5H), 6.34 (m, 1H), 4.95 (d, J=7.4 Hz, 1H), 4.54 (d, J=6.5 Hz, 1H), 4.48 (s, 2H), 3.69 (s, 6H), 2.95-3.13 (m, 2H), 1.53 (s, 9H), 1.40 (s, 9H).

[Deprotection]

To a solution of alkylated product 14-(2) (26 mg, 0.0348 mmol) in anhydrous MeOH (5.0 mL) was dropwise added 0.5 N hydrochloride solution (0.5 mL). After being stirred at the same temperature for 24 h, the reaction mixture was concentrated in vacuo. The residue was purified by column chromatography (reverse phase silica gel, MeOH or MeOH/H₂O, 10/1) to give JGK031 (12 mg, 61%); ¹H NMR (400 MHz, MeOD) δ 8.20 (s, 1H), 7.67 (s, 1H), 7.49 (t, J=7.9 Hz, 2H), 7.24-7.30 (m, 1H), 7.11-7.21 (m, 4H), 3.94 (s, 3H), 3.59 (s, 3H), 2.83-3.01 (m, 2H); HRMS-ESI [M+H]⁺ found 554.1631 [calcd for C₂₇H₂₅ClFN₅O₅ 553.1522].

### Preparation of JGK033

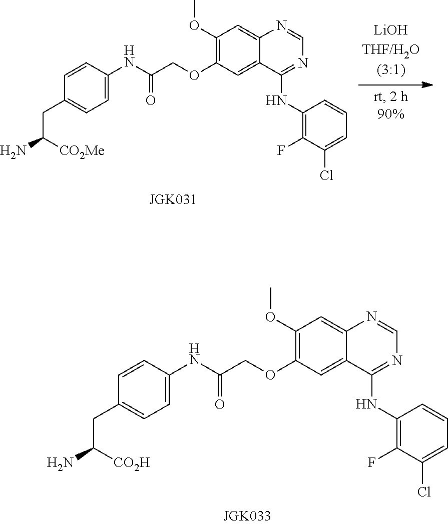 [SMILES: COC(=O)[C@@H](N)Cc1ccc(NC(=O)COc2cc3c(Nc4cccc(Cl)c4F)ncnc3cc2OC)cc1 ; COc1cc2ncnc(Nc3cccc(Cl)c3F)c2cc1OCC(=O)Nc1ccc(C[C@H](N)C(=O)O)cc1]

To a solution of JGK031 (5 mg, 0.009026 mmol) in anhydrous THF (6 mL) and H₂O (2 mL) was added lithium hydroxide.H₂O (3 mg) in one portion. After being stirred at room temperature for 2 h, the reaction mixture was neutralized with 1N hydrochloride solution and concentrated in vacuo. The residue was purified by reverse column chromatography (reverse phase silica gel, MeOH/H₂O, 5/1) to give JGK033 (4.4 mg, 90%); ¹H NMR (400 MHz, MeOD) δ 7.16 (s, 1H), 6.32 (s, 1H), 6.03 (d, J=8.2 Hz, 2H), 5.89-5.98 (m, 2H), 5.59-5.79 (m, 4H), 4.00 (s, 2H), 2.51 (s, 3H), 2.46 (t, J=5.6 Hz, 1H); ¹³C NMR (125 MHz, MeOD) δ 163.9, 159.4, 157.2, 154.3, 152.1, 149.5, 137.1, 135.4, 129.7, 126.9, 124.6, 121.5, 120.3, 108.0, 106.9, 97.8, 56.6, 53.5, 35.6; HRMS-ESI [M+H]⁺ found 540.1435 [calcd for C₂₆H₂₃ClFN₅O₅539.1366].

### Preparation of JGK008

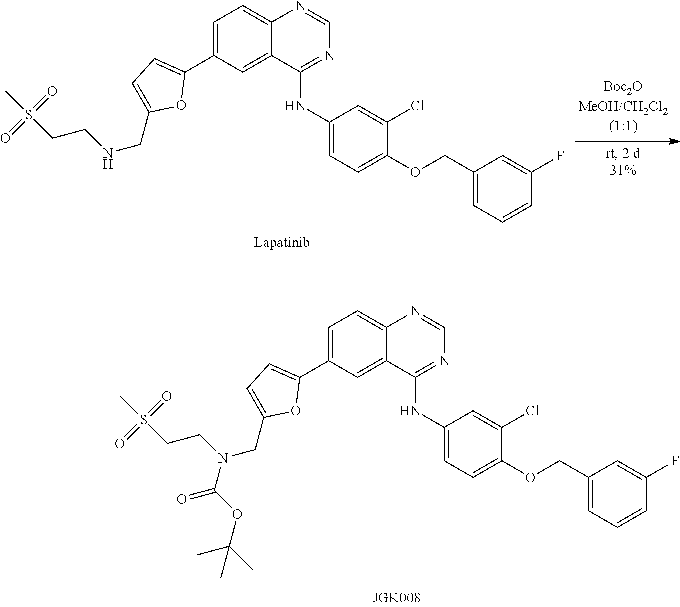 [SMILES: CS(=O)(=O)CCNCc1ccc(-c2ccc3ncnc(Nc4ccc(OCc5cccc(F)c5)c(Cl)c4)c3c2)o1 ; CC(C)(C)OC(=O)N(CCS(C)(=O)=O)Cc1ccc(-c2ccc3ncnc(Nc4ccc(OCc5cccc(F)c5)c(Cl)c4)c3c2)o1]

To a solution of lapatinib (326 mg, 0.561 mmol) in anhydrous MeOH (5.6 mL) and CH₂Cl₂ (5.6 mL) was dropwise Di-tert-butyl dicarbonate (378 mg, 2.819 mmol) in one portion under Ar. After being stirred at room temperature for 2 d, the reaction mixture was quenched with saturated aqueous H₂O (10 mL), and diluted with CH₂Cl₂ (10 mL). The layers were separated, and the aqueous layer was extracted with CH₂Cl₂ (5×50 mL). The combined organic layers were washed successively with H₂O and saturated brine, dried over anhydrous MgSO₄, filtered, and concentrated in vacuo. The residue was purified by column chromatography (silica gel, hexanes/EtOAc, 3/1 to 1/1) to give JGK008 (119 mg, 31% isolated yield); ¹H NMR (500 MHz, CDCl₃) δ 8.71 (bs, 1H), 8.64 (s, 1H), 8.44 (s, 1H), 7.85-7.95 (m, 3H), 7.68 (d, J=7.8 Hz, 1H), 7.32-7.37 (m, 1H), 7.21 (dd, J=8.4, 10.8 Hz, 2H), 6.98-7.03 (m, 1H), 6.96 (d, J=8.9 Hz, 1H), 6.39 (d, J=47.1 Hz, 1H), 5.13 (s, 2H), 4.53 (d, J=32.2 Hz, 2H), 3.99 (t, J=7.3 Hz, 2H), 3.39 (d, J=66.1 Hz, 2H), 2.89 (s, 3H), 1.49 (s, 9H); ¹³C NMR (125 MHz, CDCl₃) δ 163.9, 162.0, 158.0, 154.2, 152.7, 151.7, 151.0, 139.1 (d, J=7.3 Hz), 132.4, 130.1 (d, J=8.1 Hz), 129.1, 128.6, 128.2, 125.1, 123.1, 122.4, 122.3, 115.4, 114.9 (d, J=21.0 Hz), 114.1 (d, J=13.9 Hz), 113.9, 111.5, 110.9, 107.7, 81.3, 70.9, 45.0, 43.6, 42.3, 41.4, 41.2, 28.4; HRMS-ESI [M+H]⁺ found 681.1946 [calcd for C₃₄H₃₄ClFN₄O₆S 680.1866].

### Preparation of JGK011

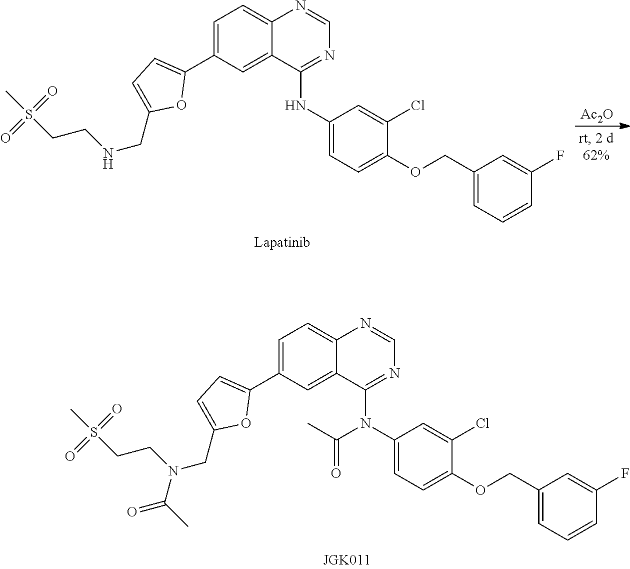 [SMILES: "Ac2O" ; CS(=O)(=O)CCNCc1ccc(-c2ccc3ncnc(Nc4ccc(OCc5cccc(F)c5)c(Cl)c4)c3c2)o1 ; CC(=O)N(CCS(C)(=O)=O)Cc1ccc(-c2ccc3ncnc(N(C(C)=O)c4ccc(OCc5cccc(F)c5)c(Cl)c4)c3c2)o1]

To a solid of lapatinib (68 mg, 0.353 mmol) was added acetic anhydride (5.0 mL) under Ar. After being stirred at room temperature for 2 d, the reaction mixture was concentrated in vacuo. The residue was purified by column chromatography (silica gel, hexanes/EtOAc, 3/1 to 1/3) to give JGK002 (48 mg, 62% isolated yield); ¹H NMR (500 MHz, CDCl₃) δ 9.18 (s, 1H), 8.10-8.17 (m, 3H), 7.50 (d, J=2.5 Hz, 1H), 7.27-7.36 (m, 2H), 7.18 (dd, J=7.6, 13.8 Hz, 2H), 6.97-7.03 (m, 2H), 6.79 (d, J=3.3 Hz, 1H), 6.44 (d, J=3.3 Hz, 1H), 5.14 (s, 2H), 4.66 (s, 2H), 3.86 (t, J=6.6 Hz, 2H), 3.30 (t, J=6.6 Hz, 2H), 2.95 (s, 3H), 2.33 (s, 3H), 2.23 (s, 3H); ¹³C NMR (125 MHz, CDCl₃) δ 171.4, 171.2, 163.9, 162.2, 162.0, 154.0, 153.7, 152.5, 151.0, 138.5 (J=7.3 Hz), 134.2, 130.8, 130.3, 130.2, 129.9, 129.1, 127.4, 123.9, 122.4 (J=2.9 Hz), 121.8, 118.0, 115.1, 115.0, 114.0 (J=5.2 Hz), 113.8, 111.1, 108.9, 70.1 (J=1.7 Hz), 52.3, 47.0, 41.4, 40.7, 23.7, 21.8; HRMS-ESI [M+H]⁻ found 665.1628 [calcd for C₃₃H₃₀ClFN₄O₆S 664.1553].

### Example 2: Preparation of Further Exemplary Compounds of the JGK Series

### General Chemistry Information

All chemicals, reagents, and solvents were purchased from commercial sources when available and were used as received. When necessary, reagents and solvents were purified and dried by standard methods. Air- and moisture-sensitive reactions were carried out under an inert atmosphere of argon in oven-dried glassware. Microwave-irradiated reactions were carried out in a single mode reactor CEM Discover microwave synthesizer. Room temperature reactions were carried out at ambient temperature (approximately 23° C.). All reactions were monitored by thin layer chromatography (TLC) on precoated Merck 60 F₂₅₄ silica gel plates with spots visualized by UV light (λ=254, 365 nm) or by using an alkaline KMnO₄ solution. Flash column chromatography (FC) was carried out on SiO₂ 60 (particle size 0.040-0.063 mm, 230-400 mesh). Concentration under reduced pressure (in vacuo) was performed by rotary evaporation at 25-50° C. Purified compounds were further dried under high vacuum or in a desiccator. Yields correspond to purified compounds, and were not further optimized. Proton nuclear magnetic resonance (¹H NMR) spectra were recorded on Bruker spectrometers (operating at 300, 400, or 500 MHz). Carbon NMR (¹³C NMR) spectra were recorded on Bruker spectrometers (either at 400 or 500 MHz). NMR chemical shifts (δ ppm) were referenced to the residual solvent signals. ¹H NMR data are reported as follows: chemical shift in ppm; multiplicity (s=singlet, d=doublet, t=triplet, q=quartet, quint=quintet, m=multiplet/complex pattern, td=triplet of doublets, ddd=doublet of doublet of doublets, br=broad signal); coupling constants (J) in Hz, integration. Data for ¹³C NMR spectra are reported in terms of chemical shift, and if applicable coupling constants. High resolution mass (HRMS) spectra were recorded on a Thermo Fisher Scientific Exactive Plus with IonSense ID-CUBE DART source mass spectrometer. Compounds 4-chloro-7,8-dihydro[1,4]dioxino[2,3-g]quinazoline (1), 4-chloroquinazoline-6,7-diol (2), and JGK010 were prepared as previously reported.

General Procedure A for the Synthesis of 4-Anilinoquinazoline Compounds JGK035-JGK041, and JKG043.

A mixture of the 4-chloroquinazoline (1 equiv) in iPrOH (0.1-0.3 M) was treated with the aniline (1 equiv), and the mixture was heated at 80° C. under microwave irradiation (60 W) for 15-20 min. The mixture was cooled to 23° C., treated with additional aniline (1 equiv), and again subjected to microwave irradiation (80° C., 60 W, 15-20 min). The mixture was either concentrated under reduced pressure, or the precipitated 4-anilinoquinazoline hydrochloride salt was isolated by filtration (washings with cold iPrOH). The residue was suspended in sat. aq. NaHCO₃, and extracted with CH₂Cl₂ (3×). The combined organic extracts were washed with water, brine, dried (Na₂SO₄), filtered, and concentrated. Purification by FC (elution with a gradient of CH₂Cl₂/EtOAc or hexanes/EtOAc) afforded the desired products typically as white to off-white, or pale-yellow solids.

### N-(2-Fluorophenyl)-7,8-dihydro[1,4]dioxino[2,3-g]quinazolin-4-amine (JGK035)

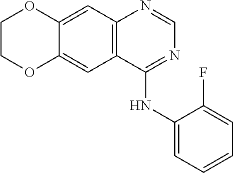 [SMILES: Fc1ccccc1Nc1ncnc2cc3c(cc12)OCCO3]

Following general procedure A, compound JGK035 was prepared from 4-chloroquinazoline 1 (51 mg, 0.23 mmol) and 2-fluoroaniline (40 μL, 0.48 mmol) in iPrOH (1.5 mL). FC (CH₂Cl₂/EtOAc 10:1→10:4) gave JGK035 (56 mg, 82%) as a white solid. ¹H NMR (500 MHz, CDCl₃): δ8.68 (s, 1H), 8.64 (td, J=8.2, 1.7 Hz, 1H), 7.38 (s, 1H), 7.36 (br, 1H), 7.31 (s, 1H), 7.22 (t, J=7.5 Hz, 1H), 7.17 (ddd, J=11.2, 8.3, 1.5 Hz, 1H), 7.10-7.05 (m, 1H), 4.44-4.37 ppm (m, 4H). ¹³C NMR (126 MHz, CDCl₃): δ156.08, 153.60, 153.50 (d, J=242.7 Hz), 149.52, 146.65, 144.34, 127.31 (d, J=9.5 Hz), 124.66 (d, J=3.7 Hz), 123.97 (d, J=7.8 Hz), 122.89, 115.06 (d, J=19.3 Hz), 114.46, 110.62, 106.10, 64.69, 64.51 ppm. HRMS (DART): m/z [M−H]⁻ calcd for C₁₆H₁₁FN₃O₂⁻, 296.0841; found, 296.0841.

### 4-Chloro(7,7,8,8-²H₄)-7,8-dihydro[1,4]dioxino[2,3-g]quinazoline (3)

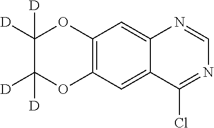 [SMILES: [2H]C1([2H])Oc2cc3ncnc(Cl)c3cc2OC1([2H])[2H]]

A solution of compound 2 (193 mg, 0.98 mmol) in dry DMF (4.8 mL) was treated with Cs₂CO₃ (788 mg, 2.42 mmol), stirred for 5 min, and treated dropwise with 1-bromo-2-chloro(²H₄)ethane (270 μL, 3.16 mmol). The mixture was stirred at 23° C. for 1 h, and then at 70° C. for 18 h. After the mixture was cooled to 23° C., all volatiles were removed in vacuo. The residue was dissolved in CH₂Cl₂ (40 mL), washed with water (2×13 mL), brine (13 mL), dried (Na₂SO₄), filtered, and evaporated. Purification by FC (CH₂Cl₂/EtOAc 1:0→10:1.5) afforded the title compound 3 (109 mg, 49%) as a white fluffy solid. ¹H NMR (400 MHz, CDCl₃): δ8.84 (s, 1H), 7.64 (s, 1H), 7.47 ppm (s, 1H). ¹³C NMR (101 MHz, CDCl₃): δ160.19, 152.52, 151.54, 147.93, 146.06, 120.10, 113.72, 110.83 ppm (two upfield carbons not observed). HRMS (DART): m/z [M+H]⁺ calcd for C₁₀H₄D₄ClN₂O₂⁺, 227.0520; found, 227.0516.

### N-(3-Chloro-2-fluorophenyl)(7,7,8,8-²H₄)-7,8-dihydro[1,4]dioxino[2,3-g]quinazolin-4-amine (JGK036)

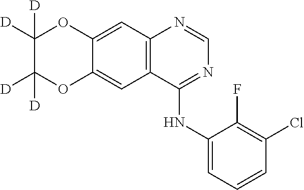 [SMILES: [2H]C1([2H])Oc2cc3ncnc(Nc4cccc(Cl)c4F)c3cc2OC1([2H])[2H]]

Following general procedure A, compound JGK036 was prepared from 4-chloroquinazoline 3 (55 mg, 0.24 mmol) and 3-chloro-2-fluoroaniline (52 μL, 0.47 mmol) in iPrOH (1.2 mL). JGK036.HCl was isolated by filtration from the crude reaction mixture, and after basification and extraction gave pure JGK036 (67 mg, 82%) as a pale-yellow solid. ¹H NMR (500 MHz, DMSO-d₆): δ 9.62 (s, 1H), 8.34 (s, 1H), 7.93 (s, 1H), 7.53-7.43 (m, 2H), 7.27 (td, J=8.1, 1.3 Hz, 1H), 7.19 ppm (s, 1H). ¹³C NMR (126 MHz, DMSO-d₆): δ 157.17, 153.10, 152.45 (d, J=249.2 Hz), 149.28, 146.04, 143.68, 128.21 (d, J=12.0 Hz), 127.27, 127.03, 124.87 (d, J=4.7 Hz), 120.11 (d, J=16.7 Hz), 112.48, 109.64, 108.35, 63.50 (m, 2C's). HRMS (DART): m/z [M+H]⁺ calcd for C₁₆H₈D₄ClFN₃O₂⁺, 336.0848; found, 336.0841.

### N-(3-Bromo-2-fluorophenyl)-7,8-dihydro[1,4]dioxino[2,3-g]quinazolin-4-amine (JGK037)

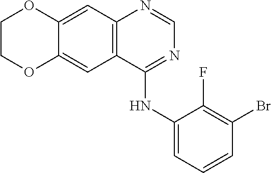 [SMILES: Fc1c(Br)cccc1Nc1ncnc2cc3c(cc12)OCCO3]

Following general procedure A, compound JGK037 was prepared from 4-chloroquinazoline 1 (100 mg, 0.45 mmol) and 3-bromo-2-fluoroaniline (100 μL, 0.89 mmol) in iPrOH (1.5 mL). FC (CH₂Cl₂/EtOAc 10:0→10:3) gave JGK037 (150 mg, 89%) as a pale-yellow solid. ¹H NMR (500 MHz, CDCl₃): δ68.68 (s, 1H), 8.65 (ddd, J=8.3, 7.4, 1.5 Hz, 1H), 7.39 (s, 1H), 7.35 (br, 1H), 7.29 (s, 1H), 7.29-7.24 (m, 1H), 7.11 (td, J=8.2, 1.6 Hz, 1H), 4.44-4.38 ppm (m, 4H). ¹³C NMR (126 MHz, CDCl₃): δ155.89, 153.37, 150.15 (d, J=242.2 Hz), 149.70, 146.75, 144.53, 128.65 (d, J=10.5 Hz), 127.24, 125.31 (d, J=4.7 Hz), 121.79, 114.53, 110.59, 108.59 (d, J=19.4 Hz), 105.93, 64.70, 64.51 ppm. HRMS (DART): m/z [M−H]⁻ calcd for C₁₆H₁₀BrFN₃O₂⁻, 373.9946; found, 373.9946.

### N-{2-Fluoro-3-[(triethylsilyl)ethynyl]phenyl}-7,8-dihydro[1,4]dioxino[2,3-g]quinazolin-4-amine (4)

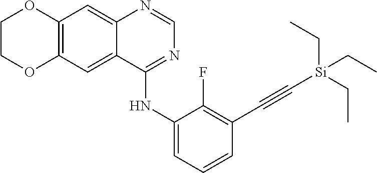 [SMILES: CC[Si](C#Cc1cccc(Nc2ncnc3cc4c(cc23)OCCO4)c1F)(CC)CC]

A 1 dram vial was charged with JGK010 (75 mg, 0.23 mmol), XPhos (19.7 mg, 0.041 mmol), Cs₂CO₃ (195 mg, 0.60 mmol), [PdCl₂.(MeCN)₂] (3.6 mg, 0.014 mmol). The vial was evacuated and backfilled with argon (repeated at least twice). Dry acetonitrile (1 mL) was added, and the orange suspension was stirred at 23° C. for 25 min, then ethynyltriethylsilane (150 μL, 0.84 mmol) was injected. The tube was sealed, and the reaction mixture stirred at 95° C. in a preheated oil bath for 3.5 h. The suspension was allowed to reach 23° C., diluted with EtOAc, filtered through a plug of SiO₂ (washings with EtOAc), and evaporated. Purification by FC (SiO₂; hexanes/EtOAc 8:2→4:6) afforded the title compound 4 (48 mg, 49%) as a yellow foamy solid. ¹H NMR (500 MHz, CDCl₃): δ8.681 (td, J=8.1, 1.9 Hz, 1H), 8.678 (s, 1H), 7.382 (s, 1H), 7.376 (br, 1H), 7.28 (s, 1H), 7.21-7.12 (m, 2H), 4.44-4.38 (m, 4H), 1.07 (t, J=7.9 Hz, 9H), 0.71 ppm (q, J=7.9 Hz, 6H). ¹³C NMR (126 MHz, CDCl₃): δ 155.95, 153.81 (d, J=248.0 Hz), 153.44, 149.62, 146.66, 144.47, 127.68, 127.60, 124.15 (d, J=4.5 Hz), 122.79, 114.49, 111.77 (d, J=14.6 Hz), 110.61, 105.97, 98.65, 98.49 (d, J=3.7 Hz), 64.70, 64.51, 7.63, 4.50 ppm. HRMS (DART): m/z [M+H]⁺ calcd for C₂₄H₂₇N₃O₂Si⁺, 436.1851; found, 436.1831.

### N-(3-Ethynyl-2-fluorophenyl)-7,8-dihydro[1,4]dioxino[2,3-g]quinazolin-4-amine (JGK038)

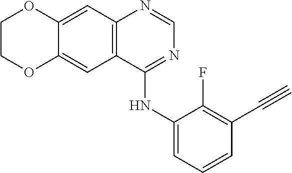 [SMILES: C#Cc1cccc(Nc2ncnc3cc4c(cc23)OCCO4)c1F]

A mixture of compound 4 (40 mg, 0.09 mmol) in wet THF (0.9 mL) was treated dropwise with a 1 M solution of TBAF in THF (450 μL, 0.45 mmol), and the mixture was stirred at 23° C. for 18 h. Water (10 mL) was added, and the mixture was extracted with EtOAc (3×15 mL). The combined organics were washed with brine (20 mL), dried (Na₂SO₄), filtered, and evaporated. Purification by FC (SiO₂; hexanes/EtOAc 7:3→3:7), followed by a second FC (SiO₂; CH₂Cl₂/EtOAc 1:0→6:4) afforded JGK038 (19 mg, 64%) as an off-white solid. ¹H NMR (500 MHz, CDCl₃): δ8.69 (td, J=8.0, 1.8 Hz, 1H), 8.67 (s, 1H), 7.38 (s, 1H), 7.36 (br, 1H), 7.29 (s, 1H), 7.24-7.15 (m, 2H), 4.43-4.38 (m, 4H), 3.34 ppm (s, 1H). ¹³C NMR (126 MHz, CDCl₃): δ 155.94, 154.04 (d, J=248.8 Hz), 153.39, 149.65, 146.70, 144.47, 127.81, 127.68 (d, J=9.1 Hz), 124.30 (d, J=4.7 Hz), 123.47, 114.49, 110.58, 110.50 (d, J=14.3 Hz), 105.99, 82.95 (d, J=3.5 Hz), 76.70 (d, J=1.6 Hz), 64.69, 64.50 ppm. HRMS (DART): m/z [M+H]⁺ calcd for C₁₈H₁₃FN₃O₂⁺, 322.0986; found, 322.0981.

### N-[2-Fluoro-3-(trifluoromethyl)phenyl]-7,8-dihydro[1,4]dioxino[2,3-g]quinazolin-4-amine (JGK039)

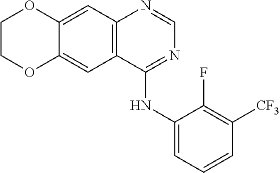 [SMILES: Fc1c(Nc2ncnc3cc4c(cc23)OCCO4)cccc1C(F)(F)F]

Following general procedure A, compound JGK039 was prepared from 4-chloroquinazoline 1 (37 mg, 0.17 mmol) and 2-fluoro-3-(trifluoromethyl)aniline (42 μL, 0.33 mmol) in iPrOH (1.5 mL). FC (CH₂Cl₂/EtOAc 1:0→10:3) gave JGK039 (35 mg, 58%) as an off-white solid. ¹H NMR (500 MHz, CDCl₃): δ9.00-8.92 (m, 1H), 8.70 (s, 1H), 7.42 (br, 1H), 7.40 (s, 1H), 7.35-7.28 (m, 2H), 7.30 (s, 1H), 4.46-4.38 ppm (m, 4H). ¹³C NMR (126 MHz, CDCl₃): δ155.77, 153.24, 150.27 (d, J=252.0 Hz), 149.81, 146.80, 144.66, 128.62 (d, J=8.5 Hz), 126.34, 124.44, 124.40, 122.66 (q, J=272.4 Hz), 120.41 (q, J=4.6 Hz), 114.58, 110.55, 105.86, 64.70, 64.51 ppm. HRMS (DART): m/z [M−H]⁻ calcd for C₁₇H₁₀F₄N₃O₂⁻, 364.0715; found, 364.0712.

### 4-Chloro-8,9-dihydro-7H-[1,4]dioxepino[2,3-g]quinazoline (5)

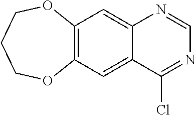 [SMILES: Clc1ncnc2cc3c(cc12)OCCCO3]

A solution of compound 2 (100 mg, 0.51 mmol) in dry DMF (10 mL) was treated with Cs₂CO₃ (460 mg, 1.41 mmol), stirred for 15 min, and treated dropwise with 1,3-dibromopropane (135 ML, 1.33 mmol). The mixture was stirred at 23° C. for 1 h, and then at 65° C. for 18 h. After cooling to 23° C., all volatiles were removed in vacuo. The residue was suspended in CH₂Cl₂ (20 mL), and washed with water (2×5 mL), dried (Na₂SO₄), filtered, and evaporated. Purification by FC (hexanes/CH₂Cl₂ 1:10→0:1→CH₂Cl₂/EtOAc 10:1.5), followed by a second FC (hexanes/EtOAc 10:1→10:3) gave the title compound 5 (41 mg, 34%) as a white solid. ¹H NMR (500 MHz, CDCl₃): δ8.87 (s, 1H), 7.75 (s, 1H), 7.55 (s, 1H), 4.46 (t, J=5.8 Hz, 2H), 4.40 (t, J=6.0 Hz, 2H), 2.34 ppm (quint, J=5.9 Hz, 2H). ¹³C NMR (126 MHz, CDCl₃): δ 160.57, 158.86, 153.10, 153.03, 148.88, 120.83, 118.19, 115.64, 70.51, 70.33, 30.51 ppm. HRMS (DART): m/z [M+H]⁺ calcd for C₁₁H₁₀ClN₂O₂⁺, 237.0425; found, 237.0416.

### N-(3-Chloro-2-fluorophenyl)-8,9-dihydro-7H-[1,4]dioxepino[2,3-g]quinazolin-4-amine (JGK040)

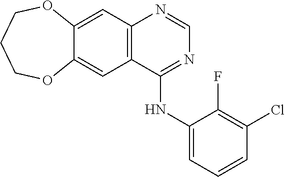 [SMILES: Fc1c(Cl)cccc1Nc1ncnc2cc3c(cc12)OCCCO3]

Following general procedure A, compound JGK040 was prepared from 4-chloroquinazoline 5 (33 mg, 0.14 mmol) and 3-chloro-2-fluoroaniline (32 μL, 0.29 mmol) in iPrOH (1.5 mL). FC (CH₂Cl₂/EtOAc 1:0→10:3.5) gave JGK040 (34 mg, 70%) as a white solid. ¹H NMR (500 MHz, DMSO-d₆): δ9.72 (s, 1H), 8.39 (s, 1H), 8.08 (s, 1H), 7.53-7.45 (m, 2H), 7.29 (s, 1H), 7.27 (td, J=8.1, 1.3 Hz, 1H), 4.32 (t, J=5.5 Hz, 2H), 4.29 (t, J=5.6 Hz, 2H), 2.22 ppm (quint, J=5.6 Hz, 2H). ¹³C NMR (126 MHz, DMSO-d₆): δ157.43, 156.67, 153.85, 152.48 (d, J=249.3 Hz), 150.74, 147.29, 128.05 (d, J=12.0 Hz), 127.41, 127.04, 124.91 (d, J=4.7 Hz), 120.13 (d, J=16.5 Hz), 117.52, 113.81, 110.77, 70.75, 70.62, 30.80 ppm. HRMS (DART): m/z [M+H]⁺ calcd for C₁₇H₁₄ClFN₃O₂⁺, 346.0753; found, 346.0740.

### 8-Chloro-2H-[1,3]dioxolo[4,5-g]quinazoline (6)

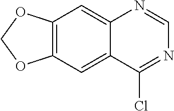 [SMILES: Clc1ncnc2cc3c(cc12)OCO3]

A solution of compound 2 (100 mg, 0.51 mmol) in dry DMF (3.4 mL) was treated with Cs₂CO₃ (335 mg, 1.03 mmol), and stirred at 23° C. for 15 min. The mixture was treated dropwise with chloroiodomethane (130 μL, 1.79 mmol), stirred for 1 h, and then at 70° C. for 17 h. After the mixture was cooled to 23° C., all volatiles were removed in vacuo. The residue was suspended in CH₂Cl₂ (30 mL), washed with water (2×7 mL), dried (Na₂SO₄), filtered, and evaporated. Purification by FC (hexanes/CH₂Cl₂3:10→0:1→CH₂Cl₂/EtOAc 10:2) gave the title compound 6 (38 mg, 36%) as a white, fluffy solid. ¹H NMR (400 MHz, CDCl₃): δ8.85 (s, 1H), 7.49 (s, 1H), 7.32 (s, 1H), 6.21 ppm (s, 2H). ¹³C NMR (126 MHz, CDCl₃): δ159.82, 154.89, 152.79, 150.94, 149.78, 121.23, 105.23, 102.89, 101.12 ppm. HRMS (DART): m/z [M+H]⁺ calcd for C₉H₆ClN₂O₂⁺, 209.0112; found, 209.0104.

### N-(3-Chloro-2-fluorophenyl)-2H-[1,3]dioxolo[4,5-g]quinazolin-8-amine (JGK041)

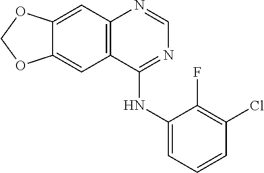 [SMILES: Fc1c(Cl)cccc1Nc1ncnc2cc3c(cc12)OCO3]

Following general procedure A, compound JGK041 was prepared from 4-chloroquinazoline 6 (35 mg, 0.17 mmol) and 3-chloro-2-fluoroaniline (38 μL, 0.35 mmol) in iPrOH (1.5 mL). FC (CH₂Cl₂/EtOAc 1:0-1:1) gave JGK041 (35 mg, 66%) as a pale-yellow solid. ¹H NMR (500 MHz, DMSO-d₆): δ9.53 (s, 1H), 8.37 (s, 1H), 7.84 (s, 1H), 7.53-7.44 (m, 2H), 7.27 (td, J=8.1, 1.3 Hz, 1H), 7.20 (s, 1H), 6.25 ppm (s, 2H). ¹³C NMR (126 MHz, DMSO-d₆): δ157.37, 153.10, 152.60, 152.43 (d, J=248.9 Hz), 148.56, 147.28, 128.30 (d, J=11.9 Hz), 127.17, 126.90, 124.88 (d, J=4.8 Hz), 120.12 (d, J=16.4 Hz), 109.82, 104.59, 102.38, 98.77 ppm. HRMS (DART): m/z [M+H]⁺ calcd for C₁₅H₁₀ClFN₃O₂⁺, 318.0440; found, 318.0435.

### [(3-Chloro-2-fluorophenyl)(7,8-dihydro[1,4]dioxino[2,3-g]quinazolin-4-yl)amino]methyl acetate (JGK043)

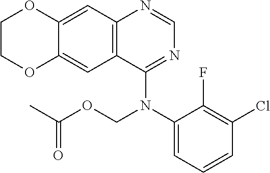 [SMILES: CC(=O)OCN(c1cccc(Cl)c1F)c1ncnc2cc3c(cc12)OCCO3]

A mixture of JGK010 (50 mg, 0.15 mmol) in dry THF (0.5 mL) was treated dropwise with a 1 M solution of LiHMDS in THF (150 μL, 0.15 mmol) at 0° C. After stirring for 15 min at that temperature, the mixture was added dropwise to a solution of chloromethyl acetate (55 μL, 0.57 mmol) in dry THF (0.5 mL). The flask which initially contained the solution of JGK010 was rinsed with 0.5 mL of dry THF and added to the reaction mixture. After stirring at 0° C. for 2 h, stirring was continued at 23° C. for 22 h. Sat. aq. NaHCO₃ (10 mL) were added, and the mixture was extracted with EtOAc (3×10 mL). The combined organics were dried (Na₂SO₄), filtered, and evaporated in vacuo. Purification by FC (CH₂Cl₂/EtOAc 1:0->1:1) afforded the title compound JGK043 (30 mg, 49%) as a pale-yellow solid. ¹H NMR (500 MHz, CDCl₃): δ7.93 (s, 1H), 7.88 (s, 1H), 7.08-6.97 (m, 2H), 6.94 (td, J=7.2, 2.1 Hz, 1H), 6.82 (s, 1H), 5.73 (s, 2H), 4.40-4.29 (m, 4H), 2.12 ppm (s, 3H). ¹³C NMR (126 MHz, CDCl₃): δ 170.33, 152.96, 150.10 (d, J=245.6 Hz), 149.37, 148.05, 143.24, 140.17 (d, J=13.1 Hz), 131.89, 124.17, 124.12 (d, J=4.8 Hz), 122.59 (d, J=2.4 Hz), 121.33 (d, J=17.1 Hz), 115.41, 114.31, 102.24, 71.19, 65.07, 64.23, 20.85 ppm. HRMS (DART): m/z [M+H]⁺ calcd for C₁₉H₁₆ClFN₃O₄⁺, 404.0808; found, 404.0792.

### Example 3: Preparation of Further Exemplary Compounds of the JGK Series

General Procedures:

All chemicals, reagents, and solvents were purchased from commercial sources when available and were used as received. When necessary, reagents and solvents were purified and dried by standard methods. Air- and moisture-sensitive reactions were carried out under an inert atmosphere of argon in oven-dried glassware. Microwave-irradiated reactions were carried out in a single mode reactor CEM Discover microwave synthesizer. Room temperature (RT) reactions were carried out at ambient temperature (approximately 23° C.). All reactions were monitored by thin layer chromatography (TLC) on precoated Merck 60 F₂₅₄ silica gel plates with spots visualized by UV light (Δ=254, 365 nm) or by using an alkaline KMnO₄ solution. Flash column chromatography (FC) was carried out on SiO₂ 60 (particle size 0.040-0.063 mm, 230-400 mesh). Concentration under reduced pressure (in vacuo) was performed by rotary evaporation at 25-50° C. Purified compounds were further dried under high vacuum or in a desiccator. Yields correspond to purified compounds, and were not further optimized. Proton nuclear magnetic resonance (¹H NMR) spectra were recorded on Bruker spectrometers (operating at 300, 400, or 500 MHz). Carbon NMR (¹³C NMR) spectra were recorded on Bruker spectrometers (either at 400 or 500 MHz). NMR chemical shifts (δ ppm) were referenced to the residual solvent signals. ¹H NMR data are reported as follows: chemical shift in ppm; multiplicity (s=singlet, d=doublet, t=triplet, q=quartet, quint=quintet, m=multiplet/complex pattern, td=triplet of doublets, ddd=doublet of doublet of doublets, br=broad signal); coupling constants (J) in Hz, integration. Data for ¹³C NMR spectra are reported in terms of chemical shift, and if applicable coupling constants. High resolution mass (HRMS) spectra were recorded on a Thermo Fisher Scientific Exactive Plus with IonSense ID-CUBE DART source mass spectrometer, or on a Waters LCT Premier mass spectrometer with ACQUITY UPLC with autosampler.

### 3-[(7,8-Dihydro[1,4]dioxino[2,3-g]quinazolin-4-yl)amino]-2-fluorobenzonitrile (JGK044)

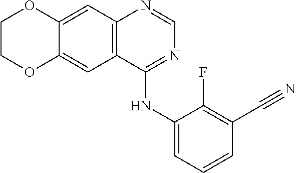 [SMILES: N#Cc1cccc(Nc2ncnc3cc4c(cc23)OCCO4)c1F]

¹H NMR (500 MHz, CDCl₃): δ=9.06-8.98 (m, 1H), 8.69 (s, 1H), 7.41 (s, 1H), 7.39 (br, 1H), 7.35-7.31 (m, 2H), 7.30 (s, 1H), 4.45-4.37 ppm (m, 4H). ¹³C NMR (126 MHz, CDCl₃): δ=155.63, 153.63 (d, J=254.6 Hz), 153.04, 149.94, 146.80, 144.76, 128.60 (d, J=7.8 Hz), 127.48, 126.58, 125.31 (d, J=4.5 Hz), 114.56, 113.80, 110.45, 105.83, 101.30 (d, J=13.9 Hz), 64.70, 64.51 ppm. HRMS (ESI): m/z [M+H]⁺ calcd for C₁₇H₁₂FN₄O₂⁺, 323.0939; found, 323.0927.

### 3-[(7,8-Dihydro[1,4]dioxino[2,3-g]quinazolin-4-yl)amino]benzonitrile (JGK045)

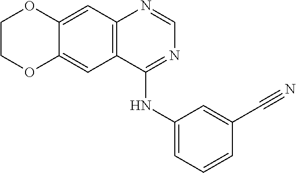 [SMILES: N#Cc1cccc(Nc2ncnc3cc4c(cc23)OCCO4)c1]

¹H NMR (500 MHz, DMSO-d₆): δ=9.68 (s, 1H), 8.52 (s, 1H), 8.46 (t, J=1.9 Hz, 1H), 8.18 (ddd, J=8.2, 2.3, 1.2 Hz, 1H), 8.08 (s, 1H), 7.58 (t, J=7.9 Hz, 1H), 7.53 (dt, J=7.6, 1.4 Hz, 1H), 7.22 (s, 1H), 4.49-4.36 ppm (m, 4H). ¹³C NMR (126 MHz, DMSO-d₆): δ=156.24, 152.69, 149.31, 146.15, 143.80, 140.52, 129.87, 126.35, 125.96, 124.15, 118.93, 112.66, 111.23, 109.96, 108.30, 64.52, 64.19 ppm. HRMS (DART): m/z [M+H]⁺ calcd for C₁₇H₁₃N₄O₂⁺, 305.1033; found, 305.1018.

### Ethyl (3-chloro-2-fluorophenyl)7,8-dihydro[1,4]dioxino[2,3-g]quinazolin-4-ylcarbamate (JGK047)

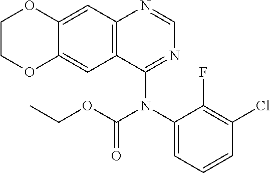 [SMILES: CCOC(=O)N(c1cccc(Cl)c1F)c1ncnc2cc3c(cc12)OCCO3]

¹H NMR (500 MHz, CDCl₃): δ=8.96 (s, 1H), 7.483 (s, 1H), 7.477 (s, 1H), 7.37 (dd, J=8.1, 6.7 Hz, 2H), 7.08 (td, J=8.1, 1.5 Hz, 1H), 4.45-4.38 (m, 4H), 4.27 (q, J=7.1 Hz, 2H), 1.22 ppm (t, J=7.1 Hz, 3H). ¹³C NMR (126 MHz, CDCl₃): 158.98, 154.43 (d, J=250.7 Hz), 153.90, 153.41, 151.17, 149.68, 145.61, 130.12, 129.85 (d, J=12.4 Hz), 128.20, 124.50 (d, J=5.1 Hz), 122.22 (d, J=16.8 Hz), 117.96, 113.51, 109.68 (d, J=2.0 Hz), 64.73, 64.36, 63.44, 14.43.ppm. HRMS (ESI): m/z [M+H]⁺ calcd for C₁₉H₁₆ClFN₃O₄⁻, 404.0808; found, 404.0800.

### (±)-4-(3-Bromo-2-fluoroanilino)-7,8-dihydro[1,4]dioxino[2,3-g]quinazolin-7-ol ((±)-JGK050)

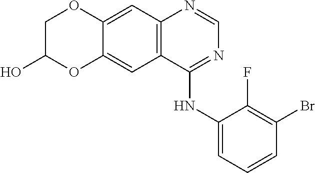 [SMILES: OC1COc2cc3ncnc(Nc4cccc(Br)c4F)c3cc2O1]

¹H NMR (500 MHz, DMSO-d₆): δ=9.61 (s, 1H), 8.34 (s, 1H), 7.91 (s, 1H), 7.72 (d, J=5.4 Hz, 1H), 7.60 (t, J=7.1 Hz, 1H), 7.55 (t, J=7.5 Hz, 1H), 7.21 (t, J=8.2 Hz, 1H), 7.19 (s, 1H), 5.70-5.59 (m, 1H), 4.27 (d, J=11.0 Hz, 1H), 4.17 ppm (d, J=10.6 Hz, 1H). ¹³C NMR (126 MHz, DMSO-d₆): δ=157.18, 153.38 (d, J=247.4 Hz), 153.13, 148.78, 146.14, 141.92, 130.12, 128.05 (d, J=13.8 Hz), 127.78, 125.45 (d, J=4.4 Hz), 111.95, 109.87, 108.71, 108.55 (d, J=20.3 Hz), 88.63, 67.23 ppm. HRMS (DART): m/z [M+H]⁺ calcd for C₁₆H₁₂BrFN₃O₃⁺, 392.0041; found, 392.0030.

### Diastereoisomeric Mixture of (±)-cis- and (±)-trans-N-(3-Bromo-2-fluorophenyl)-7,8-dimethyl-7,8-dihydro[1,4]dioxino[2,3-g]quinazolin-4-amine ((±)-cis/trans-JGK051)

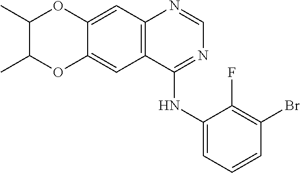 [SMILES: CC1Oc2cc3ncnc(Nc4cccc(Br)c4F)c3cc2OC1C]

¹H NMR (500 MHz, CDCl₃; (±)-cis/trans 2:1): δ=8.68 (s, 1H), 8.68-8.63 (m, 1H), 7.37 (s, 1H), 7.35 (br, 1H), 7.28 (s, 1H), 7.28-7.24 (m, 1H), 7.10 (td, J=8.2, 1.6 Hz, 1H), 4.52-4.39 (m, 1.3H), 4.09-3.98 (m, 0.7H), 1.453 (d, J=6.1 Hz, 1.1H), 1.451 (d, J=6.1 Hz, 1.1H), 1.369 (d, J=6.6 Hz, 1.9H), 1.368 ppm (d, J=6.6 Hz, 1.9H). ¹³C NMR (126 MHz, CDCl₃; (±)-cis/trans 2:1): 6=155.87, 155.84, 153.19, 150.12 (d, J=242.5 Hz), 150.09 (d, J=242.2 Hz), 149.87, 148.89, 146.77, 144.64, 143.63, 128.73 (d, J=10.0 Hz), 127.15, 127.12, 125.31 (d, J=4.7 Hz), 121.71, 121.70, 114.30, 113.93, 110.54, 110.47, 108.57 (d, J=19.4 Hz), 105.66, 105.28 ppm. HRMS (DART): m/z [M+H]⁺ calcd for C₁₈H₁₆BrFN₃O₂⁺, 404.0404; found, 404.0393.

### (±)-cis-N-(3-Bromo-2-fluorophenyl)-7,8-dimethyl-7,8-dihydro[1,4]dioxino[2,3-g]quinazolin-4-amine ((±)-JGK052)

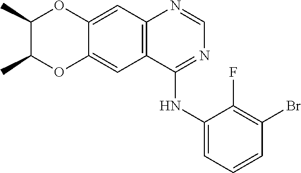 [SMILES: C[C@@H]1Oc2cc3c(Nc4cccc(Br)c4F)ncnc3cc2O[C@@H]1C]

¹H NMR (500 MHz, CDCl₃): δ=8.68 (s, 1H), 8.66 (ddd, J=8.6, 7.4, 1.6 Hz, 1H), 7.38 (s, 1H), 7.35 (br, 1H), 7.28 (s, 1H), 7.30-7.23 (m, 1H), 7.10 (td, J=8.2, 1.5 Hz, 1H), 4.50-4.41 (m, 2H), 1.369 (d, J=6.6 Hz, 3H), 1.368 ppm (d, J=6.5 Hz, 3H). ¹³C NMR (126 MHz, CDCl₃): δ=155.85, 153.19, 150.11 (d, J=242.1 Hz), 148.89, 146.77, 143.63, 128.73 (d, J=10.2 Hz), 127.14, 125.31 (d, J=4.7 Hz), 121.71, 114.30, 110.54, 108.57 (d, J=19.5 Hz), 105.65, 72.85, 72.58, 14.71, 14.55 ppm. HRMS (ESI): m/z [M+H]⁺ calcd for C₁₈H₁₆BrFN₃O₂⁺, 404.0404; found, 404.0416.

### N-(3-Bromo-4-chloro-2-fluorophenyl)-7,8-dihydro[1,4]dioxino[2,3-g]quinazolin-4-amine (JGK053)

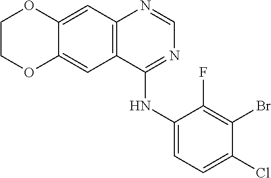 [SMILES: Fc1c(Nc2ncnc3cc4c(cc23)OCCO4)ccc(Cl)c1Br]

¹H NMR (500 MHz, DMSO-d₆): δ=9.70 (s, 1H), 8.35 (s, 1H), 7.94 (s, 1H), 7.61 (dd, J=8.8, 7.7 Hz, 1H), 7.55 (dd, J=8.7, 1.5 Hz, 1H), 7.20 (s, 1H), 4.47-4.35 ppm (m, 4H). ¹³C NMR (126 MHz, DMSO-d₆): δ=157.03, 154.14 (d, J=249.5 Hz), 153.01, 149.36, 146.08, 143.74, 130.75, 127.77 (d, J=2.9 Hz), 126.80 (d, J=13.4 Hz), 125.37 (d, J=3.8 Hz), 112.50, 110.15 (d, J=22.5 Hz), 109.66, 108.39, 64.51, 64.14 ppm. HRMS (DART): m/z [M+H]⁺ calcd for C₁₆H₁₁BrClFN₃O₂⁺, 409.9702; found, 409.9697.

### N-(3,4-Dibromo-2-fluorophenyl)-7,8-dihydro[1,4]dioxino[2,3-g]quinazolin-4-amine (JGK054)

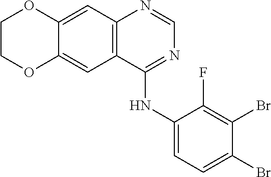 [SMILES: Fc1c(Nc2ncnc3cc4c(cc23)OCCO4)ccc(Br)c1Br]

¹H NMR (500 MHz, DMSO-d₆): δ=9.65 (s, 1H), 8.34 (s, 1H), 7.92 (s, 1H), 7.67 (d, J=8.7 Hz, 1H), 7.55 (t, J=8.2 Hz, 1H), 7.20 (s, 1H), 4.45-4.35 ppm (m, 4H). ¹³C NMR (126 MHz, DMSO-d₆): δ=156.95, 153.98 (d, J=249.1 Hz), 152.99, 149.35, 146.09, 143.74, 128.50 (d, J=3.7 Hz), 128.14, 127.21 (d, J=13.7 Hz), 120.96, 112.51, 112.33, 109.68, 108.36, 64.51, 64.14 ppm. HRMS (DART): m/z [M+H]⁺ calcd for C₁₆H₁₁Br₂FN₃O₂⁺, 453.9197; found, 453.9191.

### N-(5-Bromo-2-fluorophenyl)-7,8-dihydro[1,4]dioxino[2,3-g]quinazolin-4-amine (JGK055)

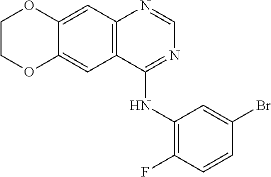 [SMILES: Fc1ccc(Br)cc1Nc1ncnc2cc3c(cc12)OCCO3]

¹H NMR (500 MHz, CDCl₃): δ=8.99 (dd, J=7.3, 2.5 Hz, 1H), 8.72 (s, 1H), 7.38 (s, 1H), 7.36 (br, 1H), 7.27 (s, 1H), 7.16 (ddd, J=8.7, 4.6, 2.5 Hz, 1H), 7.04 (dd, J=10.9, 8.7 Hz, 1H), 4.44-4.36 ppm (m, 4H). ¹³C NMR (126 MHz, CDCl₃): δ=155.59, 153.35, 152.16 (d, J=243.1 Hz), 149.69, 146.67, 144.52, 128.75 (d, J=10.5 Hz), 126.16 (d, J=7.6 Hz), 125.06, 117.19 (d, J=3.4 Hz), 116.20 (d, J=20.9 Hz), 114.52, 110.48, 105.85, 64.68, 64.50 ppm. HRMS (DART): m/z [M+H]⁺ calcd for C₁₆H₁₂BrFN₃O₂⁺, 376.0091; found, 376.0077.

### N-(3-Bromo-2,6-difluorophenyl)-7,8-dihydro[1,4]dioxino[2,3-g]quinazolin-4-amine (JGK056)

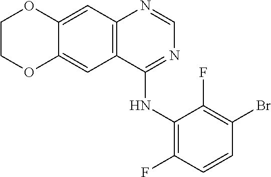 [SMILES: Fc1ccc(Br)c(F)c1Nc1ncnc2cc3c(cc12)OCCO3]

¹H NMR (500 MHz, DMSO-d₆): δ=9.60 (s, 1H), 8.32 (s, 1H), 7.94 (s, 1H), 7.74 (td, J=8.1, 5.5 Hz, 1H), 7.28 (t, J=9.3 Hz, 1H), 7.21 (s, 1H), 4.44-4.38 ppm (m, 4H). ¹³C NMR (126 MHz, DMSO-d₆): δ=157.78 (dd, J=248.8, 3.3 Hz), 157.37, 155.01 (dd, J=247.9, 4.9 Hz), 153.08, 149.47, 146.04, 143.86, 130.76 (d, J=9.3 Hz), 117.30 (t, J=17.5 Hz), 113.30 (dd, J=21.8, 3.0 Hz), 112.56, 109.45, 108.28, 103.55 (dd, J=20.4, 3.6 Hz), 64.52, 64.14 ppm. HRMS (ESI): m/z [M+H]⁺ calcd for C₁₆H₁₁BrF₂N₃O₂⁺, 393.9997; found, 394.0008.

### N-(3-Bromo-2,4-difluorophenyl)-7,8-dihydro[1,4]dioxino[2,3-g]quinazolin-4-amine (JGK057)

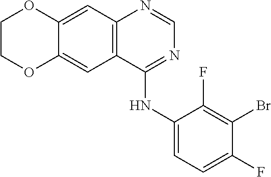 [SMILES: Fc1ccc(Nc2ncnc3cc4c(cc23)OCCO4)c(F)c1Br]

¹H NMR (500 MHz, CDCl₃): δ=8.64 (s, 1H), 8.51 (td, J=9.0, 5.6 Hz, 1H), 7.38 (s, 1H), 7.29 (s, 1H), 7.23 (br, 1H), 7.04 (ddd, J=9.2, 7.8, 2.1 Hz, 1H), 4.45-4.37 ppm (m, 4H). ¹³C NMR (126 MHz, CDCl₃): δ=156.10, 155.80 (dd, J=246.6, 3.5 Hz), 153.28, 151.25 (dd, J=245.1, 4.0 Hz), 149.74, 146.56, 144.53, 124.39 (dd, J=10.8, 3.4 Hz), 122.72 (dd, J=8.3, 1.8 Hz), 114.42, 111.49 (dd, J=22.5, 3.9 Hz), 110.34, 105.98, 97.86 (dd, J=25.7, 22.9 Hz), 64.69, 64.50 ppm. HRMS (ESI): m/z [M+H]⁺ calcd for C₁₆H₁₁BrF₂N₃O₂⁺, 393.9997; found, 394.0013.

### N-(3-Bromo-5-chloro-2-fluorophenyl)-7,8-dihydro[1,4]doxino[2,3-g]quinazolin-4-amine (JGK058)

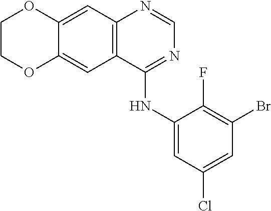 [SMILES: Fc1c(Br)cc(Cl)cc1Nc1ncnc2cc3c(cc12)OCCO3]

¹H NMR (500 MHz, CDCl₃): δ=8.88 (dd, J=6.6, 2.6 Hz, 1H), 8.73 (s, 1H), 7.41 (s, 1H), 7.37 (br, 1H), 7.26 (s, 1H), 7.28-7.23 (m, 1H), 4.44-4.39 ppm (m, 4H). ¹³C NMR (126 MHz, CDCl₃): δ=155.45, 153.13, 149.88, 148.60 (d, J=241.7 Hz), 146.76, 144.72, 130.30 (d, J=4.4 Hz), 129.26 (d, J=10.8 Hz), 126.08, 121.21, 114.60, 110.49, 108.68 (d, J=20.9 Hz), 105.71, 64.70, 64.52 ppm. HRMS (ESI): m/z [M+H]⁺ calcd for C₁₆H₁₁BrClFN₃O₂⁺, 409.9702; found, 409.9713.

### (±)-trans-N-(3-Bromo-2-fluorophenyl)-7,8-dimethyl-7,8-dihydro[1,4]dioxino[2,3-g]quinazolin-4-amine ((±)-JGK059)

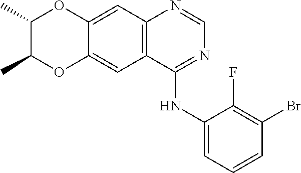 [SMILES: C[C@@H]1Oc2cc3ncnc(Nc4cccc(Br)c4F)c3cc2O[C@H]1C]

¹H NMR (500 MHz, CDCl₃): δ=8.68 (s, 1H), 8.66 (ddd, J=8.6, 7.3, 1.6 Hz, 1H), 7.376 (s, 1H), 7.375 (br, 1H), 7.28 (s, 1H), 7.28-7.24 (m, 1H), 7.10 (td, 0.1=8.2, 1.6 Hz, 1H), 4.08-3.98 (m, 2H), 1.451 (d, J=6.1 Hz, 3H), 1.448 ppm (d, J=6.1 Hz, 3H). ¹³C NMR (126 MHz, CDCl₃): δ=155.90, 153.12, 150.12 (d, J=242.5 Hz), 149.90, 146.59, 144.65, 128.70 (d, J=10.2 Hz), 127.18, 125.30 (d, J=4.5 Hz), 121.74, 113.84, 110.43, 108.57 (d, J=19.2 Hz), 105.31, 75.31, 75.05, 17.23, 17.20 ppm. HRMS (ESI): m/z [M+H]⁺ calcd for C₁₈H₁₆BrFN₃O₂⁺, 404.0404; found, 404.0405.

### N-(3,4-Dichloro-2-fluorophenyl)-7,8-dihydro[1,4]dioxino[2,3-g]quinazolin-4-amine (JGK060)

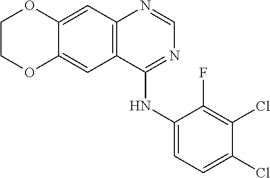 [SMILES: Fc1c(Nc2ncnc3cc4c(cc23)OCCO4)ccc(Cl)c1Cl]

¹H NMR (500 MHz, CDCl₃): δ=8.67 (s, 1H), 8.59 (t, J=8.6 Hz, 1H), 7.40 (s, 1H), 7.38 (br, 1H), 7.33 (dd, J=9.1, 2.1 Hz, 1H), 7.31 (s, 1H), 4.45-4.38 ppm (m, 4H). ¹³C NMR (126 MHz, CDCl₃): δ=155.84, 153.08, 149.98 (d, J=246.3 Hz), 149.88, 146.42, 144.67, 127.55, 127.19 (d, J=10.0 Hz), 125.30 (d, J=4.1 Hz), 121.05, 120.47 (d, J=18.2 Hz), 114.36, 110.43, 105.97, 64.71, 64.51 ppm. HRMS (ESI): m/z [M+H]⁺ calcd for C₁₆H₁₁Cl₂FN₃O₂⁺, 366.0207; found, 366.0207.

### N-(3-Bromo-2,5-difluorophenyl)-7,8-dihydro[1,4]dioxino[2,3-g]quinazolin-4-amine (JGK061)

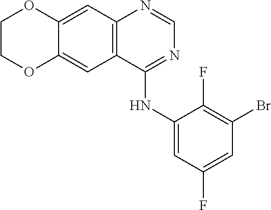 [SMILES: Fc1cc(Br)c(F)c(Nc2ncnc3cc4c(cc23)OCCO4)c1]

¹H NMR (500 MHz, DMSO-d₆): δ=9.65 (s, 1H), 8.40 (s, 1H), 7.93 (s, 1H), 7.63-7.54 (m, 2H), 7.21 (s, 1H), 4.45-4.37 ppm (m, 4H). ¹³C NMR (126 MHz, DMSO-d₆): δ=157.29 (d, J=243.5 Hz), 156.84, 152.93, 149.97 (d, J=242.9 Hz), 149.43, 146.16, 143.81, 129.22-128.44 (m), 116.30 (d, J=26.7 Hz), 113.99 (d, J=25.7 Hz), 112.53, 109.73, 108.76 (dd, J=22.5, 12.5 Hz), 108.33, 64.52, 64.15 ppm. HRMS (DART): m/z [M+H]⁺ calcd for C₁₆H₁₁BrF₂N₃O₂⁺, 393.9997; found, 393.9988.

### (±)-N-(3-Bromo-2-fluorophenyl)-7-ethenyl-7,8-dihydro[1,4]dioxino[2,3-g]quinazolin-4-amine ((±)-JGK062)

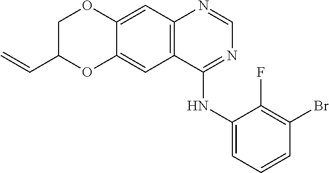 [SMILES: C=CC1COc2cc3ncnc(Nc4cccc(Br)c4F)c3cc2O1]

¹H NMR (500 MHz, CDCl₃): δ=8.68 (s, 1H), 8.65 (ddd, J=8.2, 7.3, 1.5 Hz, 1H), 7.40 (s, 1H), 7.37 (br, 1H), 7.35 (s, 1H), 7.27 (ddd, J=8.0, 6.4, 1.5 Hz, 1H), 7.10 (td, J=8.2, 1.6 Hz, 1H), 5.95 (ddd, J=17.3, 10.7, 5.8 Hz, 1H), 5.60 (dt, J=17.3, 1.2 Hz, 1H), 5.48 (dt, J=10.7, 1.1 Hz, 1H), 4.82-4.74 (m, 1H), 4.42 (dd, J=11.5, 2.5 Hz, 1H), 4.09 ppm (dd, J=11.6, 8.1 Hz, 1H). ¹³C NMR (126 MHz, CDCl₃): δ=155.90, 153.38, 150.14 (d, J=242.4 Hz), 149.12, 146.70, 144.12, 131.48, 128.64 (d, J=10.3 Hz), 127.24, 125.30 (d, J=4.7 Hz), 121.76, 120.43, 114.29, 110.69, 108.58 (d, J=19.3 Hz), 106.06, 74.03, 67.84 ppm. HRMS (DART): m/z [M+H]⁺ calcd for C₁₈H₁₄BrFN₃O₂⁺, 402.0248; found, 402.0233.

### Example 4: Bioactivity and Assay Protocol of Exemplary Compounds

The Cell Free EGFR Kinase Assay was performed using the EGFR Kinase System (Promega # V3831). 13 concentrations at 2-fold dilutions from 250 nM to 0.03052 nM, a no drug control, and a no enzyme control were used in duplicates on 25 ng of EGFR enzyme per reaction. The ADP-Glo Kinase Assay (Promega # V6930) was used to measure EGFR activity in the presence of inhibitors.

The GI50 Assays were performed using patient-derived glioblastoma cells. 13 concentrations at 2-fold dilutions from 40,000 nM to 9.77 nM (for GBM lines) or from 4,000 nM to 0.977 nM (for Lung Cancer lines (HK031)) were plated on 384-well plates in quadruplicates with 1500 cells per well. Cells were incubated for 3 days and then proliferation was assessed by Cell Titer Glo (Promega # G7570). As a reference, Eriotinib exhibited an GI₅₀ of 642 nM (HK301) and 2788 nM (GBM39).

Pharmacokinetic studies were performed on male CD-1 mice aged 8-10 weeks. Mice were dosed as indicated in duplicates. At the time points, whole blood was obtained by retro-orbital bleeding and brain tissue was harvested. Blood samples were centrifuged to obtain plasma and brain tissue was washed and homogenized. Samples were extracted with acetonitrile and supernatant was dried using a speed-vac. Dried samples were solubilized in 50:50:0.1 acetonitrile:water:formic acid and quantified on an Agilent 6400 series Triple Quadrupole LC/MS.

TABLE 3
Activity of Exemplary Compounds of the Present Invention

| | | HK301 | GBM39 |
|---|---|---|---|
| Number | Structure | GI₅₀ (nM) | GI₅₀ (nM) |
| JGK001 | 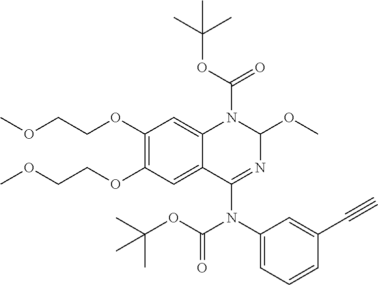 [SMILES: C#Cc1cccc(N(C(=O)OC(C)(C)C)C2=NC(OC)N(C(=O)OC(C)(C)C)c3cc(OCCOC)c(OCCOC)cc32)c1] | 2214 | 19670 |
| JGK002 | 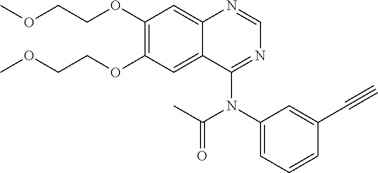 [SMILES: C#Cc1cccc(N(C(C)=O)c2ncnc3cc(OCCOC)c(OCCOC)cc23)c1] | 5448 | 19820 |
| JGK003 | 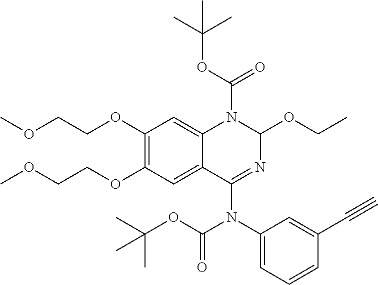 [SMILES: C#Cc1cccc(N(C(=O)OC(C)(C)C)C2=NC(OCC)N(C(=O)OC(C)(C)C)c3cc(OCCOC)c(OCCOC)cc32)c1] | 1127 | 23110 |
| JGK004 | 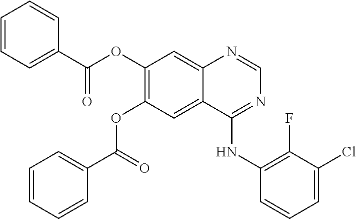 [SMILES: O=C(Oc1cc2ncnc(Nc3cccc(Cl)c3F)c2cc1OC(=O)c1ccccc1)c1ccccc1] | 23383 | 102690 |
| JGK005 | 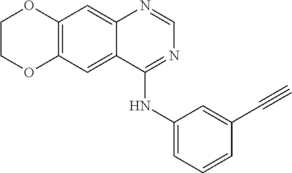 [SMILES: C#Cc1cccc(Nc2ncnc3cc4c(cc23)OCCO4)c1] | 8824 | 20536 |
| JGK006 | 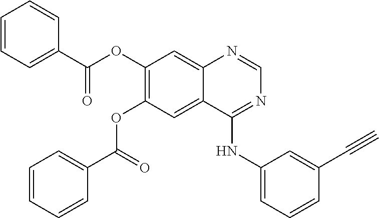 [SMILES: C#Cc1cccc(Nc2ncnc3cc(OC(=O)c4ccccc4)c(OC(=O)c4ccccc4)cc23)c1] | 11012 | 51380 |
| JGK007 | 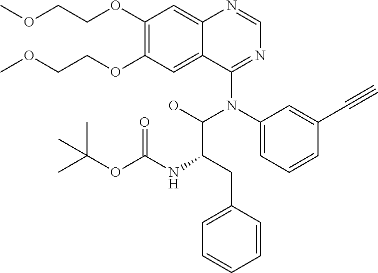 [SMILES: C#Cc1cccc(N(c2ncnc3cc(OCCOC)c(OCCOC)cc23)C(O)[C@H](Cc2ccccc2)NC(=O)OC(C)(C)C)c1] | 92147 | — |
| JGK008 | 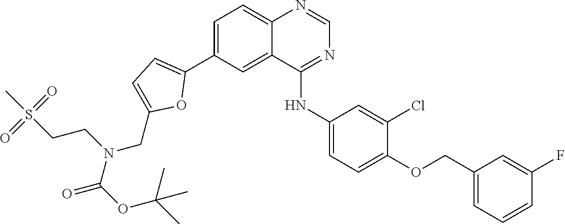 [SMILES: CC(C)(C)OC(=O)N(CCS(C)(=O)=O)Cc1ccc(-c2ccc3ncnc(Nc4ccc(OCc5cccc(F)c5)c(Cl)c4)c3c2)o1] | 81269 | — |
| JGK009 | 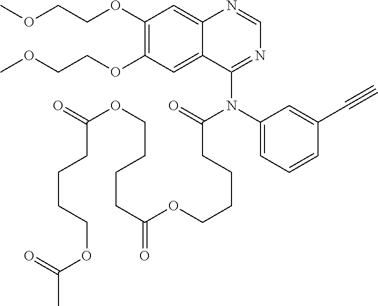 [SMILES: C#Cc1cccc(N(C(=O)CCCCOC(=O)CCCCOC(=O)CCCCOC(C)=O)c2ncnc3cc(OCCOC)c(OCCOC)cc23)c1] | 6096 | 14640 |
| JGK010 | 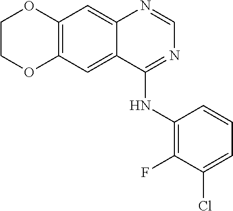 [SMILES: Fc1c(Cl)cccc1Nc1ncnc2cc3c(cc12)OCCO3] | 780.5 | 2594 |
| JGK011 | 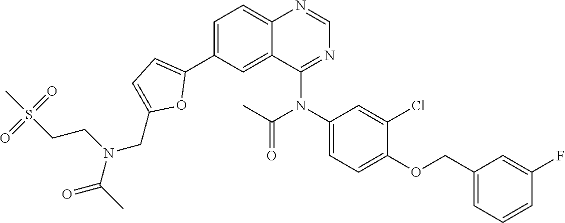 [SMILES: CC(=O)N(CCS(C)(=O)=O)Cc1ccc(-c2ccc3ncnc(N(C(C)=O)c4ccc(OCc5cccc(F)c5)c(Cl)c4)c3c2)o1] | 9206 | 3252 |
| JGK0012 | 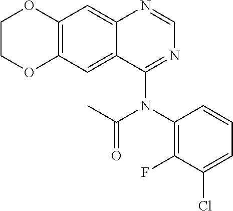 [SMILES: CC(=O)N(c1cccc(Cl)c1F)c1ncnc2cc3c(cc12)OCCO3] | 5477 | 10820 |
| JGK013 | 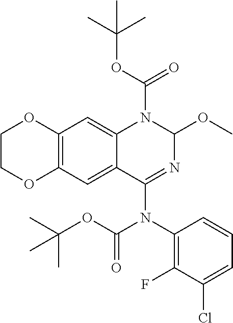 [SMILES: COC1N=C(N(C(=O)OC(C)(C)C)c2cccc(Cl)c2F)c2cc3c(cc2N1C(=O)OC(C)(C)C)OCCO3] | 1551 | 219870 |
| JGK014 | 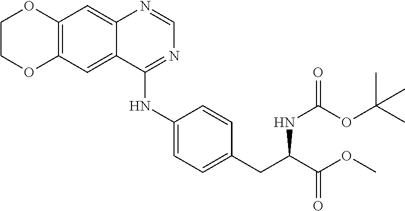 [SMILES: COC(=O)[C@@H](Cc1ccc(Nc2ncnc3cc4c(cc23)OCCO4)cc1)NC(=O)OC(C)(C)C] | 11816 | 27914 |
| JGK015 | 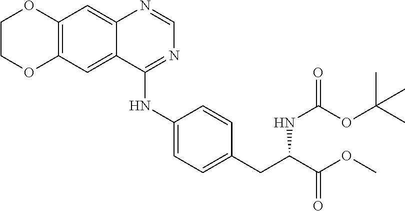 [SMILES: COC(=O)[C@H](Cc1ccc(Nc2ncnc3cc4c(cc23)OCCO4)cc1)NC(=O)OC(C)(C)C] | 2154 | 11540 |
| JGK016 | 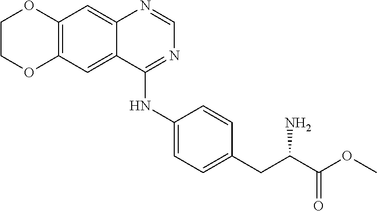 [SMILES: COC(=O)[C@@H](N)Cc1ccc(Nc2ncnc3cc4c(cc23)OCCO4)cc1] | 4052 | 61593 |
| JGK017 | 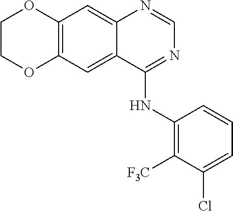 [SMILES: FC(F)(F)c1c(Cl)cccc1Nc1ncnc2cc3c(cc12)OCCO3] | 64235 | 97624 |
| JGK018 | 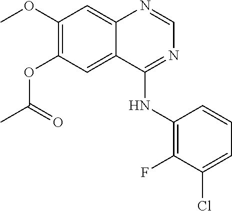 [SMILES: COc1cc2ncnc(Nc3cccc(Cl)c3F)c2cc1OC(C)=O] | 1071 | 4543 |
| JGK019 | 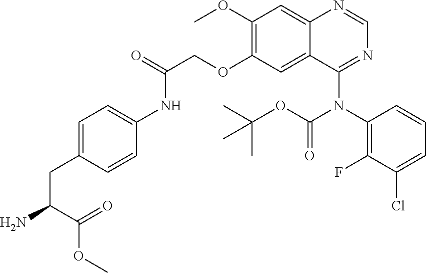 [SMILES: COC(=O)[C@@H](N)Cc1ccc(NC(=O)COc2cc3c(N(C(=O)OC(C)(C)C)c4cccc(Cl)c4F)ncnc3cc2OC)cc1] | — | — |
| JGK020 | 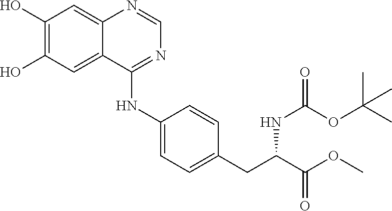 [SMILES: COC(=O)[C@H](Cc1ccc(Nc2ncnc3cc(O)c(O)cc23)cc1)NC(=O)OC(C)(C)C] | 29236 | 36179 |
| JGK021 | 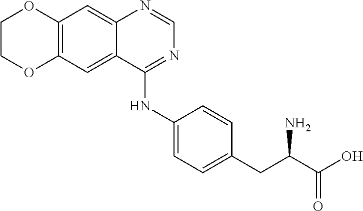 [SMILES: N[C@H](Cc1ccc(Nc2ncnc3cc4c(cc23)OCCO4)cc1)C(=O)O] | 480850 | 555780 |
| JGK022 | 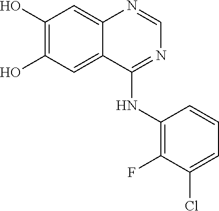 [SMILES: Oc1cc2ncnc(Nc3cccc(Cl)c3F)c2cc1O] | 31116 | 150164 |
| JGK023 | 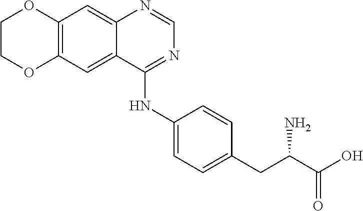 [SMILES: N[C@@H](Cc1ccc(Nc2ncnc3cc4c(cc23)OCCO4)cc1)C(=O)O] | 86230 | 96260 |
| JGK024 | 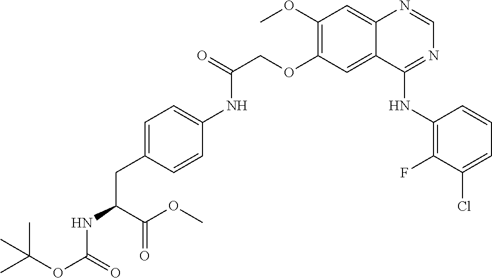 [SMILES: COC(=O)[C@H](Cc1ccc(NC(=O)COc2cc3c(Nc4cccc(Cl)c4F)ncnc3cc2OC)cc1)NC(=O)OC(C)(C)C] | 156370 | 124940 |
| JGK025 | 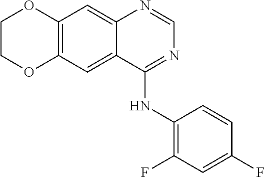 [SMILES: Fc1ccc(Nc2ncnc3cc4c(cc23)OCCO4)c(F)c1] | 10659 | 27706 |
| JGK026 | 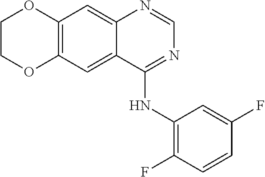 [SMILES: Fc1ccc(F)c(Nc2ncnc3cc4c(cc23)OCCO4)c1] | 6124 | 16525 |
| JGK027 | 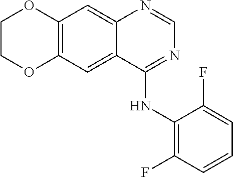 [SMILES: Fc1cccc(F)c1Nc1ncnc2cc3c(cc12)OCCO3] | 5807 | 11837 |
| JGK028 | 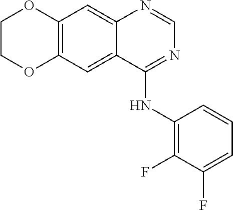 [SMILES: Fc1cccc(Nc2ncnc3cc4c(cc23)OCCO4)c1F] | 1688 | 5572 |
| JGK029 | 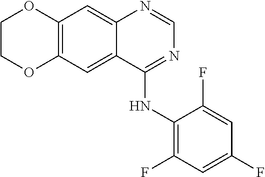 [SMILES: Fc1cc(F)c(Nc2ncnc3cc4c(cc23)OCCO4)c(F)c1] | 24395 | 33970 |
| JGK030 | 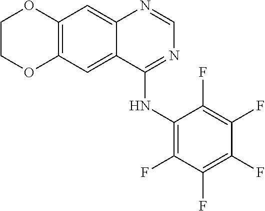 [SMILES: Fc1c(F)c(F)c(Nc2ncnc3cc4c(cc23)OCCO4)c(F)c1F] | 558742 | 961204 |
| JGK031 | 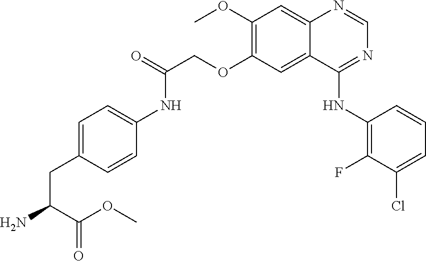 [SMILES: COC(=O)[C@@H](N)Cc1ccc(NC(=O)COc2cc3c(Nc4cccc(Cl)c4F)ncnc3cc2OC)cc1] | — | — |
| JGK032 | 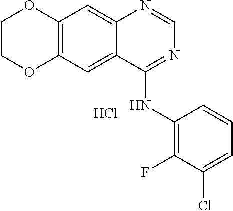 [SMILES: Fc1c(Cl)cccc1Nc1ncnc2cc3c(cc12)OCCO3 ; Cl] | 780.5 | 2594 |
| JGK033 | 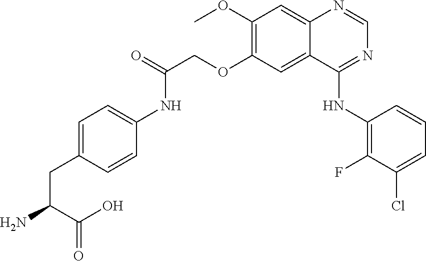 [SMILES: COc1cc2ncnc(Nc3cccc(Cl)c3F)c2cc1OCC(=O)Nc1ccc(C[C@H](N)C(=O)O)cc1] | — | — |
| JGK035 | 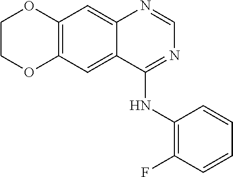 [SMILES: Fc1ccccc1Nc1ncnc2cc3c(cc12)OCCO3] | 4040 | 10721 |
| JGK036 | 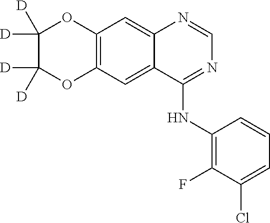 [SMILES: [2H]C1([2H])Oc2cc3ncnc(Nc4cccc(Cl)c4F)c3cc2OC1([2H])[2H]] | 1046 | 4507 |
| JGK037 | 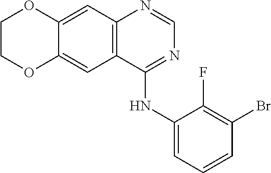 [SMILES: Fc1c(Br)cccc1Nc1ncnc2cc3c(cc12)OCCO3] | 329.3 | 1116 |
| JGK038 | 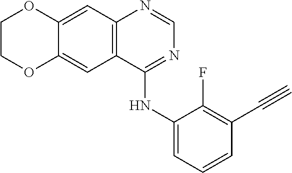 [SMILES: C#Cc1cccc(Nc2ncnc3cc4c(cc23)OCCO4)c1F] | 791.1 | 2946 |
| JGK039 | 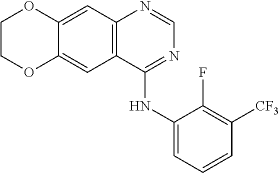 [SMILES: Fc1c(Nc2ncnc3cc4c(cc23)OCCO4)cccc1C(F)(F)F] | 3614 | 7820 |
| JGK040 | 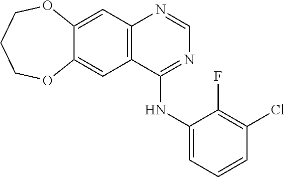 [SMILES: Fc1c(Cl)cccc1Nc1ncnc2cc3c(cc12)OCCCO3] | 1721 | 7115 |
| JGK041 | 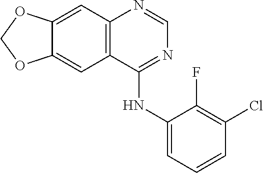 [SMILES: Fc1c(Cl)cccc1Nc1ncnc2cc3c(cc12)OCO3] | 1658 | 6042 |
| JGK042 | 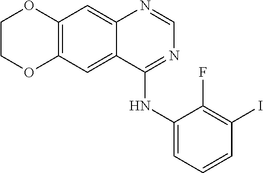 [SMILES: Fc1c(I)cccc1Nc1ncnc2cc3c(cc12)OCCO3] | 2294 | 4521 |
| JGK043 | 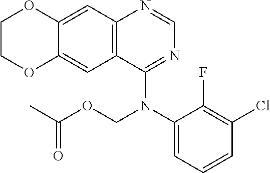 [SMILES: CC(=O)OCN(c1cccc(Cl)c1F)c1ncnc2cc3c(cc12)OCCO3] | 745 | 1778 |
| JGK044 | 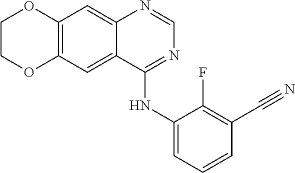 [SMILES: N#Cc1cccc(Nc2ncnc3cc4c(cc23)OCCO4)c1F] | 4522 | 5635 |
| JGK045 | 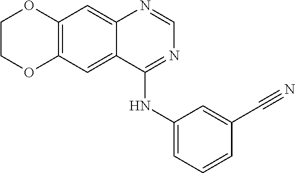 [SMILES: N#Cc1cccc(Nc2ncnc3cc4c(cc23)OCCO4)c1] | 3940 | 10939 |
| JGK047 | 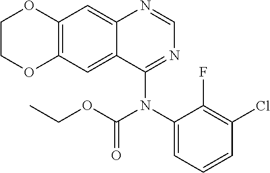 [SMILES: CCOC(=O)N(c1cccc(Cl)c1F)c1ncnc2cc3c(cc12)OCCO3] | 316000 | — |
| JGK050 | 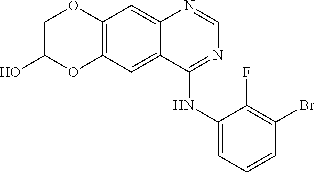 [SMILES: OC1COc2cc3ncnc(Nc4cccc(Br)c4F)c3cc2O1] | 1159 | 3568 |
| JGK051 | 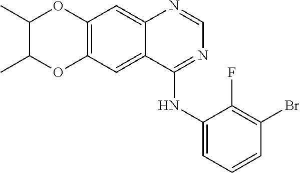 [SMILES: CC1Oc2cc3ncnc(Nc4cccc(Br)c4F)c3cc2OC1C] | 8253 | 24140 |
| JGK052 | 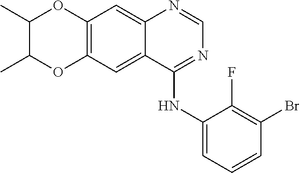 [SMILES: CC1Oc2cc3ncnc(Nc4cccc(Br)c4F)c3cc2OC1C] | 3866 | 9219 |
| JGK053 | 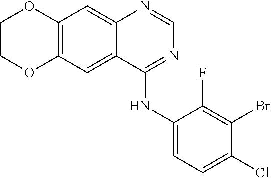 [SMILES: Fc1c(Nc2ncnc3cc4c(cc23)OCCO4)ccc(Cl)c1Br] | 2778 | 5277 |
| JGK054 | 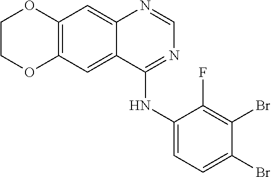 [SMILES: Fc1c(Nc2ncnc3cc4c(cc23)OCCO4)ccc(Br)c1Br] | 5723 | 7697 |
| JGK055 | 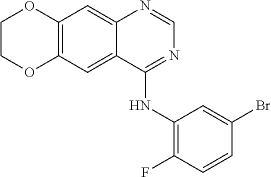 [SMILES: Fc1ccc(Br)cc1Nc1ncnc2cc3c(cc12)OCCO3] | 290.1 | 966.4 |
| JGK056 | 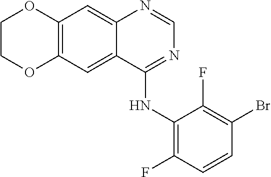 [SMILES: Fc1ccc(Br)c(F)c1Nc1ncnc2cc3c(cc12)OCCO3] | 418.7 | 1355.8 |
| JGK057 | 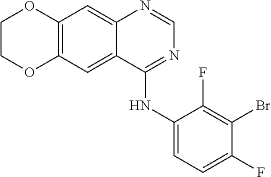 [SMILES: Fc1ccc(Nc2ncnc3cc4c(cc23)OCCO4)c(F)c1Br] | 1382.7 | 9361.5 |
| JGK058 | 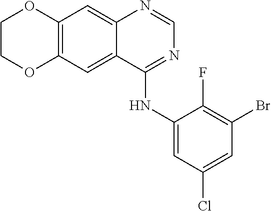 [SMILES: Fc1c(Br)cc(Cl)cc1Nc1ncnc2cc3c(cc12)OCCO3] | 1852.7 | 12974 |
| JGK059 | 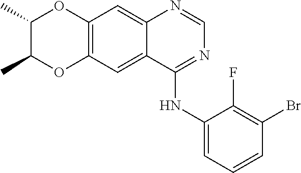 [SMILES: C[C@@H]1Oc2cc3ncnc(Nc4cccc(Br)c4F)c3cc2O[C@H]1C] | 8110 | 11218 |
| JGK060 | 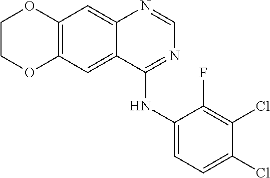 [SMILES: Fc1c(Nc2ncnc3cc4c(cc23)OCCO4)ccc(Cl)c1Cl] | 2947 | 3501 |
| JGK061 | 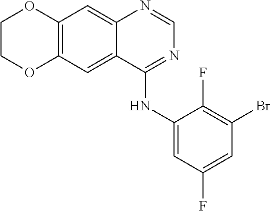 [SMILES: Fc1cc(Br)c(F)c(Nc2ncnc3cc4c(cc23)OCCO4)c1] | 1131 | 1727 |
| JGK062 | 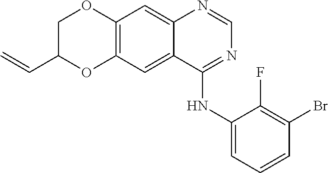 [SMILES: C=CC1COc2cc3ncnc(Nc4cccc(Br)c4F)c3cc2O1] | 3784 | 5856 |

### Example 5: Protein Binding of Erlotinib and Exemplary Compounds of the Disclosure

Illustrated below in Table 4 is the protein binding of erlotinib and several exemplary compounds of the disclosure. Fu refers to “fraction unbound”.

| | Fu (blood)/Fu | Bound (blood)/ | Kpuu |
|---|---|---|---|
| Compound | (brain) | (brain) | (Avg) |
| Erlotinib | 4.88% | 95.12% | 0.0513 |
| | 2.93% | 97.07% | |
| JGK010 | 0.52% | 99.48% | 1.151 |
| | 0.56% | 99.44% | |
| JGK037 | 1.70% | 98.30% | 1.2994 |
| | 1.04% | 98.96% | |
| AZD3759 | 5.93% | 94.07% | 1.30 |
| | 2.77% | 97.23% | |

### Example 6: Classification of EGFRi Metabolic Responders and Non-Responders

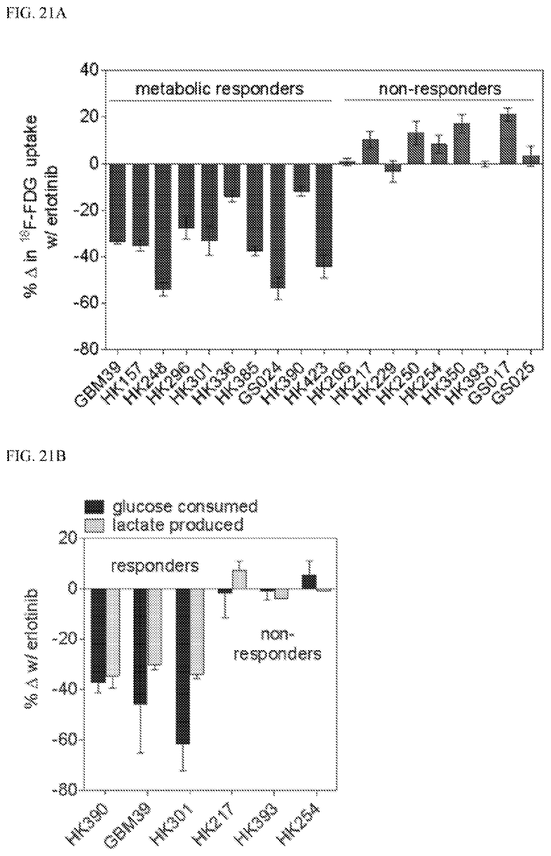

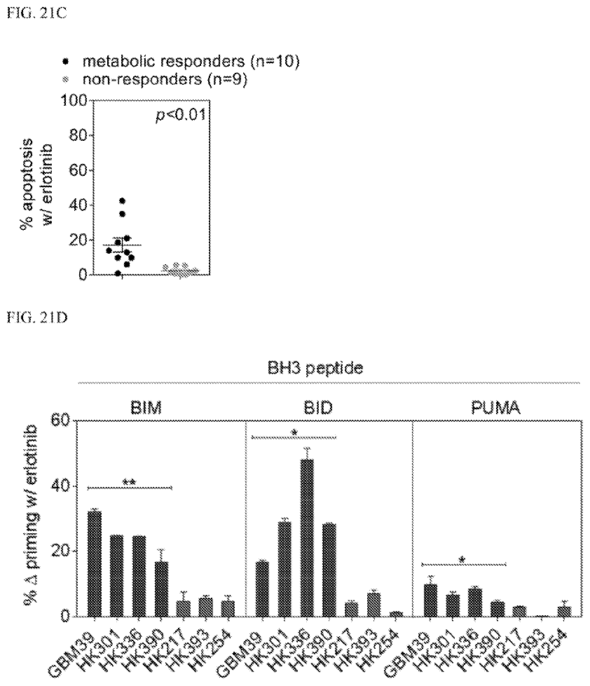

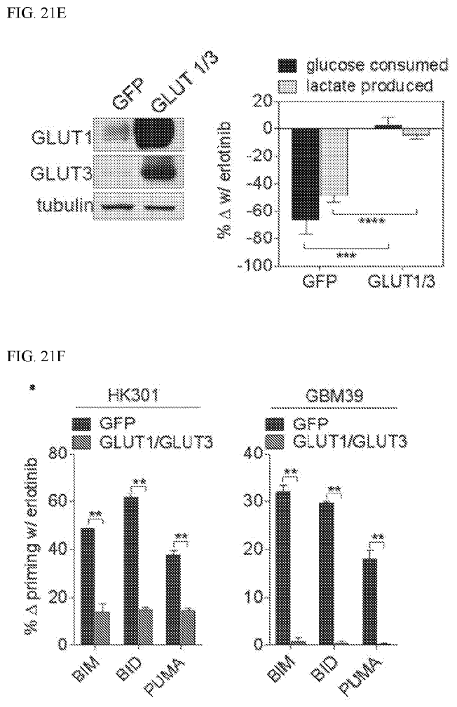

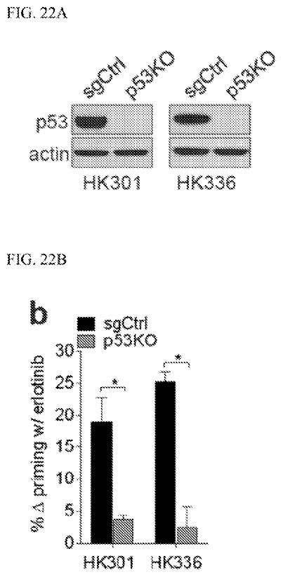

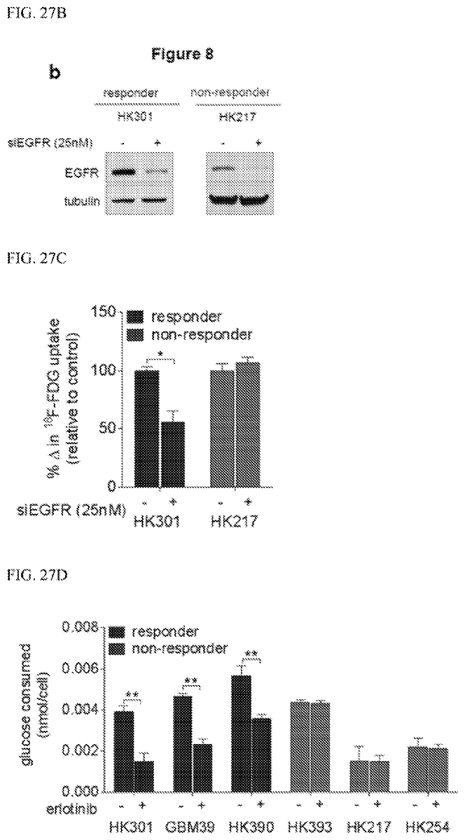

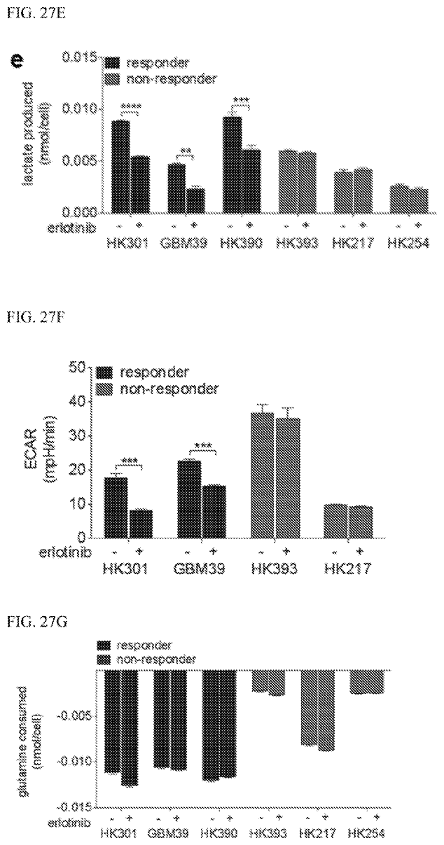

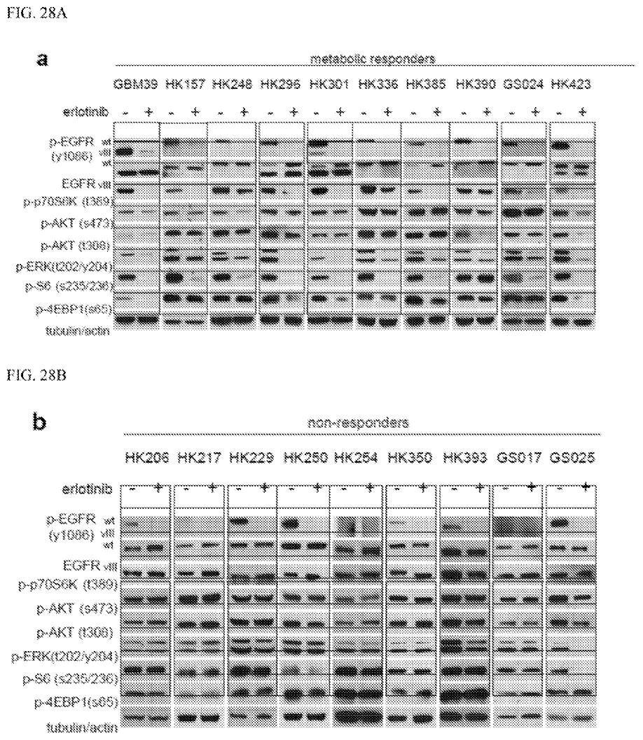

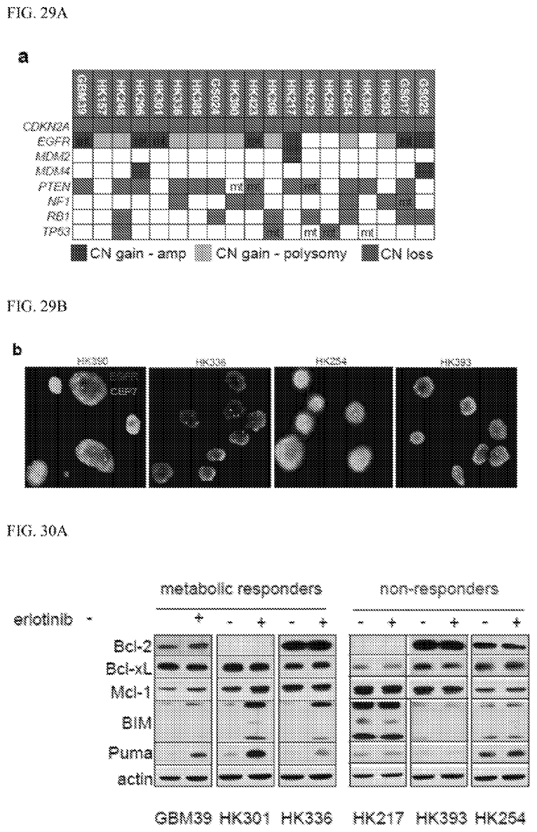

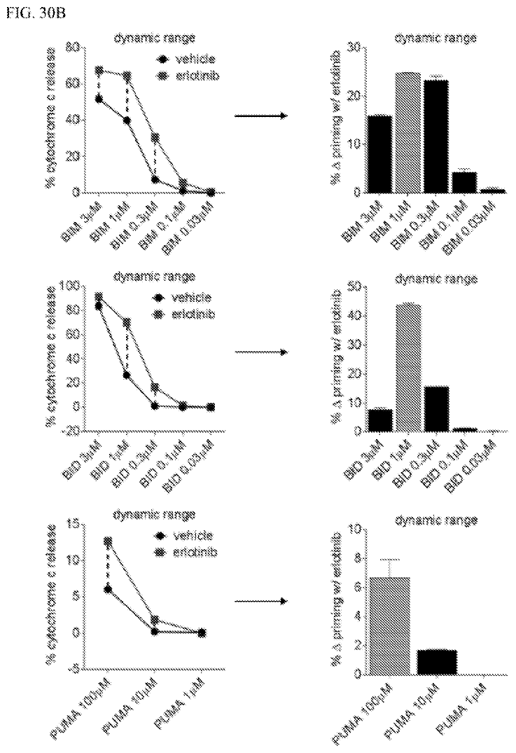

Changes in glucose consumption with acute EGFR inhibition across 19 patient-derived GBM cell lines was characterized. The cells were cultured in supplemented serum-free medium as gliomaspheres which, in contrast to serum-based culture conditions, preserve many of the molecular features of patient tumors. Treatment with the EGFR tyrosine kinase inhibitor (EGFRi) erlotinib identified a subset of GBMs whose radio-labeled glucose uptake (¹⁸F-FDG) was significantly attenuated with EGFR inhibition, hereafter termed “metabolic responders” (FIG. 21A and FIG. 27A). Silencing of EGFR using siRNA confirmed that the reduction in glucose uptake was not due to off target effects of erlotinib (FIG. 27B, 27C). Reduced ¹⁸F-FDG uptake in EGFRi treated cells was associated with decreased lactate production, glucose consumption, and extracellular acidification rate (ECAR), yet glutamine levels remained unchanged (FIG. 21B and FIG. 27D-G). Finally, decreased glucose utilization correlated with perturbations in RAS-MAPK and PI3K-AKT-mTOR signaling—each of which can regulate glucose metabolism in GBM and other cancers (FIG. 28A).

In contrast, in all “non-responders” GBMs (i.e. no change in ¹⁸F-FDG uptake with EGFRi or siRNA) (FIG. 21A and FIG. 27B, 27C), no changes in glucose consumption, lactate production, and ECAR were observed despite robust inhibition of EGFR (FIG. 21B, FIG. 27D-G, and FIG. 28B). Moreover, RAS-MAPK and PI3K-AKT-mTOR signaling were largely unaffected in these cells (FIG. 28B). Notably, while all metabolic responders had alterations in EGFR (copy number gain, mutation), 6 GBM lines without a metabolic response also contained EGFR mutations and/or copy number gains (FIG. 29A, 29B). Taken together, these data illustrate two key points. First, acute inhibition of EGFR rapidly attenuates glucose utilization in a subset of primary GBM cells, and second, genetic alterations in EGFR could not alone predict which GBMs have a metabolic response to EGFRi.

### Example 7: EGFRi Metabolic Responders are Primed for Apotosis

Perturbations in glucose metabolism can induce the expression of pro-apoptotic factors and stimulate intrinsic apoptosis, suggesting that reduced glucose uptake in response to EGFRi would stimulate the intrinsic apoptotic pathway. Indeed, acute erlotinib treatment promoted the expression of the pro-apoptotic BH3-only proteins, BIM and PUMA, only in the metabolic responder cultures (FIG. 30A). However, annexin V staining revealed that the metabolic responders had only modest (˜17%), albeit significantly higher, apoptosis compared with non-responders (˜3%), following 72 hours of erlotinib exposure (FIG. 21C).

The low level of apoptosis, despite pronounced induction of pro-apoptotic factors, led the inventors to ask if perturbing glucose uptake with EGFRi “primes” GBM cells for apoptosis; thus increasing the propensity for apoptosis without inducing significant cell death. To test this, the inventors treated both metabolic responders and non-responders with erlotinib for 24 hours and performed dynamic BH3 profiling to quantify the changes in apoptotic priming (FIG. 30B). Using multiple BH3 peptides (e.g., BIM, BID, and PUMA), we observed a significant increase in apoptotic priming—as determined by the change in cytochrome c release relative to vehicle—in the metabolic responders with erlotinib treatment (FIG. 21D—dark gray bars). Importantly, priming in the metabolic responders was significantly higher than priming in the non-responders (FIG. 21D—light gray bars), supporting the premise that attenuated glucose uptake with EGFRi triggers apoptotic priming in GBM.

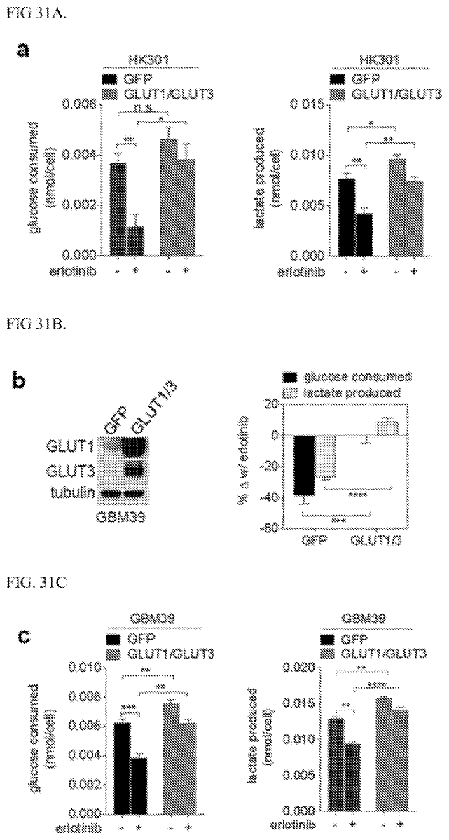

The inventors tested if reduced glucose uptake is required for apoptotic priming with EGFRi, by checking whether rescuing glucose consumption should mitigate these effects. To test this, glucose transporters 1 (GLUT1) and 3 (GLUT3) were ectopically expressed in two metabolic responders (HK301 and GBM39). Enforced expression of GLUT1 and GLUT3 (GLUT 1/3) rescued EGFRi-mediated attenuation of glucose uptake and lactate production in both cell lines (FIG. 21E and FIG. 31A-C) and, importantly, markedly suppressed apoptotic priming in response to EGFRi (FIG. 21F). Collectively, these data demonstrate that EGFRi-mediated inhibition of glucose consumption, although insufficient to induce significant cell death, lowers the apoptotic threshold potentially rendering GBM cells vulnerable to agents that exploit this primed state.

### Example 8: Cytoplasmic p53 is Required for Apoptotic Priming with EGFRi

The mechanism by which GBMs become primed for apoptosis with EGFRi was investigated. The inhibition of oncogene-driven glucose metabolism renders GBM cells synergistically susceptible to cytoplasmic p53 dependent apoptosis. Attenuated glucose metabolic flux in GBM, via targeting oncogenic signaling (e.g., EGFRi), results in cytoplasmic p53 engaging the intrinsic apoptotic pathway (“priming”). However, Bcl-xL blocks cytoplasmic p53-mediated cell death. Pharmacological p53 stabilization overcomes this apoptotic block, leading to synergistic lethality with combined targeting of oncogene-driven glucose metabolism in GBM.

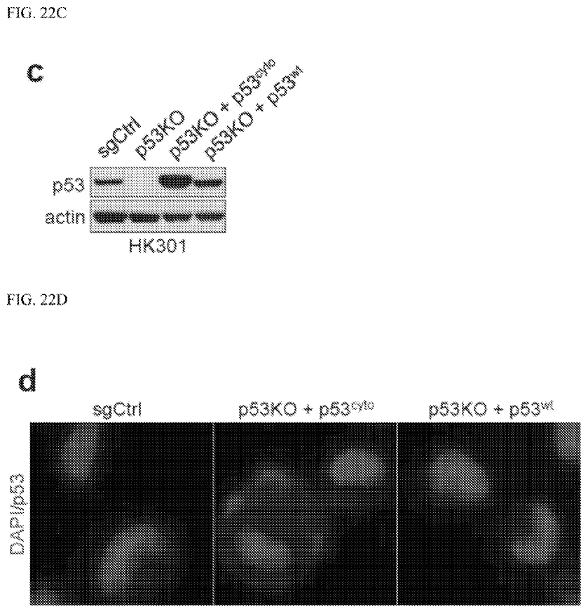

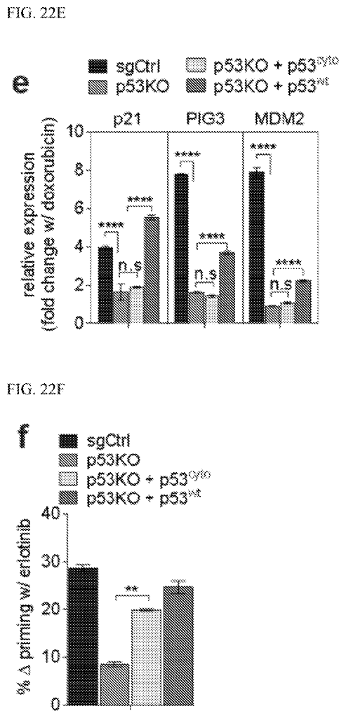

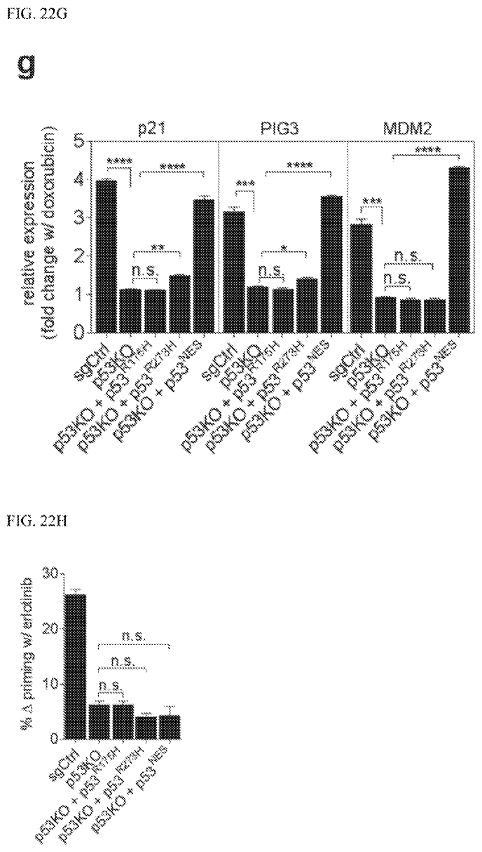

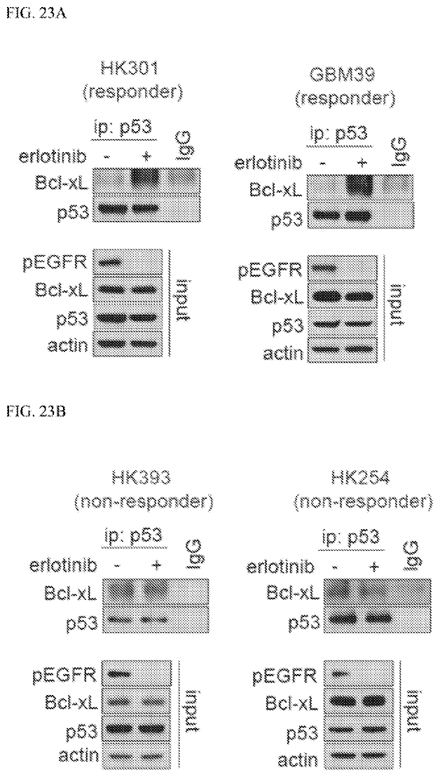

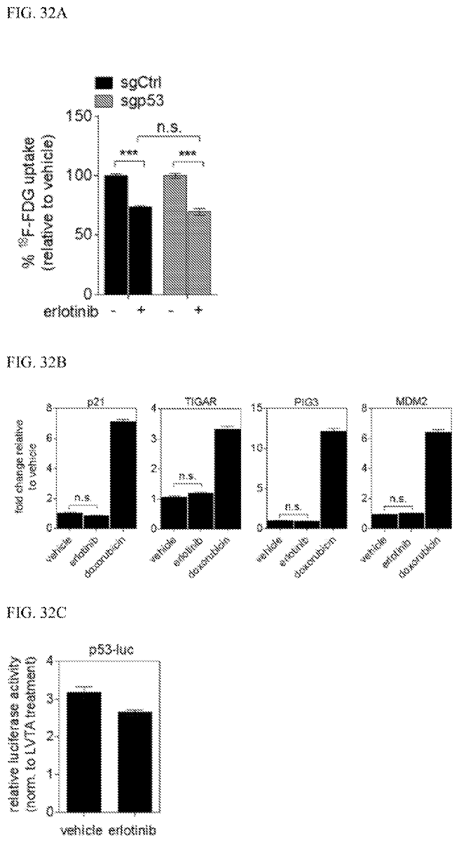

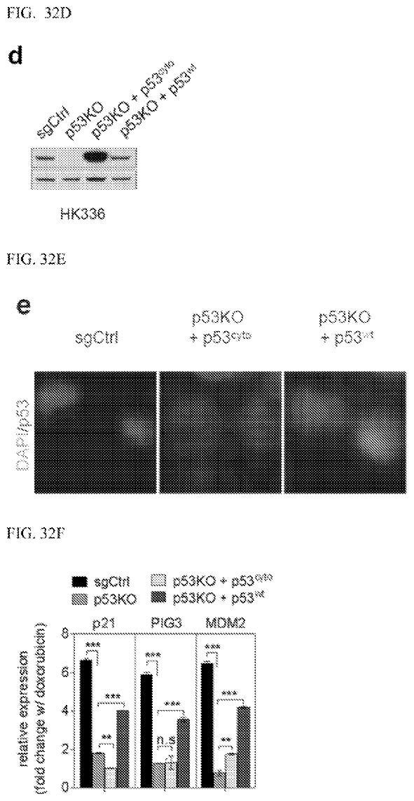

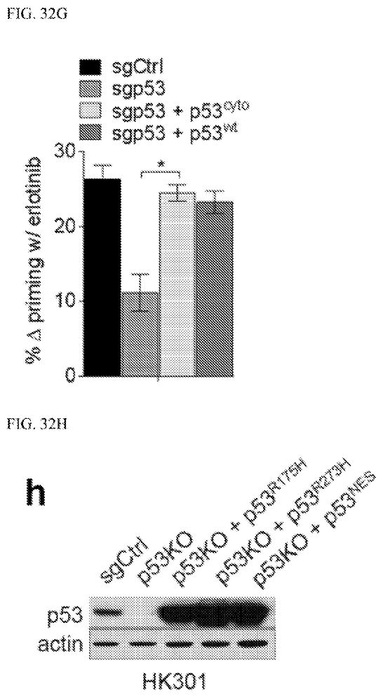

In cells that are in a primed state, the anti-apoptotic Bcl-2 family proteins (e.g. Bcl-2, Bcl-xL, Mcl-1) are largely loaded with pro-apoptotic BH3 proteins (e.g., BIM, BID, PUMA, BAD, NOXA, HRK); consequently, cells are dependent on these interactions for survival. The tumor suppressor protein, p53, is known to upregulate pro-apoptotic proteins that subsequently need to be bound by anti-apoptotic Bcl-2 proteins to prevent cell death. To examine whether p53 is required for EGFRi-induced priming, we silenced p53 with CRISPR/CAS-9 (hereafter referred to as p53KO) in two metabolic responders (HK301 and HK336, FIG. 22A). While the change in glucose consumption with EGFRi was unaffected in p53KO cells (FIG. 32A), BH3 profiling revealed p53KO nearly abolished erlotinib-induced apoptotic priming in both HK301 and HK336 cells (FIG. 22B).

As p53 transcriptional activity has been shown to be enhanced under glucose limitation, it we investigated to determine whether p53-mediated transcription was induced by EGFRi. However, erlotinib did not increase the expression of p53-regulated genes (e.g., p21, MDM2, PIG3, TIGAR) (FIG. 32B), nor induce p53-luciferase reporter activity in HK301 metabolic responder cells (FIG. 32C). These data indicate that while p53 is required for priming with EGFRi, its transcriptional activity may not be necessary.

In addition to p53's well-described nuclear functions, p53 can localize in the cytoplasm where it can directly engage the intrinsic apoptotic pathway. To evaluate whether cytoplasmic p53 is important for apoptotic priming with EGFRi, we stably introduced a p53 mutant with a defective nuclear localization signal (p53^{cyto}) into HK301 and HK336 p53KO gliomaspheres. As expected, p53^{cyto} was expressed (FIG. 22C and FIG. 32D), restricted to the cytoplasm (FIG. 22D and FIG. 32E) and had no transcriptional activity (FIG. 22E and FIG. 32F). Conversely, reconstitution of wild-type p53 (p53^{wt}) in HK301 and HK336 p53KO cells displayed similar localization as parental cells and rescued transcription of p53-regulated genes (FIG. 22C-E and FIG. 32E-G). Remarkably, stable introduction of p53C)^{cyto} significantly restored priming with erlotinib in both HK301 and HK336 p53KO cells to levels comparable to p53^{wt} (FIG. 22F and FIG. 32G), indicating that the cytoplasmic function of p53 is required for EGFRi-mediated priming. In support of this, introduction of a transcriptionally active (FIG. 2622G), yet nuclear-confined p53 mutant (p53^{NES}) into HK301 p53KO cells failed to induce EGFRi-mediated apoptotic priming (FIG. 22G, 22H and FIG. 32H). Finally, pharmacological inhibition of cytoplasmic p53 activity with pifithrin-μ (PFTμ) markedly reduced priming with erlotinib (FIG. 32I). Collectively, these results show that cytoplasmic p53 engages the intrinsic apoptotic machinery following EGFRi in GBM.

Prior work demonstrated that human tumor-derived p53 mutants—specifically those in the DNA binding domain—have diminished cytoplasmic functions in addition to transactivation deficiencies. Thus, the inventors tested whether stable expression of two of these “hotspot” p53 mutants, R175H or R273H, in HK301 p53KO would have reduced EGFRi-mediated priming (FIG. 32H). As expected, both mutants lacked transcriptional capabilities (FIG. 22G) and, consistent with reduced cytoplasmic activity, were incapable of apoptotic priming with EGFRi (FIG. 22H). Therefore, in line with previous findings, oncogenic mutations in the DNA binding domain of p53 result in “dual hits”, whereby both transactivation and cytoplasmic functions are abrogated—the latter having implications for apoptotic priming with EGFRi.

### Example 9: Inhibition of EGFR-Driven Glucose Uptake Creates an Exploitable Bcl-xL Dependency

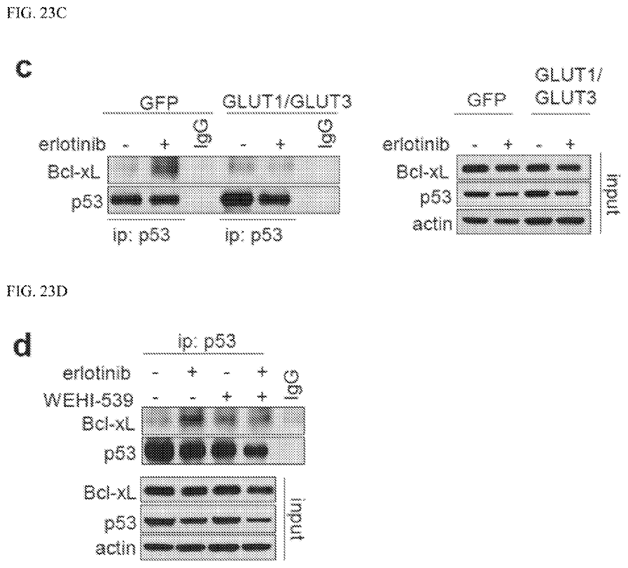

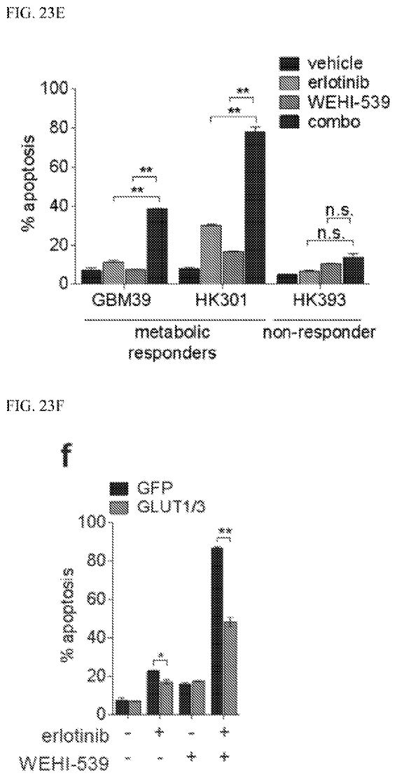

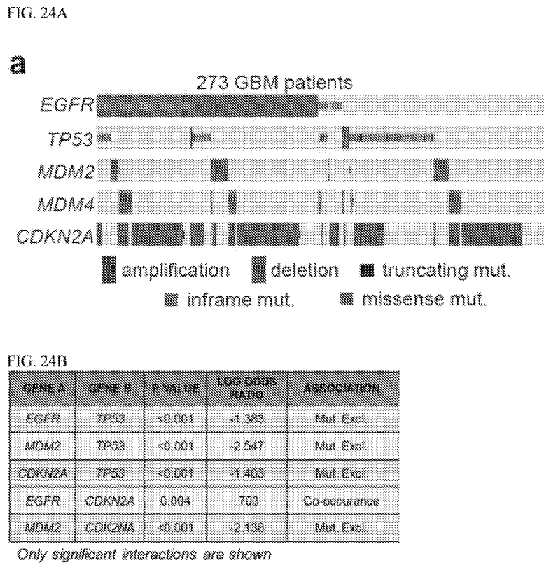

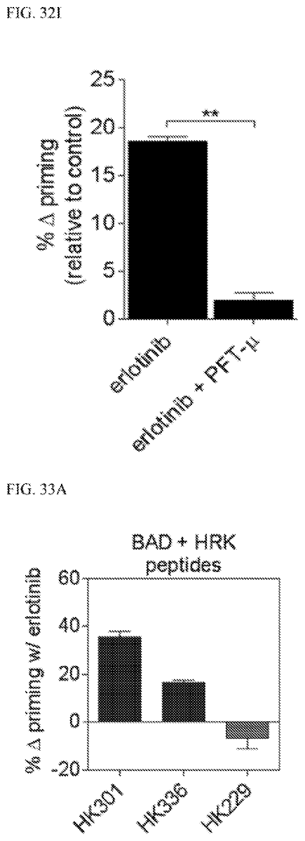

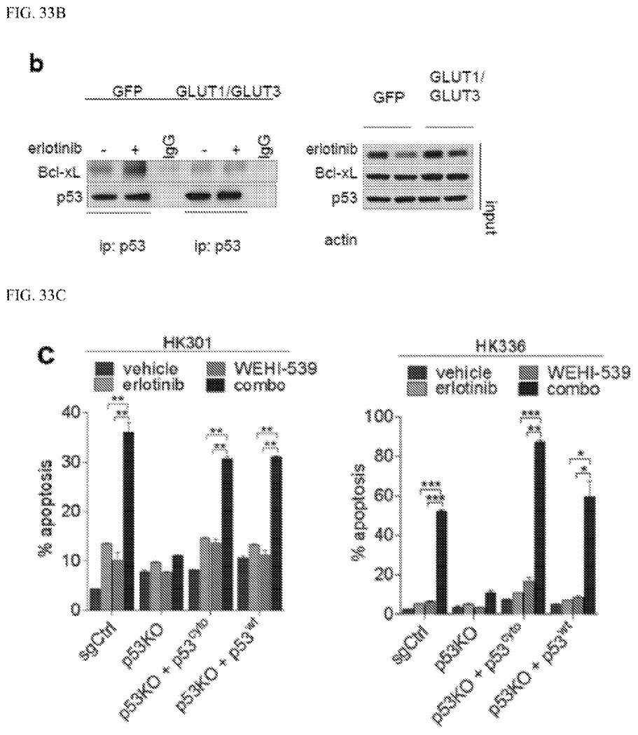

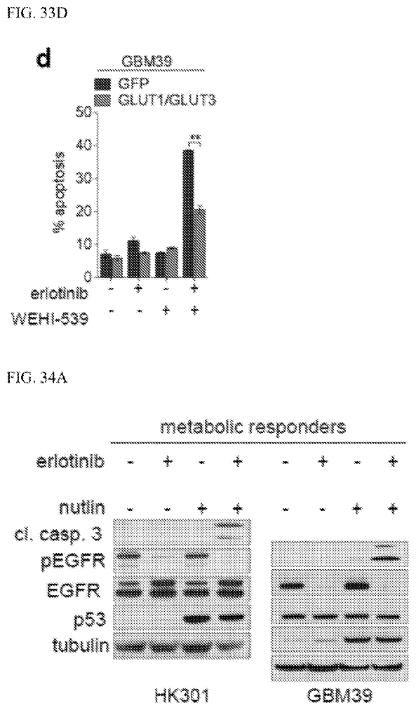

Bcl-xL can sequester cytoplasmic p53 and prevent p53-mediated apoptosis; thus creating a primed apoptotic state and a dependency on Bcl-xL for survival. Indeed, BH3 profiling revealed a dependence on Bcl-xL for cell survival in EGFRi metabolic responders (FIG. 33A). Therefore, we hypothesized that attenuated glucose consumption with EGFRi may result in sequestration of cytoplasmic p53 by Bcl-xL. To investigate this, we performed co-immunoprecipitations to examine the dynamics of p53-Bcl-xL interactions in response to EGFRi in both responders (n=2) and non-responders (n=2). Importantly, we observed markedly heightened Bcl-xL and p53 interactions following erlotinib treatment in metabolic responders (FIG. 23A) but not in non-responders (FIG. 23B). This suggests that inhibition of EGFR-dependent glucose consumption results in sequestration of p53 by Bcl-xL. Consistent with this interpretation, ectopic expression of GLUT1/3, which rescues the EGFRi-mediated reduction in glucose uptake and apoptotic priming, prevented the association of p53 with Bcl-xL (FIG. 23C and FIG. 33B). These findings strongly indicate that EGFRi-mediated inhibition of glucose uptake primes GBM cells for apoptosis by promoting an interaction between cytoplasmic p53 and Bcl-xL.

The liberation of p53 from Bcl-xL enables p53 to directly activate BAX, resulting in cytochrome c release and cell death. Once we recognized increased binding between Bcl-xL and p53 in metabolic responders in response to EGFRi, we asked whether displacement of p53 from Bcl-xL elicits apoptosis. To test this, we treated a metabolic responder (HK301) with erlotinib and the specific Bcl-xL inhibitor, WEHI-539. The addition of WEHI-539 disrupted the association of Bcl-xL with p53 under erotinib treatment (FIG. 23D), leading to synergistic lethality in HK301 and GBM39 cells (metabolic responders) (FIG. 23E). Notably, cytoplasmic p53 was sufficient for the combinatorial effects in EGFRi metabolic responder cells (FIG. 33C). However, WEHI-539 did not enhance apoptosis in a non-responder (HK393) treated with erlotinib, suggesting that attenuation of glucose uptake with EGFRi, and subsequent association between p53 and Bcl-xL, is necessary to generate a dependence on Bcl-xL for survival (FIG. 33E). In support of this, enforced expression of GLUT1/3 significantly mitigated cell death with the drug combination (FIG. 23F and FIG. 33D). Together, these observations indicate that Bcl-xL attenuates GBM cell death in response to EGFRi-mediated inhibition of glucose uptake by sequestering cytoplasmic p53 (FIG. 32G).

### Example 10: Combined Targeting of EGFR and p53 is Synergistic in EGFRi Metabolic Responders

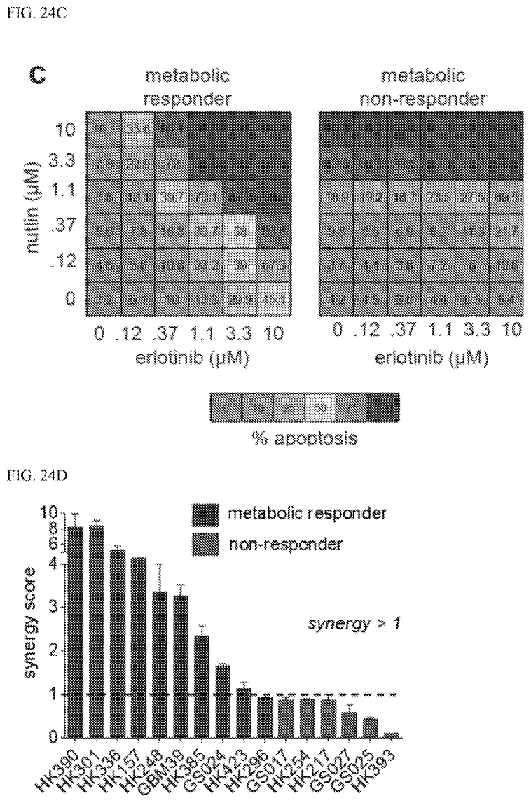

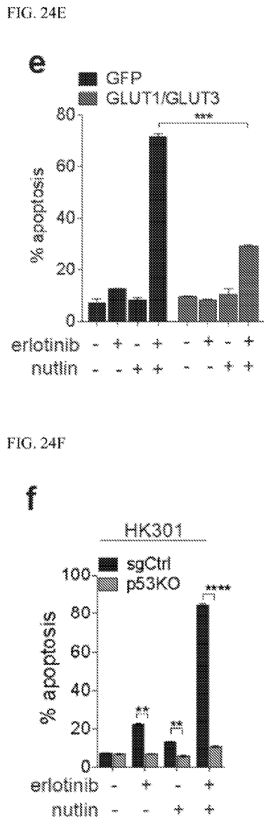

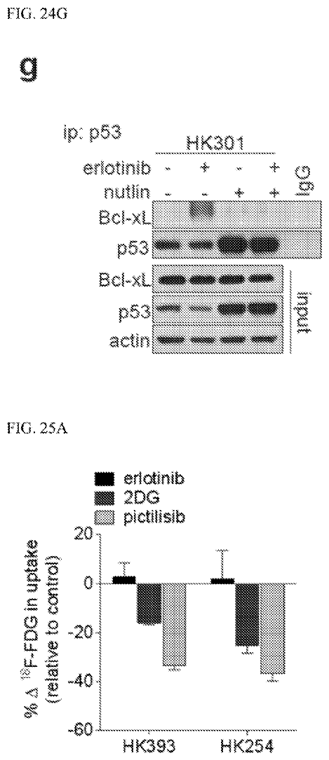

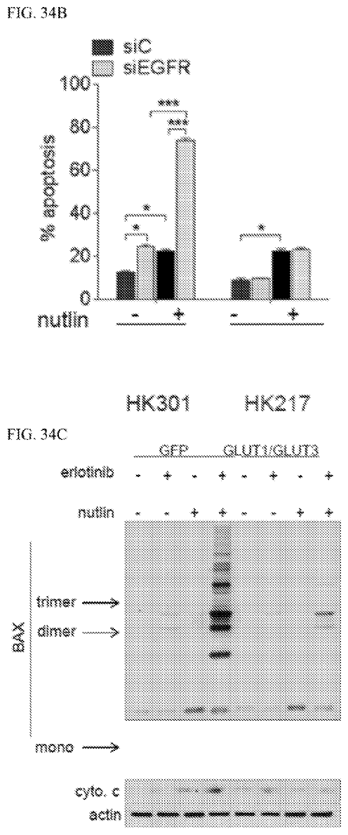

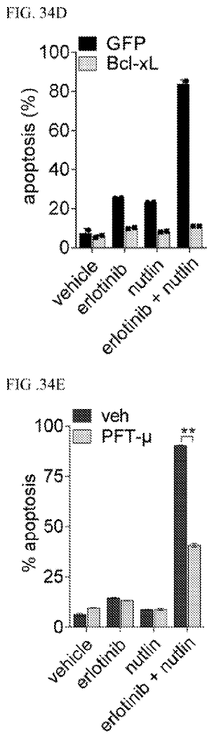

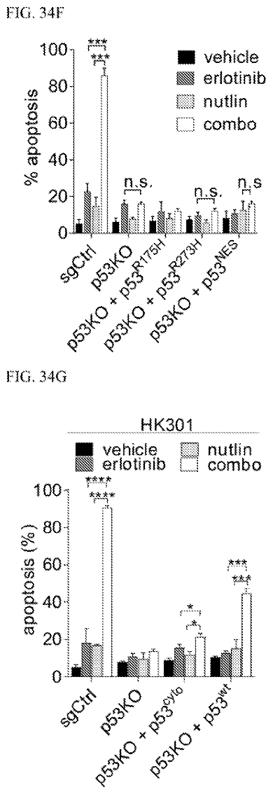

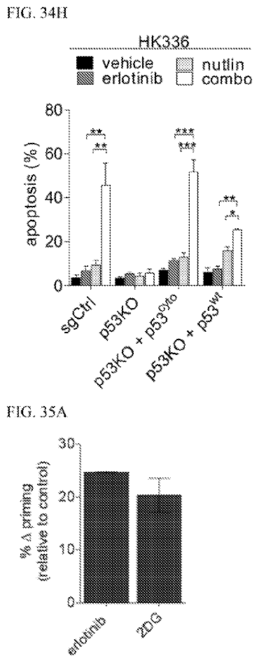

The mechanistic studies revealed a potential therapeutic opportunity in EGFR-driven GBMs that will be dependent on functional p53. While the p53 signaling axis is one of the three core pathways altered in GBM, analysis of the TCGA GBM dataset demonstrated that p53 mutations are mutually exclusive with alterations in EGFR (FIGS. 28A and 28B). Conversely, in patients with EGFR mutations or gains, the p53 pathway can be suppressed through amplification of MDM2 and/or deletions in the negative regulator of MDM2, p14 ARF, at the CDKN2A locus. Given these relationships, and the requirement of p53 for priming under EGFRi-attenuated glucose uptake, we hypothesized that stabilization of p53 via MDM2 inhibition may have similar therapeutic effects to Bcl-xL antagonism. Using nutlin—an extensively characterized inhibitor of MDM2—we found striking synergistic lethality when paired with erlotinib in a metabolic responder gliomasphere. Greater than 90% of HK301 cells underwent apoptosis with combined erlotinib and nutlin (FIG. 24C). Notably, we observed no synergy between these drugs in a metabolic non-responder (GS017, FIG. 24C). We then tested this combination across our panel of primary GBM cells (all p53 wild-type) and found synergistic lethality only in GBMs with a metabolic response to EGFRi (FIG. 24D and FIG. 34A). Genetic knockdown of EGFR confirmed synergy only in the metabolic responders (FIG. 34B). Importantly, enforced expression of GLUT1/3 significantly reduced BAX oligermization, cytochrome c release and apoptosis with combined eriotinib and nutlin (FIG. 24E and FIG. 34C), supporting the concept that inhibition of glucose metabolism with EGFRi is required for the synergistic effects of the erlotinib and nutlin combination.

The role of p53 in eliciting cell death to combined erlotinib and nutlin was then investigated. As expected, CRISPR/CAS-9 targeting of p53 in two EGFRi metabolic responders (HK301 and HK336) completely mitigated sensitivity to the drug combination (FIG. 24F). Likewise, ectopic expression of Bcl-xL markedly suppressed cell death with combined treatment, consistent with a critical function for Bcl-xL in antagonizing p53-mediated apoptosis (FIG. 34D). Moreover, similar to the results with Bcl-xL inhibition (e.g., WEHI-539), the addition of nutlin liberated p53 from Bcl-xL under erlotinib treatment (FIG. 24G). These data are in agreement with prior observations that p53 stabilization can stimulate cytoplasmic p53-mediated apoptosis. In support of the suggestion that cytoplasmic p53 activity is required for EGFRi and nutlin induced apoptosis in metabolic responders, blocking cytoplasmic p53 activity with PFTμ significantly reduced the synergistic effects of the combination (FIG. 34E), while, HK301 cells containing the nuclear-confined p53 mutant, p53^{NES}, were incapable of enhanced apoptosis with erlotinib and nutlin (FIG. 34F). Finally, the cancer “hotspot” mutants, R175H and R273H, which have both transactivation and cytoplasmic deficiencies, were completely insensitive to the drug combination (FIG. 34F).

While cytoplasmic p53 is desired to promote cell death with the drug combination, we observed in some instances that both the transcription-dependent and independent functions of p53 are needed for optimal execution of synergistic apoptosis with nutlin (FIG. 34F). These results are consistent with reports that the transcription-independent functions of p53 can alone execute intrinsic apoptosis, whereas, in other contexts, may require its transcription-dependent functions to stimulate cytoplasmic p53 mediated cell kill. Collectively, the results described herein show that combined targeting of EGFR-driven glucose metabolism and p53 can induce marked synergistic cell death in primary GBM; which is dependent on the cytoplasmic functions of p53.

### Example 11: Modulation of Glucose Metabolism Primes EGFRi Non-Responders for p53-Mediated Cell Death

The aforementioned data led the inventors to propose a model where EGFRi-mediated attenuation of glucose metabolism primes the apoptotic machinery, resulting in synergy with pro-apoptotic stimuli such as p53 activation. The synergy lies between induction of cellular stress by EGFR inhibitors, reduction of glucose uptake and the priming of the cell for apoptosis and the stabilization of p53 by antagonists of BCL-2. EGFR inhibition can rapidly attenuate glycolysis in cellular stress. This creates a tumor-specific vulnerability in which intrinsic apoptosis can be significantly enhanced by: 1) activation of p53 (such as, for example, through nutlin, analogues or others described herein) and 2) inhibition of BCL-2 (by any of several agents as described herein such as for example, ABT-263 (Navitoclax).

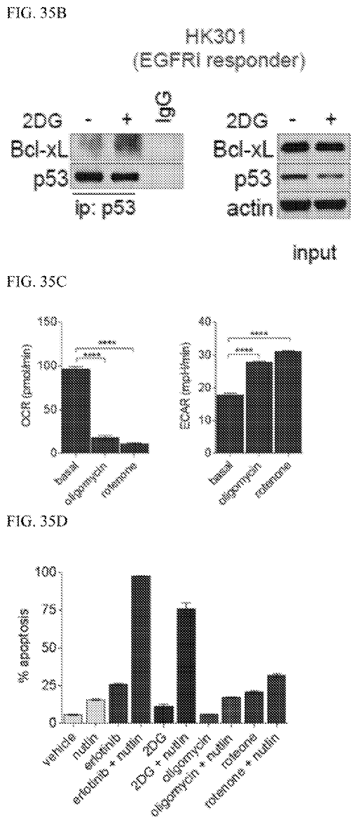

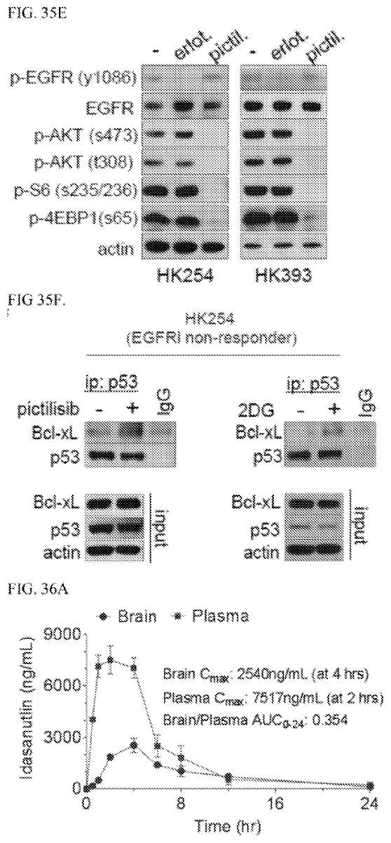

A logical prediction of this model is that direct inhibition of glucose metabolism should phenocopy the effects of EGFRi. Consistent with this, addition of the glucose metabolic inhibitor 2-deoxyglucose (2DG) stimulated apoptotic priming, binding of p53 to Bcl-xL, and synergy with nutlin in HK301 cells (an EGFRi metabolic responder) (FIGS. 40A, 40B, and 40D). Interestingly, inhibition of oxidative phosphorylation with oligomycin (complex V/ATP synthase) or rotenone (complex I) did not synergize with nutlin treatment in HK301 gliomaspheres (FIGS. 35C and 35D). Thus, reduced glucose metabolic flux alone, but not oxidative metabolism, appears to be sufficient for synergistic sensitivity to p53 activation.

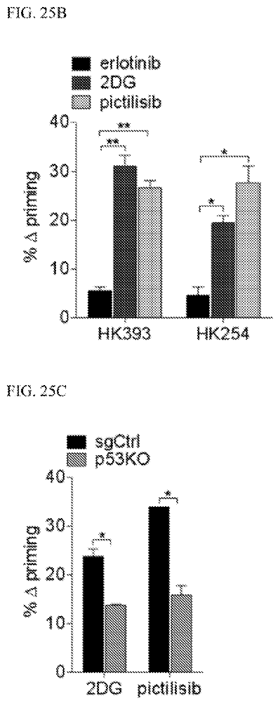

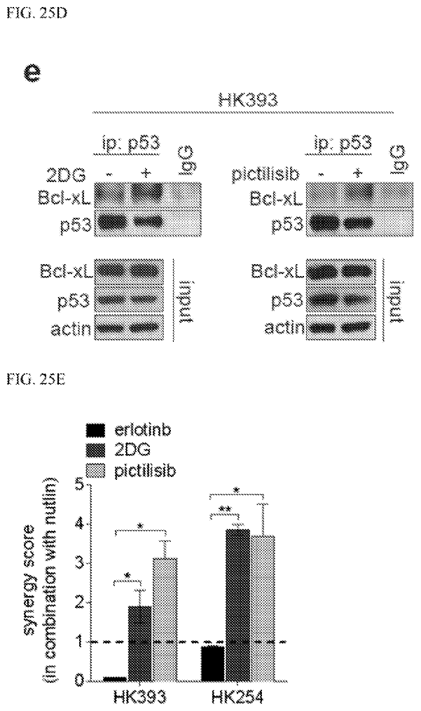

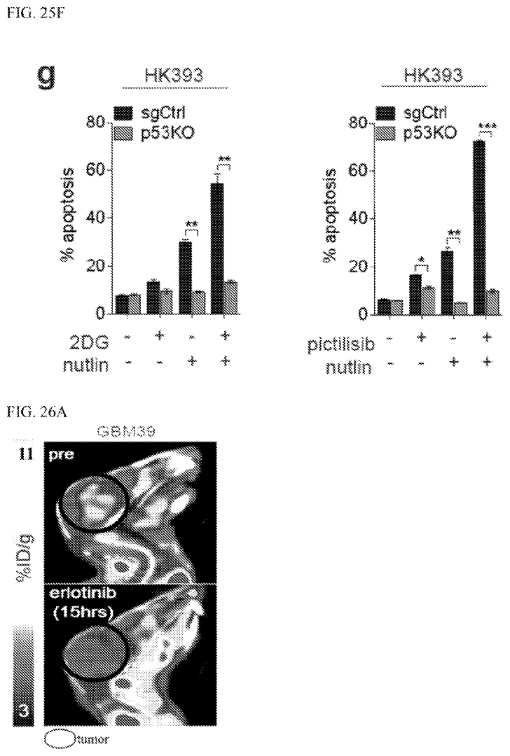

This prompted the inventors to consider whether modulating glucose consumption in EGFRi non-responders results in a similar p53-dependent vulnerability. To investigate this, they tested whether direct inhibition of glucose uptake, with 2DG, or through targeting PI3K—a well characterized driver of glucose metabolism—elicits apoptotic priming in two EGFRi metabolic non-responders (FIG. 25A). In contrast to erlotinib treatment, acute inhibition of PI3K with pictilisib abrogated PI3K-AKT-mTOR signaling (FIG. 35E), and significantly reduced ¹⁸F-FDG uptake in HK393 and HK254 cells (FIG. 25B). The decrease in glucose consumption with pictilisib was associated with significantly higher apoptotic priming and, as anticipated, 2DG completely mirrored these effects (FIGS. 25B and C). Therefore, EGFRi metabolic non-responders can be primed for apoptosis following inhibition of glucose uptake. Importantly, CRISPR/CAS-9 targeting of p53 in HK393 significantly suppressed priming mediated by 2DG or pictilisib. (FIG. 25D). Moreover, p53-dependent priming was associated with heightened Bcl-xL and p53 binding, indicative of sequestration of p53 by Bcl-xL to block apoptosis (FIG. 25E and FIG. 35F). Consistent with this interpretation, combining 2DG or pictilisib with nutlin caused significant, p53-dependent synergistic lethality in EGFRi non-responder cells (FIGS. 25F & 25G). Taken together, these data demonstrate that acute inhibition of glucose metabolism, either directly or with targeted therapy, promotes p53-dependent apoptotic priming in GBM; which, creates a targetable vulnerability for enhanced cell kill.

### Example 12: Combinatorial Therapeutic Strategy and Non-Invasive Biomarker for Targeting GBM In Vivo

The results obtained in cell culture show that combined targeting of oncogene-driven glucose metabolism and p53 has synergistic activity in primary GBM. This led the us to investigate whether this approach could be effective in orthotopic GBM xenograft models. For these studies, we employed a potent, MDM2 inhibitor, Idasanutlin, which is currently in clinical trials for many malignancies. Given the uncertainty of CNS penetration for Idasanutlin, we first demonstrated that Idasanutlin can accumulate in the brain of mice with an intact blood-brain-barrier (brain:plasma, 0.35) and stabilizes p53 in orthotopic tumor-bearing mice (FIGS. 41A & 41B).

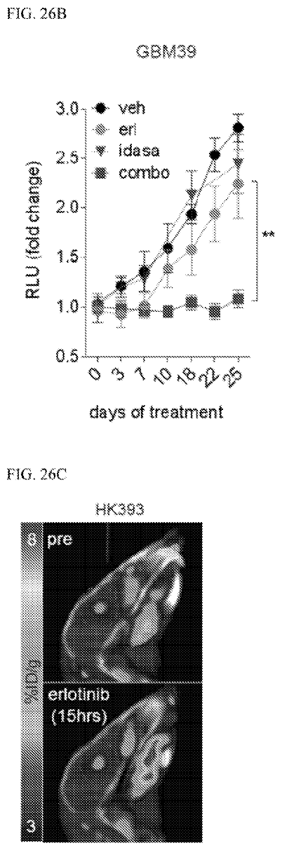

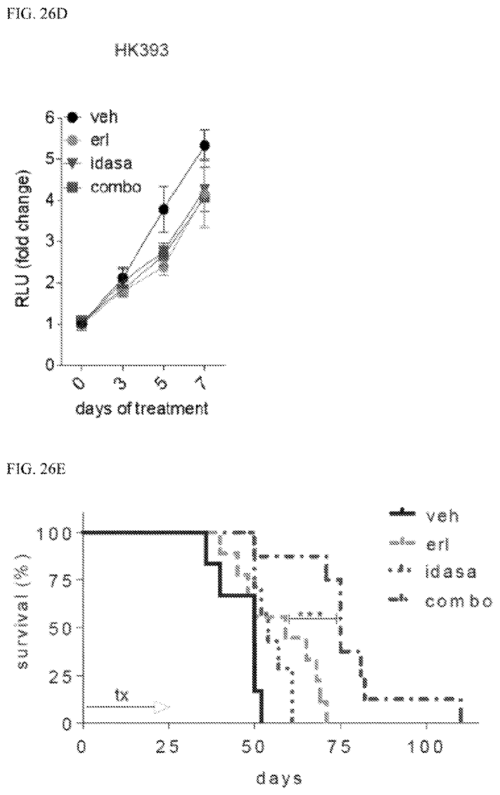

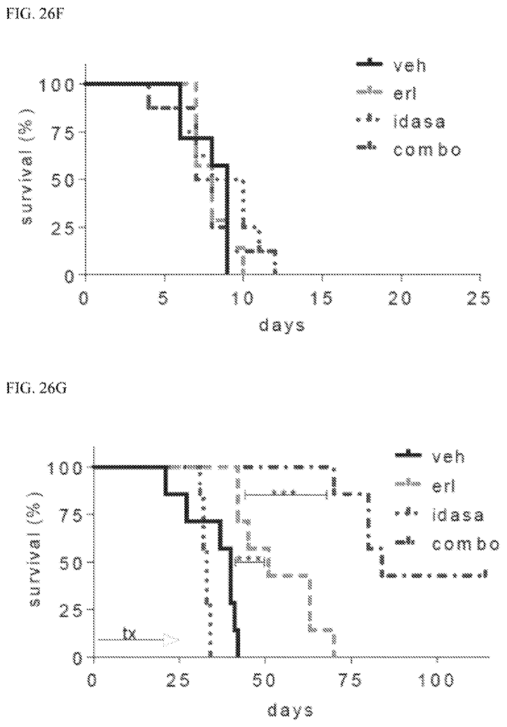

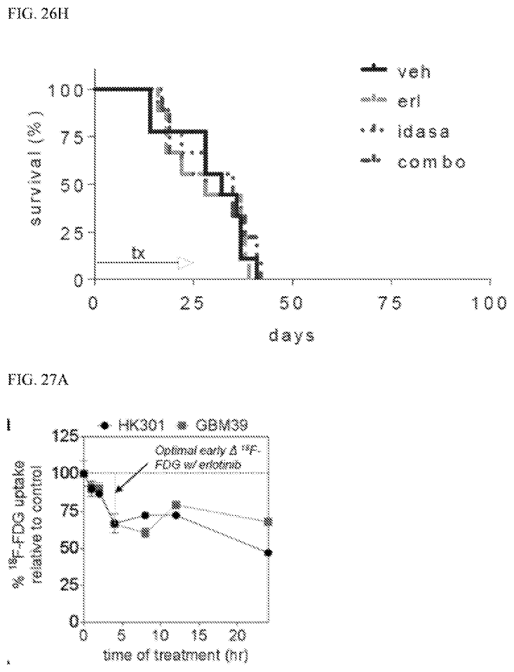

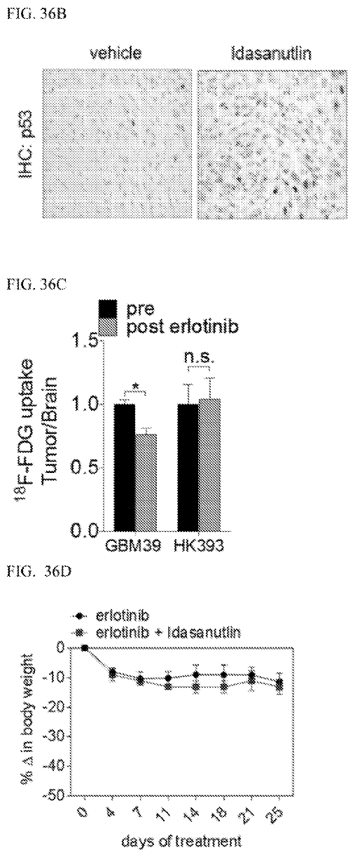

Next, as perturbations in glucose metabolism with oncogene inhibition are required for synergistic sensitivity to p53 activation, we reasoned that rapid attenuation in glucose uptake in vivo following EGFRi administration—as measured by ¹⁸F-FDG PET—could serve as a non-invasive predictive biomarker for therapeutic efficacy of combined erlotinib+Idasanutlin treatment (FIG. 26A). We observed, in orthotopic xenografts of an EGFR-metabolic responder gliomasphere (GBM39), that acute erlotinib treatment (75 mg/kg) rapidly reduced ¹⁸F-FDG uptake (15 hours post erlotinib administration) (FIG. 26B and FIG. 36C). In separate groups of mice, they tested the individual drugs and the combination of daily erlotinib (75 mg/kg) treatment and Idasanutlin (50 mg/kg). Relative to single agent controls, we observed synergistic growth inhibition—as determined by secreted gaussia luciferase—in GBM39 intracranial tumor-bearing mice, with minimal toxicity (FIG. 26B and FIG. 36D). In contrast, orthotopic xenografts of a non-metabolic responder (HK393) showed no changes in ¹⁸F-FDG uptake with acute EGFRi (FIG. 26D and FIG. 36C), nor synergistic activity with the erlotinib and Idasanutlin combination (FIG. 26E). Thus, non-invasive ¹⁸F-FDG PET, used to measure rapid changes in glucose uptake with EGFRi, was effective in predicting subsequent synergistic sensitivity to combined erlotinib and Idasanutlin.

Finally, we evaluated the effects of the drug combination on overall survival in orthotopic xenografts of either two EGFRi metabolic responders (GBM39 and HK336) or two non-responders (HK393 and GS025). All tumors were p53 wild-type (FIG. 29A). Following evidence of tumor growth (as determined by gaussia luciferase), mice were treated with vehicle, erlotinib, Idasanutlin, or the combination for up to 25 days. The drug combination led to a pronounced increase in survival only in the EGFRi metabolic responder GBM tumors (FIGS. 30F-I). Taken together, these data show that combined targeting of EGFR and p53 synergistically inhibits growth and prolongs survival in a subset of p53 wild-type GBM orthotopic xenografts. Importantly, ¹⁸F-FDG PET was valuable as a non-invasive predictive biomarker of sensitivity to this new combination therapeutic strategy.

### Example 13: Direct Inhibition of Glycolysis with 2DG or Cytocahalsin B

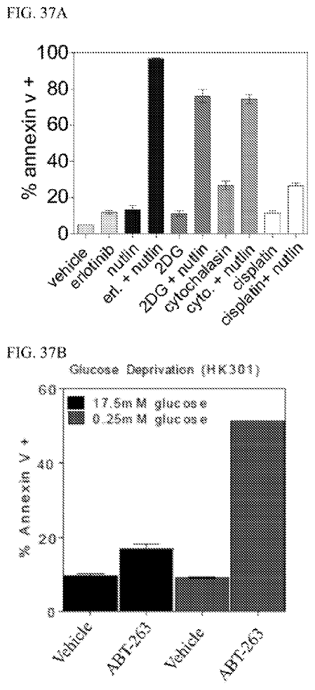

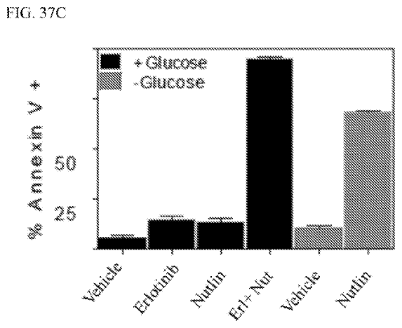

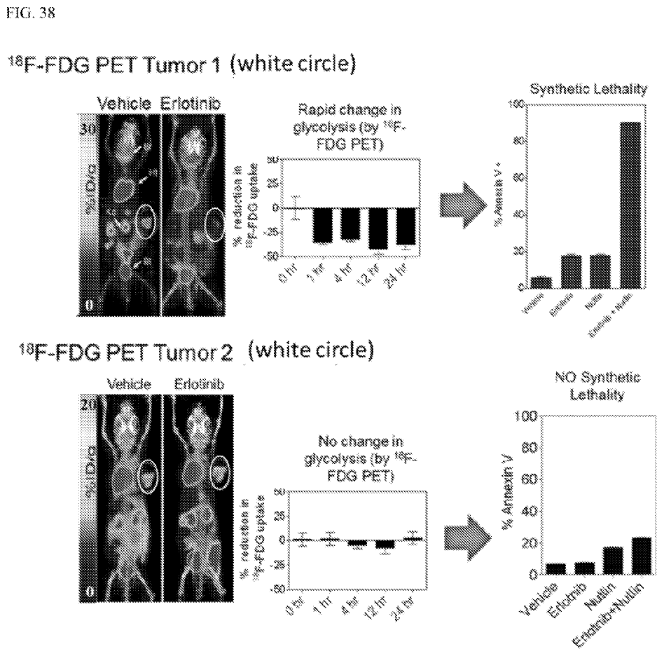

We tested how direct inhibition of glycolysis with a hexokinase inhibitor (2DG) and a glucose transporter inhibitor (cytochalasin B) affect p53 activation by nutlin. The results shown in FIG. 37 demonstrate that low glucose (0.25 mM) leads to synergistic cell kill with BCL-xL inhibition with navitoclax or nutlin. Cell death was measured using annexin V staining in gliomasphere samples treated for 72 hours with glycolytic inhibitors 2DG or cytochalasin B as single agents or in combination with p53 activator, nutlin. The same effects were recapitulated by culturing gliomaspheres in low glucose conditions (0.25 mM) and treating them with nutlin or navitoclax (ABT-263) for 72 hours.

### Example 14: Experimental Procedures

### Mice

Female NOD scid gamma (NSG), 6-8 weeks of age, were purchased from the University of California Los Angeles (UCLA) medical center animal breeding facility. Male CD-1 mice, 6-8 weeks of age, were purchased from Charles River. All mice were kept under defined flora pathogen-free conditions at the AAALAC-approved animal facility of the Division of Laboratory Animals (DLAM) at UCLA. All animal experiments were performed with the approval of the UCLA Office of Animal Resource Oversight (OARO).

### Patient-Derived GBM Cells

All patient tissue to derive GBM cell cultures was obtained through explicit informed consent, using the UCLA Institutional Review Board (IRB) protocol: 10-00065. As previously described¹², primary GBM cells were established and maintained in gliomasphere conditions consisting of DMEM/F12 (Gibco), B27 (Invitrogen), Penicillin-Streptomycin (Invitrogen), and Glutamax (Invitrogen) supplemented with Heparin (5 μg/mL, Sigma), EGF (50 ng/mL, Sigma), and FGF (20 ng/mL, Sigma). All cells were grown at 37° C., 20% O₂, and 5% CO₂ and were routinely monitored and tested negative for the presence of mycoplasma using a commercially available kit (MycoAlert, Lonza). At the time of experiments, most HK lines used were between 20-30 passages (exceptions HK385 p8, HK336 p15), while GS and GBM39 lines were less than 10 passages. All cells were authenticated by short-tandem repeat (STR) analysis

### Reagents and Antibodies

Chemical inhibitors from the following sources were dissolved in DMSO for in vitro studies: Erlotinib (Chemietek), Nutlin-3A (Selleck Chemicals), WEHI-539 (APExBIO), Pictilisib (Selleck Chemicals), Oligomycin (Sigma), Rotenone (Sigma). 2DG (Sigma) was dissolved freshly in media prior to usage. Antibodies used for immunoblotting were obtained from the listed sources: β-actin (Cell signaling, 3700), tubulin (Cell signaling, 3873), p-EGFR Y1086 (Thermo Fischer Scientific, 36-9700), t-EGFR (Millipore, 06-847), t-AKT (Cell Signaling, 4685), p-AKT T308 (Cell Signaling, 13038), p-AKT S473 (Cell Signaling, 4060), t-ERK (Cell Signaling, 4695), p-ERK T202/Y204 (Cell Signaling, 4370), t-S6 (Cell Signaling, 2217), p-S6 S235/236 (Cell Signaling, 4858), t-4EBP1 (Cell Signaling, 9644), p-4EBP1 S65 (Cell Signaling 9451), Glut3 (Abcam, ab15311), Glut1 (Millipore, 07-1401), p53 (Santa Cruz Biotechnology, SC-126), BAX (Cell Signaling, 5023), BIM (Cell Signaling, 2933), Bcl-2 (Cell Signaling, 2870), Bcl-xL (Cell Signaling, 2764), Mcl-1 (Cell Signaling, 5453), Cytochrome c (Cell Signaling, 4272), and Cleaved Caspase-3 (Cell Signaling, 9661). Antibodies used for immunoprecipitation were obtained from the listed sources: p53 (Cell Signaling, 12450) and Bcl-xL (Cell Signaling, 2764). Secondary antibodies were obtained from the listed sources: Anti-rabbit IgG HRP-linked (Cell Signaling, 7074) and Anti-mouse IgG HRP-linked (Cell Signaling, 7076). All immunoblotting antibodies were used at a dilution of 1:1000, except β-actin and tubulin, which were used at 1:10,000. Immunoprecipitation antibodies were diluted according to manufacturer's instructions (1:200 for p53 and 1:100 for Bcl-xL). Secondary antibodies were used at a dilution of 1:5000.

### ¹⁸F-Fluorodeoxyglucose (18F-FDG) Uptake Assay.

Cells were plated at 5×10 cells/ml and treated with designated drugs for indicated time points. Following appropriate treatment, cells were collected and resuspended in glucose-free DMEM/F12 (USBiological) containing ¹⁸F-FDG (radioactivity 1 μCi/mL). Cells were incubated at 37° C. for 1 hr and then washed three times with ice cold PBS. Radioactivity of each sample was then measured using a gamma counter.

### Glucose, Glutamine, and Lactate Measurements

Cellular glucose consumption and lactate production were measured using a Nova Biomedical BioProfile Basic Analyzer. Briefly, cells were plated in 1×10⁵ cells/ml in 2 mL of gliomasphere conditions and appropriate drug conditions (n=5). 12 hrs following drug treatment, 1 ml of media was removed from each sample and analyzed in the Nova BioProfile analyzer. Measurements were normalized to cell number.

### Annexin V Apoptosis Assay

Cells were collected and analyzed for Annexin V and PI staining according to manufacturer's protocol (BD Biosciences). Briefly, cells were plated at 5×10⁴ cells/ml and treated with appropriate drugs. Following indicated time points, cells were collected, trypsinized, washed with PBS, and stained with Annexin V and PI for 15 minutes. Samples were then analyzed using the BD LSRII flow cytometer.

### Immunoblotting

Cells were collected and lysed in RIPA buffer (Boston BioProducts) containing Halt Protease and Phosphatase Inhibitor (Thermo Fischer Scientific). Lysates were centrifuged at 14,000×g for 15 min at 4° C. Protein samples were then boiled in NuPAGE LDS Sample Buffer (Invitrogen) and NuPAGE Sample Reducing Agent (Invitrogen) and separated using SDS-PAGE on 12% Bis-Tris gels (Invitrogen) and transferred to nitrocellulose membrane (GE Healthcare). Immunoblotting was performed per antibody's manufacturer's specifications and as mentioned previously. Membranes were developed using the SuperSignal system (Thermo Fischer Scientific).

### Immunoprecipitation

Cells were collected, washed once with PBS, and incubated in IP lysis buffer (25 mM Tris-HCL pH 7.4, 150 mM NaCl, 1 mM EDTA, 1% NP-40, 5% Glycerol) at 4° C. for 15 minutes. 300-500 μg of each sample was then pre-cleared in Protein A/G Plus Agarose Beads (Thermo Fischer Scientific) for one hour. Following pre-clear, samples were then incubated with antibody-bead conjugates overnight according to manufacturer's specifications and as mentioned previously. The samples were then centrifuged at 1000 g for 1 min, and the beads were washed with 500 μL of IP lysis buffer for five times. Proteins were eluted from the beads by boiling in 2×LDS Sample Buffer (Invitrogen) at 95° C. for 5 min. Samples analyzed by immunoblotting as previously described. Immunoprecipitation antibodies were diluted according to manufacturer's instructions (1:200 for p53 and 1:100 for Bcl-xL).

### Dynamic BH3 Profiling

GBM gliomaspheres were first disassociated to single-cell suspensions with TrypLE (Gibco) and resuspended in MEB buffer (150 mM Mannitol 10 mM HEPES-KOH, 50 mM KCl, 0.02 mM EGTA, 0.02 mM EDTA, 0.1% BSA, 5 mM Succinate). 50l of cell suspension (3×10⁴ cells/well) were plated in wells holding 50 μL MEB buffer containing 0.002% digitonin and indicated peptides in 96-well plates. Plates were then incubated at 25° C. for 50 min. Cells were then fixed with 4% paraformaldehyde for 10 min, followed by neutralization with N2 buffer (1.7M Tris, 1.25M Glycine pH 9.1) for 5 min. Samples were stained overnight with 20 μL of staining solution (10% BSA, 2% Tween 20 in PBS) containing DAPI and anti-cytochrome c (BioLegend). The following day, cytochrome c release was quantified using BD LSRII flow cytometer. Measurements were normalized to appropriate controls that do not promote cytochrome c release (DMSO and inactive PUMA2A peptide). Delta priming refers to the difference in amount of cytochrome c release between vehicle treated cells and drug treated cells.

### BAX Oligomerization

7.5×10⁵ cells were treated with indicated drugs. Following 24 hr of treatment, cells were collected, washed once with ice cold PBS, and re-suspended in 1 mM bismaleimidohexane (BMH) in PBS for 30 min. Cells were then pelleted and lysed for immunoblotting, as described above.

### Cytochrome c Detection

5 million cells were plated at a concentration of 1×10⁵ cells/mL and treated with indicated drugs. Following 24 hr of treatment, cells were collected, washed once with ice cold PBS. Subcellular fractionation was then performed using a mitochondrial isolation kit (Thermo Fischer Scientific, 89874). Both cytoplasmic and mitochondrial fractions were subjected to immunoblotting and cytochrome c was detected using cytochrome c antibody at a dilution of 1:1000 (Cell Signaling, 4272).

### Mouse Xenograft Studies

For intracranial experiments, GBM39, HK336, HK393, and GS025 cells were injected (4×10⁵ cells per injection) into the right striatum of the brain of female NSG mice (6-8 weeks old). Injection coordinates were 2 mm lateral and 1 mm posterior to bregma, at a depth of 2 mm. Tumor burden was monitored by secreted gaussia luciferase and following three consecutive growth measurements, mice were randomized into four treatment arms consisting of appropriate vehicles, 75 mg/kg erlotinib, 50 mg/kg Idasanutlin, or a combination of both drugs. Vehicle consisted of 0.5% methylcellulose in water, which is used to dissolve erlotinib, and a proprietary formulation obtained from Roche, which is used to dissolve Idasanutlin. Tumor burden was assessed twice per week by secreted gaussia luciferase. When possible, mice were treated for 25 days and taken off treatment and monitored for survival. Drugs were administered through oral gavage. Sample sizes were chosen based off estimates from pilot experiments and results from previous literature¹². Investigators were not blinded to group allocation or assessment of outcome. All studies were in accordance with UCLA OARO protocol guidelines.

### Intracranial Delayed PET/CT Mouse Imaging

Mice were treated with indicated dose and time of erlotinib then pre-warmed, anesthetized with 2% isoflurane, and intravenously injected with 70 μCi of ¹⁸F-FDG. Following 1 hr unconscious uptake, mice were taken off anesthesia but kept warm for another 5 hr of uptake. 6 hr after the initial administration of ¹⁸F-FDG, mice were imaged using G8 PET/CT scanner (Sofie Biosciences). Per above, quantification was performed by drawing 3D regions of interest (ROI) using the AMIDE software.

### Immunohistochemistry

Immunohistochemistry was performed on 4 μm sections that were cut from FFPE (formalin-fixed, paraffin-embedded) blocks. Sections were then deparaffinised with xylene and rehydrated through graded ethanol. Antigen retrieval was achieved with a pH 9.5 Nuclear Decloaker (Biocare Medical) in a Decloaking pressure cooker at 95° C. for 40 min. Tissue sections were then treated with 3% hydrogen peroxide (LOT 161509; Fisher Chemical) and with Background Sniper (Biocare Medical, Concord, Calif., USA) to reduce nonspecific background staining. Primary antibody for p53 (Cell Signaling, 2527) was applied in a 1:150 dilution for 80 min followed by detection with the MACH 3 Rabbit HRP-Polymer Detection kit (Biocare Medical). Visualization was achieved using VECTOR NovaRED (SK-4800; Vector Laboratories, Inc.) as chromogen. Lastly, sections were counterstained with Tacha's Automated Hematoxylin (Biocare Medical).

### Quantitative RT-PCR

RNA was extracted from all cells using Purelink RNA Kit (Invitrogen). cDNA was synthesized with iScript cDNA Synthesis Kit (Bio-Rad) as per manufacturer's instructions. Quantitative PCR (qPCR) was conducted on the Roche LightCycler 480 using SYBRGreen Master Mix (Kapa Biosciences). Relative expression values are normalized to control gene (GAPDH). Primer sequences are as listed (5′ to 3′): P21 (forward GACTTTGTCACCGAGACACC, reverse GACAGGTCCACATGGTCTTC), PUMA (forward ACGACCTCAACGCACAGTACG, reverse GTAAGGGCAGGAGTCCCATGATG), GAPDH (forward TGCCATGTAGACCCCTTGAAG, reverse ATGGTACATGACAAGGTGCGG), MDM2 (forward CTGTGTTCAGTGGCGATTGG, reverse AGGGTCTCTTGTTCCGAAGC), TIGAR (forward GGAAGAGTGCCCTGTGTTTAC, reverse GACTCAAGACTTCGGGAAAGG), PIG3 (forward GCAGCTGCTGGATTCAATTA, reverse TCCCAGTAGGATCCGCCTAT).

### P53 Reporter Activity

Cells were first infected with lentivirus synthesized from a p53 reporter plasmid which codes for luciferase under the control of a p53 responsive element: TACAGAACATGTCTAAGCATGCTGTGCCTTGCCTGGACTTGCCTGGCCTTGCCTT GGG. Infected cells were then plated into a 96-well plate at 5,000 cells/50 μL and treated with indicated drugs for 24 hr and then incubated with 1 mM D-luciferin for two hours. Bioluminescence was measured using IVIS Lumina II (Perkin Elmer).

### Genetic Manipulation

In general, lentivirus used for genetic manipulation were produced by transfecting 293-FT cells (Thermo) using Lipofectamine 2000 (Invitrogen). Virus was collected 48 hours after transfection. The lentiviral sgp53 vector and sgControl vector contained the following guide RNA, respectively: CCGGTTCATGCCGCCCATGC and GTAATCCTAGCACTTTTAGG. LentiCRISPR-v2 was used as the backbone. Glut1 and Glut3 cDNA was cloned from commercially available vectors and incorporated into pLenti-GLuc-IRES-EGFP lentiviral backbone containing a CMV promoter (Glut1 was a gift from Wolf Frommer (Addgene #18085⁴⁴), Glut3 was obtained from OriGene # SC 115791, and the lentiviral backbone was obtained from Targeting Systems # GL-GFP). pMIG Bcl-xL was a gift from Stanley Korsmeyer (Addgene #8790⁴⁵) and cloned into the lentiviral backbone mentioned above (Targeting Systems). Cytoplasmic (K305A and R306A) and wild-type p53 constructs were a kind gift from R. Agami and G. Lahav. The genes of interest were cloned into a lentiviral vector containing a PGK promoter. Constructs for p53 DNA binding domain mutants (R175H) and (R273H) as well as the nuclear mutant (L348A and L350A) were generated using site-directed mutagenesis (New England Biolabs # E0554S) on the wild-type p53 construct.

For EGFR knockdown experiments, siRNA against EGFR (Thermo Fischer Scientific, s563) was transfected into cells using DharmaFECT 4 (Dharmacon). Following 48 hours, cells were harvested and used for indicated experiments.

### Immunofluorescence

For immunofluorescence, gliomaspheres were first disassociated to single cell and adhered to the 96-well plates using Cell-Tak (Corning) according to manufacturer instructions. Adhered cells were then fixed with ice-cold methanol for 10 min then washed three times with PBS. Cells were then incubated with blocking solution containing 10% FBS and 3% BSA in PBS for 1 hr and subsequently incubated with p53 (Santa Cruz, SC-126, dilution of 1:50) antibody overnight at 4° C. The following day, cells were incubated with secondary antibody (Alexa Fluor 647, dilution 1:2000) for an hour and DAPI staining for 10 min, then imaged using a Nikon TI Eclipse microscope equipped with a Cascade II fluorescent camera (Roper Scientific). Cells were imaged with emissions at 461 nM and 647 nM and then processed using NIS-Elements AR analysis software.

### Oxygen Consumption Rate (OCR) and Extracellular Acidification Rate (ECAR) Measurements

For metabolic measurements involving OCR and ECAR, gliomaspheres treated with indicated drugs were first disassociated to single cell suspensions and adhered to XF24 plates (Seahorse Bioscience) using Cell-Tak (Corning) according to manufacturer instructions. Prior to the assay, cells were supplemented with unbuffered DMEM, and incubated at 37° C. for 30 min before starting OCR and ECAR measurements. Basal ECAR measurements between control and erlotinib treated cells are shown.

### Mass-Spectroscopy Sample Preparation

Male CD-1 mice (6-8 weeks old) were treated with 50 mg/kg Idasanutlin in duplicate through oral gavage. At 0.5, 1, 2, 4, 6, 8, 12, and 24 hr after administration, mice were sacrificed, blood was harvested by retro-orbital bleeding, and brain tissue was collected. Whole blood from mice was centrifuged to isolate plasma. Idasanutlin was isolated by liquid-liquid extraction from plasma: 50 μL plasma was added to 2 μL internal standard and 100 μL acetonitrile. Mouse brain tissue was washed with 2 mL cold PBS and homogenized using a tissue homogenizer with fresh 2 mL cold PBS. Idasanutlin was then isolated and reconstituted in a similar manner by liquid-liquid extraction: 100 μL brain homogenate was added to 2 μL internal standard and 200 μL acetonitrile. After vortex mixing, the samples was centrifuged. The supernatant was removed and evaporated by a rotary evaporator and reconstituted in 100 μL 50:50 water: acetonitrile.

### Idasanutlin Detection by Mass-Spectrometry

Chromatographic separations were performed on a 100×2.1 mm Phenomenex Kinetex C18 column (Kinetex) using the 1290 Infinity LC system (Agilent). The mobile phase was composed of solvent A: 0.1% formic acid in Milli-Q water, and B: 0.1% formic acid in acetonitrile. Analytes were eluted with a gradient of 5% B (0-4 min), 5-99% B (4-32 min), 99% B (32-36 min), and then returned to 5% B for 12 min to re-equilibrate between injections. Injections of 20 μL into the chromatographic system were used with a solvent flow rate of 0.10 mL/min. Mass spectrometry was performed on the 6460 triple quadrupole LC/MS system (Agilent). Ionization was achieved by using electrospray in the positive mode and data acquisition was made in multiple reactions monitoring (MRM) mode. The MRM transition used for Idasanutlin detection was m/z 616.2→421.2 with fragmentor voltage of 114V, and collision energy of 20 eV. Analyte signal was normalized to the internal standard and concentrations were determined by comparison to the calibration curve (0.5, 5, 50, 250, 500, 2000 nM). Idasanutlin brain concentrations were adjusted by 1.4% of the mouse brain weight for the residual blood in the brain vasculature.

### Secreted Gaussia Luciferase Measurements

Cells were infected with a lentiviral vector containing secreted gaussia luciferase (sGluc) reporter gene (Targeting Systems # GL-GFP) and intracranially implanted into the right striatum of mice (4×10⁵ cells/mouse). To measure the levels of secreted Gaussia luciferase (sGluc), 6 μL of blood was collected from the tail vein of the mice and immediately mixed with 50 mM EDTA to prevent coagulation. Gluc activity was obtained by measuring chemiluminescence following injection of 100 μL of 100 μM coelentarazine (Nanolight) in a 96 well plate.

### Synergy Score Calculations

1.0×10⁵ GBM cells were plated in triplicate and treated with erlotinib, nutlin, or combination at multiple concentrations using a matrix where each drug was added to the cells at six concentrations (0-10 μM). Annexin V staining was measured following 72 hrs of treatment. Using the Chalice software, the response of the combination was compared to its single agents. The combinatorial effects were calculated using the synergy score.

### DNA Sequencing

Targeted sequencing was performed for samples HK206, HK217, HK250, HK296 for the following genes BCLIIA, BCLIIB, BRAF, CDKN2A, CHEK2, EGFR, ERBB2, IDH1, IDH2, MSH6, NF1, PIK3CA, PIK3R1, PTEN, RB1, TP53 using Illumina Miseq. There were 1 to 2 million reads per sample with average coverage of 230 per gene. Copy number variants were determined for these samples using a whole genome SNP array. The genetic profile of GBM39 has been previously reported in the literature.

Whole exome sequencing was performed for samples HK157, HK229, HK248, HK250, HK254, HK296, HK301, HK336, HK350, HK390, HK393 and carried out at SeqWright. Samples were grouped into 2 pools with separate capture reactions. Nextera Rapid capture and library preparation were used and sequencing performed on a HiSeq 2500, 2×100 bp with 100× on-target coverage, 2 full rapid runs, each with 1 normal diploid control. Copy number analysis for these samples was carried out using EXCAVATOR software.

### Annotation of TCGA Samples

273 GBM samples from the TCGA were analyzed for genetic alterations in EGFR, p53 and p53-regulated pathways. Co-occurrences of mutations were examined and only significant interactions are displayed. Data was analyzed using cBioPortal as previously described.

### Fluorescence In Situ Hybridization (FISH)

Fluorescence in situ hybridization (FISH) was performed using commercially available fluorescently labeled dual-color EGFR (red)/CEP 7(green) probe (Abbott-Molecular). FISH hybridization and analyses were performed on cell lines, following the manufacturer's suggested protocols. The cells were counterstained with DAPI and the fluorescent probe signals were imaged under a Zeiss (Axiophot) Fluorescent Microscope equipped with dual- and triple-color filters.

### Statistical Analysis.

Comparisons were made using two-tailed unpaired Student's t-tests and p values <0.05 were considered statistically significant. All data from multiple independent experiments were assumed to be of normal variance. Data represent means±s.e.m. values. All statistical analyses were calculated using Prism 6.0 (GraphPad). For all in vitro and in vivo experiments, no statistical method was used to predetermine sample size and no samples were excluded. For in vivo tumor measurements, the last data sets were used for comparisons between groups. As described above, all mice were randomized before studies.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of Formula I-a or Formula I-b: 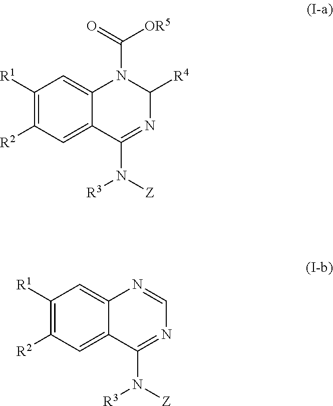
or a pharmaceutically acceptable salt or stereoisomer thereof, wherein:
Z is aryl or heteroaryl;
R¹ is hydrogen, alkyl, halo, CN, NO₂, OR⁷, cycloalkyl, heterocyclyl, aryl or heteroaryl;
R² is hydrogen, alkyl, halo, CN, NO₂, OR⁸, cycloalkyl, heterocyclyl, aryl or heteroaryl; or R¹ and R² taken together complete a carbocyclic or heterocyclic ring;
R³ is hydrogen, alkyl, or acyl;
R⁴ is alkoxy;
R⁵ is alkyl; and
R⁷ and R⁸ are, each independently, selected from hydrogen, alkyl, such as alkoxyalkyl, aralkyl, or arylacyl.

2. The compound of claim 1, wherein if R⁷ and R⁸ are alkoxyalkyl and R³ is hydrogen, then Z is not 3-ethynylphenyl.

3. The compound of any one of the preceding claims, wherein Z is optionally substituted with R⁶ selected from alkyl, alkoxy, OH, CN, NO₂, halo, alkenyl, aralkyloxy, cycloalkyl, heterocyclyl, aryl, and heteroaryl.

4. The compound of any one of the preceding claims, wherein either:
R⁷ and R⁸ are, each independently, selected from hydrogen, aralkyl, or arylacyl;
each instance of R⁶ is independently selected from alkyl, alkoxy, OH, CN, NO₂, halo, alkenyl, aralkyloxy, cycloalkyl, heterocyclyl, aryl, or heteroaryl; or
R¹ and R² taken together complete a carbocyclic or heterocyclic ring.

5. The compound of any one of claims 1-3, wherein if R⁷ and R^{B} combine to form a heterocylic ring and R³ is hydrogen, then Z is not 2-fluoro,4-bromophenyl, 3-bromophenyl, 3-methylphenyl, 3-trifluoromethylphenyl, or 3-chloro,4-fluorophenyl.

6. The compound of any one of the preceding claims, wherein the compound is a compound of Formula (II-a) or Formula (II-b):

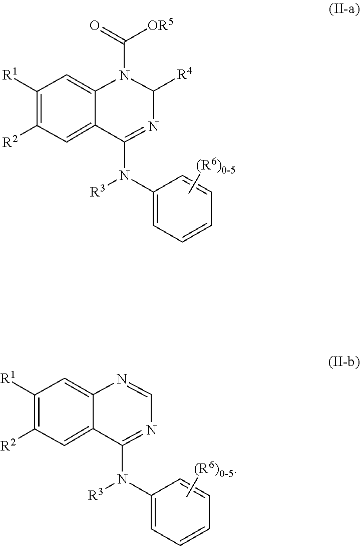

7. The compound of any one of claims 1-6, wherein R¹ is hydrogen.

8. The compound of any one of claims 1-6, wherein R¹ is OR⁷.

9. The compound of claim 8, wherein R⁷ is hydrogen.

10. The compound of claim 8, wherein R⁷ is alkyl.

11. The compound of claim 8, wherein R⁷ is alkoxyalkyl.

12. The compound of claim 8, wherein R⁷ is arylacyl.

13. The compound of any one of claims 1-12, wherein R² is heteroaryl, such as furanyl.

14. The compound of claim 13, wherein the heteroaryl is substituted with alkyl, alkoxy, OH, CN, NO₂, halo,

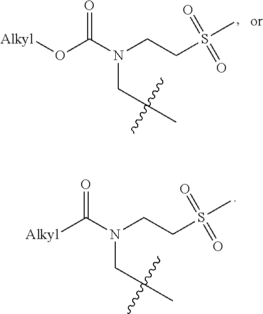

15. The compound of any one of claims 1-12, wherein R² is OR⁸.

16. The compound of claim 15, wherein R⁸ is hydrogen.

17. The compound of claim 15, wherein R⁸ is alkoxyalkyl.

18. The compound of claim 15, wherein R⁸ is alkyl substituted with

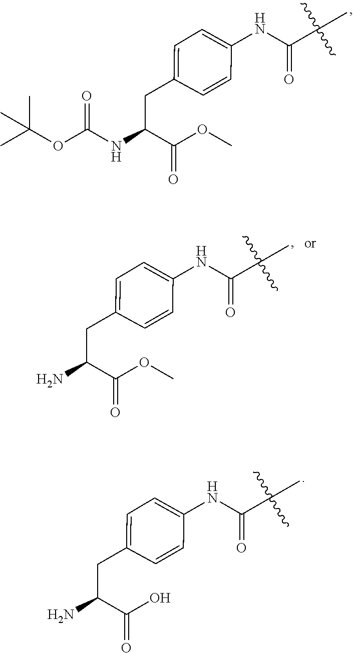 [SMILES: *C(=O)Nc1ccc(C[C@H](NC(=O)OC(C)(C)C)C(=O)OC)cc1 ; *C(=O)Nc1ccc(C[C@H](N)C(=O)OC)cc1 ; *C(=O)Nc1ccc(C[C@H](N)C(=O)O)cc1]

19. The compound of claim 15, wherein R⁸ is acyl.

20. The compound of claim 15, wherein R⁸ is arylacyl.

21. The compound of any one of claims 1-6, wherein R¹ and R² combine to form a carbocylic or heterocyclic ring, such as a 5-member, 6-member, or 7-member carbocyclic or heterocyclic ring.

22. The compound of claim 21, wherein the carbocyclic or heterocyclic ring is substituted with hydroxyl, alkyl (e.g., methyl), or alkenyl (e.g., vinyl).

23. The compound of claim 22, wherein the compound is

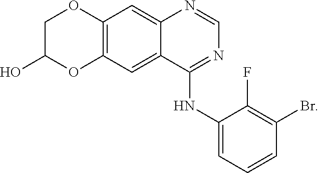 [SMILES: OC1COc2cc3ncnc(Nc4cccc(Br)c4F)c3cc2O1]

24. The compound of claim 22, wherein the carbocyclic or heterocyclic ring is substituted with alkyl (e.g., methyl) and the alkyl moieties are trans relative to each other.

25. The compound of claim 24, wherein the compound is

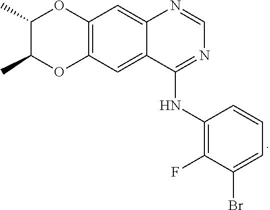 [SMILES: C[C@@H]1Oc2cc3ncnc(Nc4cccc(Br)c4F)c3cc2O[C@H]1C]

26. The compound of claim 22, wherein the carbocyclic or heterocyclic ring is substituted with alkyl (e.g., methyl) and the alkyl moieties are cis relative to each other.

27. The compound of claim 26, wherein the compound is

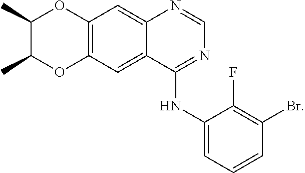 [SMILES: C[C@@H]1Oc2cc3c(Nc4cccc(Br)c4F)ncnc3cc2O[C@@H]1C]

28. The compound of claim 21, wherein the compound is a compound of Formula (III-a), (III-b), (III-c), (III-d), (III-e), or (III-f):

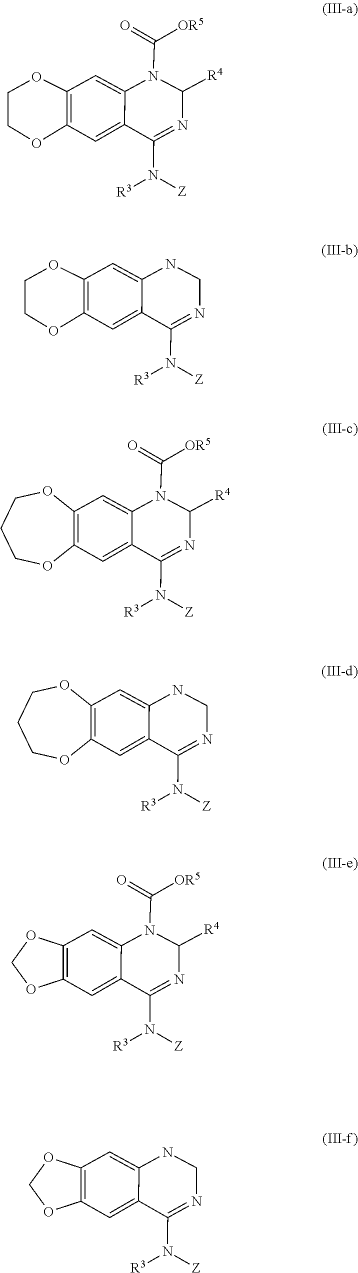

29. The compound of any one of claims 1-28, wherein R³ is hydrogen.

30. The compound of any one of claims 1-28, wherein R³ is acyl.

31. The compound of claim 30, wherein R³ is alkylacyl.

32. The compound of claim 30, wherein R³ is alkyloxyacyl.

33. The compound of claim 30, wherein R³ is acyloxyalkyl.

34. The compound of claim 30, wherein R³ is 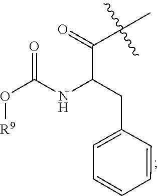
and
R⁹ is alkyl.

35. The compound of any one of the preceding claims, wherein:
Z is aryl or heteroaryl optionally substituted with one or more R⁶; and
each instance of R⁶ is independently selected from alkyl, alkoxy, OH, CN, NO₂, halo, alkenyl, alkynyl, aralkyloxy, cycloalkyl, heterocyclyl, aryl, or heteroaryl.

36. The compound of claim 35, wherein Z is phenyl substituted with 1, 2, 3, 4, or 5 R⁶.

37. The compound of claim 35 or 36, wherein each R⁶ is independently selected from halo, alkyl, alkynyl, or arylalkoxy.

38. The compound of any one of claims 35-37, wherein Z is 2-fluoro-3-chlorophenyl, 2-fluorophenyl, 2,3-difluorophenyl, 2,4-difluorophenyl, 2,5-difluorophenyl, 2,6-difluorophenyl, 2,4,6-trifluorophenyl, pentafluorophenyl, 2-fluoro-3-bromophenyl, 2-fluoro-3-ethynylphenyl, and 2-fluoro-3-(trifluoromethyl)phenyl.

39. The compound of any one of claims 35-37, wherein Z is 3-ethynylphenyl.

40. The compound of any one of claims 35-37, wherein Z is 3-chloro-4-((3-fluorobenzyl)oxy)benzene.

41. The compound of any one of claims 35-37, wherein Z is 3-chloro-2-(trifluoromethyl)phenyl.

42. The compound of any one of claims 35-37, wherein Z is 2-fluoro-3-bromophenyl.

43. The compound of any one of claims 35-37, wherein Z is 2-fluoro,5-bromophenyl.

44. The compound of any one of claims 35-37, wherein Z is 2,6-difluoro,5-bromophenyl.

45. The compound of any one of claims 35-44, wherein:
Z is substituted with one R⁶ selected from 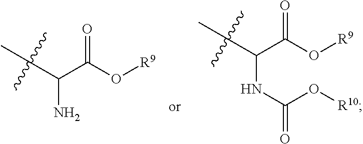
and
R⁹ and R⁰ are independently selected from alkyl.

46. The compound of any one claims 1-45, wherein the compound is a compound of Formula (IV-a): 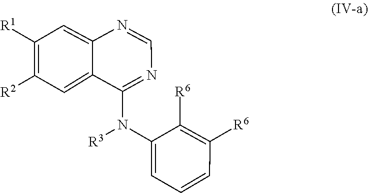
and each R⁶ is independently selected from fluoro, chloro, or bromo.

47. The compound of any one claims 1-45, wherein the compound is a compound of Formula (IV-b): 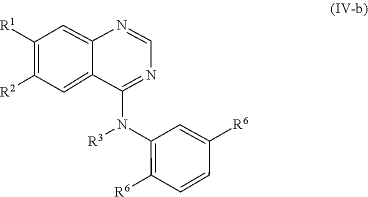
and each R⁶ is independently selected from fluoro, chloro, or bromo.

48. The compound of any one claims 1-45, wherein the compound is a compound of Formula (IV-c): 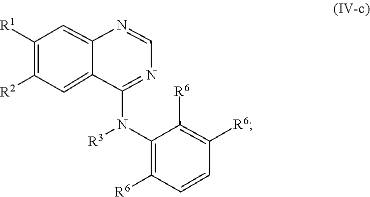
and each R⁶ is independently selected from fluoro, chloro, or bromo.

49. The compound of any one claims 1-45, wherein the compound is a compound of Formula (IV-a): 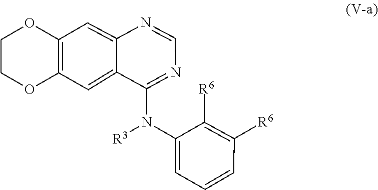
and each R⁶ is independently selected from fluoro, chloro, or bromo.

50. The compound of any one claims 1-45, wherein the compound is a compound of Formula (V-b): 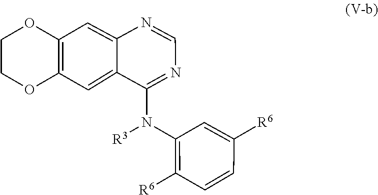
and each R⁶ is independently selected from fluoro, chloro, or bromo.

51. The compound of any one claims 1-45, wherein the compound is a compound of Formula (V-c): 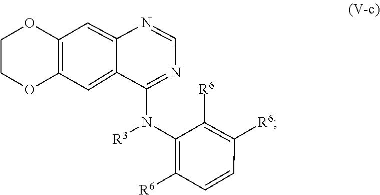
and each R⁶ is independently selected from fluoro, chloro, or bromo.

52. The compound of any one of claims 1-51, wherein the compound is: 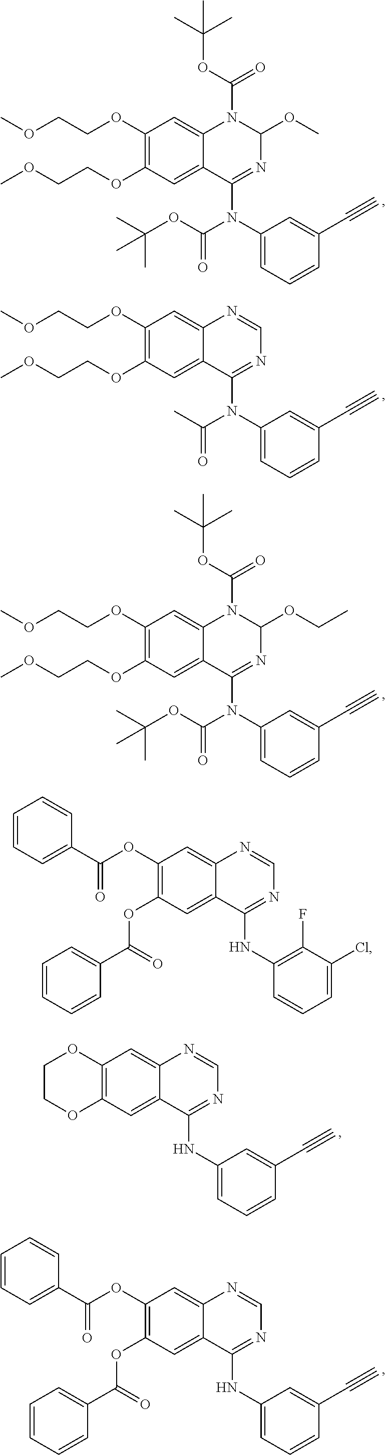 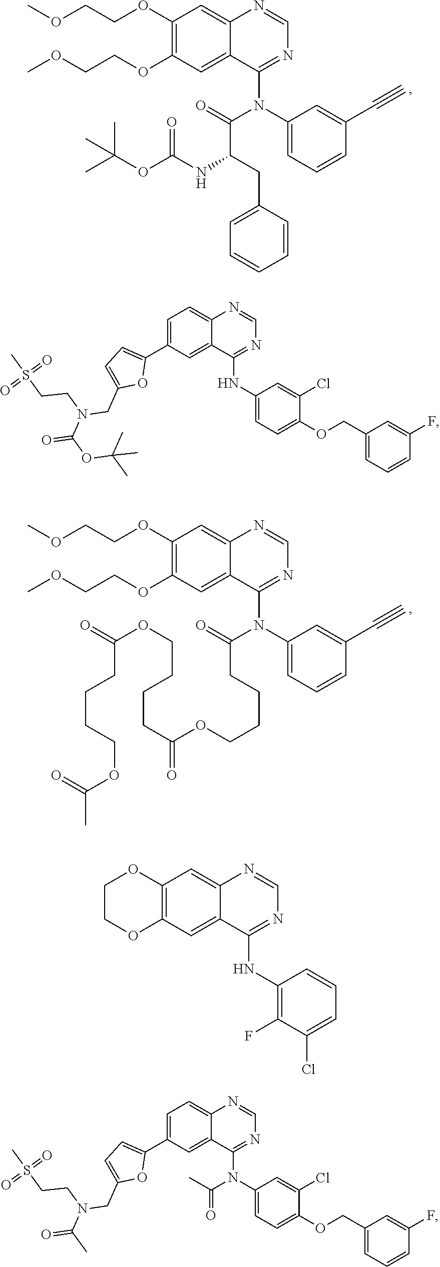 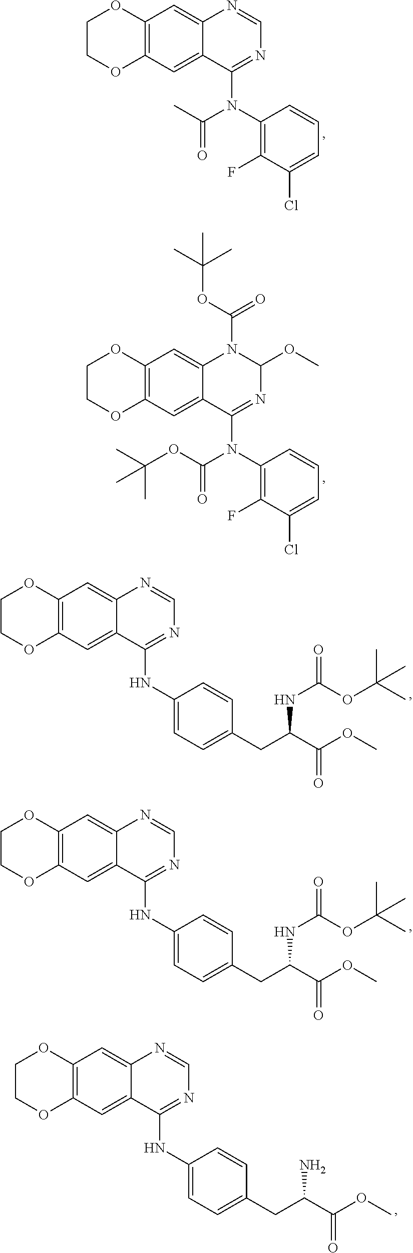 [SMILES: CC(=O)N(c1cccc(Cl)c1F)c1ncnc2cc3c(cc12)OCCO3 ; COC1N=C(N(C(=O)OC(C)(C)C)c2cccc(Cl)c2F)c2cc3c(cc2N1C(=O)OC(C)(C)C)OCCO3 ; COC(=O)[C@@H](Cc1ccc(Nc2ncnc3cc4c(cc23)OCCO4)cc1)NC(=O)OC(C)(C)C ; COC(=O)[C@H](Cc1ccc(Nc2ncnc3cc4c(cc23)OCCO4)cc1)NC(=O)OC(C)(C)C ; COC(=O)[C@@H](N)Cc1ccc(Nc2ncnc3cc4c(cc23)OCCO4)cc1] 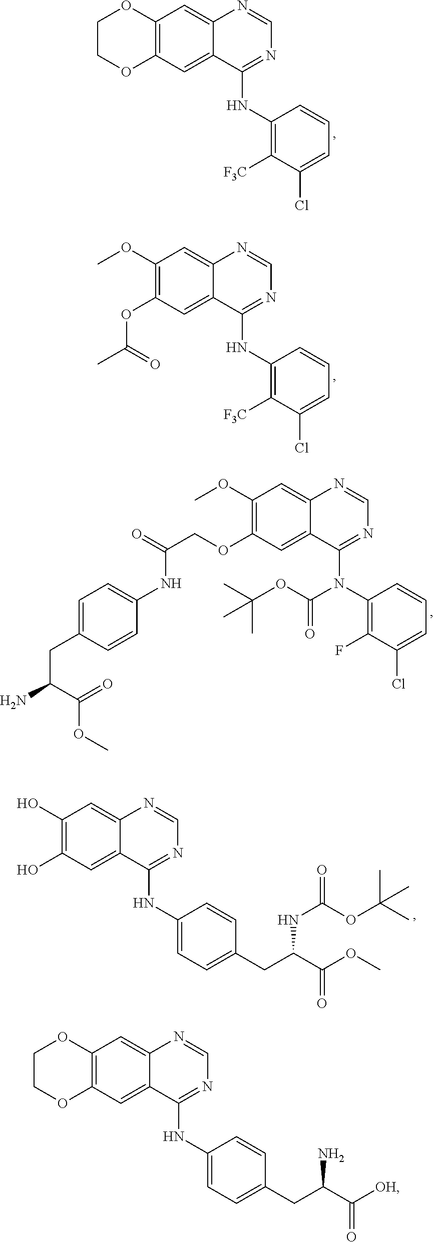 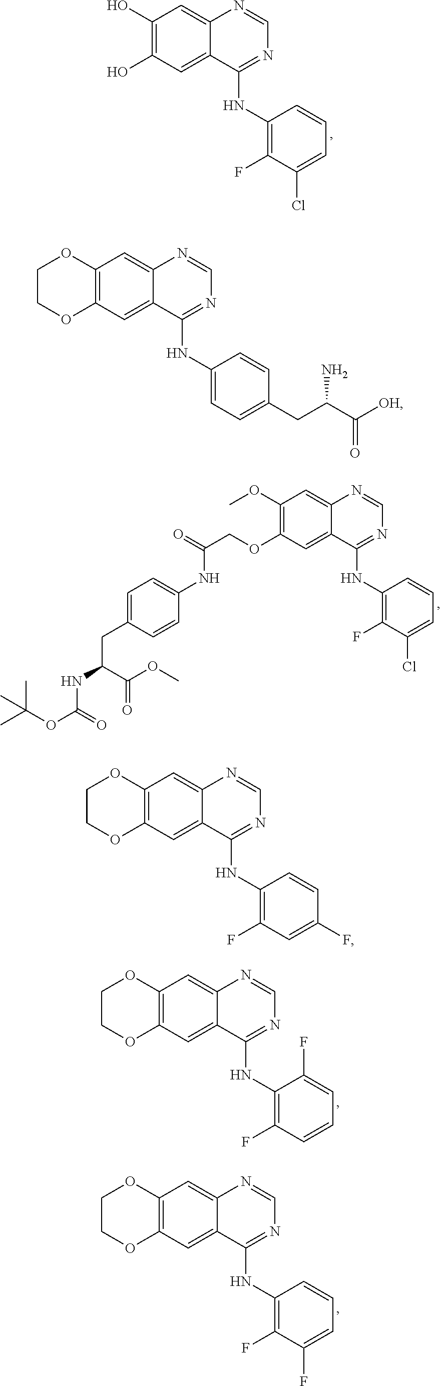 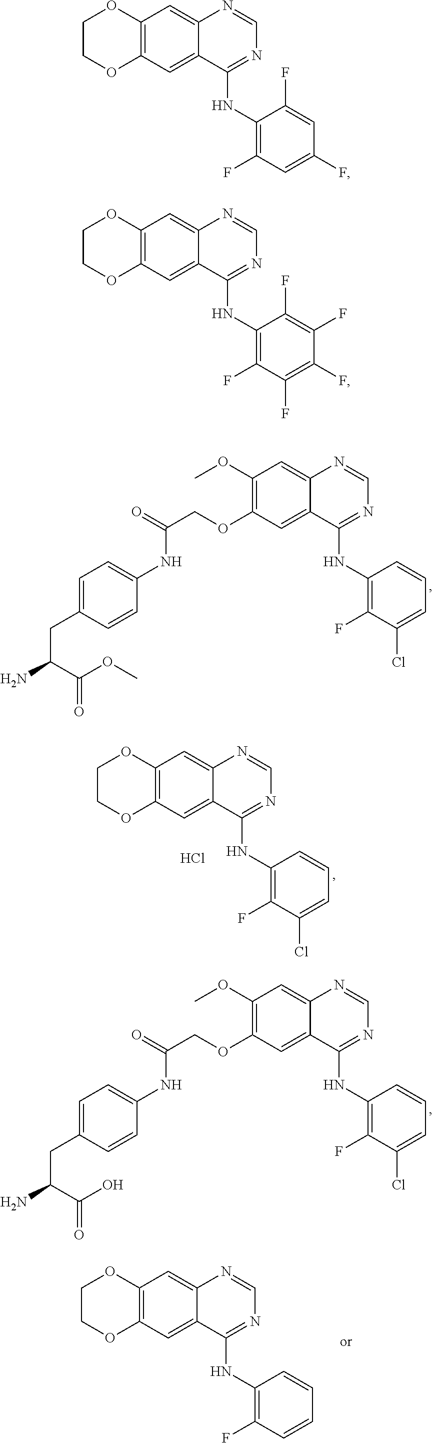 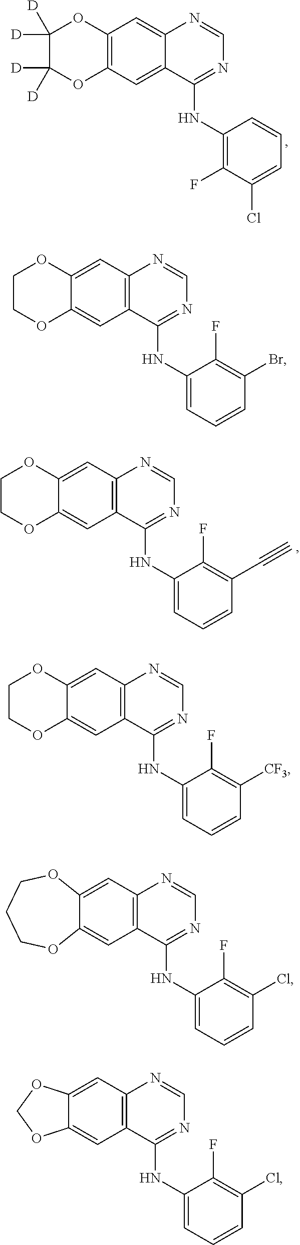 [SMILES: [2H]C1([2H])Oc2cc3ncnc(Nc4cccc(Cl)c4F)c3cc2OC1([2H])[2H] ; Fc1c(Br)cccc1Nc1ncnc2cc3c(cc12)OCCO3 ; C#Cc1cccc(Nc2ncnc3cc4c(cc23)OCCO4)c1F ; Fc1c(Nc2ncnc3cc4c(cc23)OCCO4)cccc1C(F)(F)F ; Fc1c(Cl)cccc1Nc1ncnc2cc3c(cc12)OCCCO3 ; Fc1c(Cl)cccc1Nc1ncnc2cc3c(cc12)OCO3] 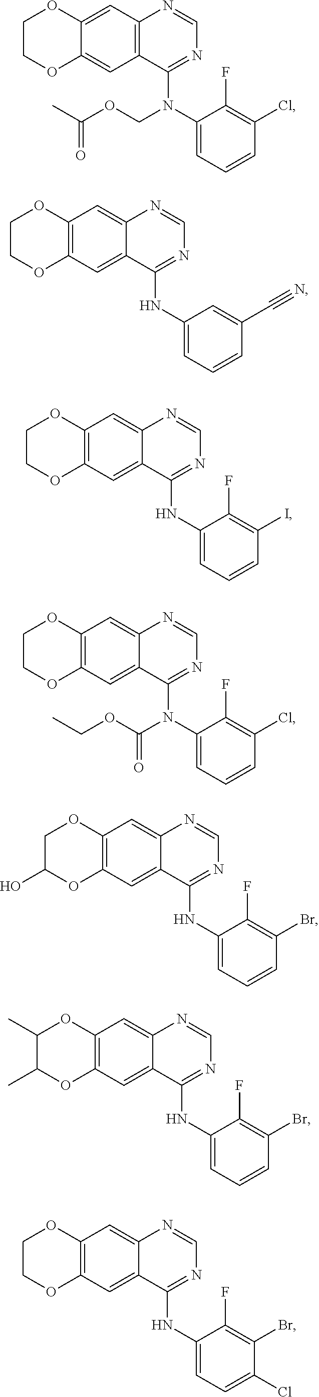 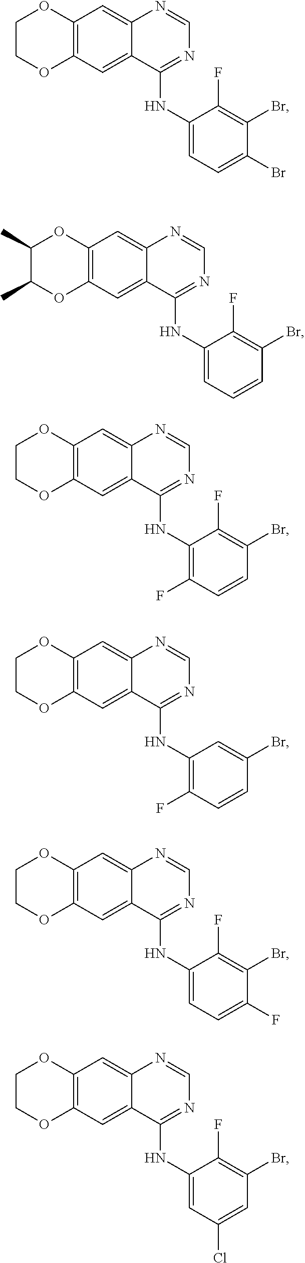 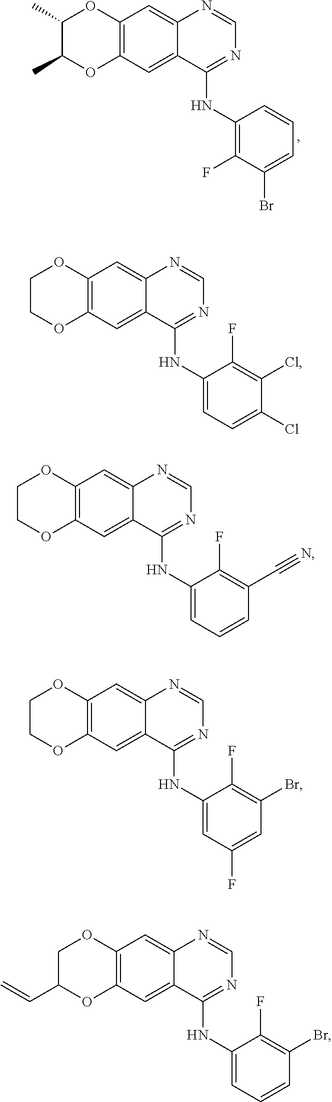 [SMILES: C[C@@H]1Oc2cc3ncnc(Nc4cccc(Br)c4F)c3cc2O[C@H]1C ; Fc1c(Nc2ncnc3cc4c(cc23)OCCO4)ccc(Cl)c1Cl ; N#Cc1cccc(Nc2ncnc3cc4c(cc23)OCCO4)c1F ; Fc1cc(Br)c(F)c(Nc2ncnc3cc4c(cc23)OCCO4)c1 ; C=CC1COc2cc3ncnc(Nc4cccc(Br)c4F)c3cc2O1]
pharmaceutically acceptable salt or stereoisomer thereof.

53. A pharmaceutical composition comprising the compound of any one of the preceding claims and a pharmaceutically acceptable excipient.

54. A method of inhibiting EGFR or a variant thereof, such as ΔEGFR, EGFR extracellular mutants, EGFR A289, EGFR T263, and/or EGFR activating mutants for example ex19 deletion, comprising administering to a subject a compound or composition of any one of claims 1-52.

55. A method of treating cancer, comprising of administering to a subject in need of a treatment for cancer a compound or composition of any one of claims 1-52.

56. The method of claim 55, wherein the cancer is bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, or prostate cancer.

57. The method of claim 56, wherein the cancer is glioma, astrocytoma or glioblastoma.

58. A method of classifying a subject diagnosed with glioma or GBM, the method comprising:
a. obtaining a biological sample from the subject;
b. treating the biological sample with a glucose metabolism inhibitor; and
c. determining if glucose metabolism is attenuated by the glucose metabolism inhibitor.

59. The method of claim 58, wherein the biological sample is from a GBM tumor.

60. The method of claim 58 or 59, wherein the method further comprises comparing the level of glucose attenuation to a control.

61. The method of claim 60, wherein the control comprises a non-cancerous sample, a cancerous sample with a different phenotype, a cancer sample with a wildtype EFGR expression level or a cancerous sample taken from the biological sample before the biological sample is subjected to a glucose metabolism inhibitor.

62. The method of any of claims 58-61 wherein the method further comprises classifying the subject as a metabolic responder if glucose metabolism is attenuated by the glucose metabolism inhibitor in the biological sample.

63. The method of claim 63, wherein the method further comprises treating the subject classified as a metabolic responder with a glucose metabolism inhibitor and a cytoplasmic p53 stabilizer.

64. A method of treating cancer in a subject, the method comprising administering to the subject a glucose metabolism inhibitor and a cytoplasmic p53 stabilizer.

65. The method of claim 64, wherein the cancer is glioma, astrocytoma or glioblastoma.

66. A method of treating glioblastoma in a subject, the method comprising administering to the subject an amount of a glucose uptake inhibitor and a cytoplasmic p53 stabilizer after determining that the subject is susceptible to reduced glucose metabolism by an EGFR inhibitor.

67. A method of treating glioblastoma in a subject, the method comprising administering to the subject a therapeutically effective amount of a glucose uptake inhibitor and a cytoplasmic p53 stabilizer after determining that the subject is susceptible to reduced glucose metabolism by an EGFR inhibitor.

68. A method of reducing glioblastoma proliferation in a subject, the method comprising administering to the subject an amount of an EGFR inhibitor and a MDM2 inhibitor.

69. A method of reducing glioblastoma proliferation in a subject, the method comprising administering to the subject an effective amount of an EGFR inhibitor and a MDM2 inhibitor after determining that the glucose metabolism in a sample taken from the subject is susceptible to EGFR inhibitors.

70. A method for treating cancer or reducing cancer cell proliferation in a subject, comprising administering to the subject an amount of a glucose metabolism inhibitor and a p53 stabilizer.

71. A method for treating cancer or reducing cancer cell proliferation in a subject that has been determined to have cancer that is responsive to glucose metabolism inhibitors, comprising administering to the subject a therapeutically effective amount of a glucose metabolism inhibitor and a p53 stabilizer.

72. The method of claim 71, where the cancer is glioblastoma multiforme, glioma, low-grade astrocytoma, mixed oligoastrocytoma, pilocytic astrocytoma, pleomorphic xanthoastrocytoma, subependymal giant cell astrocytoma, anaplastic astrocytoma, CNS cancer, non-CNS cancer, or CNS metastases or lung cancer.

73. A method of treating malignant glioma or glioblastoma in a subject, the method comprising administering to the subject an amount of the glucose metabolism inhibitor and a cytoplasmic p53 stabilizer.

74. A method of treating malignant glioma or glioblastoma in a subject, the method comprising administering to the subject after the subject has been determined to be susceptible to a glucose metabolism inhibitor an amount of the glucose metabolism inhibitor and a cytoplasmic p53 stabilizer.

75. The method of any of claims 58-74, wherein the subject has been determined to be susceptible to the glucose metabolism inhibitor by a method comprising:
a. obtaining a tumor biopsy from the subject;
b. measuring the level of glucose uptake by the tumor cells in the presence of the glucose metabolism inhibitor;
c. comparing the level of glucose uptake by the tumor cells obtained in step b. to the level of glucose uptake by a control, and
d. determining that the subject is susceptible to the glucose metabolism inhibitor if the level of glucose uptake by the tumor cells is attenuated compared to the control.

76. The method of claim 75, wherein glucose uptake is measured by the uptake of radio-labelled glucose 2-deoxy-2-[fluorine-18]fluoro-D-glucose (¹⁸F-FDG).

77. The method of claim 76, further comprising detecting the ¹⁸F-FDG by positron emission tomography (PET).

78. The method of any of claims 58-74, wherein the subject has been determined to be susceptible to the glucose metabolism inhibitor by a method comprising:
a. obtaining a first blood sample from the subject;
b. placing the subject on a ketogenic diet;
c. obtaining a second blood sample from the subject after being placed on a ketogenic diet for a period of time;
d. measuring glucose level in the first and in the second blood sample;
e. comparing the glucose level in the second blood sample with the glucose level in the first blood sample; and
f. determining that the subject is susceptible if the glucose level in the second blood sample is reduced as compared to glucose levels in the first blood sample.

79. The method of claim 78, wherein the reduction in the glucose level between the second blood sample and the control blood sample is about or greater than 0.15 mM.

80. The method of claim 78, wherein the reduction in the glucose level between the second blood sample and the control blood sample is about or greater than 0.20 mM.

81. The method of claim 78, wherein the reduction in the glucose level between the second blood sample and the control blood sample is in the range of 0.15 mM-2.0 mM.

82. The method of claim 78, wherein the reduction in the glucose level between the second blood sample and the control blood sample is in the range of 0.25 mM-1.0 mM.

83. A method of assessing the sensitivity of cancer cells or tumor to treatment with a glucose metabolism inhibitor and a cytoplasmic p53 stabilizer, the method comprising measuring or detecting the level of glucose uptake by the cancer cells and comparing the level of glucose uptake with a control.

84. The method of claim 83, wherein the glucose is radio-labelled.

85. The method of claim 84 wherein the radio-labelled glucose is 2-deoxy-2-[fluorine-18]fluoro-D-glucose (¹⁸F-FDG).

86. The method of claim 84, wherein measuring and detecting radio labeled glucose uptake is quantified by positron emission tomography (PET).

87. The method of any one of claims 83-86, wherein the control comprises a non-cancerous sample, a cancerous sample with a different phenotype, a cancer sample with a wildtype EGFR expression level.

88. The method of any one of claims 58-87, wherein the glucose metabolism inhibitor comprises a glucose uptake inhibitor, a glucose transporter inhibitor, a glycolysis inhibitor, or an epidermal growth factor receptor (EGFR) inhibitor.

89. The method of claim 88, wherein the EGFR inhibitor is erlotinib, gefitinib, lapatinib, cetuximab, panitumumab, vandetanib, necitumumab, or osimertinib.

90. The method of claim 88, wherein the EGFR inhibitor is a compound of any one of claims 1-43.

91. The method of claim 88, wherein the glucose metabolism inhibitor is a phosphatidylinositol 3-kinase PI3K inhibitor.

92. The method of claim 88, wherein the PI3K inhibitor is pictilisib, dactolisib, wortmannin, LY294002, Idelalisib, duvelisib, buparlisib, IPI-549, SP2523, GDC-0326, TGR-1202, VPS34 inhibitor 1, GSK2269557, GDC-0084, SAR405, AZD8835, LY3023414, PI-103, TGX-221, NU7441, IC-87114, wortmannin, XL147 analogue, ZSTK474, Alpelisib, PIK-75 HCl, A66, AS-605240, 3-Methyladenine (3-MA), PIK-93, PIK-90, AZD64822, PF-04691502, Apitolisib, GSK1059615, Duvelisib, Gedatolisib, TG100-115, AS-252424, BGT226, CUDC-907, AS-604850, PIK-294, GSK2636771, Copanlisib, YM201636, CH5132799, CAY10505, PIK-293, PKI-402, TG100713, VS-5584, Taselisib, CZC24832, AMG319, GSK2292767, HS-173, Quercetin, Voxtalisib, PIK-93, Omipalisib, PIK-90, GNE-317, Pilaralisib, PF-4989216, AZD8186, 740 Y-P, Vps34-IN1, PIK-III, PI-3065 or analogs thereof.

93. The method of claim 88, wherein the glucose metabolism inhibitor is 2-deoxyglucose (2DG) or cytochalasin B.

94. The method of any one of claims 63-93, wherein the cytoplasmic p53 stabilizer is an MDM2 inhibitor.

95. The method of claim 94, wherein the MDM2 inhibitor is a nutlin.

96. The method of claim 94, wherein the MDM2 inhibitor is nutlin-3 or idasanutlin.

97. The method of claim 94, wherein the MDM2 inhibitor is RO5045337, RO5503781, RO6839921, SAR405838, DS-3032, DS-3032b, or AMG-232.

98. The method of any one of claims 63-93, wherein the cytoplasmic p53 stabilizer is a BCL-2 inhibitor.

99. The method of claim 85, wherein the BCL-2 inhibitor is antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252.

100. The method of any one of claims 63-93, wherein the cytoplasmic p53 stabilizer is a Bcl-xL inhibitor.

101. The method of claim 100, wherein the Bcl-xL inhibitor is WEHI 539, ABT-263, ABT-199, ABT-737, sabutoclax, AT101, TW-37, APG-1252, or gambogic acid.

102. The method of any one of claims 63-101, wherein the glucose metabolism inhibitor and the cytoplasmic p53 stabilizer are administered in the same composition.

103. The method of any one of claims 63-101, wherein the glucose metabolism inhibitor and the p53 stabilizer are administered conjointly.

104. The method of any one of claims 63-101, wherein the glucose metabolism inhibitor and the p53 stabilizer are administered within 24 hours of each other.

105. The method of any one of claims 63-101, wherein the glucose metabolism inhibitor and the p53 stabilizer are administered within 6 hours of each other.

106. The method of any one of claims 63-101, wherein the glucose metabolism inhibitor and the p53 stabilizer are administered within 2 hours of each other.

107. The method of any one of claims 63-101, wherein the glucose metabolism inhibitor and the p53 stabilizer are administered within 1 hour of each other.

108. The method of any one of claims 63-101, wherein the glucose metabolism inhibitor and the p53 stabilizer are administered within 30 min of each other.

109. The method of any one of claims 63-101, wherein the glucose metabolism inhibitor and the p53 stabilizer are administered at the same time to the subject.

110. The method of any one of claims 57-89 and 94-109, wherein the subject is administered 1 mg to 250 mg of erlotinib.

111. The method of any one of claims 57-89 and 94-109, wherein the subject is administered 25 mg of erlotinib.

112. The method of any one of claims 57-89 and 94-109, wherein the subject is administered 100 mg of erlotinib.

113. The method of any one of claims 57-89 and 94-109, wherein the subject is administered 150 mg of erlotinib.

114. The method of any one of claims 57-96 and 102-113, wherein the subject is administered 50 mg to 1600 mg idasanutlin.

115. The method of any one of claims 57-96 and 102-113, wherein the subject is administered 100 mg of idasanutlin.

116. The method of any one of claims 57-96 and 102-113, wherein the subject is administered 150 mg of idasanutlin.

117. The method of any one of claims 57-96 and 102-113, wherein the subject is administered 300 mg of idasanutlin.

118. The method of any one of claims 57-96 and 102-113, wherein the subject is administered 400 mg of idasanutlin.

119. The method of any one of claims 57-96 and 102-113, wherein the subject is administered 600 mg of idasanutlin.

120. The method of any one of claims 57-96 and 102-113, wherein the subject is administered 1600 mg of idasanutlin.

121. The method of any of claims 54-120, wherein the subject has been diagnosed with glioblastoma multiforme.

122. The method of any of claims 54-121, wherein the subject has been previously treated for glioblastoma with a prior treatment.

123. The method of any of claims 54-122, wherein the subject has been determined to be resistant to the prior treatment.

124. The method of any of claims 54-123, wherein the method further comprises administration of an additional therapy.

125. A pharmaceutical composition comprising a glucose metabolism inhibitor and a cytoplasmic p53 stabilizer.

126. The pharmaceutical composition of claim 125, wherein the glucose metabolism inhibitor comprises a glucose uptake inhibitor, a glucose transporter inhibitor, a glycolysis inhibitor, or an epidermal growth factor receptor (EGFR) inhibitor.

127. The pharmaceutical composition of claim 125 or 126, wherein the EGFR inhibitor is erlotinib, gefitinib, lapatinib, cetuximab, panitumumab, vandetanib, necitumumab, or osimertinib.

128. The pharmaceutical composition of claim 125 or 126, wherein the EGFR inhibitor is a compound of any one of claims 1-52.

129. The pharmaceutical composition of claim 125 or 126, wherein the glucose metabolism inhibitor is a phosphatidylinositol 3-kinase PI3K inhibitor.

130. The pharmaceutical composition of claim 129, wherein the PI3K inhibitor is pictilisib, dactolisib, wortmannin, LY294002, Idelalisib, duvelisib, buparlisib, IPI-549, SP2523, GDC-0326, TGR-1202, VPS34 inhibitor 1, GSK2269557, GDC-0084, SAR405, AZD8835, LY3023414, PI-103, TGX-221, NU7441, IC-87114, wortmannin, XL147 analogue, ZSTK474, Alpelisib, PIK-75 HCl, A66, AS-605240, 3-Methyladenine (3-MA), PIK-93, PIK-90, AZD64822, PF-04691502, Apitolisib, GSK1059615, Duvelisib, Gedatolisib, TG100-115, AS-252424, BGT226, CUDC-907, AS-604850, PIK-294, GSK2636771, Copanlisib, YM201636, CH5132799, CAY10505, PIK-293, PKI-402, TG100713, VS-5584, Taselisib, CZC24832, AMG319, GSK2292767, HS-173, Quercetin, Voxtalisib, PIK-93, Omipalisib, PIK-90, GNE-317, Pilaralisib, PF-4989216, AZD8186, 740 Y-P, Vps34-IN1, PIK-III, PI-3065 or analogs thereof.

131. The pharmaceutical composition of claim 125 or 126, wherein the glucose metabolism inhibitor is 2-deoxyglucose (2DG) or cytochalasin B.

132. The pharmaceutical composition of any one of claims 125-131, wherein the cytoplasmic p53 stabilizer is an MDM2 inhibitor or antagonist.

133. The pharmaceutical composition of claim 132, wherein the MDM2 inhibitor is a nutlin.

134. The pharmaceutical composition of claim 132, wherein the MDM2 inhibitor is nutlin-3 or idasanutlin.

135. The pharmaceutical composition of claim 132, wherein the MDM2 inhibitor is RO5045337, RO5503781, RO6839921, SAR405838, DS-3032, DS-3032b, or AMG-232.

136. The pharmaceutical composition of any one of claims 125-131, wherein the cytoplasmic p53 stabilizer is a BCL-2 inhibitor.

137. The pharmaceutical composition of claim 136, wherein the BCL-2 inhibitor is antisense oligodeoxynucleotide G3139, mRNA antagonist SPC2996, venetoclax (ABT-199), GDC-0199, obatoclax, paclitaxel, navitoclax (ABT-263), ABT-737, NU-0129, S 055746, or APG-1252.

138. The pharmaceutical composition of any one of claims 125-131, wherein the cytoplasmic p53 stabilizer is a Bcl-xL inhibitor.

139. The pharmaceutical composition of claim 138, wherein the Bcl-xL inhibitor is WEHI 539, ABT-263, ABT-199, ABT-737, sabutoclax, AT101, TW-37, APG-1252, or gambogic acid.
